(19) United States
(12) Patent Application Publication
(10) Pub. No.: US 2023/0293706 A1
(43) Pub. Date: Sep. 21, 2023

(54) Multicomponent Delivery Systems for Polyanionic Cargo Compound Delivery

(71) Applicant: NUTCRACKER THERAPEUTICS, INC., Emeryville, CA (US)

(72) Inventor: Colin James McKinlay, FRemont, CA (US)

(21) Appl. No.: 18/020,246

(22) PCT Filed: Aug. 6, 2021
(86) PCT No.: PCT/US21/44885

§ 371 Date: Feb. 7, 2023

Related U.S. Application Data
(60) Provisional application No. 63/062,749, filed on Aug. 7, 2020.

(30) Foreign Application Priority Data
Nov. 4, 2020 (NL) 2026825

(51) Int. Cl. A61K 47/64; A61K 39/12; A61K 47/69

(52) U.S. Cl. CPC A61K 47/6455; A61K 39/12; A61K 47/646; A61K 47/6929; A61K 2039/53

(57) ABSTRACT

The present disclosure relates to multicomponent delivery systems for delivering polyanionic cargo compounds, such as nucleic acids. The present disclosure also relates to methods of preparing and using the multicomponent delivery systems.

### CROSS REFERENCE TO RELATED APPLICATIONS

The present application claims priority to and benefit of U.S. provisional patent application No. 63/062,749, filed on Aug. 7, 2020, and Dutch patent application no., NL 2026825, filed on Nov. 4, 2020, the disclosure of each of which is incorporated by reference in its entirety.

### BACKGROUND

Therapeutic nucleic acids include, e.g., mRNA, small interfering RNA (siRNA), small activating RNA (saRNA), micro RNA (miRNA), antisense oligonucleotides, ribozymes, plasmids, and immune stimulating nucleic acids. These nucleic acids act via a variety of mechanisms. A safe and effective delivery system is required for the nucleic acids to be therapeutically useful. Viral vectors are relatively efficient gene delivery systems, but suffer from a variety of limitations, such as the potential for reversion to the wild-type as well as immune response concerns. Furthermore, viral systems are rapidly cleared from the circulation, limiting transfection to organs such as the lungs, liver, and spleen. In addition, these systems induce immune responses that compromise delivery with subsequent injections. Thus, nonviral gene delivery systems are being developed such as reverse micelles, anionic liposomes, cationic liposomes, and polymer liposomes.

### BRIEF SUMMARY

The present disclosure generally relates to multicomponent delivery systems for delivering polyanionic cargo compounds, such as nucleic acids, to target cells, and methods of making and using the multicomponent delivery systems. In some implementations, the present disclosure provides multicomponent delivery systems comprising at least one lipidated cationic peptoid component for the delivery of polyanionic compounds, such as nucleic acids, with improved efficiency. These polyanionic compounds are also referred to as polyanionic cargo compounds or cargos of the multicomponent delivery system. Other components in the multicomponent delivery systems may include non-cationic components, such as anionic or zwitterionic components, lipid components, and shielding components.

In yet another aspect of the present disclosure, provided herein are methods for delivering a polyanionic cargo compound to a cell comprising contacting the cell with a complex formed by the multicomponent delivery system and the polyanionic cargo compound.

An additional aspect of this disclosure provides methods of preparing the multicomponent delivery system and the complex comprising the multicomponent delivery system and polyanionic compounds.

Another aspect of the disclosure relates to methods of eliciting an immune response and/or treating diseases and conditions with the multicomponent delivery systems of the disclosure.

The disclosure includes the following sub-sections:
1. A system for the delivery of polyanionic compounds to target cells, the delivery system comprising at least one cationic component, wherein the at least one cationic component comprises a lipidated peptoid (“lipidated cationic peptoid”).
2. The system of claim 1 further comprising one or more of the following: (i) an anionic or zwitterionic component, (ii) a non-cationic lipid component, and (iii) a shielding component.
3. The system of claim 2, wherein the anionic or zwitterionic component comprises a phospholipid, a lipitoid, or a mixture thereof.
4. The system of claim 3, wherein the anionic or zwitterionic component is a DOPE or DSPC
5. The system of any one of claims 2-4, wherein the non-cationic lipid component comprises a sterol and/or a neutral peptoid.
6. The system of claim 5, wherein the sterol is cholesterol.
7. The system of any one of claims 2-6, wherein the shielding component comprises a poly(ethylene glycol) (PEG) moiety.
8. The system of claim 7, wherein the shielding component is a PEGylated lipid or a PEGylated lipidated peptoid.
9. The system of claim 8, wherein the shielding component is DMG-PEG.
10. The system of claim 9, wherein the shielding component is DMG-PEG 2000.
11. The system of any one of claims 5-10, wherein the non-cationic lipid component comprises Compound 90, or wherein the cationic lipid component comprises Compound 89.
12. The system according to any of the preceding claims 2-6 wherein the cationic component comprises Compound 112, and the non-cationic lipid component comprises Compound 90.
13. The system according to claim 12, wherein the mol % of the cationic component is between about 20 to about 50; the mol % of the anionic or zwitterionic component is between about 0 to about 30; the mol % of the non-cationic lipid component is between about 30 to about 80; and the mol % of the shielding component is between about 0 to about 10.
14. The system according to claim 12 or 13, wherein the mol % of the cationic component is between about 30 to about 50; the mol % of the anionic or zwitterionic component is between about 0 to about 10; the mol % of the non-cationic lipid component is between about 40 to about 70; and the mol % of the shielding component is between about 0 to about 5.
15. The system according to any of the claims 13-14 wherein the mol % of the cationic component is about 38; the mol % of the anionic or zwitterionic component is 0; the mol % of the non-cationic lipid component is about 59.7; and the mol % of the shielding component is about 2.3.
16. The system according to claim 15, wherein the anionic or zwitterionic component comprises DOPE, and wherein the shielding component comprises DMG-PEG2K.
17. The system according to any of the claims 2-16, wherein the cationic component comprises Compound 89 or Compound 93, and the non-cationic lipid component comprises cholesterol, Compound 90, or any combination thereof.
18. The system according to any of the claims 1-10, wherein the cationic component comprises Compound 79, and the non-cationic lipid component comprises Compound 90.
19. The system according to any of the claims 1-10, wherein the cationic component comprises Compound 24 or Compound 89, and the non-cationic lipid component comprises cholesterol, Compound 90, or any combination thereof.
20. The system of any of the preceding claims 2-10 comprising at least about 99 mol % cationic component and less than about 1 mol % shielding component.
21. The system according to claim 20 wherein the cationic component comprises Compound 24, Compound 79, Compound 93, Compound 112, or any combination thereof.
22. The system according to claim 20 or 21 wherein the mol % of the cationic component is about 99.1, and the mol % of the shielding component comprising PEG is about 0.9.
23. The system according to any of the preceding claims 2-10 comprising less than about 20 mol % of the cationic component, less than about 5 mol % of the shielding component, and more than about 75 mol % of a mixture of the zwitterionic component and the non-cationic lipid component.
24. The system of claim 23, wherein the cationic component comprises Compound 24, Compound 79, Compound 93, Compound 112, or any combination thereof.
25. The system of claim 23 or 24, wherein the mol % of the cationic component is about 17.9, the mol % of the shielding component comprising PEG is about 2.8, the mol % of the non-cationic lipid component is about 62.9, and the mol % of the anionic or zwitterionic component is about 16.4.
26. The system of claim 23 or 24, wherein the mol % of the cationic component is about 17.1, the mol % of the shielding component comprising PEG is about 2.7, the mol % of the non-cationic lipid component is about 80.2.
27. The system according to any of the claims 2-10 comprising between about 30 and about 45 mol % of the cationic component, between about 50 and about 70 mol % of a mixture of the anionic or zwitterionic component and the non-cationic lipid component, and between about 1.5 about 4.5 mol % of the shielding component.
28. The system of claim 27 wherein the cationic component comprises Compound 24, Compound 79, Compound 93, Compound 112, or any combination thereof.
29. The system of claim 27 or 28 wherein the mol % of the cationic component is about 32.9, the mol % of the shielding component comprising PEG is about 2.0, the mol % of the non-cationic lipid component is about 51.7, and the mol % of the anionic or zwitterionic component is about 13.4.
30. The system of claim 27 or 28 wherein the mol % of the cationic component is about 42.3, the mol % of the shielding component comprising PEG is about 4.4, the mol % of the non-cationic lipid component is 0, and the mol % of the anionic or zwitterionic component is about 53.3.
31. The system according to any of the claims 2-10 comprising between about 15 and about 35 mol % of the cationic component, between about 60 and about 80 mol % of a mixture of the zwitterionic component and the non-cationic lipid component, and between about 1.5 and about 3.0 mol % of the shielding component.
32. The system of claim 31 wherein the cationic component comprises Compound 24, Compound 79, Compound 93, Compound 112, or any combination thereof.
33. The system of claim 31 or 32, wherein the mol % of the cationic component is about 17.9, the mol % of the shielding component comprising PEG is about 2.8, the mol % of the non-cationic lipid component is about 62.9, and the mol % of the anionic or zwitterionic component is about 16.4.
34. The system of claim 31 or 32 wherein the mol % of the cationic component is about 32.9, the mol % of the shielding component comprising PEG is about 2.0, the mol % of the non-cationic lipid component is about 51.7, and the mol % of the anionic or zwitterionic component is about 13.4.
35. A complex comprising the system of any one of claims 1 to 34 and a polyanionic compound.
36. A delivery vehicle composition comprising a lipidated cationic peptoid component.
37. The delivery vehicle composition of claim 36, wherein the lipidated cationic peptoid component comprises a compound of Formula (Ia):

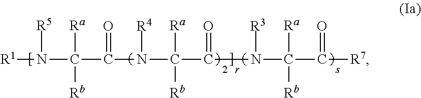

wherein:
r is an integer from 1 to 5;
s is an integer from 1 to 8;
R¹ is alkyl or CH₃;
each R³ independently is C₈-C₂₄alkyl or C₈-C₂₄-alkenyl;
each R⁴ independently is selected from the group consisting of C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalky is: (i) unsubstituted or (ii) substituted with one or more substituents selected from —OH, halo, or alkoxy;
each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl;
R⁷ is NH₂; and
each of R^{a} and R^{b} independently is H.
   38. The delivery vehicle composition of claim 37, wherein the lipidated cationic peptoid component comprises one or more compounds selected from Compounds 1-12 and 19-36.
   39. The delivery vehicle composition of claim 38, wherein the lipidated cationic peptoid component comprises Compound 24.
   40. The delivery vehicle composition of claim 36, wherein the lipidated cationic peptoid component comprises a compound of Formula (Ib):

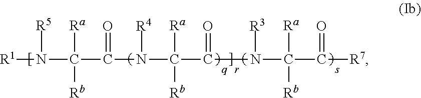

wherein:
q is 0 or 1;
r is an integer from 1 to 10;
s is an integer from 1 to 8;
R¹ is alkyl or CH₃;
each R³ independently is C₈-C₂₄alkyl or C₈-C₂₄-alkenyl;
each R⁴ independently is selected from the group consisting of C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalky is: (i) unsubstituted or (ii) substituted with one or more substituents selected from —OH, halo, or alkoxy;
each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl;
R⁷ is NH₂; and
each of R^{a} and R^{b} independently is H.
   41. The delivery vehicle composition of claim 40, wherein the lipidated cationic peptoid component comprises one or more compounds selected from Compounds 13-16, 76, and 99.
   42. The delivery vehicle composition of claim 36, wherein the lipidated cationic peptoid component comprises a compound of Formula (Ic):

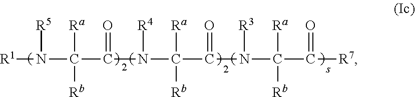

wherein:
s is 3 or 4;
R¹ is alkyl or CH₃;
each R³ independently is C₈-C₂₄alkyl or C₈-C₂₄-alkenyl;
each R⁴ independently is selected from the group consisting of C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalky is: (i) unsubstituted or (ii) substituted with one or more substituents selected from —OH, halo, or alkoxy;
each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl;
R⁷ is NH₂; and
each of R^{a} and R^{b} independently is H.
   43. The delivery vehicle composition of claim 42, wherein the lipidated cationic peptoid component comprises one or more compounds selected from Compounds 49-55.
   44. The delivery vehicle composition of claim 36, wherein the lipidated cationic peptoid component comprises a compound of Formula (Id):

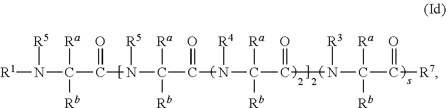

wherein:
s is 3 or 4;
R¹ is alkyl or CH₃;
each R³ independently is C₈-C₂₄alkyl or C₈-C₂₄-alkenyl;
each R⁴ independently is selected from the group consisting of C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalky is: (i) unsubstituted or (ii) substituted with one or more substituents selected from —OH, halo, or alkoxy;
each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl;
R⁷ is NH₂; and
each of R^{a} and R^{b} independently is H.
   45. The delivery vehicle composition of claim 44, wherein the lipidated cationic peptoid component comprises one or more compounds selected from Compounds 57-63 and 65-71.
   46. The delivery vehicle composition of claim 36, wherein the lipidated cationic peptoid component comprises a compound of Formula (Ie):

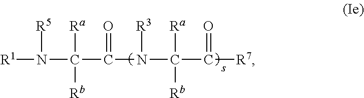

wherein:
s is 1 to 8;
R¹ is H or alkyl;
each R³ independently is C₈-C₂₄alkyl or C₈-C₂₄-alkenyl;
R⁵ is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl;
R⁷ is NH₂; and
each of R^{a} and R^{b} independently is H.
   47. The delivery vehicle composition of claim 46, wherein the lipidated cationic peptoid component comprises one or more compounds selected from Compounds 41-47, 73-75, 77-80, 85, 86, 91-94, 97, 98, 111-118, 137, 138, 140, 145, 146, 148, 149, 151-154, and 160-162.
   48. The delivery vehicle composition of claim 47, wherein the lipidated cationic peptoid component comprises Compound 73, Compound 79, Compound 85, Compound 93, Compound 112, Compound 115, Compound 137, Compound 152, or combinations thereof.
   49. The delivery vehicle composition of claim 48, wherein the lipidated cationic peptoid component comprises Compound 112.
   50. The delivery vehicle composition of claim 36, wherein the lipidated cationic peptoid component comprises a compound of Formula (If):

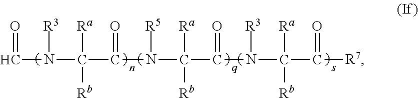

wherein:
n is an integer from 0 to 4;
q is 1 or 2;
s is an integer from 1 to 4;
each R³ independently is C₈-C₂₄alkyl or C₈-C₂₄-alkenyl;
R⁵ is selected from the group consisting of aminoalkyl, alkylaminoalkyl, aminoalkylaminoakyl, guanidinoalkyl, N-heterocyclylalkyl, and N-heteroaryl;
R⁷ is NH₂; and
each of R^{a} and R^{b} independently is H.
   51. The delivery vehicle composition of claim 50, wherein the lipidated cationic peptoid component comprises one or more compounds selected from Compounds 81, 82, 83, 95, 96, 142, 144, 155, 156, 163, 164, and 165.
   52. The delivery vehicle composition of claim 51, wherein the lipidated cationic peptoid component comprises Compound 81, Compound 155, Compound 163, Compound 164, or combinations thereof.
   53. The delivery vehicle composition of claim 52, wherein the lipidated cationic peptoid component comprises Compound 81, Compound 155, or combinations thereof.
   54. The delivery vehicle composition of claim 36, wherein the lipidated cationic peptoid component comprises one or more compounds selected from Compounds 17, 18, 84, 87-89, 100, 101, 102, 103, 113, 114, 127, 130, 132, 134, 139, 141, 147, and 159.
   55. The delivery vehicle composition of claim 36, wherein the lipidated cationic peptoid component comprises Compound 127.
   56. The delivery vehicle composition of claim 36, wherein the lipidated cationic peptoid component is selected from a compound listed in Table 1A.
   57. The delivery vehicle composition of any one of claims 36-56 further comprising a non-cationic lipidated peptoid component.
   58. The delivery vehicle composition of claim 57, wherein the non-cationic lipidated peptoid component comprises a neutral lipidated peptoid component.
   59. The delivery vehicle composition of claim 58, wherein the neutral lipidated peptoid component comprises a compound Formula (IVa):

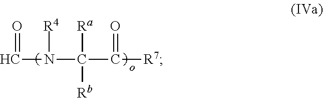

wherein o is integer from 3 to 10;
each R⁴ independently is C₈-C₂₄alkyl, or C₁-C₄-alkyl substituted with cycloalkyl, heterocyclylalkyl, aryl, alkylaryl, arylalkyl, alkylheteroaryl, heteroarylalkyl, alkoxy, alkoxyalkyl, or hydroxyalkyl;
R⁷ is —NH₂; and
each R^{a} and R^{b} independently is —H.
   60. The delivery vehicle composition of claim 59, wherein the neutral lipidated peptoid component comprises Compound 90, Compound 119, Compound 120, Compound 121, Compound 122, Compound 123, Compound 128, Compound 129, or combinations thereof.
   61. The delivery vehicle composition of claim 60, wherein the neutral lipidated peptoid component comprises Compound 90, Compound 119, or a combination thereof.
   62. The delivery vehicle composition of claim 58, wherein the neutral lipidated peptoid component comprises Compound 150.
   63. The delivery vehicle composition of claim 58, wherein the neutral lipidated peptoid component is selected from a compound listed in Table 1C.
   64. The delivery vehicle composition of any one of claims 36-63, further comprising an anionic or zwitterionic (“anionic/zwitterionic”) lipidated peptoid component.
   65. The delivery vehicle composition of claim 64, wherein the anionic/zwitterionic lipidated peptoid component comprises a compound of Formula (IIIa)

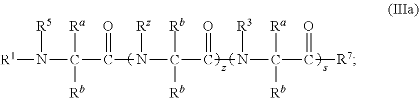

wherein:
s is an integer from 1 to 6;
z is 1 or 2;
R¹ is H or alkyl;
each R³ independently is C₈-C₂₄alkyl or C₈-C₂₄-alkenyl;
R⁵ is selected from the group consisting of aminoalkyl, alkylaminoalkyl, aminoalkylaminoakyl, guanidinoalkyl, N-heterocyclylalkyl, and N-heteroaryl;
R⁷ is —NH₂;
each R^{a} and R^{b} independently is —H; and
each R^{z} independently is C₂₋₅ alkylenecarboxylic acid.
   66. The delivery vehicle composition of claim 65, wherein the anionic/zwitterionic lipidated peptoid component comprises Compound 104, Compound 105, or a combination thereof.
   67. The delivery vehicle composition of claim 64, wherein the anionic/zwitterionic lipidated peptoid component comprises a compound of Formula (IIIb):

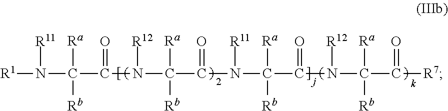

wherein:
j is 1, 2, 3, 4, 5, or 6;
k is 1, 2, 3, or 4;
R¹ is —H, alkyl, alkylaryl, —COR^{1a} or a lipid moiety, wherein R^{1a} is —H, —OH, alkyl, aryl, alkylaryl, —O-alkyl, or —O-alkylaryl;
each R¹¹ independently is aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, hydroxylheteroalkyl, or C₂₋₅alkylenecarboxylic acid;
each R¹² independently is C₈-C₂₄alkyl, C₈-C₂₄-alkenyl, C₁-C₄aralkyl, or C₁-4heteroaralkyl;
R⁷ is —H, alkyl, acyl, —OH, —OR^{7a}, —NH₂, —NHR^{7a}, or a lipid moiety, wherein R^{7a} is alkyl, acyl, or a lipid moiety; and
each R^{a} and R^{b} independently is —H, C₁-C₄-alkyl, or a side chain moiety found on a naturally- or non-naturally-occurring amino acid.
   68. The delivery vehicle composition of claim 67, wherein the non-cationic lipidated peptoid component comprises Compound 124, Compound 126, Compound 134, or a combination thereof.
   69. The delivery vehicle composition of claim 64, wherein the anionic/zwitterionic lipidated peptoid component comprises one or more compounds selected from Compounds 125, 131, 133, 135, 136.
   70. The delivery vehicle composition of claim 64, wherein the non-cationic lipidated peptoid component comprises Compound 124, Compound 125, Compound 126, or combinations thereof.
   71. The delivery vehicle composition of claim 64, wherein the anionic/zwitterionic lipidated peptoid component is selected from a compound listed in Table 1B.
   72. The delivery vehicle composition of any one of claims 36-71, further comprising a PEGylated lipidated peptoid component.
   73. The delivery vehicle composition of claim 72, wherein the PEGylated lipidated peptoid component comprises a compound of Formula (Va), a compound of Formula (Vb), or a combination thereof:

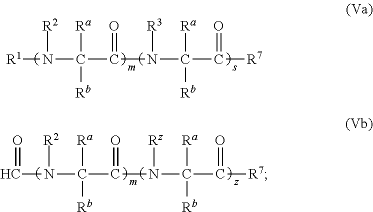

wherein:
m is an integer from 1 to 15;
s is an integer from 1 to 6;
z is an integer from 1 to 6;
R¹ is H or alkyl;
each R² independently is an ethylene glycol moiety of the formula —CH₂CH₂O(CH₂CH₂O)ᵤCH₃, and wherein each u is independently an integer from 2 to 200;
each R³ independently is C₈-C₂₄alkyl or C₈-C₂₄-alkenyl;
R⁷ is —NH₂;
each R^{a} and R^{b} independently is —H; and
each R^{z} independently is C₂₋₅alkylenecarboxylic acid.
   74. The delivery vehicle composition of claim 73, wherein the PEGylated lipidated peptoid component comprises Compound 106, Compound 107, Compound 108, Compound 109, or combinations thereof.
   75. The delivery vehicle composition of claim 72, wherein the PEGylated lipidated peptoid component comprises one or more of Compounds 56, 64, 72.
   76. The delivery vehicle composition of claim 72, wherein the PEGylated lipidated peptoid component is selected from a compound listed in Table 1D.
   77. The delivery vehicle composition of any one of claims 36-76, further comprising Compound 135.
   78. The delivery vehicle composition of claim 36, wherein:
   (a) the cationic lipidated peptoid component comprises Compound 24, Compound 73, Compound 79, Compound 81, Compound 85, Compound 93, Compound 112, Compound 115, Compound 127, Compound 137, Compound 152, Compound 155, Compound 163, Compound 164, or combinations thereof; and
   (b) the non-cationic lipidated peptoid component comprises Compound 90, Compound 119, Compound 120, Compound 121, Compound 122, Compound 123, Compound 124, Compound 125, Compound 126, Compound 128, Compound 129, or combinations thereof.
   79. The delivery vehicle composition of claim 78, wherein:
   (a) the cationic lipidated peptoid component comprises Compound 81, Compound 112, Compound 155, Compound 163, Compound 164, or combinations thereof; and
   (b) the non-cationic lipidated peptoid component comprises Compound 90, Compound 119, Compound 124, Compound 125, Compound 126, or combinations thereof.
   80. The delivery vehicle composition of claim 79, wherein:
   (a) the cationic lipidated peptoid component comprises Compound 81, Compound 155, or combinations thereof; and
   (b) the non-cationic lipidated peptoid component comprises Compound 90, Compound 119, or combinations thereof.
   81. The delivery vehicle composition of any one of claims 36-80, further comprising a PEGylated lipid.
   82. The delivery vehicle composition of claim 81, wherein the PEGylated lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, a PEG-modified sterol, a PEG-modified phospholipid, and combinations thereof.
   83. The delivery vehicle composition of claim 82, wherein the PEGylated lipid comprises dimyristoylglycerol-polyethylene glycol 2000 (DMG-PEG 2000).
   84. The delivery vehicle composition of any one of claim 36-83, wherein the delivery vehicle composition further comprises a phospholipid.
   85. The delivery vehicle composition of claim 84, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE),1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and combinations thereof.
   86. The delivery vehicle composition of claim 85, wherein the phospholipid comprises DOPE, DSPC, or combinations thereof.
   87. The delivery vehicle composition of any one of claims 36-86, further comprising a sterol.
   88. The delivery vehicle composition of claim 87, wherein the sterol comprises cholesterol.
   89. The delivery vehicle composition of any one of claims 57-88, wherein the composition comprises about 20 mol % to about 50 mol % of the lipidated cationic component; about 40 mol % to about 80 mol % of the non-cationic lipidated peptoid component, about 1 mol % to about 5 mol % of the PEGylated lipid, and 0 mol % to about 20 mol % of the phospholipid.
   90. The delivery vehicle composition of claim 89, wherein the composition comprises about 30 mol % to about 45 mol % of the lipidated cationic component; about 50 mol % to about 70 mol % of the non-cationic lipidated peptoid component, about 1 mol % to about 3 mol % of the PEGylated lipid, and 0 mol % of the phospholipid.
   91. The delivery vehicle composition of claim 90, wherein the composition comprises about 35 mol % to about 40 mol % of the lipidated cationic component; about 55 mol % to about 65 mol % of the non-cationic lipidated peptoid component, about 1.5 mol % to about 2.5 mol % of the PEGylated lipid, and 0 mol % of the phospholipid.
   92. The delivery vehicle composition of claim 91, wherein the composition comprises about 38 mol % of the lipidated cationic component; about 60 mol % of the non-cationic lipidated peptoid component, about 2.3 mol % of the PEGylated lipid, and 0 mol % of the phospholipid.
   93. A delivery vehicle complex comprising the delivery vehicle composition of any one of claims 36-92, and a polyanionic compound.
   94. The delivery vehicle complex of claim 93, wherein the lipidated cationic peptoid component is complexed to the polyanionic compound.
   95. The delivery vehicle complex of claim 93 or 94, wherein the mass ratio of the lipidated cationic peptoid component to the polyanionic compound is between about 5:1 and about 20:1.
   96. The delivery vehicle complex of claim 95, wherein the mass ratio of the lipidated cationic peptoid component to the polyanionic compound is between about 7:1 and about 15:1.
   97. The delivery vehicle complex of claim 96, wherein the mass ratio of the lipidated cationic peptoid component to the polyanionic compound is between about 10:1 and about 11:1.
   98. The delivery vehicle complex of claim 97, wherein the mass ratio of the lipidated cationic peptoid component to the polyanionic compound is about 10:1.
   99. The delivery vehicle complex of any one of claims 93-98, wherein the mass ratio of the non-cationic lipidated peptoid to the polyanionic compound is between about 4:1 and about 6.5:1.
   100. The delivery vehicle complex of claim 99, wherein the mass ratio of the non-cationic lipidated peptoid to the polyanionic compound is between about 5:1 and about 6:1.
   101. The delivery vehicle complex of claim 100, wherein the mass ratio of the non-cationic lipidated peptoid to the polyanionic compound is about 5.4:1.
   102. The delivery vehicle complex of any one of claims 93-101, wherein the mass ratio of the PEGylated lipid to the polyanionic compound is between about 1:1 to about 2:1.
   103. The delivery vehicle complex of claim 102, wherein the mass ratio of the PEGylated lipid to the polyanionic compound is about 1.4:1.
   104. The delivery vehicle complex of any one of claims 93-103, wherein the mass ratio of the lipidated cationic peptoid to the polyanionic compound is about 10:1, the mass ratio of the non-cationic lipidated peptoid to the polyanionic compound is about 5.4:1, and the mass ratio of the PEGylated lipid to the polyanionic compound is about 1.4:1.
   105. The delivery vehicle complex of any one of claims 93-104, wherein:
   (a) the cationic lipidated peptoid component comprises Compound 24, Compound 73, Compound 79, Compound 81, Compound 85, Compound 93, Compound 112, Compound 115, Compound 137, Compound 152, Compound 155, Compound 163, Compound 164, or combinations thereof;
   (b) the non-cationic lipidated peptoid component comprises Compound 90, Compound 119, Compound 120, Compound 121, Compound 122, Compound 123, Compound 124, Compound 125, Compound 126, Compound 128, Compound 129, or combinations thereof; and

(c) the PEGylated lipid is DMG-PEG 2000.
106. The delivery vehicle composition of claim 105, wherein:
(a) the cationic lipidated peptoid component comprises Compound 81, Compound 112, Compound 155, Compound 163, Compound 164, or combinations thereof; and
(b) the non-cationic lipidated peptoid component comprises Compound 90, Compound 119, Compound 124, Compound 125, Compound 126, or combinations thereof.
107. The delivery vehicle composition of claim 106, wherein:
(a) the cationic lipidated peptoid component comprises Compound 81, Compound 155, or combinations thereof; and
(b) the non-cationic lipidated peptoid component comprises Compound 90, Compound 119, or combinations thereof.
108. The delivery vehicle complex of claim 107, wherein the lipidated cationic component comprises Compound 81, and the non-cationic lipidated peptoid component comprises Compound 90.
109. The delivery vehicle complex of claim 107, wherein the lipidated cationic component comprises Compound 81, and the non-cationic lipidated peptoid component comprises Compound 119.
110. The delivery vehicle complex of claim 107, wherein the lipidated cationic component comprises Compound 155, and the non-cationic lipidated peptoid component comprises Compound 90.
111. The delivery vehicle complex of claim 107, wherein the lipidated cationic component comprises Compound 155, and the non-cationic lipidated peptoid component comprises Compound 119.
112. The delivery vehicle complex of any one of claims 93-111, further comprising Compound 135.
113. The delivery vehicle complex of any one of claims 93-112, wherein the complex exhibits a particle size of less than about 200 nm and/or a polydispersity index (PDI) of less than 0.25.
114. The delivery vehicle complex of claim 113, wherein the complex exhibits a particle size of about 50 nm to about 95 nm.
115. The delivery vehicle complex of claim 113, wherein the complex exhibits a particle size of about 105 nm to about 200 nm.
116. The delivery vehicle complex of any one of claims 93-115, wherein at least 80% of the polyanionic compound is retained after storage at 4° C. for 48 days, or the delivery vehicle complex retains at least 80% of its original size after storage at 4° C. for 48 days, or both.
117. The delivery vehicle complex of any one of claims 93-116, wherein the polyanionic compound comprises at least one nucleic acid.
118. The delivery vehicle complex of claim 117, wherein the at least one nucleic acid comprises RNA, DNA, or a combination thereof.
119. The delivery vehicle complex of claim 118, wherein the at least one nucleic acid comprises RNA.
120. The delivery vehicle complex of claim 119, wherein the RNA is mRNA encoding a peptide, a protein, or a functional fragment of any the foregoing.
121. The delivery vehicle complex of claim 120, wherein the mRNA encodes for a viral peptide, a viral protein, or functional fragment of any of the foregoing.
122. The delivery vehicle complex of claim 121, wherein the mRNA encodes for a human papillomavirus (HPV) protein or a functional fragment thereof.
123. The delivery vehicle complex of claim 122, wherein the mRNA encodes for the HPV E6 protein and/or the HPV E7 protein, or a functional fragment of any of the foregoing.
124. The delivery vehicle complex of claim 121, wherein the mRNA encodes for a viral spike protein or a functional fragment thereof.
125. The delivery vehicle complex of claim 124, wherein the mRNA encodes for a SARS-CoV spike (S) protein or a functional fragment thereof.
126. The delivery vehicle complex of claim 121, wherein the mRNA encodes for influenza hemagglutinin (HA), or a functional fragment thereof.
127. The delivery vehicle complex of claim 121, comprising an mRNA that encodes for a SARS-CoV spike (S) protein and an mRNA that encodes for influenza hemagglutinin (HA), or a functional fragment of the foregoing.
128. A pharmaceutical composition comprising the delivery vehicle complex of any one of claims 93-127, and a pharmaceutically acceptable excipient.
129. The pharmaceutical composition of claim 128 as an intratumoral (IT) or intramuscular (MI) composition.
130. A method of inducing an immune response in a subject in need thereof, comprising administering to the subject in need thereof an effective amount of the delivery vehicle complex of any one of claims 93-127, or the pharmaceutical formulation of claim 128 or 129, thereby inducing an immune response in the subject.
131. A method of treating a viral infection in a subject in need thereof, comprising administering to the subject an effective amount of the delivery vehicle complex of any one of claims 93-127, or the pharmaceutical formulation of any of claim 128 or 129, thereby treating the viral infection in the subject.
132. A method of treating cancer in a subject in need thereof, comprising administering to the subject an effective amount of the delivery vehicle complex of any one of claims 93-127, or the pharmaceutical formulation of any of claim 128 or 129, thereby treating the cancer in the subject.
133. The method of claim 132, wherein the cancer is cervical cancer, head and neck cancer, B-cell lymphoma, T-cell lymphoma, prostate cancer, lung cancer, or a combination thereof.
134. The method of any one of claims 130-132, wherein the administering is by intramuscular, intratumoral, intravenous, intraperitoneal, or subcutaneous delivery.
135. A method of delivering a polyanionic compound to a cell comprising contacting the cell with the delivery vehicle complex of any one of claims 93-127 or the pharmaceutical composition of claim 128 or 129.
136. The method of claim 135, wherein the polyanionic compound is an mRNA that encodes for a peptide, a protein, or a fragment of any of the foregoing, and the cell expresses the peptide, the protein, or the fragment after being contacted with the delivery vehicle complex.
137. A method of forming the delivery vehicle complex of any one of claims 93-127, comprising contacting the lipidated cationic peptoid component with the polyanionic compound.
138. The method of claim 137, comprising admixing a solution comprising the lipidated cationic peptoid component with a solution comprising the polyanionic compound.
139. A compound of Formula (If):

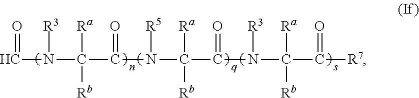

wherein:
n is an integer from 0 to 4;
q is 1 or 2;
s is an integer from 1 to 4;
each R³ independently is C₈-C₂₄alkyl or C₈-C₂₄-alkenyl;
R⁵ is selected from the group consisting of aminoalkyl, alkylaminoalkyl, aminoalkylaminoakyl, guanidinoalkyl, N-heterocyclylalkyl, and N-heteroaryl;
R⁷ is NH₂; and
each of R^{a} and R^{b} independently is H.
   140. The compound of claim 139 selected from Compounds 81, 82, 83, 95, 96, 142, 144, 155, 156, 163, 164, and 165.
   141. A compound of Formula (IVa):

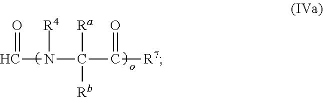

wherein o is integer from 3 to 10;
each R⁴ independently is C₈-C₂₄alkyl, or C₁-C₄-alkyl substituted with cycloalkyl, heterocyclylalkyl, aryl, alkylaryl, arylalkyl, alkylheteroaryl, heteroarylalkyl, alkoxy, alkoxyalkyl, or hydroxyalkyl;
R⁷ is —NH₂; and
each R^{a} and R^{b} independently is —H.
   142. The compound of claim 141 selected from Compounds 90, 119, 120, 121, 122, 123, 128, and 129.
   143. A compound of Formula (IIIa)

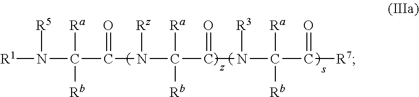

wherein:
s is an integer from 1 to 6;
z is 1 or 2;
R¹ is H or alkyl;
each R³ independently is C₈-C₂₄alkyl or C₈-C₂₄-alkenyl;
R⁵ is selected from the group consisting of aminoalkyl, alkylaminoalkyl, aminoalkylaminoakyl, guanidinoalkyl, N-heterocyclylalkyl, and N-heteroaryl;
R⁷ is —NH₂;
each R^{a} and R^{b} independently is —H; and
each R^{z} independently is C₂₋₅ alkylenecarboxylic acid.
   144. The compound of claim 143 selected from Compound 104 and Compound 105.
   145. A compound of Formula (IIIb):

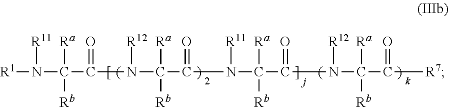

wherein:
j is 1, 2, 3, 4, 5, or 6;
k is 1, 2, 3, or 4;
R¹ is —H, alkyl, alkylaryl, —COR^{1a} or a lipid moiety, wherein R^{1a} is —H, —OH, alkyl, aryl, alkylaryl, —O-alkyl, or —O-alkylaryl;
each R¹¹ independently is aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, hydroxylheteroalkyl, or C₂₋₅ alkylenecarboxylic acid;
each R¹² independently is C₈-C₂₄alkyl, C₈-C₂₄-alkenyl, C₁-C₄aralkyl, or C₁₋₄heteroaralkyl;
R⁷ is —H, alkyl, acyl, —OH, —OR^{7a}, —NH₂, —NHR^{7a}, or a lipid moiety, wherein R^{7a} is alkyl, acyl, or a lipid moiety; and
each R^{a} and R^{b} independently is —H, C₁-C₄-alkyl, or a side chain moiety found on a naturally- or non-naturally-occurring amino acid.
   146. The compound of claim 145 selected from Compound 124, Compound 126, and Compound 134.
   147. A compound listed in Table 1A, 1B, 1C, or 1D selected from Compound 73-165.

It should be appreciated that all combinations of the foregoing concepts and implementations, and additional concepts and implementations, discussed in greater detail below are contemplated as being part of the inventive subject matter disclosed herein, and may be employed in any suitable combination to achieve the benefits as described here. In particular, all combinations of claimed subject matter appearing at the end of this disclosure are contemplated as being part of the inventive subject matter disclosed herein.

Further aspects and advantages will be apparent to those of ordinary skill in the art from a review of the following detailed description, taken in conjunction with the drawings. While the compounds and methods disclosed herein are susceptible of implementations in various forms, the description hereafter includes specific implementations with the understanding that the disclosure is illustrative, and is not intended to limit the invention to the specific implementations described herein.

### BRIEF DESCRIPTION OF THE DRAWINGS

FIG. 1A and FIG. 1B show the in vivo expression of firefly luciferase (Fluc) in C57BI/6 mice treated via intramuscular injection (“IM”) with the indicated mixed peptoid delivery systems (as described in Table 4), complexed with mRNA coding for firefly luciferase (“Fluc mRNA”), compared to a control. The data show that the multicomponent delivery vehicles of the disclosure elicit high luciferase expression in vivo when complexed with Fluc mRNA.

FIG. 2 shows the cellular immune response in C57BI/6 mice treated (via IM) with the indicated multicomponent delivery systems (as described in Table 4) complexed with mRNA coding for Ovalbumin (OVA), compared to a control. The data show that the multicomponent delivery vehicles of the disclosure elicit a strong T cell vaccine response.

FIG. 3A shows the relative bioluminescence following transfection of peripheral blood mononuclear cells (PBMCs) with the indicated multicomponent delivery vehicle systems (112-F2, 79-F2, and 112-MP1-90 (each as described herein) as compared to controls (untreated PBMCs or commercial lipid nanoparticle (LNP)). FIG. 3B shows the percentage of particular cell populations within the parent PBMC population that were transfected with composition 112-MP1-90. FIG. 3C shows the viability of macrophages and dendritic cells (DCs) following administration of 5× working concentration of composition 112-MP1-90.

FIG. 4 shows the in vivo expression of Fluc mRNA in C57BI/6 mice treated intratumorally (“IT”) with multicomponent delivery systems of the disclosure, such as 112-MP1-90 and as disclosed in Table 4, complexed with Fluc mRNA, compared to a delivery system comprising a commercial cationic component (DLIN-MC3-DMA, MC3). The data demonstrate that the delivery vehicle complexes of the disclosure elicit strong Fluc expression via intratumoral injection when complexed with mRNA encoding for Fluc.

### DETAILED DESCRIPTION

The following description sets forth example methods, parameters and the like. It should be recognized, however, that such description is not intended as a limitation on the scope of the present disclosure but is instead provided as a description of example implementations.

The cellular and in vivo delivery of oligonucleotides remains a significant hurdle in the field of gene therapy. Therefore, chemical strategies to enhance the bioavailability of this class of drugs remain in significant demand. Notably, prior systems in the area of chemical agents for nucleic acid delivery have focused on cationic lipid nanoparticles (LNPs), and polycationic polymers. LNPs have traditionally included 4 lipid-like components that utilize hydrophobic interactions to self-assemble into lamellar or non-lamellar particles and encapsulate anionic oligonucleotides. Polymeric delivery agents typically utilize molecules with numerous cationic charges to electrostatically bind the nucleic acid into a nano-sized coacervate. These cationic materials sometimes include additional elements such as hydrophilic shielding elements (e.g., poly(ethylene glycol) (PEG)) or lipid elements through block co-polymer structures. These domains are most commonly incorporated onto the same polymer in what can be described as a “jack of all trades” strategy in an attempt to balance the many necessary functions for nucleic acid delivery. However, while lipid nanoparticle formulations have often melded multiple functions (such as cationic nucleic acid binding, PEG shielding, lipid fluidity, etc.) by lipid anchoring into the LNP, relatively little innovation has taken place to systematically incorporate multiple polycationic elements with specific functions into a single particle. There is a need in the art for methods and compositions that can be optimized for delivering various types of nucleic acid material to a range of different target cells.

Accordingly, disclosed herein, in some implementations, are multicomponent delivery systems comprising, in some examples, one or more lipidated peptoid components, such as a lipidated cationic peptoid component (“lipidated cationic peptoid”), and additionally in some instances, a non-cationic lipididated peptoid component (“non-cationic lipidated peptoid”). The multicomponent delivery systems disclosed herein can effectively and efficiently deliver polyanionic cargo, such a one or more nucleic acids (e.g., mRNA), to a cell where uptake and expression of the nucleic acid(s) can occur. When the multicomponent delivery system described herein are complexed with polyanionic cargo (e.g., mRNA), they can be used as vaccines to elicit strong cellular and humoral immune responses. In some instances, the multicomponent delivery systems of the disclosure have a benefit of providing nanoparticles that are stable and monodisperse. Further, the multicomponent delivery systems of the disclosure can be administered through a variety of routes (e.g., intravenous, intratumoral, subcutaneous, and intramuscular). As used herein the term “multicomponent delivery vehicle system” is interchangeable with “delivery vehicle composition” or “delivery vehicle.”

### Components of the Multicomponent Delivery System

The present disclosure provides examples related to a multicomponent polyanionic compound delivery vehicle system utilizing a plurality of components to contribute specific features to the resulting complex. “Multicomponent” as used herein refers to a composition or complex with more than one structurally different compound (also referred to as component). The multicomponent delivery system comprises complexes of at least one peptoid compound. “Peptoid” as used herein refers to peptidomimetic compounds wherein the nitrogen atoms of the peptide backbone are substituted with side chains. “Lipidated peptoids” refer to peptoids in which the side chains on the nitrogen atoms comprise lipids. “Nucleic acids” as used herein refers to naturally occurring oligonucleotide compounds, such as but not limited to DNA, RNA, and/or hybrids thereof, as well as unnaturally occurring variations thereof. Unnaturally occurring nucleic acids can comprise, for example, an unnatural backbone, one or more modified backbone linkages such as phosphorothioate, unnatural and modified bases, unnatural and modified termini, and combinations thereof. Non-limiting examples of nucleic acids include mRNA, small interfering RNA (siRNA), small activating RNA (saRNA), micro RNA (miRNA), antisense oligonucleotides, ribozymes, plasmids, and immune stimulating nucleic acids.

In some implementations, the multicomponent delivery system comprises lipidated cationic peptoid compounds. The lipidated cationic peptoid compounds have one or more cationic anchoring groups that can associate with polyanionic compounds, such as nucleic acids, through electrostatic interactions between the cationic anchoring group(s) and negative charges on the polyanionic compounds, such as the negative phosphodiester backbone of nucleic acids.

In some implementations, the multicomponent delivery system comprises cationic peptoid compounds in combination with further components. These further components each serve a specific function or functions in the delivery vehicle, such that all functionality for cellular delivery does not need to be accomplished by a single entity. These functions can be isolated, or can be served in concert through cooperation between two or more components. The further components may comprise additional peptoid compounds including cationic peptoids, neutral lipidated peptoids, anionic peptoids, zwitterionic peptoids, and/or shielding (or PEGylated) peptoids. The delivery vehicles can further comprise lipids, such as structural lipids, phospholipids, and/or shielding lipids to prevent aggregation. The further components may comprise other peptoids that function as structural lipids, phospholipids, and/or shielding lipids.

In some cases, the multicomponent delivery systems disclosed herein are mixed peptoid delivery systems, or mixed peptoid delivery vehicles. A “mixed peptoid delivery system” or “mixed peptoid delivery vehicle” refers to a multicomponent delivery system that includes at least two types of peptoid components, such as a lipidated cationic peptoid and a non-cationic lipididated peptoid. Examples of the non-cationic lipidated peptoid include neutral lipidated peptoids, anionic and/or zwitterionic lipidated peptoids (“anionic/zwitterionic lipidated peptoids”), and shielding (or PEGylated) peptoids, each as described in detail below. In various cases, the multicomponent delivery systems disclosed herein include one peptoid component, such as a lipidated cationic peptoid, and are referred to as “single peptoid delivery systems,” or “single peptoid delivery vehicles.”

### Cationic Component

### Cationic Anchor

In some implementations, the multicomponent delivery systems described herein include a cationic component, such as a lipidated cationic peptoid. The cationic component of the multicomponent delivery system of the present disclosure comprises one or more compounds that have a cationic anchoring portion, which contributes to their association with polyanionic (e.g., nucleic acid) cargos and other components. The compounds that have a cationic anchoring portion may be lipidated cationic peptoids that can form complexes to counterbalance the negative charge on the polyanionic cargoes, such as nucleic acids, thus promoting uptake of the cargo into a target cell. The lipidated cationic peptoids as described herein have a net zero charge or a net positive charge. In some implementations wherein the multicomponent delivery system has one or more cationic compounds, each of the one or more lipidated cationic peptoids independently has a net zero charge or a net positive charge. In certain implementations, each of the one or more lipidated cationic peptoids independently has a net positive charge of at least +1. It should be recognized that the net charge present on the one or more lipidated cationic peptoids may vary depending upon environmental conditions. For example, in some implementations, each of the one or more lipidated cationic peptoids independently has a stable, net positive charge at physiologically relevant pH ranges. For example, physiological pH is at least about 5.5 and typically at least about 6.0. More typically, physiological pH is at least about 6.5. Usually, physiological pH is less than about 8.5 and typically less than about 8.0. More typically, physiological pH is less than about 7.5 (e.g., 7.4). Thus, physiological pH can range from about 5.5 to about 8.5, or about 5.5 to about 8.0, or about 6.0 to about 7.5, or about 6.5 to about 8.0, or about 7.35 to about 7.45, such as about 7.4.

In some variations of the foregoing, the multicomponent delivery system comprises about 10.0 to about 99.5 mol % (molar percentage) of lipidated cationic peptoids of the total number of moles of the multicomponent delivery system components. In the present disclosure, when the mol % of a component is discussed, the polyanionic cargo compound is not calculated in the total number of moles. The mol % of a component is the number of moles of a particular component divided by the total number of moles of all the components of the multicomponent delivery system. The polyanionic cargo is not calculated in the total number of moles of the delivery vehicle composition.

In certain variations, the multicomponent delivery system comprises between about 10.1 to about 20.0 mol %, about 20.1 to about 30.0 mol %, about 30.1 to about 40.0 mol %, about 40.1 to about 50.0 mol %, about 50.1 to about 60.0 mol %, about 60.1 to about 70.0 mol %, about 70.1 to about 80.0 mol %, about 80.1 to about 90.0 mol %, or about 90.1 to about 99.5 mol % of lipidated cationic peptoids of the total number of moles of the multicomponent delivery system. In some cases, the multicomponent delivery system comprises between about 10 to about 20 mol %, about 20 to about 30 mol %, about 30 to about 40 mol %, about 40 to about 50 mol %, about 50 to about 60 mol %, about 60 to about 70 mol %, about 70 to about 80 mol %, about 80 to about 90 mol %, or about 90 to about 99.5 mol % of lipidated cationic peptoids of the total number of moles of the multicomponent delivery system. In various cases, the multicomponent delivery system comprises between about 10 to about 55 mol %, about 15 to about 50 mol %, about 20 to about 45 mol %, about 25 to about 40 mol %, about 30 to about 50 mol %, about 35 to about 45 mol %, about 40 to about 48 mol %, about 43 to about 49.5 mol %, or about 20 to about 35 mol % of lipidated cationic peptoids of the total number of moles of the multicomponent delivery system. In various cases, the multicomponent delivery system comprises between about 20 to about 50 mol %, about 15 to about 50 mol %, about 20 to about 45 mol %, about 25 to about 40 mol %, about 30 to about 45 mol %, about 35 to about 40 mol %, about 40 to about 48 mol %, about 43 to about 49.5 mol %, or about 20 to about 35 mol % of lipidated cationic peptoids of the total number of moles of the multicomponent delivery system. In some implementations, the multicomponent delivery system comprises less than about 50 mol % of lipidated cationic peptoids, such as less than about 49 mol %, less than about 48 mol %, less than about 47 mol %, less than about 46 mol %, less than about 45 mol %, less than about 44 mol %, less than about 43 mol %, less than about 42 mol %, less than about 41 mol %, less than about 40 mol %, less than about 39 mol %, less than about 38 mol %, less than about 37 mol %, less than about 36 mol %, less than about 35 mol %, less than about 34 mol %, less than about 33 mol %, less than about 32 mol %, less than about 31 mol %, less than about 30 mol %; and greater than about 20 mol % of lipidated cationic peptoids, such as greater than about 21 mol %, greater than about 22 mol %, greater than about 23 mol %, greater than about 24 mol %, greater than about 25 mol %, greater than about 26 mol %, greater than about 27 mol %, greater than about 28 mol %, greater than about 29 mol %, greater than about 30 mol %, greater than about 31 mol %, greater than about 33 mol %, greater than about 34 mol %, greater than about 35 mol %, greater than about 36 mol %, greater than about 38 mol %, greater than about 39 mol %, or greater than about 40 mol of lipidated cationic peptoids of the total number of moles of the multicomponent delivery system.

In some implementations, the lipidated cationic peptoids used in the compositions as provided herein may include cationic peptoid-phospholipid conjugate constructs, also known as “lipitoids.” In some implementations, the lipidated cationic peptoids used in the multicomponent delivery system may include N-substituted cationic peptide compounds possessing lipid moieties and/or (oligo- and/or poly)ethylene glycol moieties throughout the peptide backbone, herein referred to as “tertiary amino lipidated and/or PEGylated cationic peptoids.” In some implementations, the multicomponent delivery system of the present disclosure comprises complexes comprising one or more tertiary amino lipidated and/or PEGylated cationic peptoids. In other implementations, the multicomponent delivery system comprises complexes comprising one or more lipitoids. In still other implementations, the multicomponent delivery system comprises complexes comprising one or more tertiary amino lipidated and/or PEGylated cationic peptoids, one or more lipitoids, or any combinations thereof.

In some implementations, the tertiary amino lipidated and/or PEGylated cationic peptoids comprise an oligopeptide backbone, wherein the oligopeptide backbone comprises one subunit or repeating subunits of N-substituted cationic amino acid residues that can be, in some examples, interleaved with N-substituted neutral (“spacer”) and/or lipid amino acid residues. In some implementations, the oligopeptide backbone can be further capped at the N- and/or C-terminus by amino acid residues that are N-substituted with lipid moieties (“N-lipidated”) and/or N-substituted with oligoethylene glycol and/or polyethylene glycol (“N-PEGylated”).

In some implementations, the cationic components are tertiary amino lipidated and/or PEGylated cationic peptoids selected from any tertiary amino lipidated and/or PEGylated cationic peptoid compounds disclosed in WO2020/069442 or WO2020/069445, each of which is incorporated by reference in its entirety, such as tertiary amino lipidated and/or PEGylated cationic peptoid compounds of formula (I) or salts thereof:

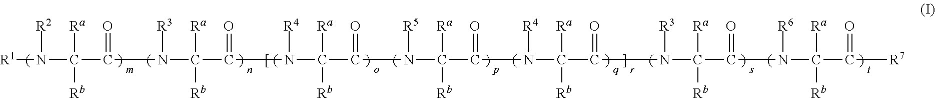

wherein:
m is an integer from 0 to 10;
n is an integer from 0 to 8, such as 0 to 5;
s is an integer from 0 to 8, such as 0 to 5;
t is an integer from 0 to 10;

wherein at least one of m, n, s, and t is nonzero;
r is an integer from 1 to 20;
each o is independently an integer 0, 1, or 2;
each q is independently an integer 0, 1, or 2;
each p is independently an integer 1 or 2;
R¹ is —H, alkyl, alkylaryl, —COR^{1a} or a lipid moiety,

wherein R^{1a} is —H, —OH, alkyl, aryl, alkylaryl, —O-alkyl, or —O-alkylaryl;
each R² is independently an ethylene glycol moiety of the formula —CH₂CH₂O(CH₂CH₂O)ᵤCH₃, and wherein each u is independently an integer from 2 to 200;
each R³ is independently a lipid moiety;
each R⁴ is independently a neutral spacer moiety or a lipid moiety;
each R⁵ is independently a cationic moiety;
each R⁶ is independently an ethylene glycol (EG) moiety of the formula —CH₂CH₂O(CH₂CH₂O)ᵥCH₃, and wherein each v is independently an integer from 2 to 200;
R⁷ is —H, alkyl, acyl, —OH, —OR^{7a}, —NH₂, —NHR^{7a}, or a lipid moiety, wherein R^{7a} is alkyl, acyl, or a lipid moiety; and
each R^{a} and R^{b} are independently —H, C₁-C₄-alkyl, or a side chain moiety found on a naturally- or non-naturally-occurring amino acid.

In some implementations, R¹ is H. In various implementations, R¹ is alkyl. Contemplated R¹ groups include, for example, —CH₃,

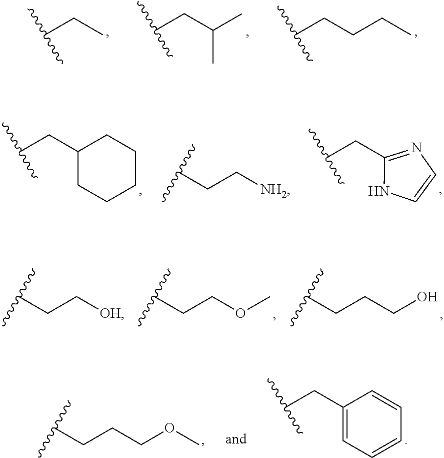

Variable R⁵ of the cationic component is a cationic moiety. The cationic moiety R⁵ can include, for example, nitrogen-based substituents, such as those containing the following functional groups: amino, guanidino, hydrazido, and amidino. These functional groups can be either aromatic, saturated cyclic, or aliphatic. In some implementations, each R⁵ independently is aminoalkyl, alkylaminoalkyl, aminoalkylaminoakyl, guanidinoalkyl, N-heterocyclylalkyl, or N-heteroaryl. In some implementations, each R⁵ independently is

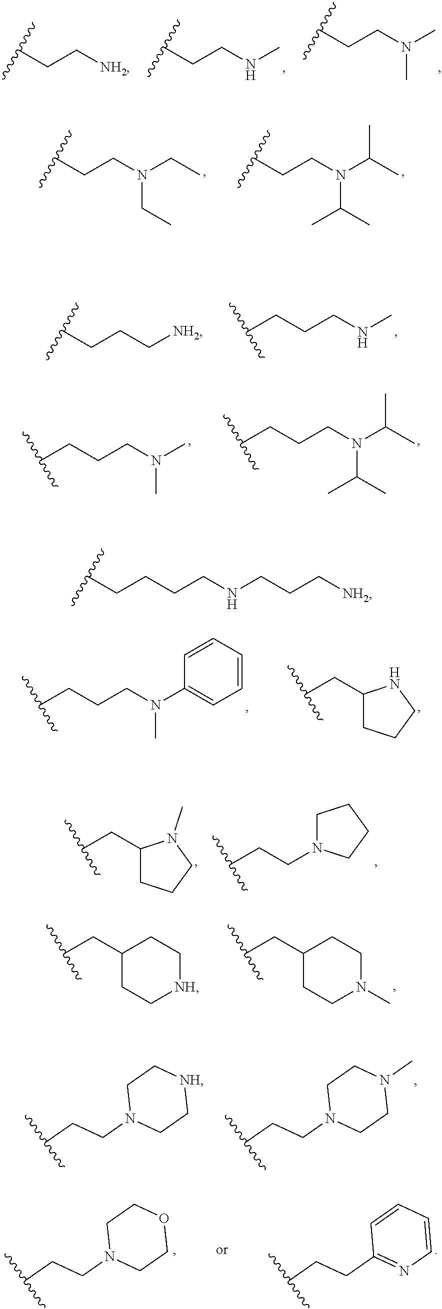

In some implementations, each R⁵ is independently selected from the group consisting of:

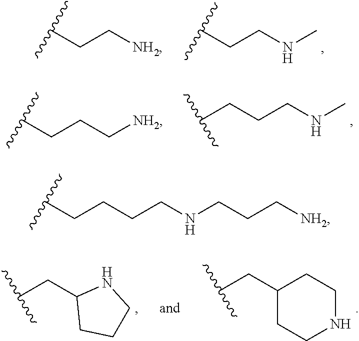

In various implementations, each R⁵ independently is

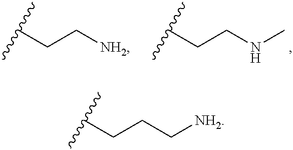

In other implementations, each R⁵ is

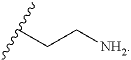 [SMILES: *CCN]

In some cases, each R⁵ is

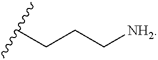 [SMILES: *CCCN]

In some implementations wherein a cationic residue is the terminal residue of the entire peptoid compound, additional cationic moieties R⁵ which are not compatible with the synthesis or deprotection conditions (such as acid-labile linkers) or for which a suitable protecting group strategy is not available (e.g. polyamines) may be used. For example, in some implementations, the cationic moiety R⁵ of the terminal cationic residue is a polyamine. Contemplated polyamines can include, for example,

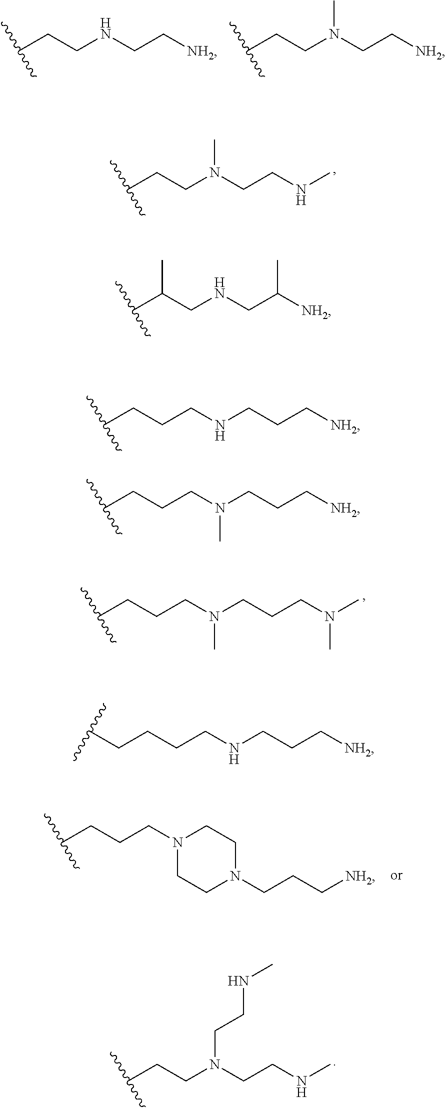

In certain implementations, the polyamine is selected from the group consisting of

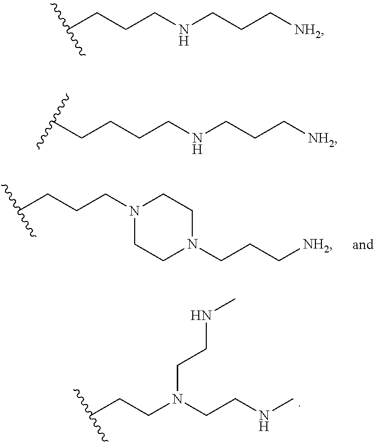

In some implementations, each R⁵ is independently selected from the group consisting of:

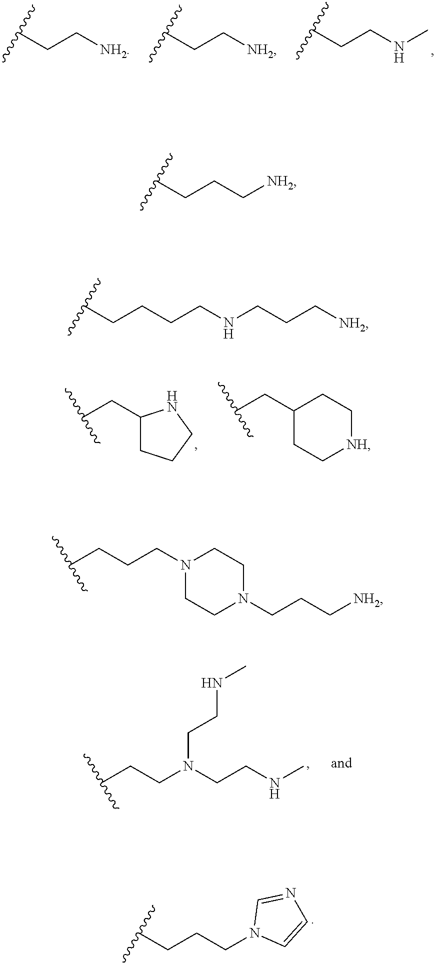

In other implementations, the cationic moiety R⁵ of the terminal cationic residue is a hydroxyalkyl, a hydroxyether, an alkoxyalkyl, or a hydroxylheteroalkyl. In certain implementations, the cationic moiety R⁵ of the terminal cationic residue is

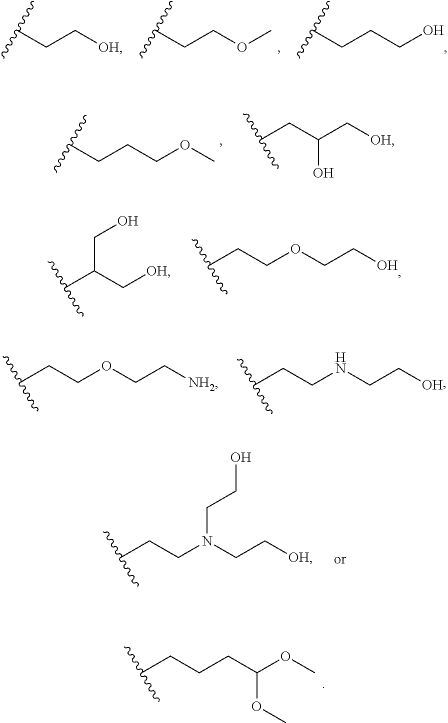

In some implementations, R⁵ is

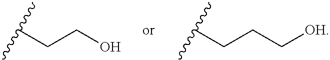

In some cases, R⁵ is

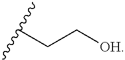 [SMILES: *CCO]

In some cases, R⁵ is

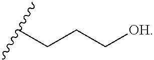 [SMILES: *CCCO]

In still further implementations, the cationic moiety R⁵ of the terminal cationic residue is

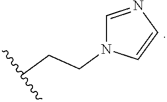 [SMILES: *CCn1ccnc1]

It should be further recognized that an unsubstituted nitrogen atom in the peptide chain, that is, wherein R⁵ is hydrogen, may also serve as an ionizable cationic moiety under physiological conditions. In some implementations, the cationic moiety R⁵ is a hydrogen atom,

### Neutral Spacer Moieties

Within the oligopeptide backbone of tertiary amino lipidated and/or PEGylated cationic peptoids, the cationic amino acid residues may be, in some examples, interleaved with neutral spacer amino acid residues, possessing a neutral spacer moiety at the N-position. The neutral amino acid residues may be useful for modulating the spatial distribution of the positive charge in the tertiary amino lipidated and/or PEGylated cationic peptoid compounds for improved electrostatic interactions with the polyanionic cargo compounds, including polynucleotides, to be complexed with the lipidated cationic peptoid compounds.

In each repeating subunit of the tertiary amino lipidated and/or PEGylated cationic peptoids, a neutral amino acid residue may present on either the N- or C-terminal end of the cationic amino acid residue as one or more R⁴ groups. In some implementations wherein a subunit r comprises a neutral spacer moiety R⁴, the corresponding o and/or q for each neutral spacer moiety present represent the respective numbers of neutral spacer residues bonded to the N- and C-terminal ends of the cationic amino acid residue(s) within the subunit r. In some implementations, each o is independently an integer 0, 1, or 2. In other implementations, each q is independently an integer 0, 1, or 2.

Each neutral spacer amino acid residue comprises a neutral spacer moiety R⁴ at the N-position. As with the cationic moieties described herein, it should be recognized that each neutral spacer moiety R⁴ is independently selected within the repeating subunit of the cationic peptoid compounds as well as amongst the repeating subunits r of the oligopeptide backbone.

It should also be recognized that neutral spacer moieties may include any substituents that are neutral, or have zero net charge, at physiologically relevant pH ranges. In some implementations, each neutral moiety R⁴ is independently a C₁-C₄-alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is, in some cases, substituted with one or more substituents —OH, halo, or alkoxy. In still some implementations, each neutral spacer moiety R⁴ is independently selected from the group consisting of: —CH₃,

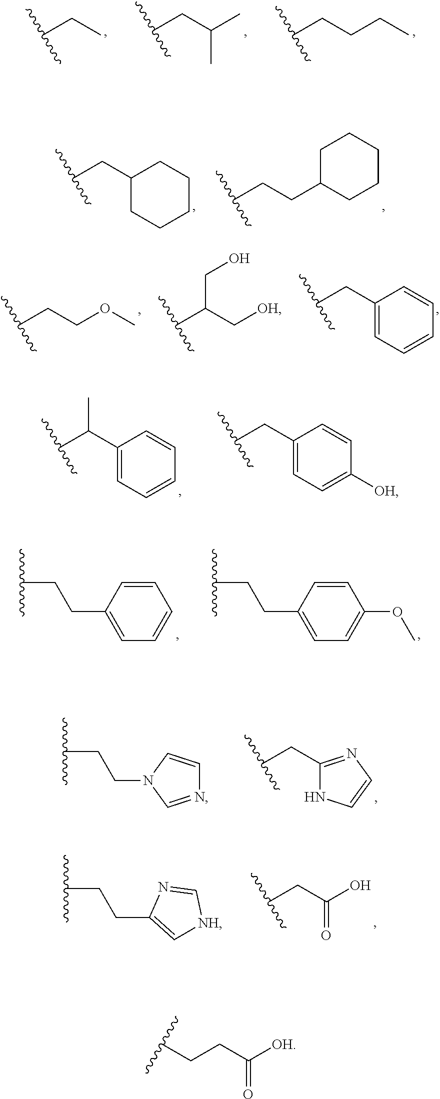

In certain implementations, each neutral spacer moiety R⁴ independently is

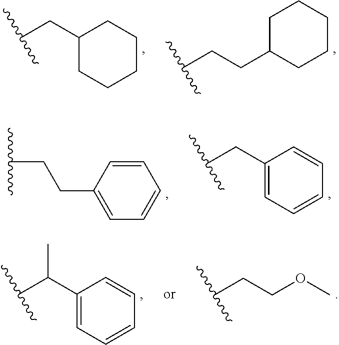

### Lipid Moieties

In addition to the interleaving of neutral amino acid residues with cationic amino acid residues within the oligopeptide backbone of tertiary amino lipidated and/or PEGylated cationic peptoids that can occur in some instances, N-lipidated amino acid residues, possessing a lipid moiety at the N-position, may also, in some cases, be interleaved with the cationic (and neutral spacer) amino acid residues. In some implementations wherein the tertiary amino lipidated and/or PEGylated cationic peptoid comprises N-lipidated amino acid residues, the tertiary amino lipidated and/or PEGylated cationic peptoid is N-lipidated. Similar to the neutral amino acid residues, the N-lipidated amino acid residues within the oligopeptide backbone may be useful in modulating the spatial distribution of the positive charge(s) in the tertiary amino lipidated and/or PEGylated cationic peptoids as well as augment their lipophilicity for improved encapsulation of polyanionic materials and endocellular delivery. The spacing of lipids along the peptoid backbone may also influence the lipid fluidity/crystallinity of the peptoid, which is known to influence cellular uptake and endosomal release.

As with the neutral spacer residues, in each repeating subunit of the tertiary amino lipidated and/or PEGylated cationic peptoids, the N-lipidated amino acid residue may present on either N- or C-terminal end or both ends of the cationic amino acid residue (containing R⁵) as one or more R⁴ groups. In some implementations wherein a subunit r comprises a lipid moiety R⁴, the corresponding o and/or q for each lipid moiety present may also represent the respective numbers of lipidated residues bonded to the N- and C-terminal ends of the cationic amino acid residue(s) within the subunit r. In some implementations, each o is independently an integer 0, 1, or 2. In other implementations, each q is independently an integer 0, 1, or 2.

Each N-lipidated amino acid residue comprises a lipid moiety R⁴ at the N-position. As with the cationic and neutral moieties described herein, it should be recognized that each lipid moiety R⁴ is independently selected within the repeating subunit of the cationic peptoid compounds as well as amongst the repeating subunits r of the oligopeptide backbone.

Suitable lipid moieties may include, for example, optionally substituted branched or straight chain aliphatic moieties, or optionally substituted moieties derived from natural lipid compounds, including fatty acids, sterols, and isoprenoids.

In some implementations, the lipid moieties may include branched or straight chain aliphatic moieties having from about 6 to about 50 carbon atoms or from about 10 to about 50 carbon atoms. In some cases, the aliphatic moities can comprise, in some examples, one or more heteroatoms, and/or one or more double or triple bonds (i.e., saturated or mono- or poly-unsaturated). In certain implementations, the lipid moieties may include optionally substituted aliphatic, straight chain or branched moieties, each hydrophobic tail independently having from about 8 to about 30 carbon atoms or from about 6 to about 30 carbon atoms. In certain implementations, the lipid moieties may include, for example, aliphatic carbon chains derived from fatty acids and fatty alcohols. In some implementations, each lipid moiety R⁴ is independently C₈-C₂₄-alkyl or C₈-C₂₄-alkenyl, wherein the C₈-C₂₄-alkenyl can be, in some instances, mono- or poly-unsaturated. In some implementations, each lipid moiety R⁴ is a C₆-C₁₈ alkyl or C₆-C₁₈ alkenyl. In certain implementations, each lipid moiety R⁴ is C₈-C₁₂ alkyl. In still other implementations, each lipid moiety R⁴ is a C₁₀-alkyl, such as n-decyl. In other implementations, each lipid moiety R⁴ is independently selected from the group consisting of 2-ethylhex-1-yl, caproyl, oleyl, stearyl, linoleyl, myristyl, and lauryl.

In yet other implementations which may be combined with any of the preceding implementations, each lipid moiety R⁴ is independently

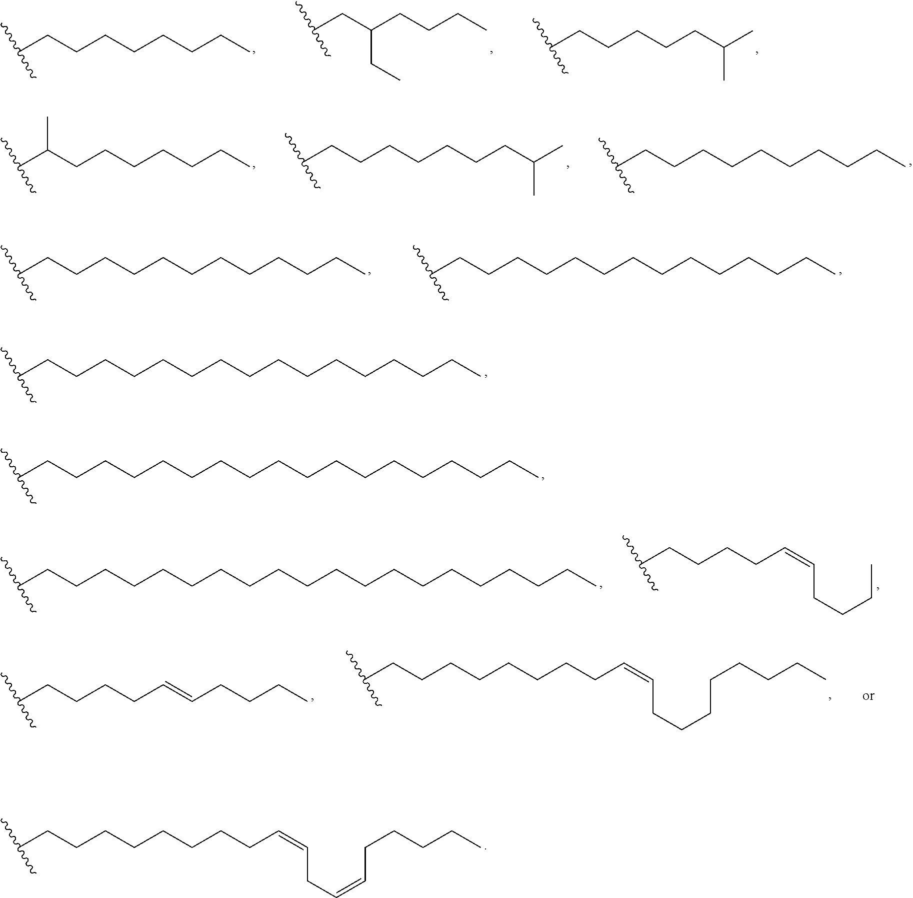

In still yet other implementations which may be combined with any of the preceding implementations, each lipid moiety R⁴ is independently a lipid of the formula C₁-C₈alkylester. In some implementations, each R⁴ is independently a lipid of the formula

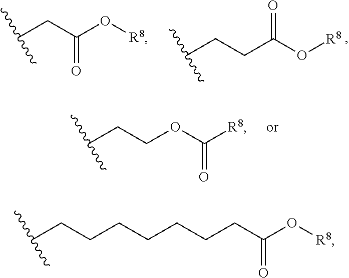

wherein R⁸ is a branched or straight chain aliphatic moieties having from about 6 to about 50 carbon atoms or from about 10 to about 50 carbon atoms. In some instances, the aliphatic moieties can include, for example, one or more heteroatoms, and/or one or more double or triple bonds. In certain implementations, each lipid moiety R⁴ is independently

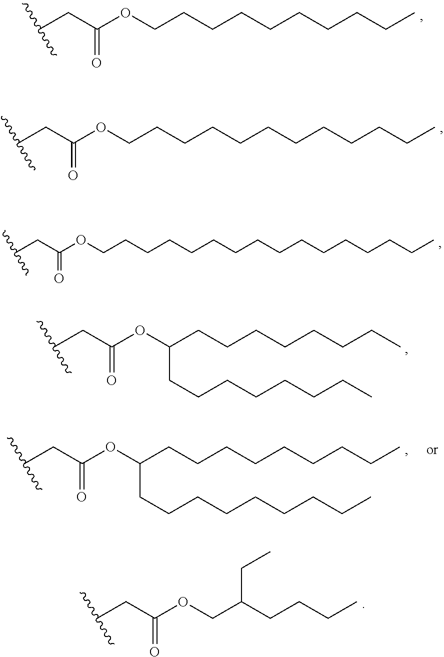

In some implementations, each R⁴ moiety is independently C₈-C₂₄-alkyl, C₈-C₂₄-alkenyl, or C₁-C₈alkylester, each as previously described herein.

Natural lipid moieties employed in the practice of the present disclosure can be derived from, for example, phospholipids, glycerides (such as di- or tri-glycerides), glycosylglycerides, sphingolipids, ceramides, and saturated and unsaturated sterols, isoprenoids, and other like natural lipids.

Other suitable lipid moieties may include lipophilic carbocyclic or aromatic groups such as optionally substituted aryl, cycloalkyl, cycloalkylalkyl, or arylalkyl moieties, including for example naphthalenyl or ethylbenzyl, or lipids comprising ester functional groups including, for example, sterol esters and wax esters. In still other implementations, the lipid moiety R⁴ is

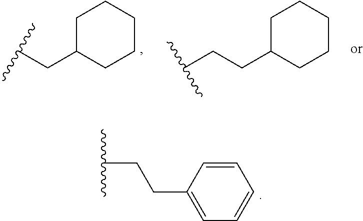 [SMILES: *CC1CCCCC1 ; *CCC1CCCCC1 ; *CCc1ccccc1]

In some cases, the lipid moiety also functions as a neutral spacer.

Additionally or alternatively, the tertiary amino lipidated and/or PEGylated cationic peptoid compounds may comprise amino acid residues at the N-terminus and/or the C-terminus, wherein the amino acid residues are N-substituted with a lipid moiety, or “N-lipidated”. The incorporation of N-lipidated amino acid residues at the N- and/or C-terminus of the cationic peptoid compounds described herein increase the lipophilicity of the compounds. The increased lipophilicity of the cationic peptoid compounds enhances their affinity for hydrophobic environments, such as the lipid bilayer of the cell membrane, thus increasing the propensity of the tertiary amino lipidated and/or PEGylated cationic peptoid compounds, and any complexes thereof with polyanionic compounds, to be transported into the cell.

In some implementations, the tertiary amino lipidated and/or PEGylated cationic peptoid compound is N-lipidated. In some implementations, the tertiary amine lipidated and/or PEGylated cationic peptoid compounds of the present disclosure comprise N-lipidated amino acid residues at the N-terminus. In various implementations, the tertiary amine lipidated and/or PEGylated cationic peptoid compounds of the present disclosure comprise N-lipidated amino acid residues at the C-terminus. In some implementations, the tertiary amine lipidated and/or PEGylated cationic peptoid compounds of the present disclosure comprise N-lipidated amino acid residues at the N- and C-termini.

In some implementations, the number of N-lipidated amino acid residues at the N-terminus of the cationic peptoid compounds described herein is represented by n. In other implementations, the number of N-lipidated amino acid residues at the N-terminus of the cationic peptoid compounds described herein is represented by s.

In some implementations, n is an integer from 0 to 8. In certain implementations, n is an integer from 0 to 5. In other implementations, n is an integer 0, 1, 2, 3, 4, 5, 6, or 7. In yet other implementations, n is an integer 1, 2, 3, or 4. In some implementations, s is an integer from 0 to 8. In various implementations, s is an integer from 0 to 5. In other implementations, v is an integer 0, 1, 2, 3, 4, 5, 6, or 7. In still other implementations, s is an integer 1, 2, 3, or 4. In some implementations wherein the tertiary amino lipidated and/or PEGylated cationic peptoid compound is N-lipidated, at least one of n or s is nonzero. In some implementations, n is nonzero. In other implementations, s is nonzero. In certain implementations, both n and s are nonzero. In some implementations, the sum of n and v is an integer from 1 to 8, from 2 to 7, or from 4 to 6. In other implementations, the sum of n and s is at least 2, at least 3, or at least 4, such as, in some cases, 2, 3, or 4.

In some implementations, the tertiary amino lipidated and/or PEGylated cationic peptoid compound comprises a block of N-lipidated residues, or“N-lipid block” wherein the tertiary amino lipidated and/or PEGylated cationic peptoid comprises at least two, at least three, or at least four N-lipidated residues adjacent to one another (e.g., R^{lipid}R^{lipid}R^{lipid}). In some implementations, the tertiary amino lipidated and/or PEGylated cationic peptoid compound comprises a block of N-lipidated residues, wherein n is at least 2, at least 3, or at least 4. In other implementations, the tertiary amino lipidated and/or PEGylated cationic peptoid compound comprises a block of N-lipidated residues wherein s is at least 2, at least 3, or at least 4.

The tertiary amino lipidated and/or PEGylated cationic peptoid compounds of the disclosure also can include lipidated amino acids at the R³ position of Formula (I). Accordingly, the N-lipidated amino acid residues in the tertiary amino lipidated and/or PEGylated cationic peptoids of the present disclosure can be N-substituted with lipid moieties R³. Lipid moieties R³ of the present disclosure may include hydrophobic or lipophilic moieties that are neutral (i.e., having no charge or a net charge of zero). The lipid moieties of the tertiary amino lipidated and/or PEGylated cationic compounds described herein may be either naturally or synthetically derived. Each R³ is independently a lipid moiety, which may be the same or different. In some implementations, each R³ is the same. In other implementations, each R³ is different.

It should be recognized that particular sequences or arrangements of N-lipidated amino acid residues may be especially useful for improving complexation with and delivery of polyanionic compounds, such as nucleic acids. For example, in some implementations wherein the tertiary amino lipidated and/or PEGylated cationic compounds comprise a set of mixed N-lipidated amino acid residues having one of two different lipid R³ moieties (e.g., R^{3a} and R^{3b}), the N-lipidated amino acid residues may be arranged on either N- or C-terminus in an alternating or block sequences.

One example of an alternating sequence of N-lipidated amino acid residues may be represented by R^{3a}—R^{3b}—R^{3a}—R^{3b} or R^{3b}—R^{3a}—R^{3b}—R^{3a}. An example of a block sequence may be represented by R^{3a}—R^{3a}—R^{3b}—R^{3b} or R^{3b}—R^{3b}—R^{3a}—R^{3a}. In other implementations, the sequence of N-lipidated amino acid residues may be ordered at random. It should be recognized that the above examples of sequences or arrangements of two N-lipidated amino acid residues are not intended to be limiting. Moreover, the tertiary amino lipidated and/or PEGylated cationic peptoid compounds of the present disclosure may comprise two or more different lipid moieties of R³, which may be present in random, alternating or block sequences as generally described above.

Suitable lipid R³ moieties may include, for example, optionally substituted branched or straight chain aliphatic moieties, or optionally substituted moieties derived from natural lipid compounds, including fatty acids, sterols, and isoprenoids.

In some implementations, the lipid R³ moieties may include branched or straight chain aliphatic moieties having from about 6 to about 50 carbon atoms or from about 10 to about 50 carbon atoms. The aliphatic moieties can, in some instances, comprises one or more heteroatoms, and/or one or more double or triple bonds (i.e., saturated or mono- or poly-unsaturated). In various implementations, the lipid R³ moieties may include optionally substituted aliphatic, straight chain or branched moieties, each hydrophobic tail independently having from about 8 to about 30 carbon atoms or from about 6 to about 30 carbon atoms. In some implementations, the lipid moieties may include, for example, aliphatic carbon chains derived from fatty acids and fatty alcohols. In some implementations, each R³ is independently C₂-C₂₄-alkyl or C₂-C₂₄-alkenyl, wherein the C₂-C₂₄-alkenyl can be, in some instances, mono- or poly-unsaturated. In some implementations, each R³ is a C₆-C₁₈ alkyl or C₆-C₁₈ alkenyl. In certain implementations, each R³ is C₈-C₁₂ alkyl. In still other implementations, each R³ is a C₁₀-alkyl, such as n-decyl. In some implementations, each R³ is independently selected from the group consisting of 3-ethylhex-1-yl, caprylyl, caproyl, oleyl, stearyl, linoleyl, myristyl, and lauryl. In other implementations, each R³ is independently selected from the group consisting of oleyl, stearyl, linoleyl, myristyl, and lauryl.

In yet other implementations which may be combined with any of the preceding implementations, each R³ is independently

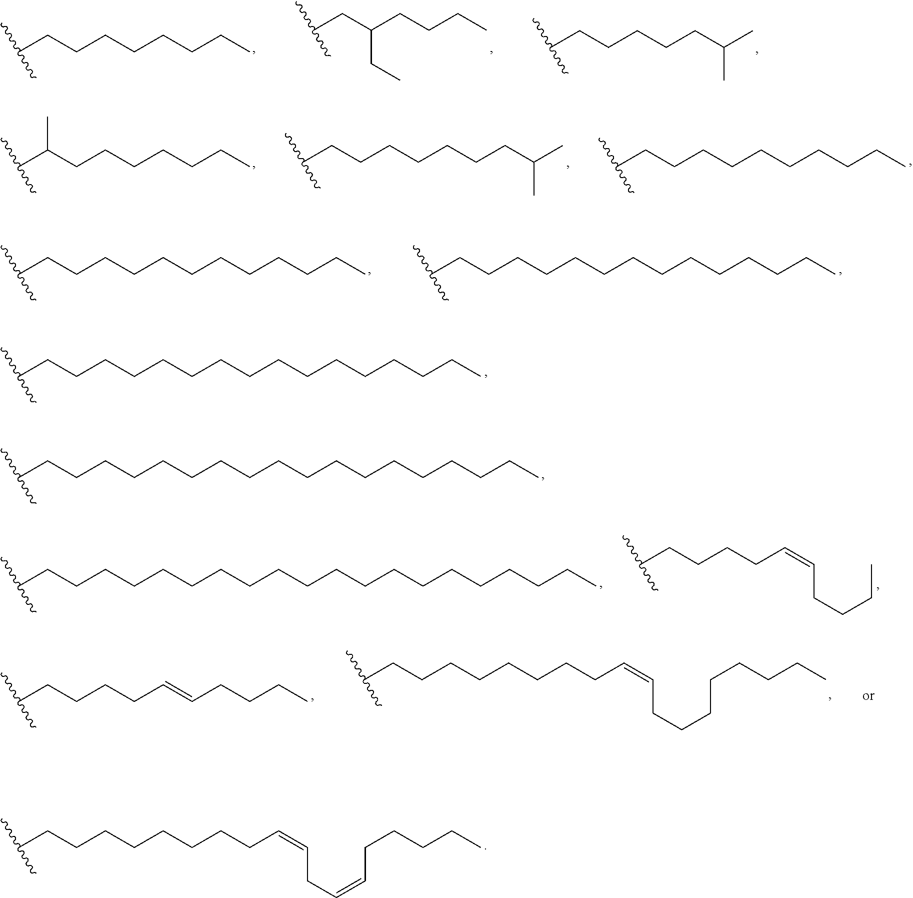

In still yet other implementations which may be combined with any of the preceding implementations, each R³ is independently a lipid of the formula

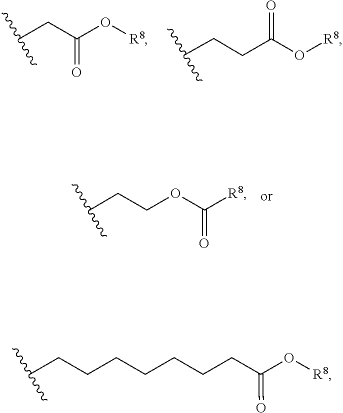

wherein R⁸ is a branched or straight chain aliphatic moieties having from about 6 to about 50 carbon atoms or from about 10 to about 50 carbon atoms, wherein the aliphatic moieties, can comprises, in some examples, one or more heteroatoms and/or one or more double or triple bonds. In some implementations, each R³ is independently

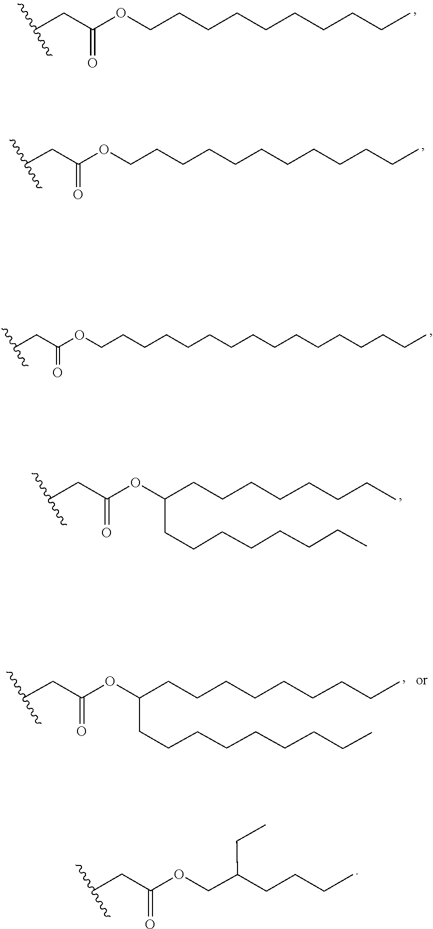 [SMILES: *CC(=O)OCCCCCCCCCC ; *CC(=O)OCCCCCCCCCCCC ; *CC(=O)OCCCCCCCCCCCCCCCC ; *CC(=O)OC(CCCCCCCC)CCCCCCCC ; *CC(=O)OC(CCCCCCCCC)CCCCCCCCC ; *CC(=O)OCC(CC)CCCC]

In some implementations, each R⁴ moiety is independently C₈-C₂₄-alkyl, C₈-C₂₄-alkenyl, or C₁-C₈alkylester, each as previously described herein

Natural lipid moieties employed in the practice of the present disclosure can be derived from, for example, phospholipids, glycerides (such as di- or tri-glycerides), glycosylglycerides, sphingolipids, ceramides, and saturated and unsaturated sterols, isoprenoids, and other like natural lipids.

Other suitable lipid moieties may include lipophilic carbocyclic or aromatic groups such as optionally substituted aryl, cycloalkyl, cycloalkylalkyl, or arylalkyl moieties, including for example naphthalenyl or ethylbenzyl, or lipids comprising ester functional groups including, for example, sterol esters and wax esters. In still other implementations, the lipid moiety of R³ and/or R⁴ can be

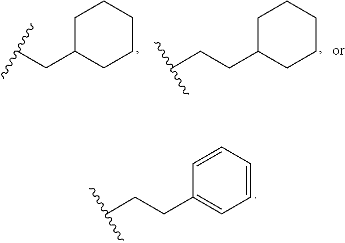

### Ethylene Glycol Moieties

The tertiary amino lipidated and/or PEGylated cationic peptoids of the present disclosure may comprise capping amino acid residues at the N- and/or C-terminus which are N-substituted by oligomers or polymers of ethylene glycol, that is, N-substituted with oligoethylene glycol and/or polyethylene glycol. The incorporation of oligo- and/or polyethylene glycol moieties into the tertiary amino lipidated and/or PEGylated cationic peptoid compounds described herein may facilitate particle stability of complexes formed with nucleic acids and prevent particle aggregation in vivo.

It should be recognized that the term“PEGylated” is used herein to describe cationic peptoid compounds comprising terminal or internal amino acid residues which may be N-substituted with oligoethylene glycol, or polyethylene glycol, or a combination thereof. In some implementations, the tertiary amino lipidated and/or PEGylated cationic peptoid compounds provided herein are N-PEGylated.

In certain implementations wherein the tertiary amino lipidated and/or PEGylated cationic peptoid compound comprises N-PEGylated amino acid residues at the N-terminus, m represents the number of N-PEGylated amino acid residues at the N-terminus, and each R² is independently an ethylene glycol moiety of the formula —CH₂CH₂O(CH₂CH₂O)ᵤR^{2a}, wherein R^{2a} is —H or C₁-C₄-alkyl. In some implementations, R^{2a} is —H, —CH₃, or —CH₂CH₃. In certain implementations, R^{2a} is —H. In other implementations, R^{2a} is —CH₃. In still yet other implementations, R^{2a} is —CH₂CH₂.

In some implementations, m is an integer from 0 to 10, an integer from 0 to 3, or an integer from 4 to 10. In some implementations, each u is independently an integer from 2 to 200, an integer 2 to 100, an integer from 2 to 50, an integer from 50 to 200, an integer from 50 to 100, an integer from 100 to 200, or an integer from 150 to 200.

In certain implementations, m is an integer from 0 to 3, and each u is an integer from 20 to 200, such as from 30 to 50. In certain implementations, m is an integer from 0 to 3, and u is an integer from 40 to 45. In still yet other implementations, m is 1, and u is an integer from 40 to 45. In other implementations, m is an integer from 4 to 10, and each u is an integer from 2 to 10. In certain implementations, m is an integer from 4 to 10, and u is an integer from 2 to 5. In still yet other implementations, m is an integer from 7 to 10, and u is 3.

In certain implementations wherein the tertiary amino lipidated and/or PEGylated cationic peptoid compound comprises N-PEGylated amino acid residues at the C-terminus, t represents the number of N-PEGylated amino acid residues at the N-terminus, and each R⁶ is independently an ethylene glycol moiety of the formula —CH₂CH₂O(CH₂CH₂O)CH₂CH₂CvR^{6a}, wherein R^{6a} is —H or C₁-C₄alkyl. In some implementations, R^{6a} is —H, —CH₃, or CH₂CH₃. In certain implementations, R^{6a} is —H. In other implementations, R^{6a} is —CH₃. In still yet other implementations, R^{6a} is —CH₂CH₃.

In some implementations, t is an integer from 0 to 10, an integer from 0 to 3, or an integer from 4 to 10. In some implementations, each u is independently an integer from 2 to 200, an integer 2 to 100, an integer from 2 to 50, an integer from 50 to 200, an integer from 50 to 100, an integer from 100 to 200, or an integer from 150 to 200.

In some implementations, t is an integer from 0 to 3, and each v is an integer from 30 to 50. In certain implementations, t is an integer from 0 to 3, and v is an integer from 40 to 45. In still yet other implementations, t is 1, and v is an integer from 40 to 45. In other implementations, t is an integer from 4 to 10, and each v is an integer from 2 to 10. In certain implementations, t is an integer from 4 to 10, and v is an integer from 2 to 5. In still yet other implementations, t is an integer from 7 to 10, and v is 3.

In some implementations wherein the tertiary amino lipidated and/or PEGylated cationic peptoid compound is N-PEGylated, at least one of m or t is nonzero. In some implementations, m is nonzero. In other implementations, t is nonzero. In certain implementations, both m and t are nonzero.

In some implementations, the cationic component of the multicomponent systems is a lipidated cationic peptoid (“lipidated cationic peptoid”) having a structure of Formula (Ia):

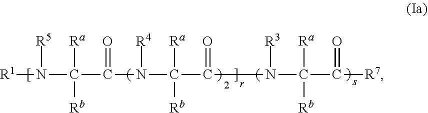

wherein r is 1 to 5, s is 1 to 8, and the remaining variables are as described herein for Formula (I). In some cases, r is 2 to 4. In various implementations, r is 3 or 4. In some implementations, r is 1. In various cases, r is 2. In some implementations, r is 3. In various implementations, r is 4, In some cases, r is 5. In some implementations, s is 2 to 4. In various implementations, s is 2 or 4. In some implementations, s is 5 to 7. In various cases, s is 1. In some implementations, s is 2. In various implementations, s is 3. In some cases, s is 4, In various cases, s is 5. In some implementations, s is 6. In some cases, s is 7. In various cases, s is 8 In some implementations, R¹ is H. In various implementations, R¹ is alkyl. In some implementations, R¹ is H or CH₃. In some implementations, each R³ independently is C₈-C₂₄alkyl or C₈-C₂₄-alkenyl. In some cases, each R³ independently is C₈-C₁₂ alkyl, such as n-decyl. In various implementations, each R³ independently is

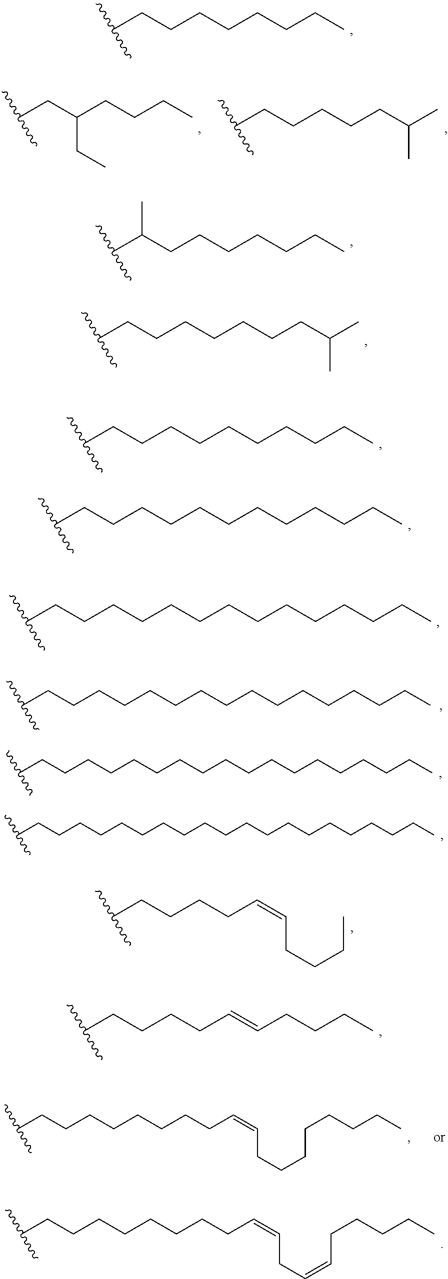

In some implementations, each R³ independently is

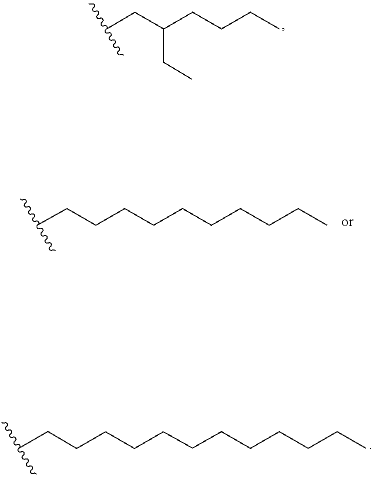

In various implementations, each R⁴ independently is a neutral spacer moiety selected from the group consisting of C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy. In some cases, each R⁴ independently is selected from the group consisting of

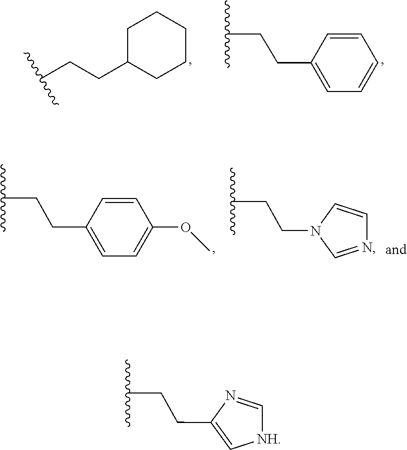

In various cases, each R⁴ independently is

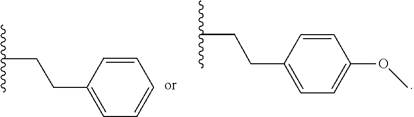 [SMILES: *CCc1ccccc1 ; *CCc1ccc(OC)cc1]

In some implementations, each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl. In some implementations, each R⁵ independently is selected from the group consisting of

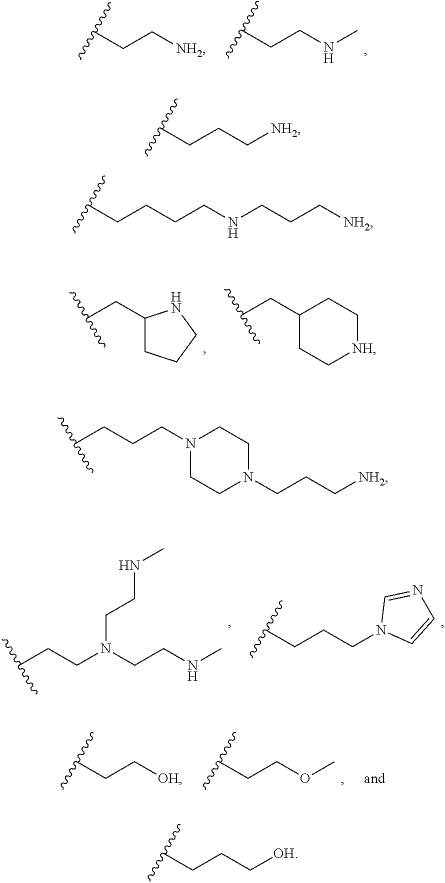

In various implementations, each R⁵ independently is

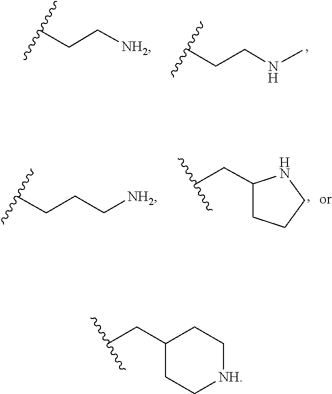

In various cases, R⁷ is NH₂. In some implementations each of R^{a} and R^{b} is H. In some cases, r is 3 or 4; s is 2 or 4; R¹ is H; each R³ independently is

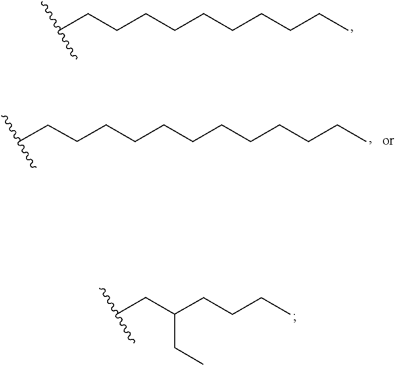

each R⁴ independently is

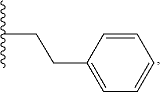 [SMILES: *CCc1ccccc1]

each R⁵ independently is

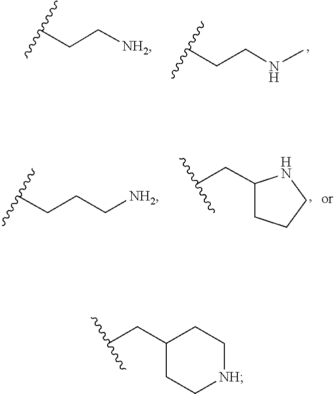

each R⁷ is NH₂; and each R^{a} and R^{b} independently is H. Examples of lipidated cationic components having a Formula (Ia) can be found in Table 1A (e.g., Compounds 1-12 and 19-36). In some examples the lipidated cationic peptoid comprises Compound 24.

In some implementations, the cationic component is a lipidated cationic peptoid having a Formula (Ib):

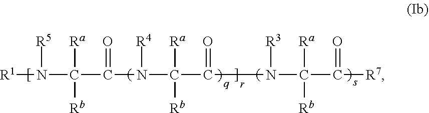

wherein q is 0 or 1, r is 1 to 10, s is 1 to 8, and the remaining variables are as described herein for Formula (I). In some implementations, q is 0. In various implementations, q is 1. In some cases, r is 1 to 5. In various cases, r is 5 to 10. In some cases, r is 2 to 4. In various cases, r is 1. In some cases, r is 2. In various cases, r is 3. In some implementations, s is 2 to 4. In various implementations, s is 2 or 4. In some implementations, R¹ is H. In various implementations, R¹ is alkyl. In some implementations, R¹ is H or CH₃. In some implementations, each R³ independently is C₈-C₂₄alkyl or C₈-C₂₄-alkenyl. In some cases, each R³ independently is C₈-C₁₂ alkyl, such as n-decyl. In various implementations, each R³ independently is

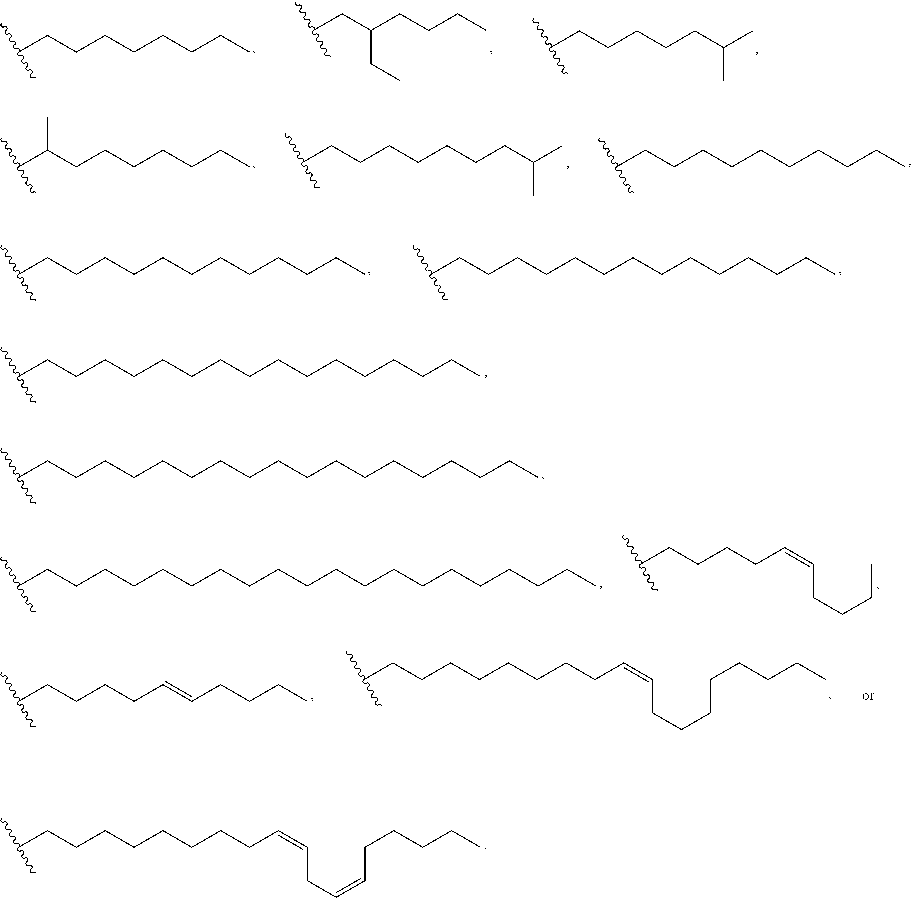

In some implementations, each R³ independently is

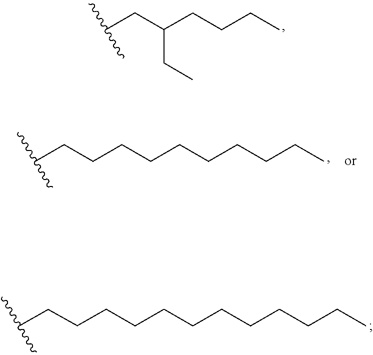

In some implementations, each R⁵ independently is selected from the group consisting of

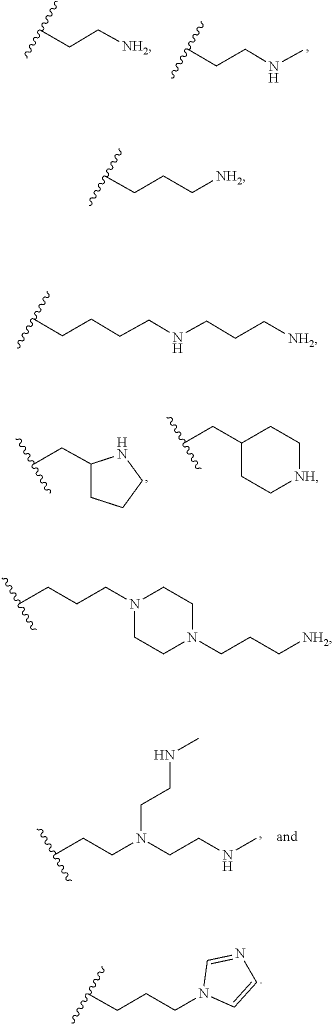

In various implementations, each R⁵ independently is

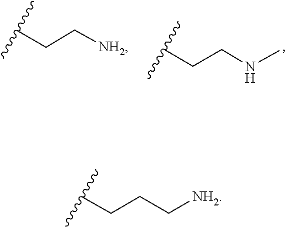

In some cases, each R⁵ is selected from the group consisting of

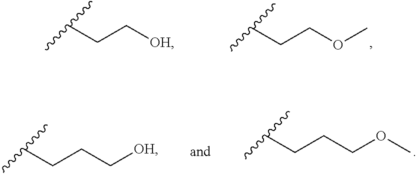

In various cases, R⁷ is NH₂. In some implementations each of R^{a} and R^{b} is H. In some cases, q is 0, s is 2 or 4, each R³ independently is

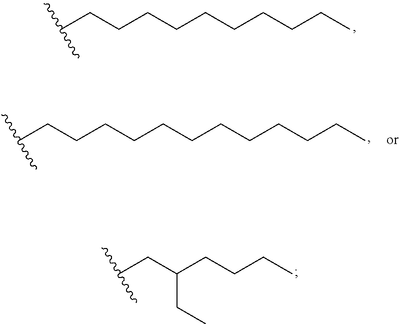

each R⁵ independently is

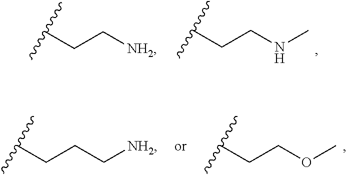

each R⁷ is NH₂; and each R^{a} and R^{b} independently is H. Examples of lipidated cationic peptoids having a Formula (Ib) can be found in Table 1A (e.g., Compounds 13-16, 76, and 99).

In some implementations, the cationic component is a lipidated cationic peptoid having a Formula (Ic):

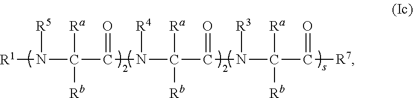

wherein s is 3 or 4 and the remaining variables are as described herein for Formula (I). In some cases, s is 3. In various cases, s is 4. In some implementations, R¹ is H. In various implementations, R¹ is alkyl. In some implementations, R¹ is H or CH₃. In some implementations, each R³ independently is C₈-C₂₄alkyl or C₈-C₂₄-alkenyl. In some cases, each R³ independently is C₈-C₁₂ alkyl, such as n-decyl. In various implementations, each R³ independently is

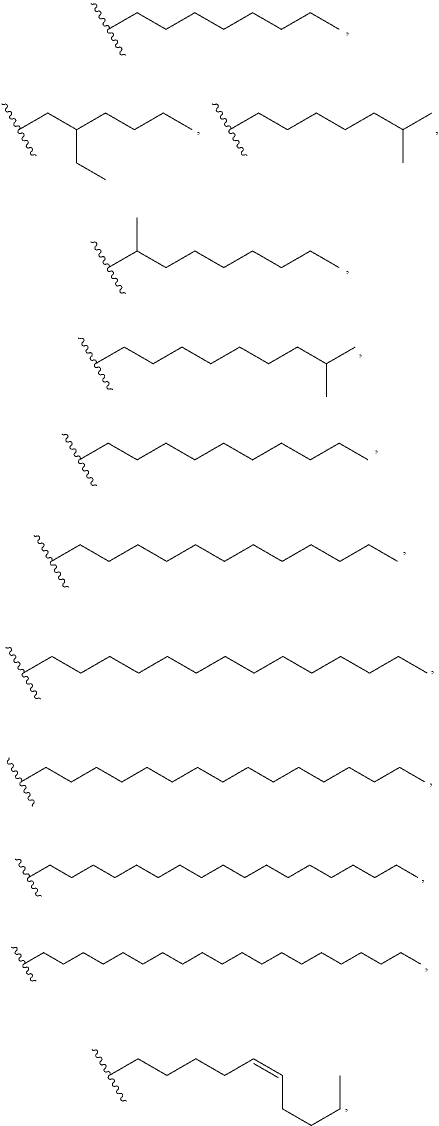

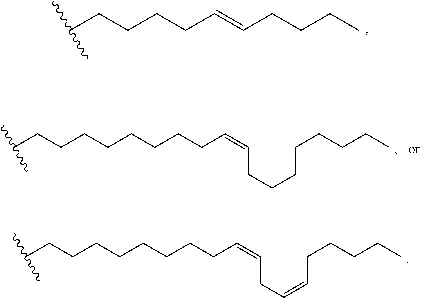 [SMILES: *CCCC/C=C/CCCC ; *CCCCCCCC/C=C\CCCCCCCC ; *CCCCCCCC/C=C\C/C=C\CCCCC]

In some implementations, each R³ independently is

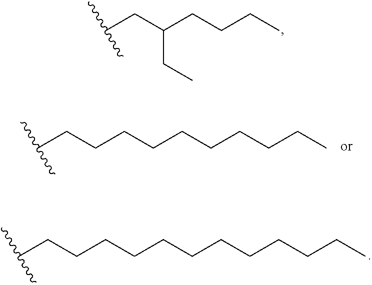

In various implementations, each R⁴ independently is a neutral spacer moiety selected from the group consisting of is a C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy. In some cases, each R⁴ independently is selected from the group consisting of

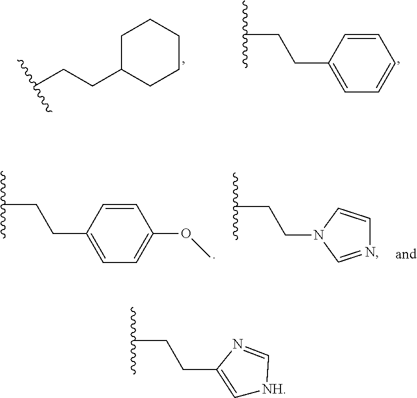 [SMILES: *CCC1CCCCC1 ; *CCc1ccccc1 ; *CCc1ccc(OC)cc1 ; *CCn1ccnc1 ; *CCc1c[nH]cn1]

In various cases, each R⁴ independently is

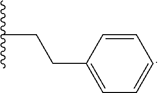 [SMILES: *CCc1ccccc1]

In some implementations, each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl. In some implementations, each R⁵ independently is selected from the group consisting of

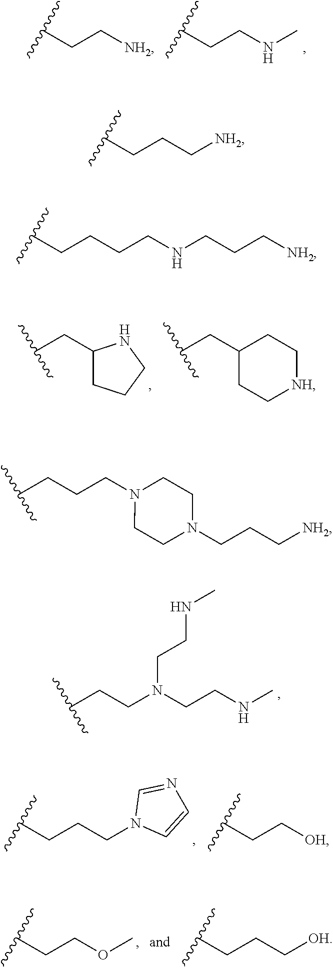

In various implementations, each R⁵ independently is

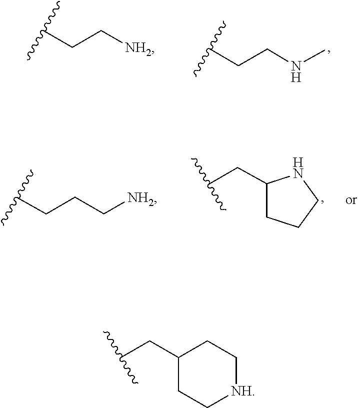

In various cases, R⁷ is NH₂. In some implementations each of R^{a} and R^{b} is H. In some cases, R¹ is H or CH₃; each R³ independently is

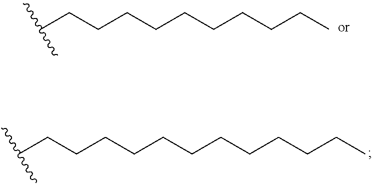 [SMILES: *CCCCCCCCCC ; *CCCCCCCCCCCC]

each R⁴ independently is

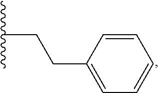 [SMILES: *CCc1ccccc1]

each R⁷ is NH₂; and each R^{a} and R^{b} independently is H. Examples of lipidated cationic peptoids having a Formula (Ic) can be found in Table 1A (e.g., Compounds 49-55).

In some implementations, the cationic component is a lipidated cationic peptoid having a Formula (Id):

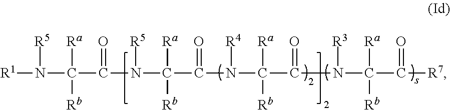

wherein s is 3 or 4 and the remaining variables are as described herein for Formula (I). In some cases, s is 3. In various cases, s is 4. In some implementations, R¹ is H. In various implementations, R¹ is alkyl. In some implementations, R¹ is H or CH₃. In some implementations, each R³ independently is C₈-C₂₄alkyl or C₈-C₂₄-alkenyl. In some cases, each R³ independently is C₈-C₁₂ alkyl, such as n-decyl. In various implementations, each R³ independently is

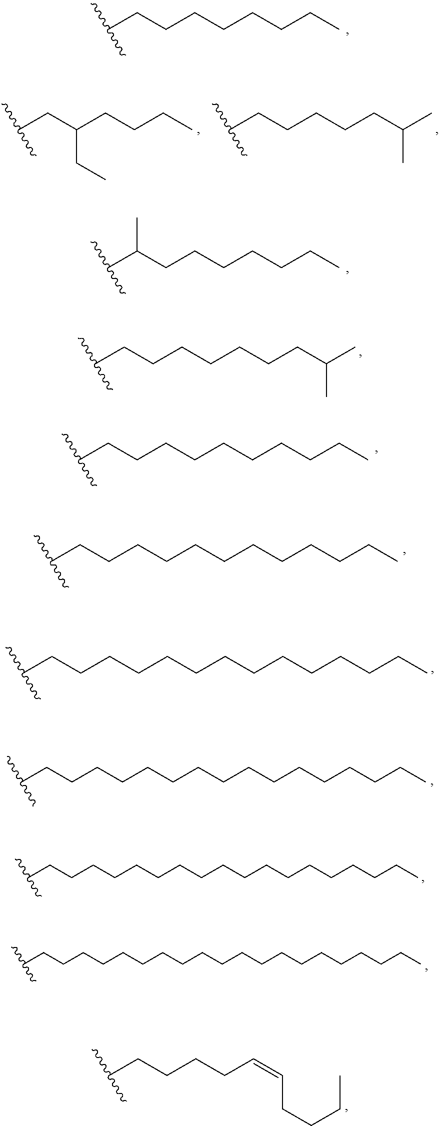

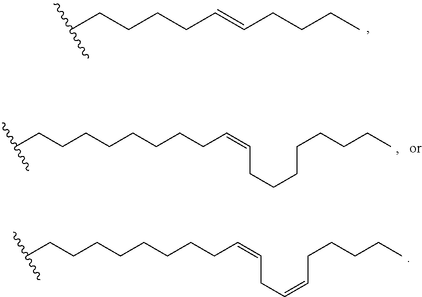 [SMILES: *CCCC/C=C/CCCC ; *CCCCCCCC/C=C\CCCCCCCC ; *CCCCCCCC/C=C\C/C=C\CCCCC]

In some implementations, each R³ independently is

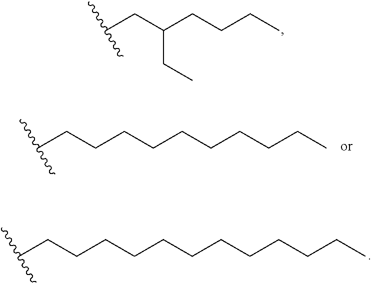

In various implementations, each R⁴ independently is a neutral spacer moiety selected from the group consisting of is a C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy. In some cases, each R⁴ independently is selected from the group consisting of

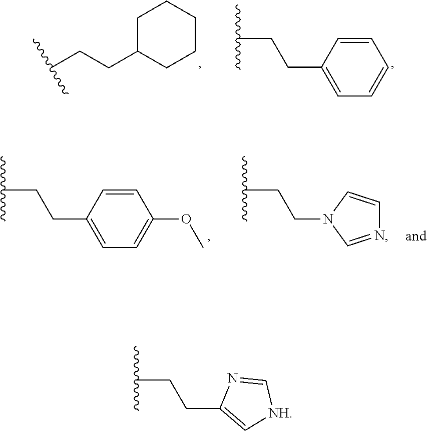

In various cases, each R⁴ independently is

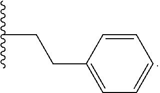 [SMILES: *CCc1ccccc1]

In some implementations, each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl. In some implementations, each R⁵ independently is selected from the group consisting of

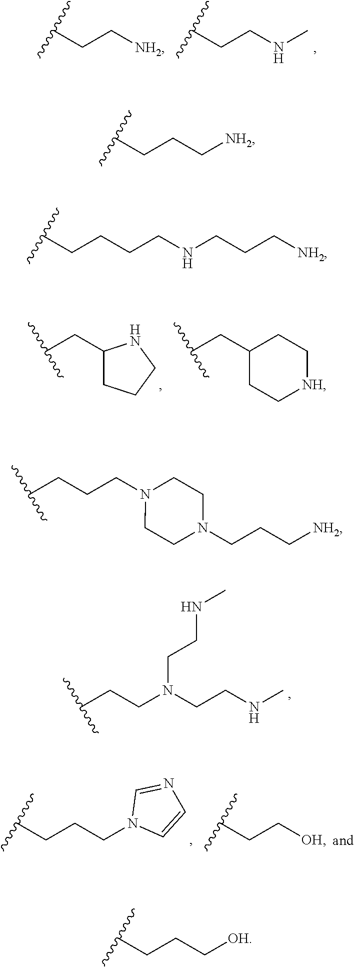

In various implementations, each R⁵ independently is

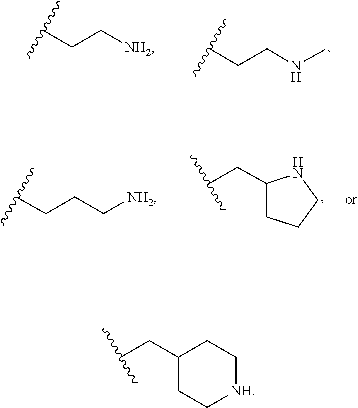

In various cases, R⁷ is NH₂. In some implementations each of R^{a} and R^{b} is H. In some cases, R¹ is H; each R³ independently is

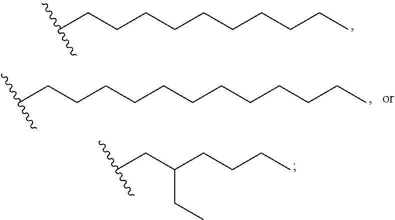

each R⁴ independently is

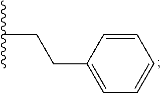 [SMILES: *CCc1ccccc1]

each R⁷ is NH₂; and each R^{a} and R^{b} independently is H. Examples of lipidated cationic peptoids having a Formula (Id) can be found in Table 1A (e.g., 57-63 and 65-71).

In some implementations, the cationic component is a lipidated cationic peptoid having a Formula (Ie):

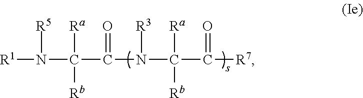

wherein s is 1 to 8 and the remaining variables are as previously described herein for Formula (I). In some implementations, s is 2 to 6. In various implementations, s 3, 4, or 5. In some cases, s is 1. In various cases, s is 2. In some implementations, s is 3. In various implementations, s is 4. In some cases, s is 5. In various cases, s is 7. In some implementations, s is 7. In some implementations, R¹ is H. In various implementations, R¹ is alkyl. In some implementations, R¹ is CH₃, or CH₂CH₃. In some implementations, R¹ is CH₂CH₂OH. In some implementations, each R³ independently is C₈-C₂₄alkyl, C₈-C₂₄-alkenyl, or C₁-C₈alkylester. In some implementations, each R³ independently is C₈-C₂₄alkyl or C₈-C₂₄-alkenyl. In some cases, each R³ independently is C₈-C₁₂ alkyl, such as n-decyl. In some cases, at least one or at least two R³ independently are C₈-C₁₂ alkyl, such as n-decyl. In various implementations, each R³ independently is

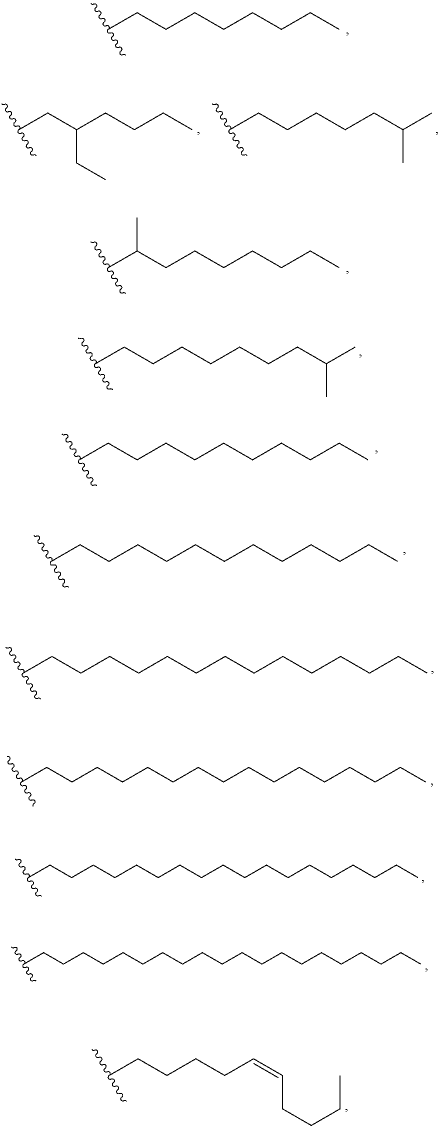

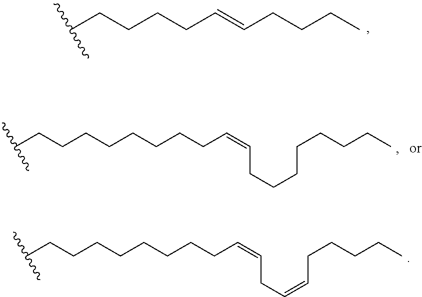 [SMILES: *CCCC/C=C/CCCC ; *CCCCCCCC/C=C\CCCCCCCC ; *CCCCCCCC/C=C\C/C=C\CCCCC]

In some implementations, at least one or at least two R³ is

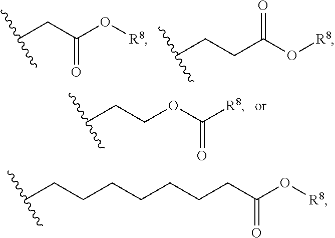

wherein R⁸ is as previously described herein. In some cases, at least one or at least two R³ independently is selected from the group consisting of

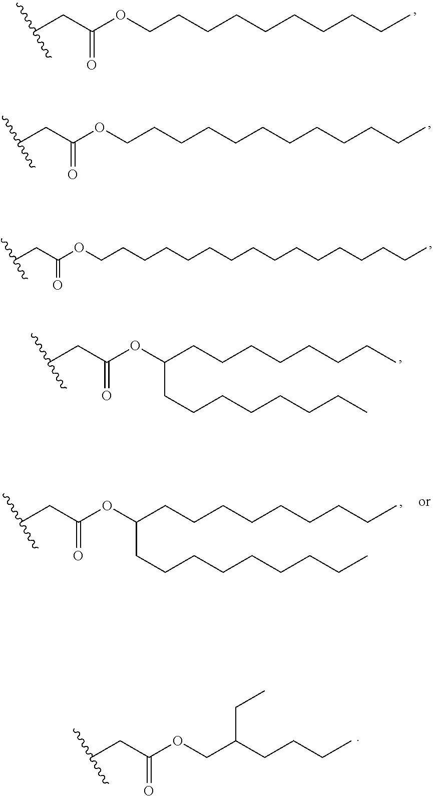 [SMILES: *CC(=O)OCCCCCCCCCC ; *CC(=O)OCCCCCCCCCCCC ; *CC(=O)OCCCCCCCCCCCCCCCC ; *CC(=O)OC(CCCCCCCC)CCCCCCCC ; *CC(=O)OC(CCCCCCCCC)CCCCCCCCC ; *CC(=O)OCC(CC)CCCC]

In some implementations, each R³ independently is

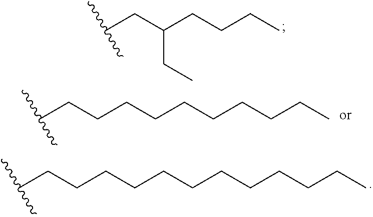

In some cases, each R³ independently is

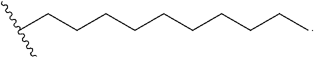 [SMILES: *CCCCCCCCCC]

In some examples, R⁵ is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl. In some implementations, s is 4, two R³ are

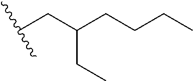 [SMILES: *CC(CC)CCCC]

### and two R³ are

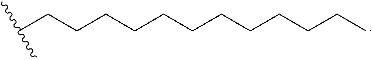 [SMILES: *CCCCCCCCCCCC]

In various cases, R⁷ is NH₂. In some implementations each of R^{a} and R^{b} is H.

In various implementations when R⁵ of Formula (Ie) is selected from the group consisting of aminoalkyl, alkylaminoalkyl, aminoalkylaminoakyl, guanidinoalkyl, N-heterocyclylalkyl, and N-heteroaryl, Formula (Ie) is designated Formula (Ie′). In various cases, R⁵ is selected from the group consisting of

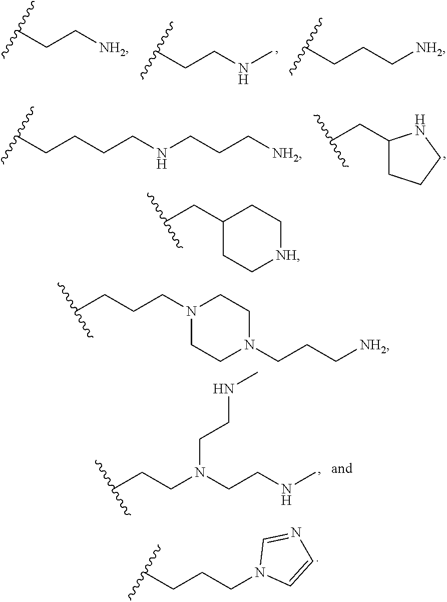

In some implementations, R⁵ is

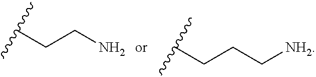

In various cases, R⁵ is

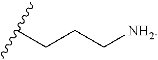 [SMILES: *CCCN]

In some implementations, each of R^{a} and R^{b} is H. In some cases, s is 4; R¹ is H; each R³ independently is

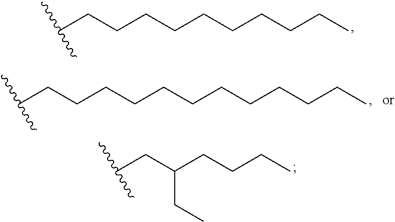

### R⁵ is

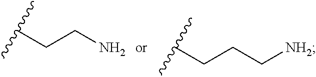

each R⁷ is NH₂; and each R^{a} and R^{b} independently is H. In some implementations each of R^{a} and R^{b} is H. In some cases, s is 4; R¹ is H; each R³ independently is

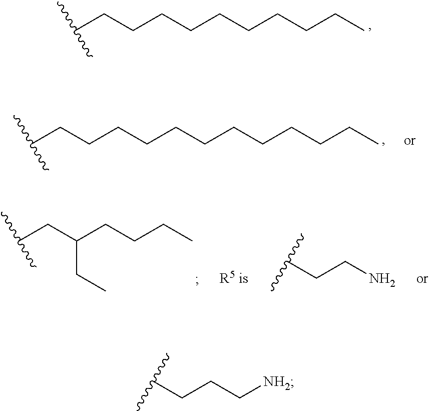

each R⁷ is NH₂; and each R^{a} and R^{b} independently is H. Examples of lipidated cationic peptoids having a Formula (Ie′) can be found in Table 1A (e.g., Compounds 41, 42, 44-47, 73-75, 77-80, 85, 86, 91-94, 97, 98, 111-118, 153, and 154). In some implementations, the lipidated cationic peptoid is Compound 79, 93, or 112. In various implementations, the lipidated cationic peptoid is Compound 112. In some cases, when R⁵ of Formula (Ie) is selected from the group consisting of hydroxyalkyl, hydroxyether, alkoxyalkyl, and hydroxylheteroalkyl, Formula (Ie) is designated Formula (Ie″). In some implementations, R⁵ is selected from the group consisting of

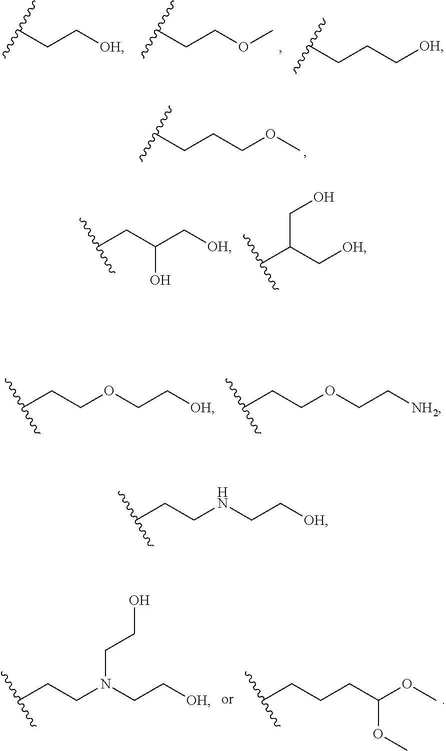

In some cases, R⁵ is selected from the group consisting of

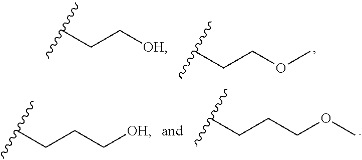

Examples of lipidated cationic peptoids having a Formula (Ie″) can be found in Table 1A (e.g., Compounds 43, 137, 138, 140, 145, 146, 148, 149, 151, 152, 160, 161, and 162).

In some implementations, the cationic component is a lipidated cationic peptoid having a Formula (If):

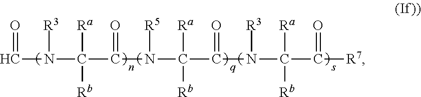

wherein n is an integer from 0 to 4; q is 1 or 2; s is an integer from 1 to 4; and the remaining substituents are as described herein for Formula (I). In some implementations, n is 0. In various cases, n is an integer from 1 to 4, or from 1 to 3. In various cases, n is 1. In some cases, n is 2. In some implementations, n is 3. In various implementations, n is 4. In some cases, q is 1. In various cases, q is 2. In some implementations, s is 1. In various implementations, s is 2. In some cases, s is 3. In various implementations, s is 4. In some implementations, each R³ independently is C₈-C₂₄alkyl or C₈-C₂₄-alkenyl. In some cases, each R³ independently is C₈-C₁₂ alkyl, such as n-decyl. In various implementations, each R³ independently is

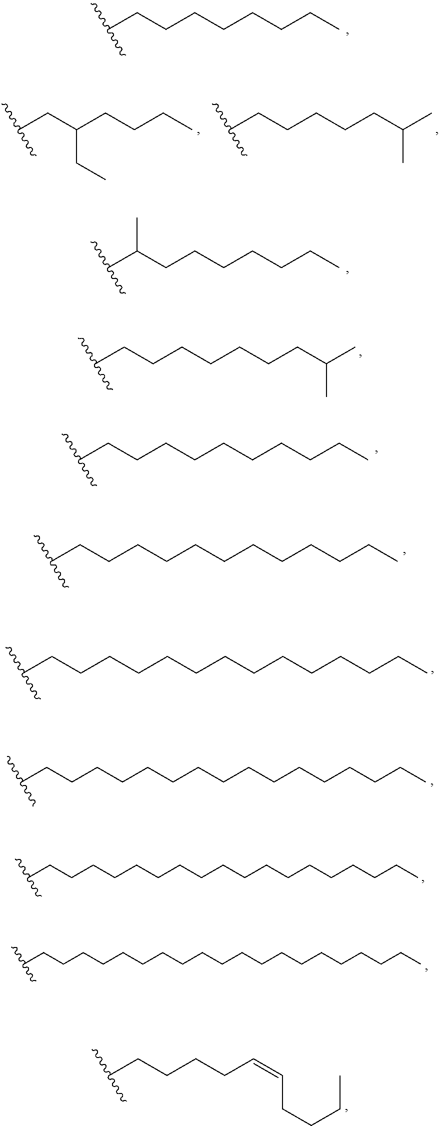

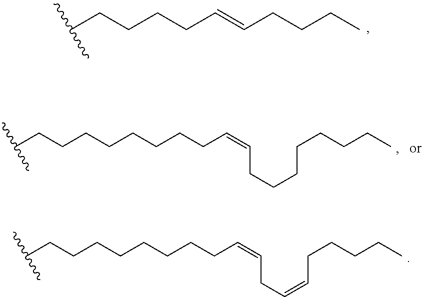 [SMILES: *CCCC/C=C/CCCC ; *CCCCCCCC/C=C\CCCCCCCC ; *CCCCCCCC/C=C\C/C=C\CCCCC]

In some implementations, each R³ independently is

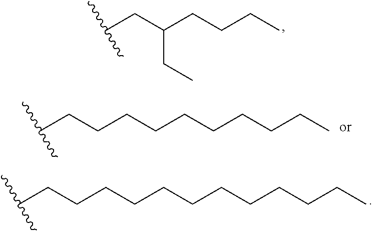

In some cases, each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl. In some implementations, each R⁵ independently is selected from the group consisting of

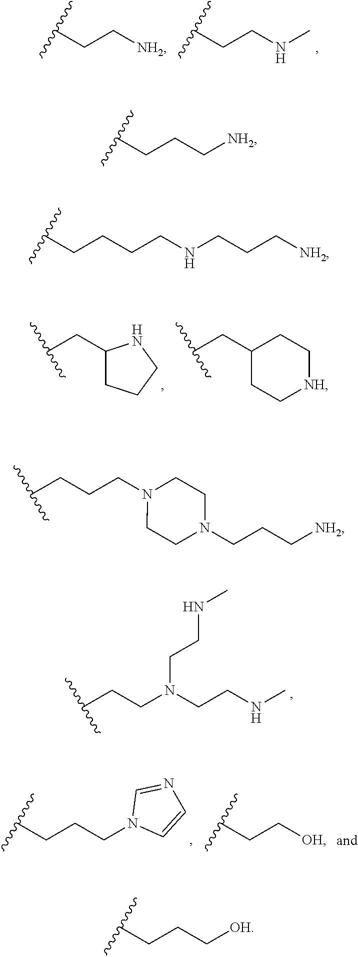

In various implementations, each R⁵ independently is

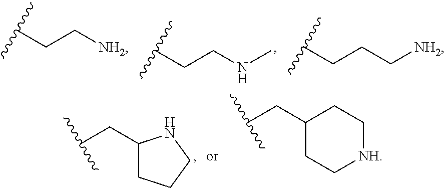

In various cases, R⁷ is NH₂. In some implementations each of R^{a} and R^{b} is H. In some cases, n is 2; q is 1 or 2; s is 2, each R³ independently is

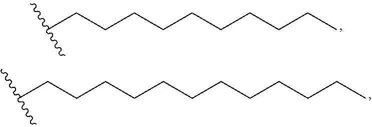 [SMILES: *CCCCCCCCCC ; *CCCCCCCCCCCC]

R⁵ independently is

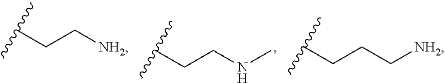

each R⁷ is NH₂; and each R^{a} and R^{b} independently is H. Examples of lipidated cationic peptoids having a Formula (If) can be found in Table 1A (e.g., Compounds 81, 82, 83, 95, 96, 142, 144, 155, 156, 163, 164, 165).

In some implementations, the lipidated cationic peptoid is a compound listed in Table 1A. In some implementations, the lipidated cationic peptoid comprises Compound 127. In some implementations, the lipidated cationic peptoid is selected from the group consisting of Compounds 17, 18, 84, 87-89, 100, 101, 102, 103, 113, 114, 127, 130, 132, 134, 139, 141, 147, and 159.

In some implementations, the lipidated cationic peptoid comprises Compound 24, 73, 79, 81, 85, 93, 112, 115, 137, 152, 155, 163, 164, or combinations thereof. In various implementations, the lipidated cationic peptoid comprises Compound 81, 112, 155, 163, 164, or combinations thereof. In some cases, the lipidated cationic peptoid comprises Compound 81. In various cases, the lipidated cationic peptoid comprises Compound 112. In some implementations, the lipidated cationic peptoid comprises Compound 155. In various implementations, the lipidated cationic peptoid comprises Compound 164. In various implementations, the lipidated cationic peptoid comprises Compound 165.

In some implementations, the cationic components are lipitoids selected from any lipitoids disclosed in WO2020/069445, such as lipitoids of formula (II):

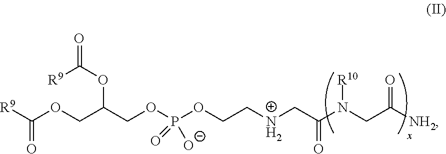

wherein:
x is a integer from 1 to 100;
each R⁹ is independently a lipid moiety; and
each R¹⁰ is independently a cationic or neutral spacer moiety.

### Electrostatic Binding

In some implementations, the cationic component of the multicomponent delivery system, such as a lipidated cationic peptoid, has electrostatic binding portions. The electrostatic binding portions can contain much of the same elements as the cationic anchor, but typically a larger number of those groups to facilitate oligonucleotide condensation. In some examples, the lipidated cationic peptoids contain spacer groups or other functionality specifically to modulate interaction with oligonucleotide. The modulation can occur through incorporation of aromatic groups for pi-pi stacking interactions, hydrophobic or hydrophilic groups, or elements to buffer cationic charge. In some cases, the lipidated cationic peptoids have the ability to degrade through either a hydrolytic or self-immolative mechanism to facilitate cargo release.

Anionic and/or Zwitterionic Component(s)

In some implementations, the multicomponent delivery system comprises anionic components, zwitterionic components, or a mixture thereof (“anionic/zwitterionic component”). The functions of the anionic components include, but not limited to, buffering the particle zeta potential without affecting cargo ratios and/or contributing to particle endosomal escape through protonation at low pH in the endosome. The zwitterionic components have similar functions. Further, the zwitterionic components can hold particles together by interacting with both the cationic peptoid as well as the polyanionic cargo compounds. Including anionic components in the multicomponent delivery systems also allows for the creation of core-shell structures in which a net positive zeta potential particle is made (e.g., by mixing lipidated cationic peptoid and the cargo at a positive+/−charge ratio), which is then coated with the anionic components. These negatively charged multicomponent system particles would avoid reticuloendothelial system (RES) clearance better than positively charged ones.

In some implementations, the anionic and/or zwitterionic components are peptoids. In some implementations, the anionic and/or zwitterionic components are lipidated peptoids (“anionic/zwitterionic lipidated peptoid”). Thus, in some cases, the non-cationic lipidated peptoid of the multicomponent delivery system disclosed herein comprises an anionic/zwitterionic lipidated peptoid.

In some implementations, the anionic/zwitterionic lipidated peptoid is a compound of Formula (IIIa),

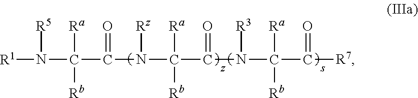

wherein:
s is 1, 2, 3, 4, 5, or 6;
z is 1 or 2;
R¹ is —H, alkyl, alkylaryl, —COR^{1a} or a lipid moiety, wherein R^{1a} is —H, —OH, alkyl, aryl, alkylaryl, —O-alkyl, or —O-alkylaryl;
each R³ independently is a lipid moiety, as previously described herein;
each R⁵ independently is a cationic moiety, as previously described herein;
R⁷ is —H, alkyl, acyl, —OH, —OR^{7a}, —NH₂, —NHR^{7a}, or a lipid moiety, wherein R^{7a} is alkyl, acyl, or a lipid moiety;
each R^{a} and R^{b} independently is —H, C₁-C₄-alkyl, or a side chain moiety found on a naturally- or non-naturally-occurring amino acid; and
R^{z} is an anionic moiety.

In some implementations, z is 1. In various implementations, z is 2. In some implementations, s is 2 to 4. In various implementations, s is 3 or 4. In some cases, s is 2. In various cases, s is 3. In some implementations, s is 4. In various implementations, s is 5. In some cases, s is 6. In some implementations, R¹ is H. In various implementations, R¹ is alkyl (e.g., methyl or ethyl). In some implementations, R¹ is H or CH₃. In some implementations, each R³ independently is C₈-C₂₄alkyl or C₈-C₂₄-alkenyl. In some cases, each R³ independently is C₈-C₁₂ alkyl, such as n-decyl or 2-ethylhexyl. In various implementations, each R³ independently is

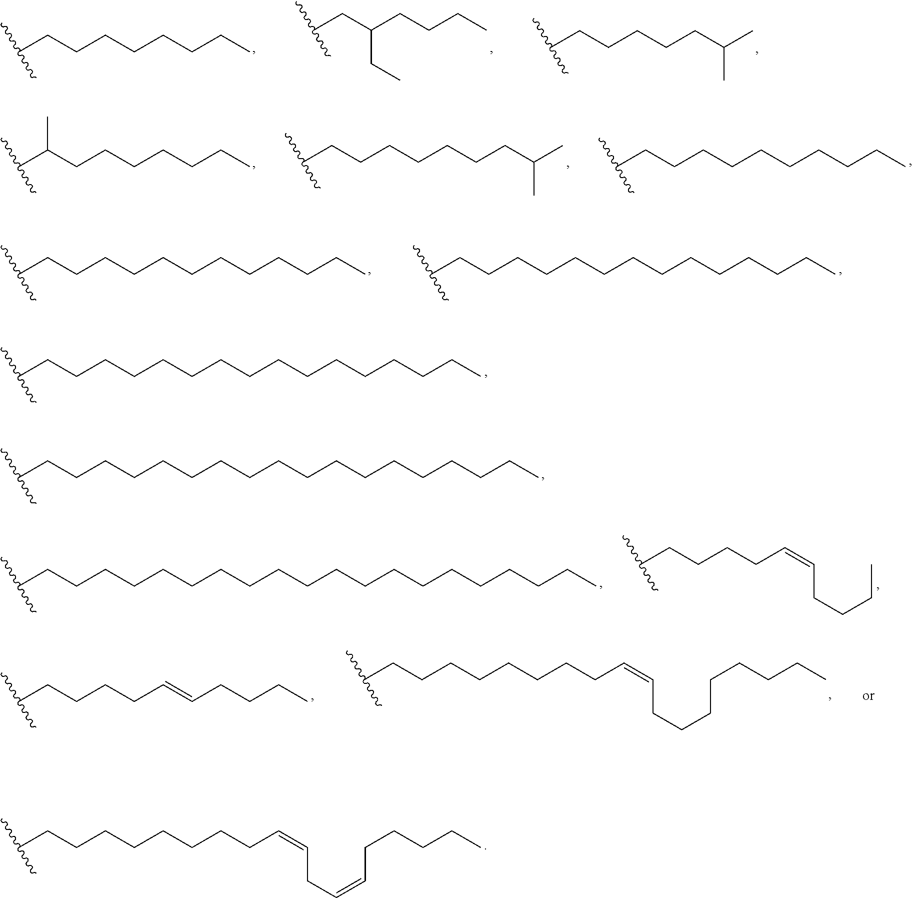

In some implementations, each R³ independently is

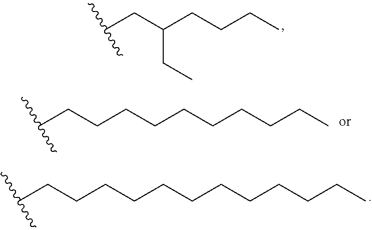

In some cases, R⁵ is selected from the group consisting of aminoalkyl, alkylaminoalkyl, aminoalkylaminoakyl, guanidinoalkyl, N-heterocyclylalkyl, and N-heteroaryl. In some implementations, each R⁵ independently is selected from the group consisting of

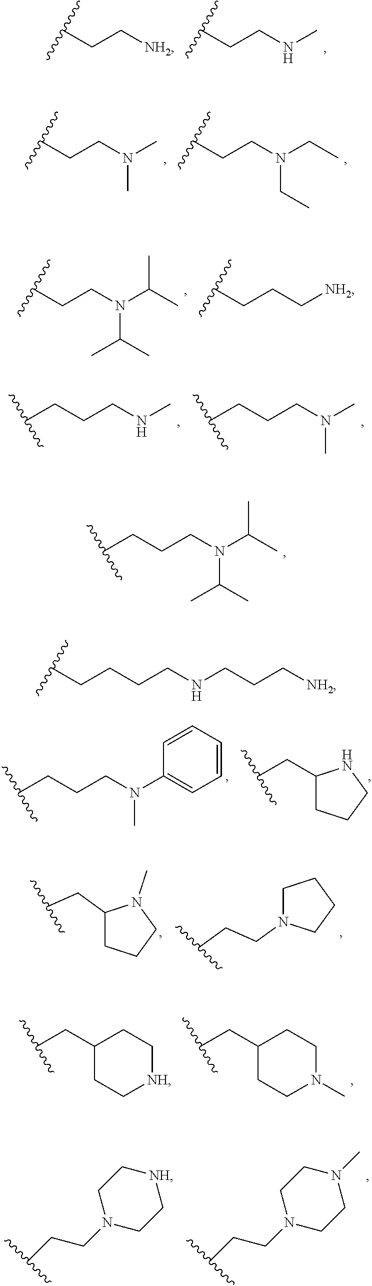

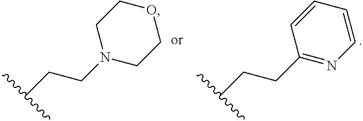 [SMILES: *CCN1CCOCC1 ; "or" ; *CCc1ccccn1]

In various implementations, each R⁵ independently is selected from the group consisting of

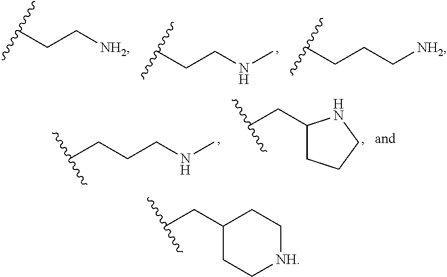

In some cases, each R⁵ independently is

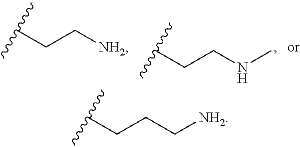

In some implementations, each R⁵ is

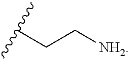 [SMILES: *CCN]

In some cases, R⁷ is —NH₂. In various cases, each R^{a} and R^{b} independently is —H. In some implementations, each R^{z} independently is C₂₋₅ alkylenecarboxylic acid. In various implementations, each R^{z} is selected from

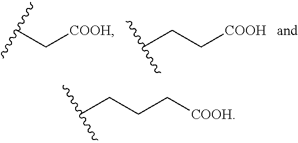 [SMILES: *CC(=O)O ; *CCC(=O)O ; "and" ; *CCCC(=O)O]

In some cases, each R^{z} is

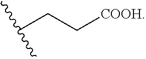 [SMILES: *CCC(=O)O]

In some implementations, z is 1 or 2; s is 3 or 4; R¹ is H or CH₃; each R³ independently is

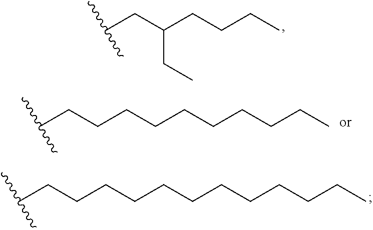

each R⁵ independently is

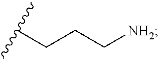 [SMILES: *CCCN]

R⁷ is —NH₂; each R^{a} and R^{b} independently is —H; and each R^{z} independently is

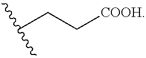 [SMILES: *CCC(=O)O]

Examples of zwitterionic/anionic lipidated peptoids having a Formula (Ma) can be found in Table 1B (e.g., Compounds 104 and 105). In some implementations, the anionic/zwitterionic lipidated peptoid is Compound 104. In some implementations, the anionic/zwitterionic lipidated peptoid is Compound 105.

In some implementations, the anionic/zwitterionic lipidated peptoid is a compound of Formula (IIIb):

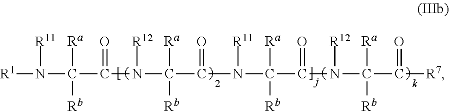

wherein:
j is 1, 2, 3, 4, 5, or 6;
k is 1, 2, 3, or 4;
R¹ is —H, alkyl, alkylaryl, —COR^{1a} or a lipid moiety, wherein R^{1a} is —H, —OH, alkyl, aryl, alkylaryl, —O-alkyl, or —O-alkylaryl;
Each R¹¹ independently is a hydrophilic moiety;
Each R¹² independently is a hydrophobic moiety;
R⁷ is —H, alkyl, acyl, —OH, —OR^{7a}, —NH₂, —NHR^{7a}, or a lipid moiety, wherein R^{7a} is alkyl, acyl, or a lipid moiety; and
each R^{a} and R^{b} independently is —H, C₁-C₄-alkyl, or a side chain moiety found on a naturally- or non-naturally-occurring amino acid.

In some implementations, j is 1. In various implementations, j is 2. In some implementations, j is 2 or 3. In various implementations, j is 3. In some cases, j is 4. In various cases, j is 5. In some implementations, j is 6. In some cases k is 1. In various cases, k is 2. In some implementations, k is 3. In various implementations, k is 4. In some implementations, R¹ is H. In various implementations, R¹ is alkyl (e.g., methyl or ethyl). In some implementations, R¹ is H or CH₃. In some implementations, each R¹¹ independently is aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, hydroxylheteroalkyl, or C₂₋₅ alkylenecarboxylic acid. In some cases, each R¹¹ independently is

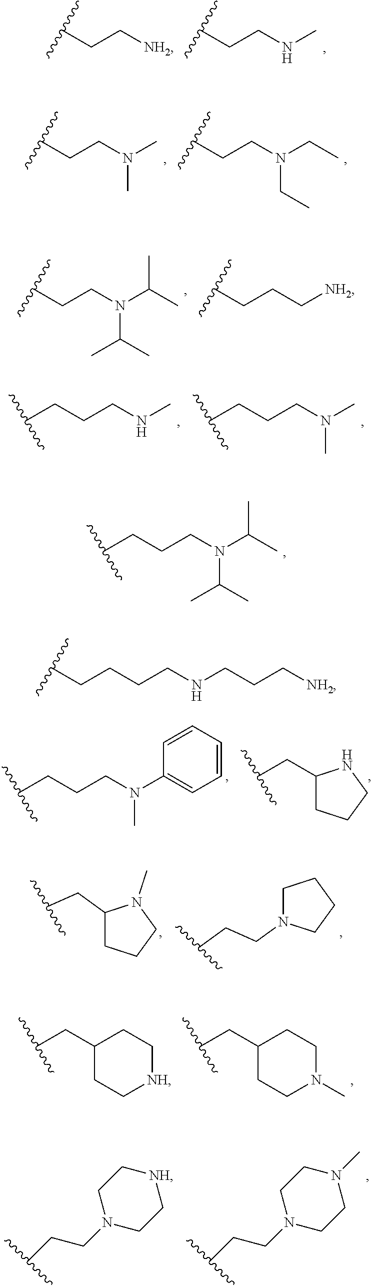

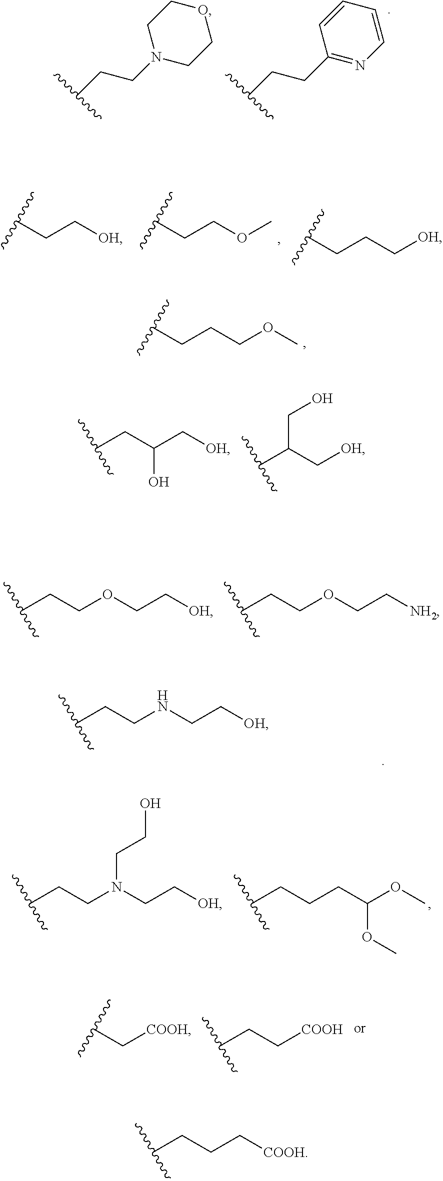

In some cases, each R¹¹ independently is

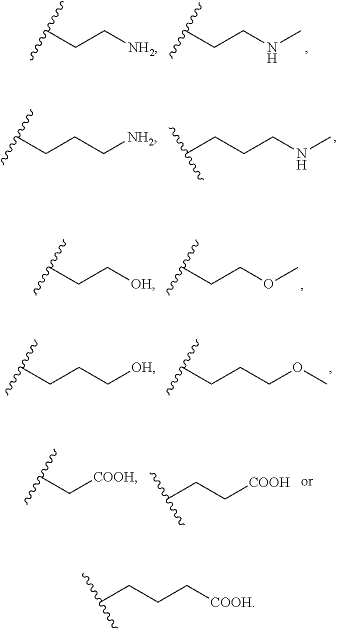

In some cases, each R¹¹ independently is

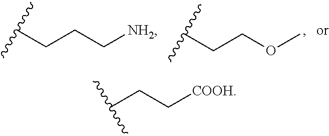

In some cases, each R¹² independently is C₈-C₂₄alkyl, C₈-C₂₄-alkenyl, C₁-C₄aralkyl, or a C₁₋₄heteroaralkyl. In some cases, each R¹² independently is

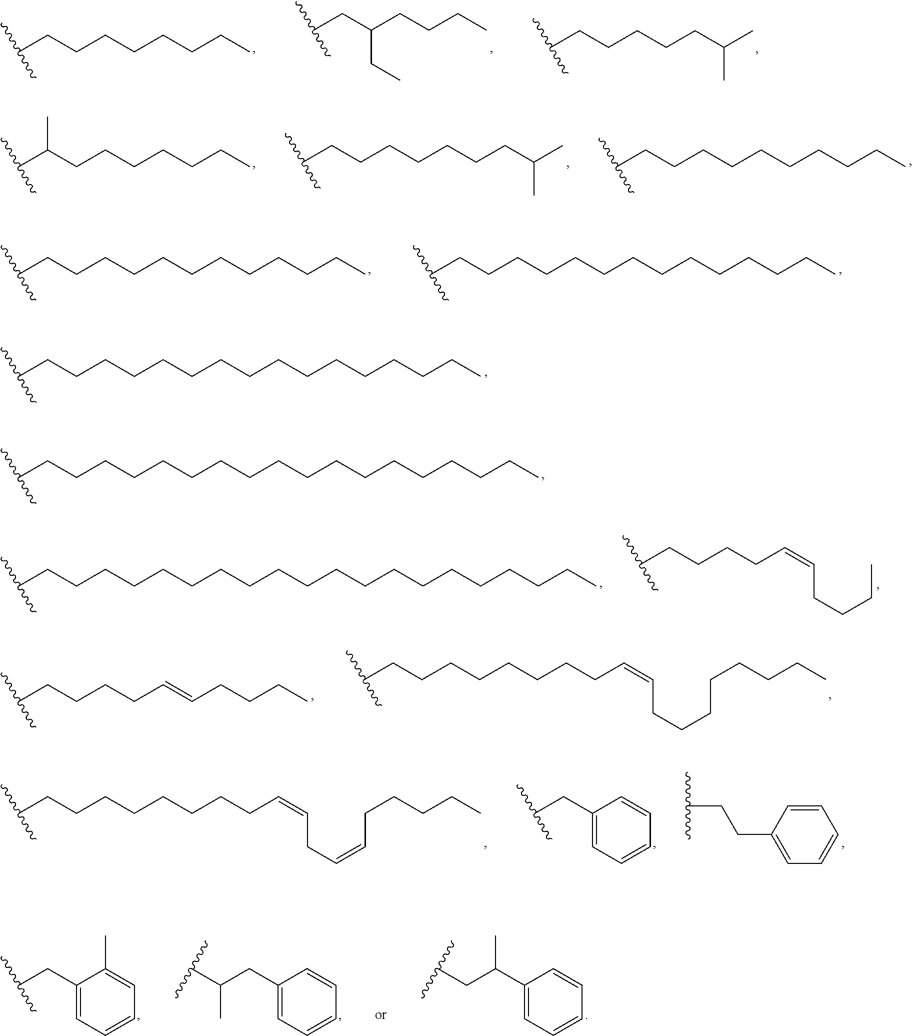

In some implementations, each R¹² independently is

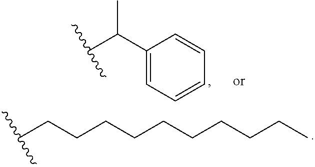

In some cases, at least one R¹¹ is chiral. In some implementations, each chiral R¹¹ has the same stereochemistry. In some cases, j is 2, 3, or 4; k is 2; R¹ is H; R⁷ is —NH₂; each R¹¹ independently is

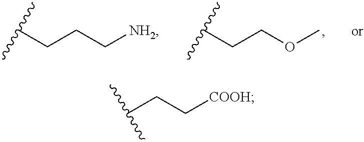

each R¹² independently is

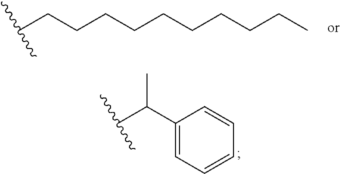

and each R^{a} and R^{b} independently is —H.

Examples of zwitterionic/anionic lipidated peptoids having a Formula (IIIb) can be found in Table 1B (e.g., Compounds 124, 126, and 134). In some implementations, the anionic/zwitterionc lipidated peptoid comprises Compound 125, 131, 133, 135, or 136. In some cases, the anionic/zwitterionc lipidated peptoid is selected from Compound 124, 125, 126, and combinations thereof. In some implementations, the anionic/zwitterionic lipidated peptoid is Compound 124. In some implementations, the anionic/zwitterionic lipidated peptoid is Compound 125. In some implementations, the anionic/zwitterionic lipidated peptoid is Compound 126.

In some implementations, the multicomponent delivery system comprises about 10.0 to about 60.0 mol % of one or more of the anionic/zwitterionic lipidated peptoid of the total moles of the multicomponent delivery system. In some variations, the multicomponent delivery system comprises 0 to about 10.0 mol %, about 10.1 to about 20.0 mol %, about 20.1 to about 30.0 mol %, about 30.1 to about 40.0 mol %, about 40.1 to about 50.0 mol %, or about 50.1 to about 60.0 mol % of one or more of the anionic/zwitterionic lipidated peptoid of the total moles of the multicomponent delivery system. In some implementations, the multicomponent delivery system comprises about 15.0 to about 60.0 mol % of one or more of the anionic/zwitterionic lipidated peptoid of the total moles of the multicomponent delivery system. In certain variations, the multicomponent delivery system comprises 0 to about 20 mol %, about 15 to about 35 mol %, about 20 to about 40 mol %, about 20 to about 35 mol %, about 30 to about 60 mol %, about 40 to about 50 mol %, or about 50 to about 60 mol % of one or more of the anionic/zwitterionic lipidated peptoid of the total moles of the multicomponent delivery system.

### Phospholipids

Phospholipids are zwitterionic compounds and may be incorporated into the multicomponent delivery system of the present disclosure. Phospholipids provide further stabilization to complexes in solution as well as facilitate cell endocytosis, by virtue of their amphipathic character and ability to disrupt the cell membrane. In some implementations, the compositions provided herein comprise complexes that include one or more phospholipids as zwitterionic components.

In some implementations, the multicomponent delivery system as described herein comprises one or more phospholipids. In some implementations, the multicomponent delivery system comprises about 10.0 to about 60.0 mol % of one or more phospholipids of the total moles of the multicomponent delivery system. In certain variations, the multicomponent delivery system comprises 0 to about 10.0 mol %, about 10.1 to about 20.0 mol %, about 20.1 to about 30.0 mol %, about 30.1 to about 40.0 mol %, about 40.1 to about 50.0 mol %, or about 50.1 to about 60.0 mol % of one or more phospholipids of the total moles of the multicomponent delivery system. In some variations, the multicomponent delivery system comprises about 0 to about 10.0 mol %, about 10 to about 20.0 mol %, about 20 to about 30.0 mol %, about 30 to about 40 mol %, about 40 to about 50 mol %, or about 50 to about 60 mol % of one or more phospholipids of the total moles of the multicomponent delivery system. In some implementations, the multicomponent delivery system comprises about 15 to about 60 mol % of one or more of the phospholipids of the total moles of the multicomponent delivery system. In certain variations, the multicomponent delivery system comprises 0 to about 20 mol %, 15 to about 35 mol %, 20 to about 40.0 mol %, 20 to about 35 mol %, 30 to about 60 mol %, 40 to about 50 mol %, or 50 to about 60 mol % of one or more of the phospholipids of the total moles of the multicomponent delivery system. In some implementations, the one or more phospholipids are selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE),1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinoleoyl-sn-glycero-3 phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In certain implementations, the phospholipid is DOPE. In some cases, the phospholipid is DSPC.

Alternatively, the multicomponent delivery system as described herein comprises lipidated peptoids, wherein the lipid moieties of the lipidated peptoids are phospholipids. In some implementations, the lipid components are lipitoids. In some implementations, the multicomponent delivery system comprises about 10.0 to about 60.0 mol % of one or more lipitoids of the total moles of the multicomponent delivery system. In certain variations, the multicomponent delivery system comprises 0 to about 10.0 mol %, about 10.1 to about 20.0 mol %, about 20.1 to about 30.0 mol %, about 30.1 to about 40.0 mol %, about 40.1 to about 50.0 mol %, or about 50.1 to about 60.0 mol % of one or more lipitoids of the total moles of the multicomponent delivery system. In some variations, the multicomponent delivery system comprises 0 to about 10.0 mol %, about 10 to about 20.0 mol %, about 20 to about 30.0 mol %, about 30 to about 40 mol %, about 40 to about 50 mol %, or about 50 to about 60 mol % of one or more one or more lipitoids of the total moles of the multicomponent delivery system. In some implementations, the multicomponent delivery system comprises about 15 to about 60 mol % of one or more lipitoids of the total moles of the multicomponent delivery system. In certain variations, the multicomponent delivery system comprises 0 to about 20 mol %, about 15 to about 35 mol %, about 20 to about 40.0 mol %, about 20 to about 35 mol %, about 30 to about 60 mol %, about 40 to about 50 mol %, or about 50 to about 60 mol % of one or more lipitoids of the total moles of the multicomponent delivery system. In some implementations, the phospholipid moiety of the lipitoids is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE),1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In certain implementations, the phospholipid is DOPE. In some cases, the phospholipid is DSPC.

### Non-Cationic (Neutral) Lipid Component for Membrane Association

In some implementations, the multicomponent delivery system includes one or more lipid components comprising lipid moieties. The lipid component can be designed to degrade or hydrolyze to facilitate in vivo clearance of the multicomponent delivery system. The lipid moieties of the lipid components can be either straight-chain or branched, saturated, unsaturated, or aromatic. The lipid moieties of the structural lipid components may be naturally-occurring lipids. In some implementations, the lipid moieties of the lipid components are non-cationic and/or neutral. Alternatively, the lipid component may be a lipidated peptoid comprising lipid moieties at the N-position (“neutral lipidated peptoid”).

In some implementations, the multicomponent delivery system includes one or more neutral lipid components, such as a neutral lipidated peptoid. In some implementations which may be combined with any of the foregoing implementations, the multicomponent delivery system comprises 0 about 85.0 mol % one or more neutral lipid components (e.g., neutral lipidated peptoid(s)) of the total moles of the multicomponent delivery system. In certain variations, the multicomponent delivery system comprises between 0 to about 10.0 mol %, about 10.1 to about 20.0 mol %, about 20.1 to about 30.0 mol %, about 30.1 to about 40.0 mol %, about 40.1 to about 50.0 mol %, about 50.1 to about 60.0 mol %, about 60.1 to about 70.0 mol %, about 70.1 to about 80.0 mol %, or about 80.1 to about 85.0 mol % of neutral lipid component (e.g., neutral lipidated peptoid(s)) of the total number of moles of the multicomponent delivery system. In some variations, the multicomponent delivery system comprises between 0 to about 10 mol %, about 10 to about 20 mol %, about 20 to about 30 mol %, about 30 to about 40 mol %, about 40 to about 50 mol %, about 50 to about 60 mol %, about 60 to about 70 mol %, about 70 to about 80 mol %, or about 80 to about 85 mol % of neutral lipid component (e.g., neutral lipidated peptoid(s)) of the total number of moles of the multicomponent delivery system. In some variations, the multicomponent delivery system comprises between 0 to about 10 mol %, about 5 to about 20 mol %, about 10 to about 30 mol %, about 15 to about 40 mol %, about 20 to about 50 mol %, about 30 to about 60 mol %, about 55 to about 70 mol %, about 60 to about 75 mol %, or about 70 to about 85 mol % of neutral lipid component (e.g., neutral lipidated peptoid(s)) of the total number of moles of the multicomponent delivery system. In some variations, the multicomponent delivery system comprises between about 40 to about 80 mol %, about 50 to about 70 mol %, or about 55 to about 65 mol % of neutral lipid component (e.g., neutral lipidated peptoid(s)) of the total number of moles of the multicomponent delivery system.

In some implementations, the non-cationic lipidated peptoid of the multicomponent delivery system is a neutral lipidated peptoid. In some implementations, the non-cationic lipid component is a compound of Formula (IVa):

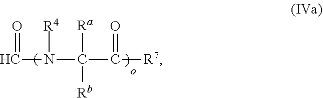

wherein o is integer from 3 to 10; each R⁴ independently is a neutral spacer moiety or a lipid moiety, as previously described herein; R⁷ is —NH₂; each R^{a} and R^{b} independently is —H. In some implementations, o is 3 or 4. In various implementations, o is 5 to 10. In some implementations, o is 3. In some cases, o is 4. In various cases, o is 5. In some implementations, o is 6. In various implementations, o is 7. In some cases o is 8. In various cases, o is 9. In some cases, o is 10. In some cases, each R⁴ independently is selected from the group consisting of R⁴ is independently C₈-C₂₄alkyl, C₁-C₄-alkyl substituted by cycloalkyl, heterocyclylalkyl, aryl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is, in some cases, substituted with one or more substituents —OH, halo, or alkoxy. In some implementations, each R⁴ is a lipid moiety selected from the group consisting of

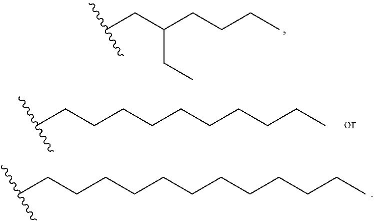

In some implementations, each R⁴ is

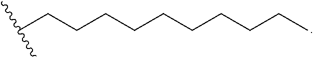 [SMILES: *CCCCCCCCCC]

In some implementations, each R⁴ is

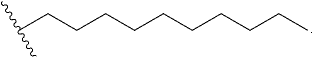 [SMILES: *CCCCCCCCCC]

In some cases, each R⁴ is selected from

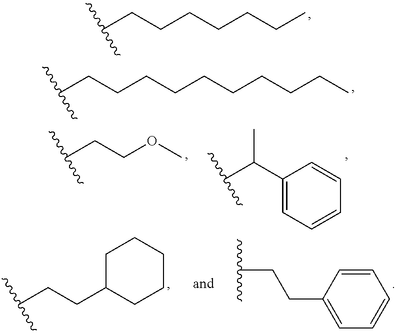

In some implementations, the peptoid backbone has alternating lipid moieties (e.g.,

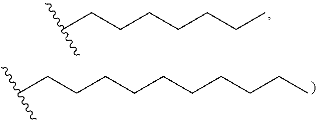

and neutral moieties (e.g.,

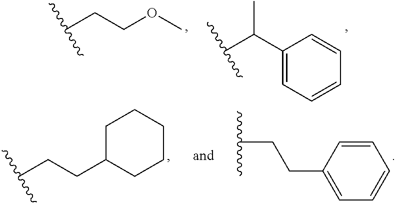

In some implementations, o is 3 or 4; each R⁴ is

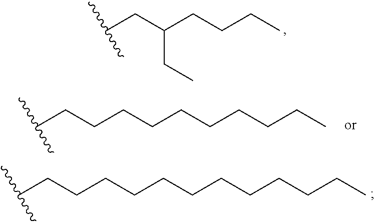

R⁷ is —NH₂; and each R^{a} and R^{b} independently is —H. Examples of neutral lipidated peptides can be found in Table 1C (e.g., Compounds 90, 119, 120, 121, 122, 123, 128, 129). In some cases, the neutral lipidated peptoid is selected from the group consisting of Compound 90, Compound 119, Compound 120, Compound 121, Compound 122, Compound 123, Compound 128, Compound 129, and combinations thereof. In some cases, the non-cationic lipid component is selected from the group consisting of Compound 90, Compound 119, and combinations thereof. In various cases, the neutral lipidated peptoid comprises Compound 90. In some implementations, the neutral lipidated peptoid comprises Compound 119. In some cases, the neutral lipidate peptoid component comprises Compound 150.

### Sterols

In some implementations, the multicomponent delivery system as described herein comprises one or more sterols. In some implementations, the one more sterols are selected from the group consisting of cholesterol, fecosterol, sitosterol, ergosterol, campesterol, stigmasterol, brassicasterol, tomatidine, ursolic acid, alpha-tocopherol, and mixtures thereof. In certain implementations, the sterol is cholesterol. In some implementations, the multicomponent delivery system comprises 0 about 85.0 mol % cholesterol of the total moles of the multicomponent delivery system. In some implementations, the multicomponent delivery system comprises about 50.0 to about 85.0 mol % cholesterol of the total moles of the multicomponent delivery system. In certain variations, the multicomponent delivery system comprises 0 to about 10.0 mol %, about 10.1 to about 20.0 mol %, about 20.1 to about 30.0 mol %, about 30.1 to about 40.0 mol %, about 40.1 to about 50.0 mol %, about 50.1 to about 60.0 mol %, about 60.1 to about 70.0 mol %, about 70.1 to about 80.0 mol %, or about 80.1 to about 85.0 mol % of cholesterol of the total moles of the multicomponent delivery system. In some variations, the multicomponent delivery system comprises 0 to about 10 mol %, about 10 to about 20 mol %, about 20 to about 30 mol %, about 30 to about 40 mol %, about 40 to about 50 mol %, about 50 to about 60 mol %, about 60 to about 70 mol %, about 70 to about 80 mol %, or about 80 to about 85 mol % of cholesterol of the total moles of the multicomponent delivery system. In some variations, the multicomponent delivery system comprises about 25 to about 85 mol %, about 30 to about 64 mol %, about 40 to about 60 mol %, about 35 to about 65 mol %, about 50 to about 70 mol %, about 45 to about 70 mol %, about 40 to about 70 mol %, about 60 to about 70 mol %, or about 75 to about 85 mol % of cholesterol of the total moles of the multicomponent delivery system. In certain variations, the multicomponent delivery system comprises at least about 50 mol % of cholesterol of the total moles of the multicomponent delivery system.

In some implementations, the multicomponent delivery system as described herein comprises one or more lipidated peptoids, wherein the lipid moieties of the lipidated peptoids are sterols. In some implementations, the sterols are selected from the group consisting of cholesterol, fecosterol, sitosterol, ergosterol, campesterol, stigmasterol, brassicasterol, tomatidine, ursolic acid, alpha-tocopherol, and mixtures thereof. In certain implementations, the lipid moieties of the lipidated peptoids are cholesterol. Such peptoids are referred to as “cholesteroids.” In some implementations, the multicomponent delivery system comprises 0-85.0 mol % cholesteroids of the total moles of the multicomponent delivery system. In certain variations, the multicomponent delivery system comprises 0 to 10.0 mol %, about 10.1 to about 20.0 mol %, about 20.1 to about 30.0 mol %, about 30.1 to about 40.0 mol %, about 40.1 to about 50.0 mol %, about 50.1 to about 60.0 mol %, about 60.1 to about 70.0 mol %, about 70.1 to about 80.0 mol %, or about 80.1 to about 85.0 mol % of cholesteroids of the total moles of the multicomponent delivery system. In certain variations, the multicomponent delivery system comprises at least 50 mol % of cholesteroids of the total moles of the multicomponent delivery system. In some variations, the multicomponent delivery system comprises 0 to about 10 mol %, about 10 to about 20 mol %, about 20 to about 30 mol %, about 30 to about 40 mol %, about 40 to about 50 mol %, about 50 to about 60 mol %, about 60 to about 70 mol %, about 70 to about 80 mol %, or about 80 to about 85 mol % of cholesteroids of the total moles of the multicomponent delivery system. In some variations, the multicomponent delivery system comprises about 25 to about 85 mol %, about 30 to about 64 mol %, about 40 to about 60 mol %, about 35 to about 65 mol %, about 50 to about 70 mol %, about 45 to about 70 mol %, about 40 to about 70 mol %, about 60 to about 70 mol %, or about 75 to about 85 mol % of cholesteroids of the total moles of the multicomponent delivery system. In certain variations, the multicomponent delivery system comprises at least about 50 mol % of cholesterol of the total moles of the multicomponent delivery system.

In some implementations, the multicomponent delivery system comprises one or more phospholipids, cholesterol, lipitoids, cholesteroids, or a mixture thereof as structural lipid component.

In some implementations, the multicomponent delivery system comprises molecules similar to cholesterol or phospholipid, such as sphingosine or phosphoinositides.

Particle Stabilization and/or Shielding Component

Stabilization of the delivery vehicles can be accomplished through anchoring of hydrophilic polymers or other molecules to the surface of the delivery vehicles, including but not limited to polyalkylene oxide compounds (e.g, poly(ethylene glycol), poly(propylene glycol)), polysaccharides, and/or poly(phosphate)s. These groups can vary in molecular weight and/or length to modulate shielding properties. The shielding component can also be anchored into the particle via a lipid, or an anionic component to attach to a positive zeta potential multicomponent delivery system.

In some implementations, the shielding component is cationic. In some implementations, the shielding component is neutral. In some implementations, the shielding component is zwitterionic. In some implementations, the shielding component is anionic. In certain implementations, the shielding component is a peptoid.

### PEGylated Lipids and PEGylated Lipidated Peptoids

In some implementations, the shielding component of the multicomponent delivery system described herein comprises one or more PEGylated compounds. The one or more PEGylated compounds may be PEGylated lipids or PEGylated lipidated peptoids. It should be recognized that the term “PEGylated lipid” may also be interchangeably referred to as a “PEG lipid” or “PEG-modified lipid”. As described herein, PEGylated lipid may be understood to include any lipid or lipid-like compound(s) covalently bound to a polyethylene glycol moiety. “PEGylated lipidated peptoids” may also be interchangeably referred to as a “PEG lipidated peptoids” or “PEG-modified lipidated peptoids.”

In some implementations, the multicomponent delivery system comprise about 0.5 to about 5 mol % (such as about 0.5 to about 1.0 mol %, about 1.1 to about 2.0 mol %, about 2.1 to about 3.0 mol %, about 3.1 to about 4.0 mol %, about 4.1 to about 5.0 mol %) one or more shielding component of the total weight of the multicomponent delivery system. In certain implementations, the multicomponent delivery system comprises about 0.5 to about 5 mol % (such as about 0.5 to about 1.0 mol %, about 1.1 to about 2.0 mol %, about 2.1 to about 3.0 mol %, about 3.1 to about 4.0 mol %, about 4.1 to about 5.0 mol %) one or more PEGylated compounds of the total moles of the multicomponent delivery system. In still other implementations, the multicomponent delivery system therein comprises about 0.5 to about 5 mol % (such as about 0.5 to about 1.0 mol %, about 1.1 to about 2.0 mol %, about 2.1 to about 3.0 mol %, about 3.1 to about 4.0 mol %, about 4.1 to about 5.0 mol %) 1,2-dimyristoyl-rac-glycero-3-methoxypolyethylene glycol (DMG-PEG) of the total moles of the multicomponent delivery system. In some implementations, the multicomponent delivery system comprises about 0.5 to about 5 mol % (such as about 0.5 to about 1.0 mol %, about 1 to about 2 mol %, about 2 to about 3 mol %, about 3 to about 4 mol %, about 4 to about 5 mol %) one or more shielding component of the total weight of the multicomponent delivery system. In certain implementations, the multicomponent delivery system comprises about 0.5 to about 5 mol % (such as about 0.5 to about 1 mol %, about 1 to about 2 mol %, about 2 to about 3 mol %, about 3 to about 4 mol %, about 4 to about 5 mol %) one or more PEGylated compounds of the total moles of the multicomponent delivery system. In still other implementations, the multicomponent delivery system therein comprises about 0.5 to about 5 mol % (such as about 0.5 to about 1 mol %, about 1 to about 2. mol %, about 2 to about 3 mol %, about 3 to about 4 mol %, about 4 to about 5 mol %) 1,2-dimyristoyl-rac-glycero-3-methoxypolyethylene glycol (DMG-PEG) of the total moles of the multicomponent delivery system. In some cases, the multicomponent delivery system therein comprises about 1 to about 5 mol %, or about 1 to about 3 mol %, or about 1.5 to about 2.5 mol % of the PEGylated lipid, such as 1,2-dimyristoyl-rac-glycero-3-methoxypolyethylene glycol (DMG-PEG), of the total moles of the multicomponent delivery system.

It should be recognized that particular molecular weights of the PEG chain in the foregoing PEGylated lipids and/or PEGylated lipidated peptoids may be especially advantageous for incorporation into the complexes of the present disclosure. For example, in some implementations, the PEG chain has a molecular weight between about 350 and about 6,000 g/mol, between about 1,000 and about 5,000 g/mol, or between about 2,000 and about 5,000 g/mol, or between about 1,000 and about 3,000 g/mol, or between about 1,500 and about 4,000 g/mol. In certain implementations, the PEG chain of the PEG lipid has a molecular weight of about 350 g/mol, about 500 g/mol, about 600 g/mol, about 750 g/mol, about 1,000 g/mol, about 2,000 g/mol, about 3,000 g/mol, about 5,000 g/mol, or about 10,000 g/mol. In certain other implementations, the PEG chain of the PEGylated lipid has a molecular weight of about 500 g/mol, about 750 g/mol, about 1,000 g/mol, about 2,000 g/mol or about 5,000 g/mol. The PEG chain can be branched or linear. For example, in certain implementations, the PEGylated lipid is dimyristoylglycerol-polyethylene glycol 2000 (DMG-PEG 2000).

Suitable lipid moieties for the PEGylated lipid or the PEGylated lipidated peptoids may include, for example, optionally substituted branched or straight chain aliphatic moieties, or optionally substituted moieties derived from natural lipid compounds, including fatty acids, sterols, and isoprenoids.

In some implementations, the lipid moieties may include branched or straight chain aliphatic moieties having from about 6 to about 50 carbon atoms or from about 10 to about 50 carbon atoms. The aliphatic moieties may, in some examples, comprise one or more heteroatoms, and/or one or more double or triple bonds (i.e., saturated or mono- or poly-unsaturated). In certain implementations, the lipid moieties may include optionally substituted aliphatic, straight chain or branched moieties, each hydrophobic tail independently having from about 8 to about 30 carbon atoms or from about 6 to about 30 carbon atoms. In certain implementations, the lipid moieties may include, for example, aliphatic carbon chains derived from fatty acids and fatty alcohols. In some implementations, each lipid moiety is independently C₈-C₂₄-alkyl or C₈-C₂₄-alkenyl, wherein the C₈-C₂₄-alkenyl is, in some cases mono- or poly-unsaturated.

Natural lipid moieties employed in the practice of the present disclosure can be derived from, for example, phospholipids, glycerides (such as di- or tri-glycerides), glycosylglycerides, sphingolipids, ceramides, and saturated and unsaturated sterols, isoprenoids, and other like natural lipids.

Other suitable lipid moieties may include lipophilic aromatic groups such as optionally substituted aryl or arylalkyl moieties, including for example naphthalenyl or ethylbenzyl, or lipids comprising ester functional groups including, for example, sterol esters and wax esters.

In some implementations, the one or more PEGylated lipids are selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and any combinations thereof.

In some implementations, the PEGylated lipids comprise a PEG-modified sterol. In certain implementations, the PEGylated lipids comprises PEG-modified cholesterol.

In some implementations, the PEGylated lipid is a PEG-modified ceramide. In certain implementations, the PEG-modified ceramine is selected from the group consisting of N-octanoyl-sphingosine-1-{succinyl[methoxy(polyethylene glycol)]} and N-palmitoyl-sphingosine-1-{succinyl[methoxy(polyethylene glycol)]}, and any combination thereof.

In some implementations, the PEGylated lipids are PEG-modified phospholipid, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE),1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In certain implementations, the phospholipid is DOPE.

In some implementations, the one or more PEGylated lipids comprise a PEG-modified phosphatidylethanol. In some implementations, the PEGylated lipid is a PEG-modified phosphatidylethanol selected from the group consisting of PEG-modified DMPE (DMPE-PEG), PEG-modified DSPE (DSPE-PEG), PEG-modified DPPE (DPPE-PEG), and PEG-modified DOPE (DOPE-PEG).

In certain implementations, the PEGylated lipid is selected from the group consisting of dimyristoylglycerol-polyethylene glycol (DMG-PEG), distearoylglycerol-polyethylene glycol (DSG-PEG), dipalmitoylglycerol-polyethylene glycol (DPG-PEG), and dioleoylglycerol-polyethylene glycol (DOG-PEG). In certain implementations, the PEG lipid is DMG-PEG.

In still further implementations, the one or more PEGylated lipidated peptoids can comprise a PEGylated peptoid encompassed by formula (I), comprising at least one oligo- or polyethylene glycol moiety. For example, the PEGylated lipidated peptoids may be Compound 48, 56, 64, 72, 106, 107, 108 and 109 in Table 1D. By virtue of their flexibility in accommodating both lipid and PEG moieties along the backbone of the peptoid chain and depending upon the nature of their specific substituents, the tertiary amino lipidated and/or PEGylated cationic peptoids of formula (I) may serve not only as the cationic components but also as suitable PEGylated compounds to stabilize the multicomponent delivery system. It should be recognized that tertiary amino PEGylated cationic peptoids of formula (I) may be combined with other classes of cationic compounds such as lipitoids or lipid-like compounds as well as other tertiary amino lipidated and/or PEGylated cationic peptoid compounds of formula (I).

In some implementations, the non-cationic lipidated peptoid comprises PEGylated lipididated peptoid. In some cases, the PEGylated lipidated peptoid comprises a compound of Formula (Va):

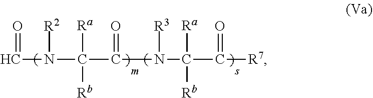

wherein:
m is an integer from 1 to 15;
s is an integer from 1 to 6;
each R² independently is an ethylene glycol moiety of the formula —CH₂CH₂O(CH₂CH₂O)ᵤCH₃, and wherein each u is independently an integer from 2 to 200, as previously described herein;
R³ is a lipid moiety, as previously described herein;
R⁷ is —H, alkyl, acyl, —OH, —OR^{7a}, —NH₂, —NHR^{7a}, or a lipid moiety, wherein R^{7a} is alkyl, acyl, or a lipid moiety; and
each R^{a} and R^{b} independently is —H, C₁-C₄-alkyl, or a side chain moiety found on a naturally- or non-naturally-occurring amino acid.

In some implementations, m is 2 to 15. In some implementations, m is 5 to 15. In some cases, m is 5 to 10. In various cases, m is 10 to 15. In some implementations, m is 10. In some cases, s is 2 to 6. In various implementations, s is 2. In some implementations, s is 3 or 4. In some cases, s is 3. In various cases, s is 4. In various implementations s is 5. In various cases, s is 6. In some implementations, each R² independently is

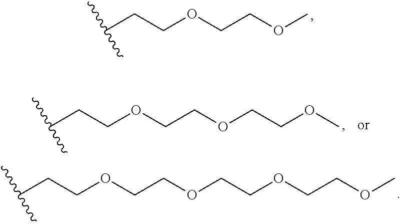

In some cases, each R² independently is

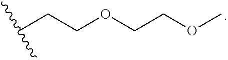 [SMILES: *CCOCCOC]

In various cases, each R² independently is

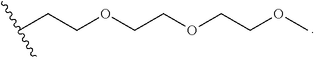 [SMILES: *CCOCCOCCOC]

In some cases, R⁷ is —NH₂. In various cases, each R^{a} and R^{b} independently is —H. In various implementations, each R³ independently is C₈-C₁₂ alkyl, such as n-decyl or 2-ethylhexyl. In various implementations, each R³ independently is

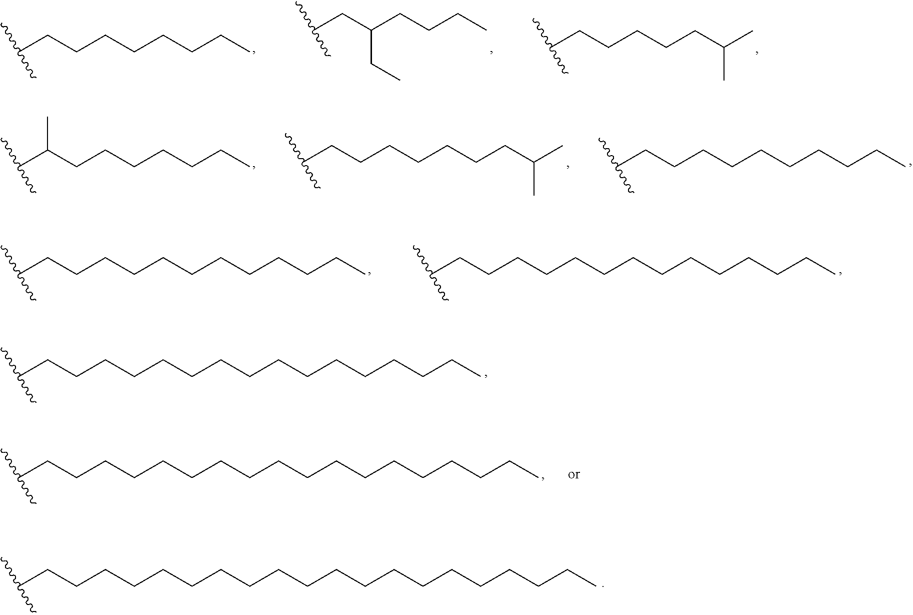

In some implementations, each R³ independently is

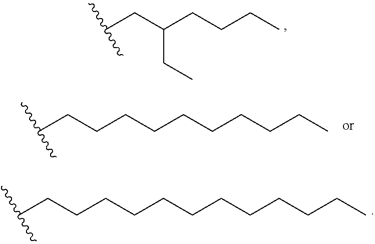

In some implementations, m is 5 to 15; s is 3, 4, or 5; R⁷ is —NH₂; each R^{a} and R^{b} independently is —H; and each R³ independently is

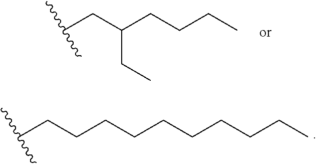

Examples of PEGylated lipididated peptoid components having a Formula (Va) can be found in Table 1D (e.g., Compounds 106 and 107).

In some implementations, the PEGylated component is a compound of Formula (Vb):

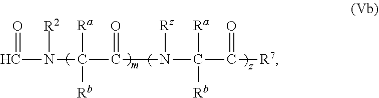

wherein:
m is an integer from 1 to 15;
z is an integer from 1 to 6;
each R² independently is any ethylene glycol is an ethylene glycol moiety of the formula —CH₂CH₂O(CH₂CH₂O)ᵤCH₃, and wherein each u is independently an integer from 2 to 200, as previously described herein;
R⁷ is —H, alkyl, acyl, —OH, —OR^{7a}, —NH₂, —NHR^{7a}, or a lipid moiety, wherein R^{7a} is alkyl, acyl, or a lipid moiety;
each R^{a} and R^{b} independently is —H, C₁-C₄-alkyl, or a side chain moiety found on a naturally- or non-naturally-occurring amino acid; and
R^{z} is an anionic moiety.

In some implementations, m is 2 to 15. In some implementations, m is 5 to 15. In some cases, m is 5 to 10. In various cases, m is 10 to 15. In some implementations, m is 10. In some cases, z is 2 to 6. In various implementations, z is 2. In some implementations, z is 3. In some cases, z is 4. In various cases, z is 3 or 4. In various implementations z is 5. In various cases, z is 6. In some implementations, each R² independently is

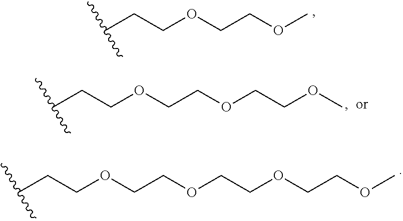

In some cases, each R² independently is

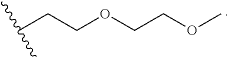 [SMILES: *CCOCCOC]

In various cases, R² independently is

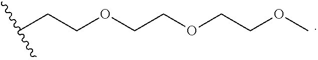 [SMILES: *CCOCCOCCOC]

In some cases, R⁷ is —NH₂. In various cases, each R^{a} and R^{b} independently is —H. In some implementations, each R^{z} independently is C₂₋₅ alkylenecarboxylic acid. In various implementations, each R^{z} is selected from

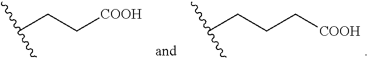 [SMILES: *CCC(=O)O ; *CCCC(=O)O]

In some cases, each R^{z} is

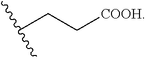 [SMILES: *CCC(=O)O]

In some implementations, m is 5 to 15; z is 3, 4, or 5; R⁷ is —NH₂; each R^{a} and R^{b} independently is —H; and each R^{z} independently is

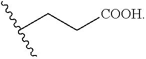 [SMILES: *CCC(=O)O]

Examples of PEGylated lipididated peptoid components having a Formula (Vb) can be found in Table 1D (e.g., Compounds 108 and 109).

In some implementations, the PEGylated component is a compound of Formula (Vc):

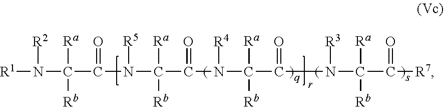

wherein s is 3 or 4, r is 1 or 2, q is 2, and the remaining variables are as previously described herein for Formula (I). Examples of PEGylated lipididated peptoid components having a Formula (Vc) can be found in Table 1D (e.g., Compounds 56, 64, and 72).

### Other Peptoid Components

The multicomponent delivery system can comprise various peptoids components that have different functions. Thus, fine tuning of the multicomponent delivery system can be achieved to deliver different polyanionic cargo (e.g, nucleic acids such as RNA and/or DNA) to cells. In some implementations, one or more of these additional peptoids are cationic lipidated peptoids. In some cases, one or more of these additional peptoids are non-cationic lipidated peptoids (e.g., a neutral lipidated peptoid, an anionic lipidated peptoid, a zwitterionic lipidated peptoid, or a PEGylated peptoid). In some implementations, one or more of these additional peptoids are neutral lipidated peptoids. In some implementations, one or more of these additional peptoids are zwitterionic lipidated peptoids. In some implementations, one or more of these additional peptoids are anionic lipidated peptoids. In some implementations, one or more of these additional peptoids are PEGylated peptoids. In some implementations, at least one of these additional peptoids is not a cationic lipidated peptoid.

Lipid fluidity/crystallinity of the components is known to influence cellular uptake and endosomal release. In some implementations, the multicomponent delivery system comprises end-capped cholesteroids. In some implementations, the lipidated peptoids (cationic or non-cationic) comprise aliphatic side chains such as cyclohexane, decalin, adamantane, (+)-dehydroabietylamine, or (−)-cis-myrtanylamine.

In some implementations, peptoid components with sheets and helices in their structures can be included in the multicomponent delivery systems to provide more/less structure to domains. The helices may have hydrophobic and/or hydrophilic faces. As a non-limiting example, low density lipoprotein (LDL) peptoid mimics may be included as a component in the multicomponent delivery system.

In some implementations, the multicomponent delivery system comprises sugar-functionalized peptoids such as mannose-lipid, galactose, or phosphoinositide.

In some implementations, the multicomponent delivery system comprises phosphoinositol compounds. The phosphoinositol compounds may be located on the outside of the multicomponent delivery system. The phosphoinositol compounds may be Phosphatidylinositol monophosphates, such as Phosphatidylinositol 3-phosphate, also known as PtdIns3P or PI(3)P, Phosphatidylinositol 4-phosphate, also known as PtdIns4P or PI(4)P, or Phosphatidylinositol 5-phosphate, also known as PtdIns5P or PI(5)P; phosphatidylinositol bisphosphates, such as Phosphatidylinositol 3,4-bisphosphate, also known as PtdIns(3,4)P₂ or PI(3,4)P₂, Phosphatidylinositol 3,5-bisphosphate, also known as PtdIns(3,5)P₂ or PI(3,5)P₂, or Phosphatidylinositol 4,5-bisphosphate, also known as PtdIns(4,5)P₂, PI(4,5)P₂ or PIP₂; Phosphatidylinositol trisphosphate, such as Phosphatidylinositol 3,4,5-trisphosphate, also known as PtdIns(3,4,5)P₃ or PI(3,4,5)P₃.

### Endosomal Escape/Disruption Component

The multicomponent delivery system may comprise, in some examples, components that facilitate endosomal escape, including but not limited to, buffering amines or polyamines; nitrogen-containing heterocycle groups and/or nitrogen-containing heteroaryl groups such as imidazoles, pyrroles, pyridines, pyrimidines; maleic acid derivatives; or membrane-lytic peptides.

### Targeting Components

The multicomponent delivery system may comprise targeting moeities on the surface of the system. Targeting moieties can be peptides, antibody mimetics, nucleic acids (e.g., aptamers), polypeptides (e.g., antibodies), glycoproteins, small molecules, carbohydrates, or lipids. Non-limiting examples of the targeting moiety include a peptide such as somatostatin, octreotide, LHRH, an EGFR-binding peptide, RGD-containing peptides, a protein scaffold such as a fibronectin domain, an aptide or bipodal peptide, a single domain antibody, a stable scFv, or a bispecific T-cell engagers, nucleic acid (e.g., aptamer), polypeptide (e.g., antibody or its fragment), glycoprotein, small molecule, carbohydrate, or lipid. The targeting moiety may be an aptamer being either RNA or DNA or an artificial nucleic acid; small molecules; carbohydrates such as mannose, galactose and arabinose; vitamins such as ascorbic acid, niacin, pantothenic acid, carnitine, inositol, pyridoxal, lipoic acid, folic acid (folate), riboflavin, biotin, vitamin B12, vitamin A, E, and K; a protein or peptide that binds to a cell-surface receptor such as a receptor for thrombospondin, tumor necrosis factors (TNF), annexin V, interferons, cytokines, transferrin, GM-CSF (granulocyte-macrophage colony-stimulating factor), or growth factors such as vascular endothelial growth factor (VEGF), hepatocyte growth factor (HGF), (platelet-derived growth factor (PDGF), basic fibroblast growth factor (bFGF), and epidermal growth factor (EGF).

### Therapeutic Components

Small molecule drugs or other biologics can also be incorporated into the multicomponent delivery system. Non-limiting examples include incorporating drugs that disrupt the blood-brain-barrier or enhance cellular uptake; drugs that affect intracellular trafficking or endosomal escape; or drugs that are immunomodulators to affect antigen presentation when the multicomponent delivery system is used in as a vaccine.

### Non-Limiting Examples of Multicomponent Delivery System Formulations

Tables 1A, 1B, 1C, 1D, and 1E show non-limiting examples of lipidated peptoids of the disclosure. Table 1A provides example lipidated cationic peptoids, such as Compound 24, 73, 79, 81, 85, 93, 112, 115, 127, 137, 152, 155, 163, and 164, which may be used as the cationic lipidated peptoid in the multicomponent delivery system. Table 1B provides example anionic/zwitterionic lipidated peptoids, such as 105, 124, 125, 126, and 135, which can be used as the non-cationic lipidated peptoids of the multicomponent delivery system. Table 1C provides example neutral lipidated peptoids, such as Compound 90, 119, 120, 121, 122, 123, 128, and 129, which can be used as the non-cationic lipidated peptoids of the multicomponent delivery system. Table 1D provides example PEGylated peptoids (average molecular weight 2000 g/mol), such as Compound 48, 56, 64, 72, 106, 107, 108, and 109, which can be used as a shielding component in the multicomponent delivery system.

TABLE 1A
Non-Limiting Lipidated Cationic Peptoids

| # | Compound Structure |
|---|---|
| 1 | 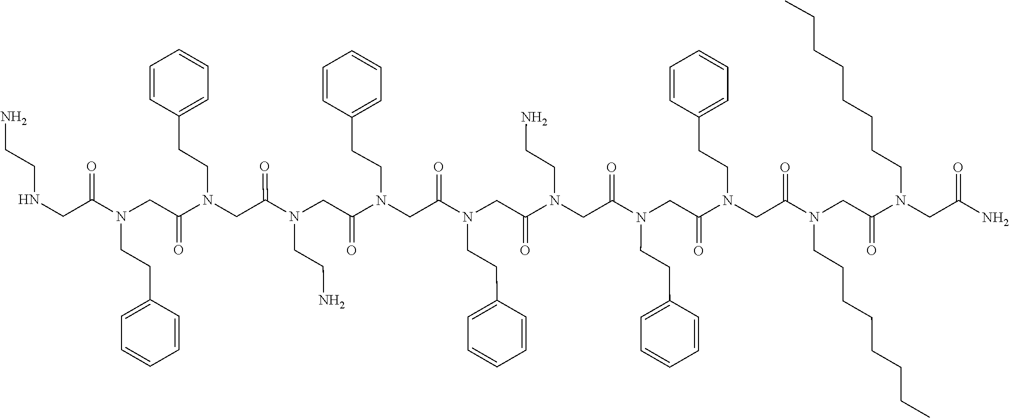 [SMILES: CCCCCCCCN(CC(N)=O)C(=O)CN(CCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 2 | 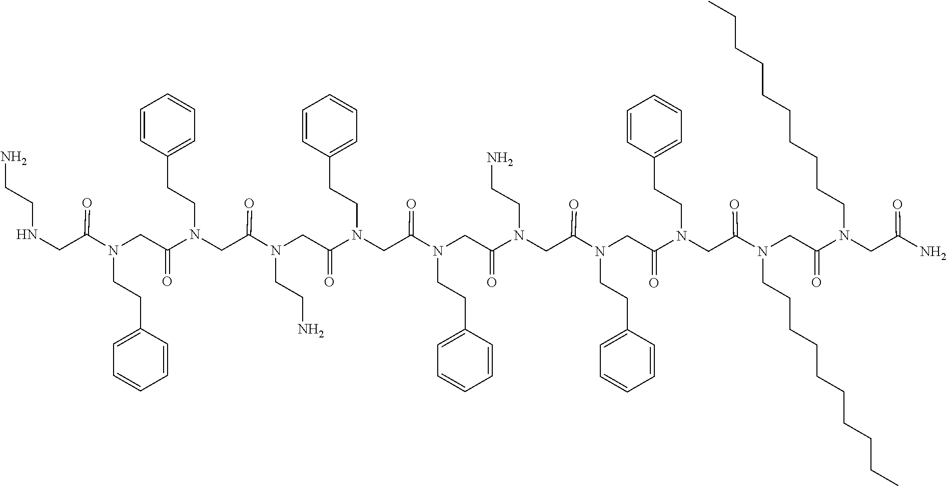 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 3 | 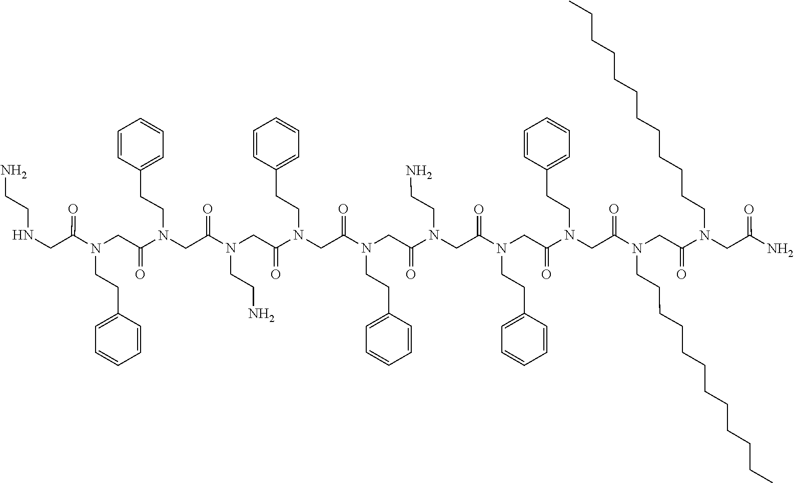 [SMILES: CCCCCCCCCCCCN(CC(N)=O)C(=O)CN(CCCCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 4 | 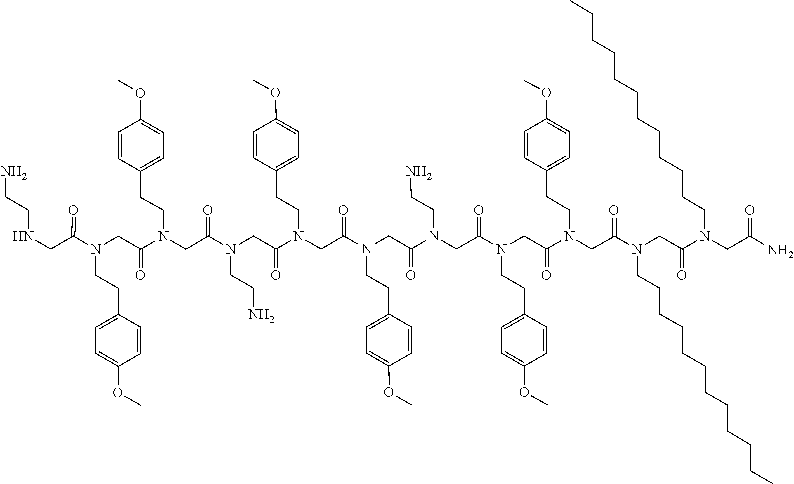 [SMILES: CCCCCCCCCCCCN(CC(N)=O)C(=O)CN(CCCCCCCCCCCC)C(=O)CN(CCc1ccc(OC)cc1)C(=O)CN(CCc1ccc(OC)cc1)C(=O)CN(CCN)C(=O)CN(CCc1ccc(OC)cc1)C(=O)CN(CCc1ccc(OC)cc1)C(=O)CN(CCN)C(=O)CN(CCc1ccc(OC)cc1)C(=O)CN(CCc1ccc(OC)cc1)C(=O)CNCCN] |
| 5 | 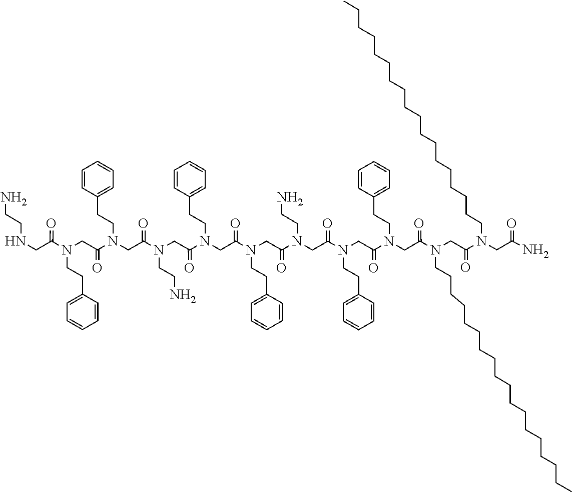 [SMILES: CCCCCCCCCCCCCCCCCCN(CC(N)=O)C(=O)CN(CCCCCCCCCCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 6 | 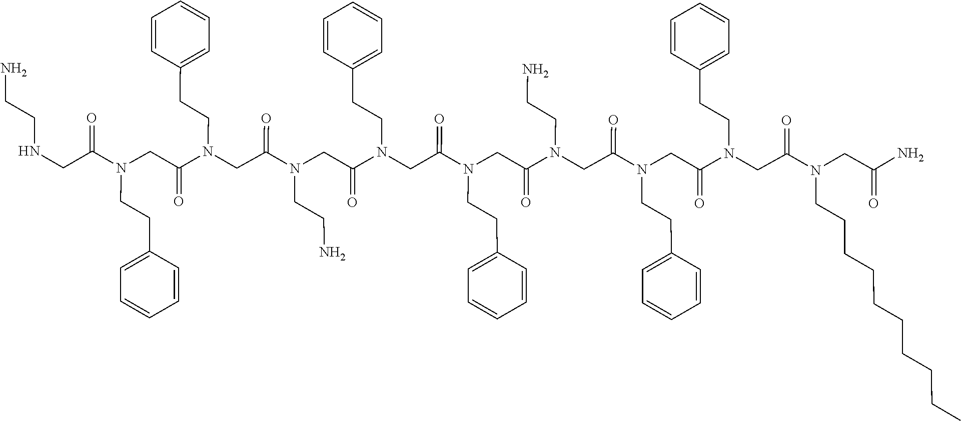 [SMILES: CCCCCCCCCCN(CC(N)=O)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 7 | 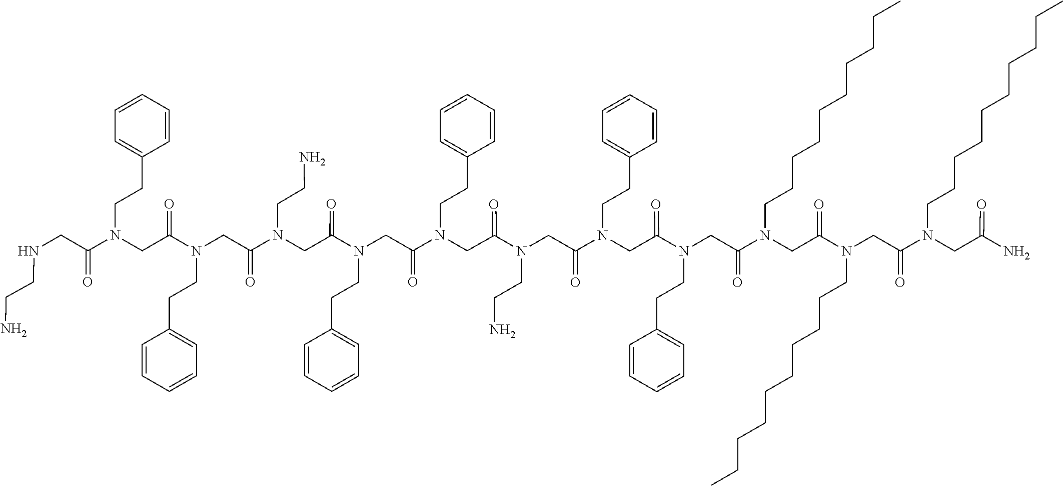 [SMILES: CCCCCCCCCCN(CC(N)=O)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 8 | 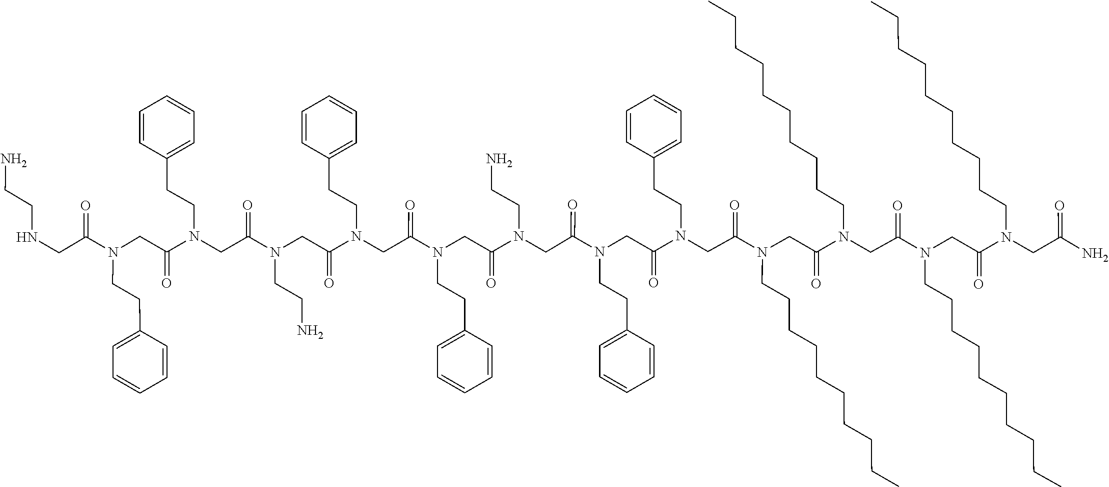 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 9 | 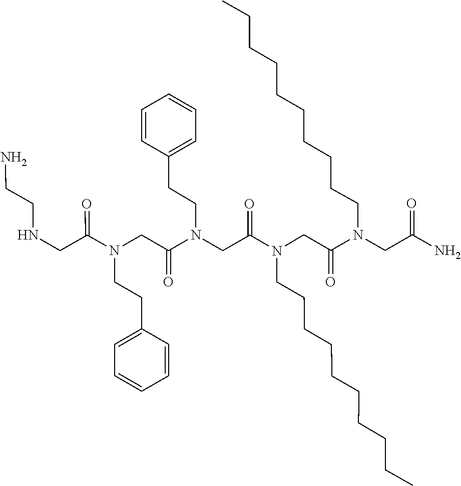 [SMILES: CCCCCCCCCCN(CC(N)=O)C(=O)CN(CCCCCCCCCC)C(=O)CN(CCc1ccccc1)C(=O)CN(CCc1ccccc1)C(=O)CNCCN] |
| 10 | 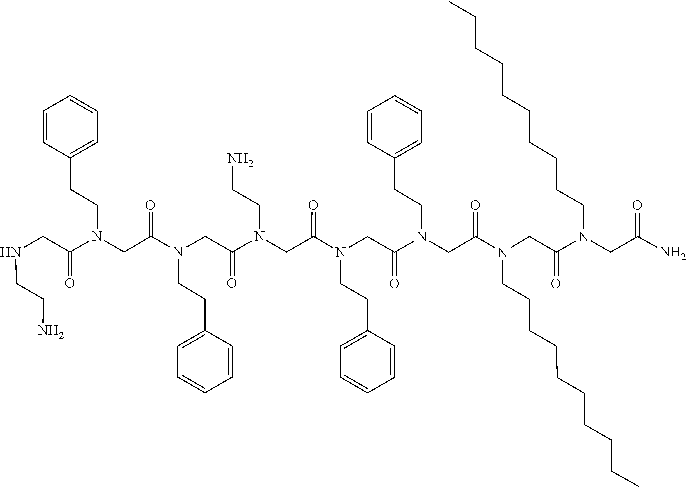 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 11 | 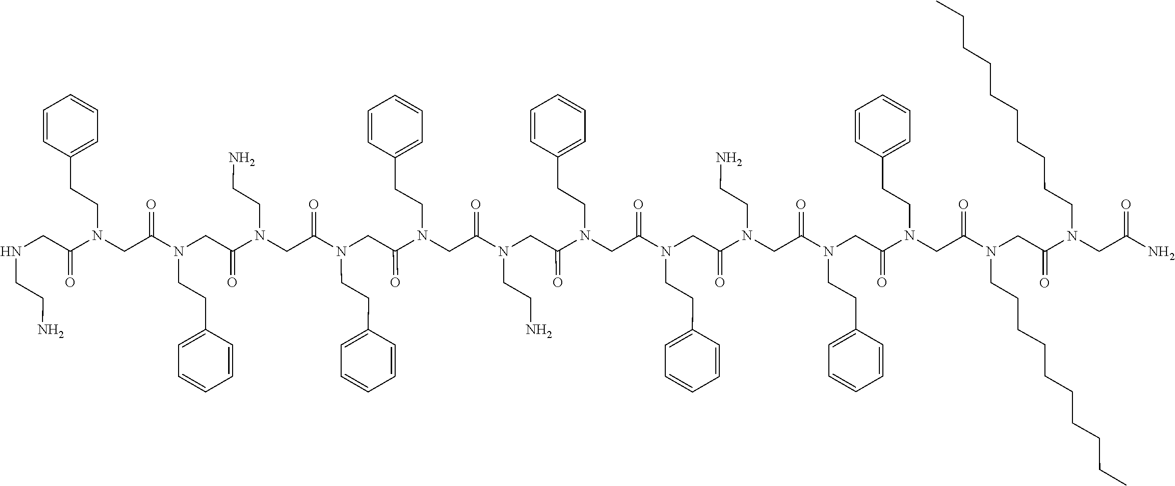 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 12 | 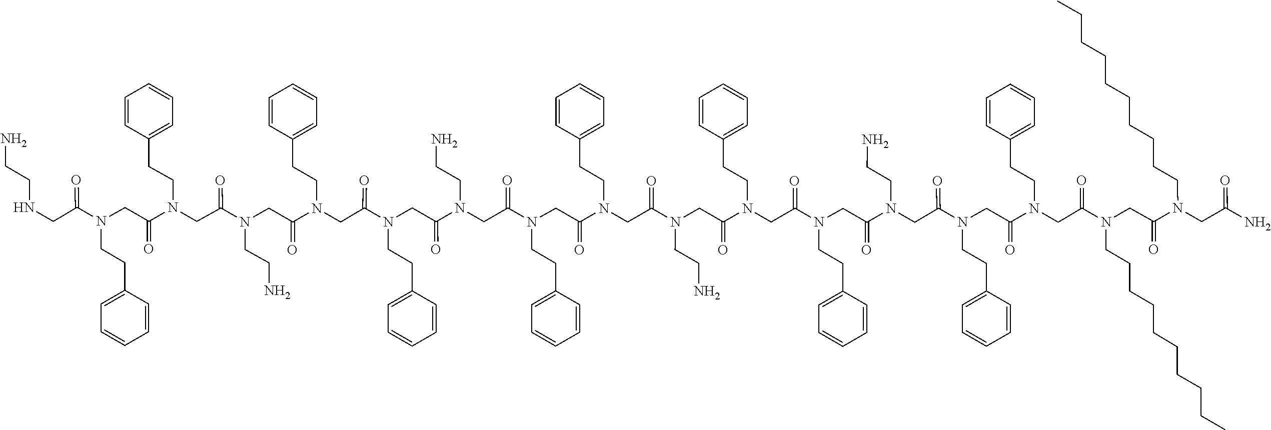 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 13 | 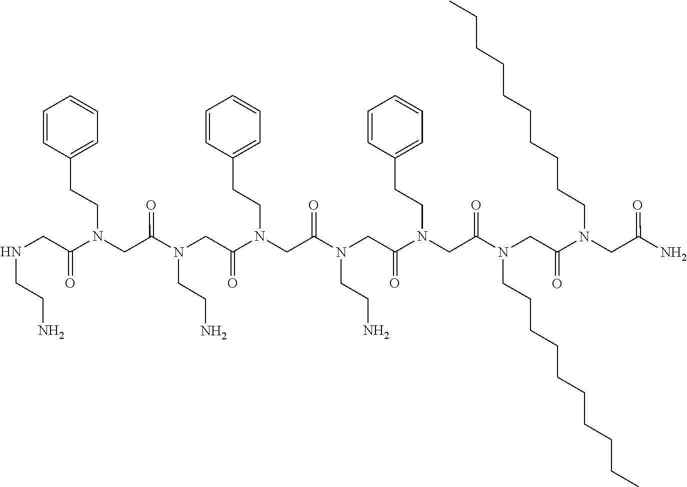 [SMILES: CCCCCCCCCCN(CC(N)=O)C(=O)CN(CCCCCCCCCC)C(=O)CN(CCc1ccccc1)C(=O)CN(CCN)C(=O)CN(CCc1ccccc1)C(=O)CN(CCN)C(=O)CN(CCc1ccccc1)C(=O)CNCCN] |
| 14 | 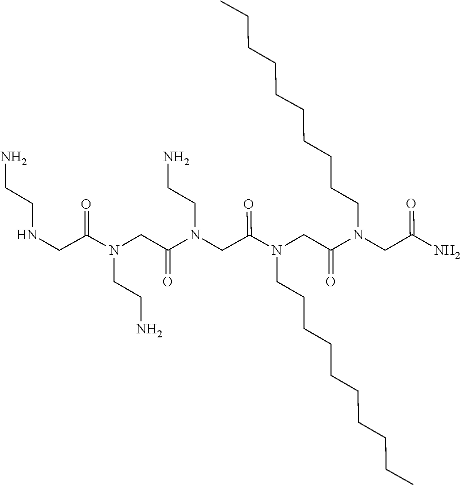 [SMILES: CCCCCCCCCCN(CC(N)=O)C(=O)CN(CCCCCCCCCC)C(=O)CN(CCN)C(=O)CN(CCN)C(=O)CNCCN] |
| 15 | 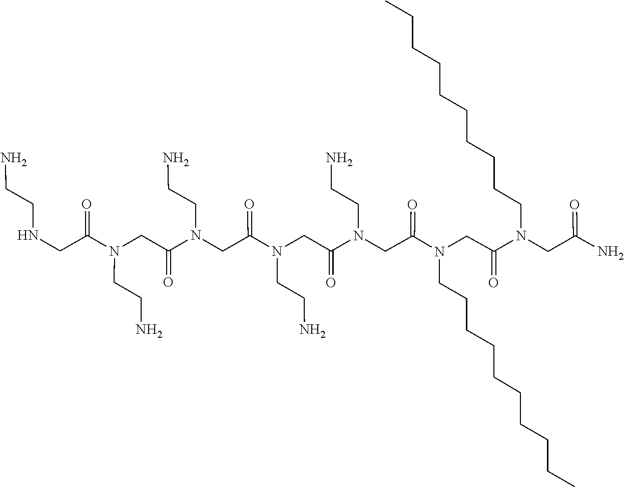 [SMILES: CCCCCCCCCCN(CC(N)=O)C(=O)CN(CCCCCCCCCC)C(=O)CN(CCN)C(=O)CN(CCN)C(=O)CN(CCN)C(=O)CN(CCN)C(=O)CNCCN] |
| 16 | 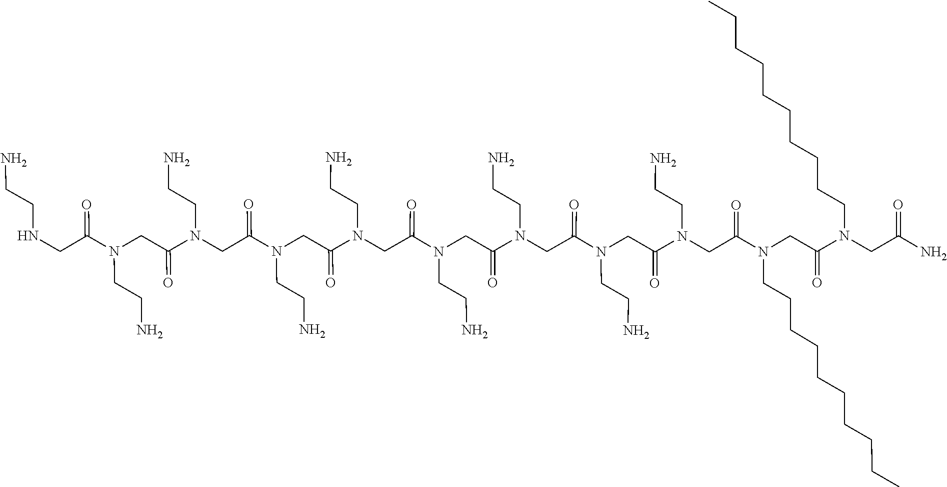 [SMILES: CCCCCCCCCCN(CC(N)=O)C(=O)CN(CCCCCCCCCC)C(=O)CN(CCN)C(=O)CN(CCN)C(=O)CN(CCN)C(=O)CN(CCN)C(=O)CN(CCN)C(=O)CN(CCN)C(=O)CN(CCN)C(=O)CN(CCN)C(=O)CNCCN] |
| 17 | 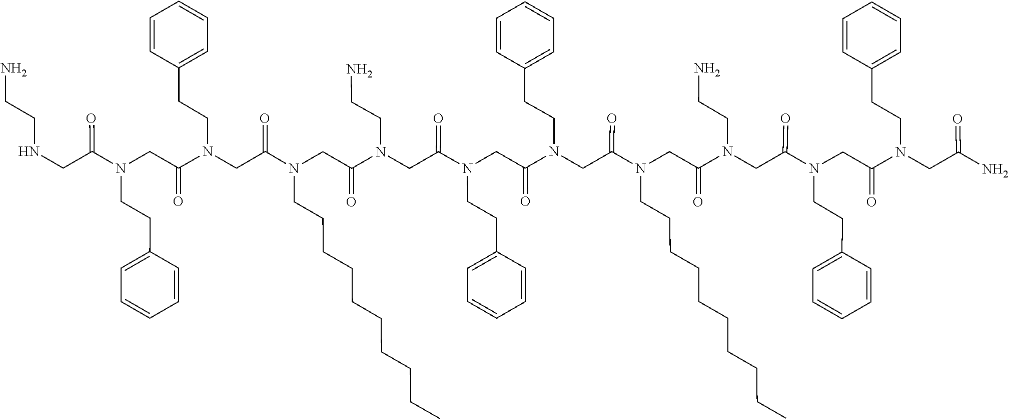 [SMILES: CCCCCCCCCCN(CC(=O)N(CCN)CC(=O)N(CCc1ccccc1)CC(=O)N(CCc1ccccc1)CC(=O)N(CCCCCCCCCC)CC(=O)N(CCN)CC(=O)N(CCc1ccccc1)CC(=O)N(CCc1ccccc1)CC(N)=O)C(=O)CN(CCc1ccccc1)C(=O)CN(CCc1ccccc1)C(=O)CNCCN] |
| 18 | 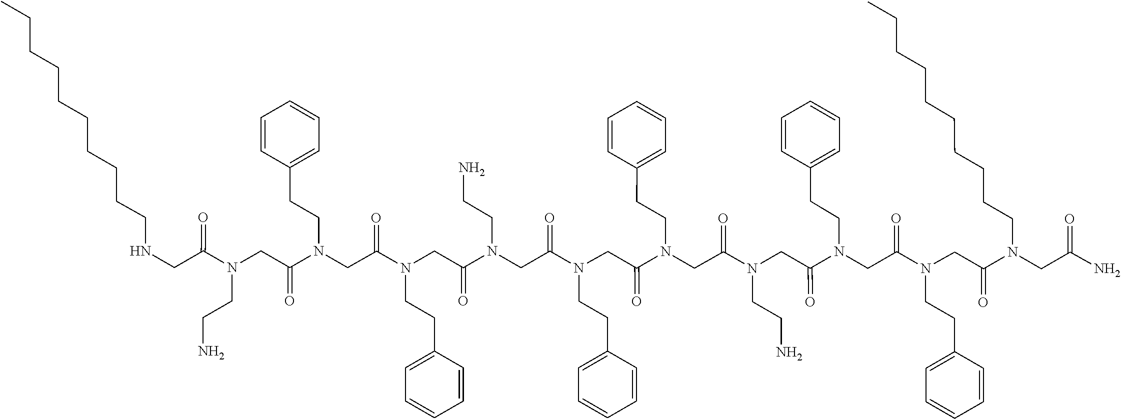 [SMILES: CCCCCCCCCCNCC(=O)N(CCN)CC(=O)N(CCc1ccccc1)CC(=O)N(CCc1ccccc1)CC(=O)N(CCN)CC(=O)N(CCc1ccccc1)CC(=O)N(CCc1ccccc1)CC(=O)N(CCN)CC(=O)N(CCc1ccccc1)CC(=O)N(CCc1ccccc1)CC(=O)N(CCCCCCCCCC)CC(N)=O] |
| 19 | 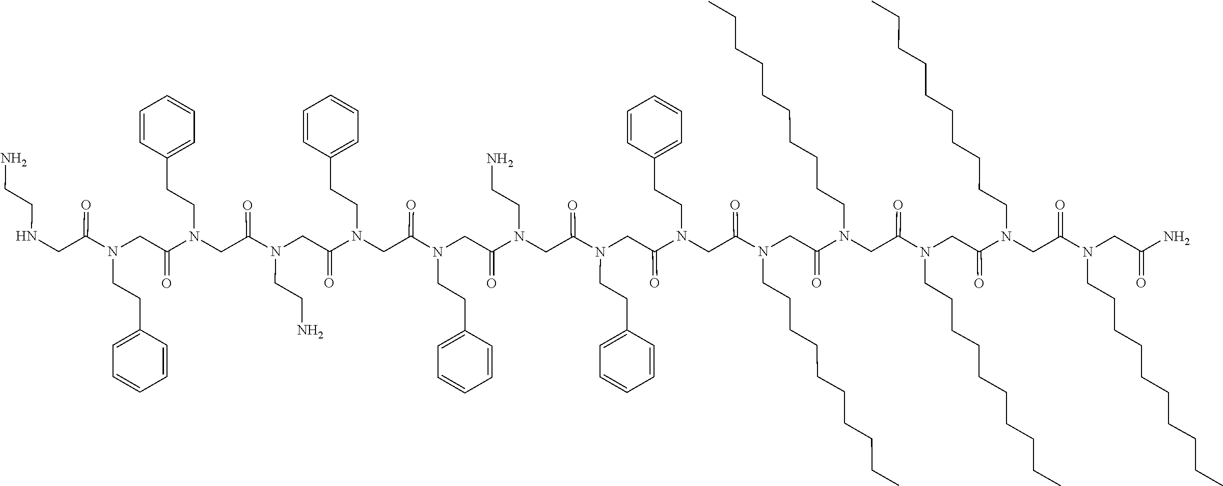 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 20 | 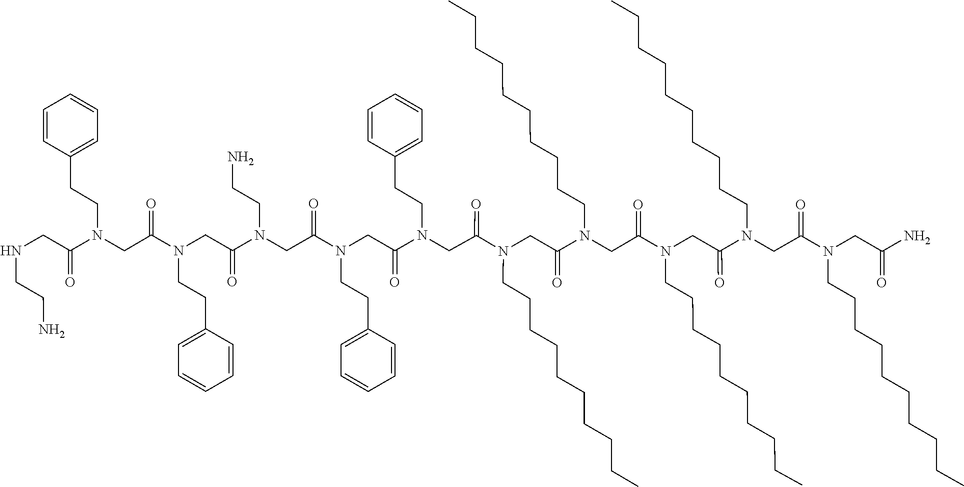 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 21 | 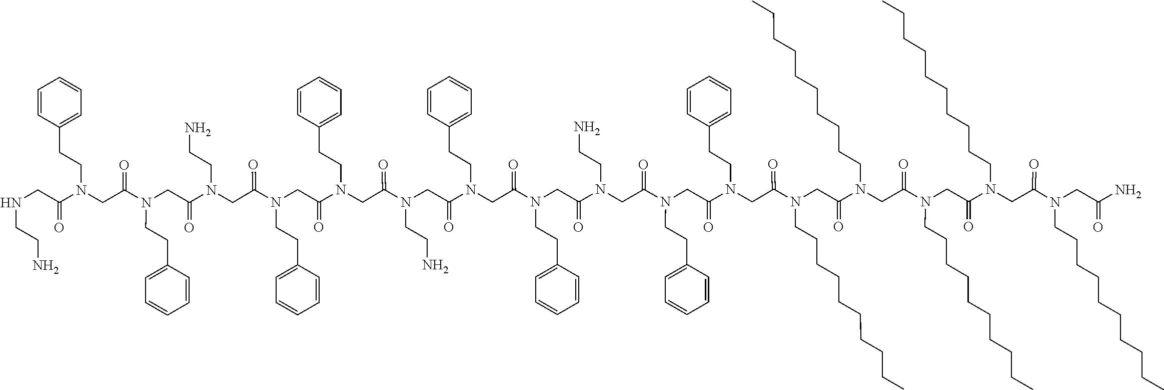 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 22 | 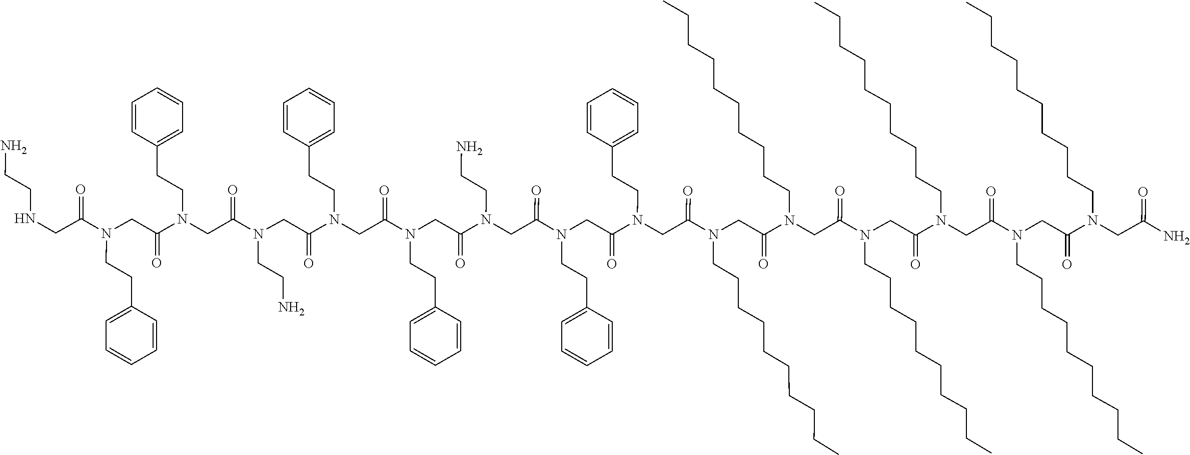 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 23 | 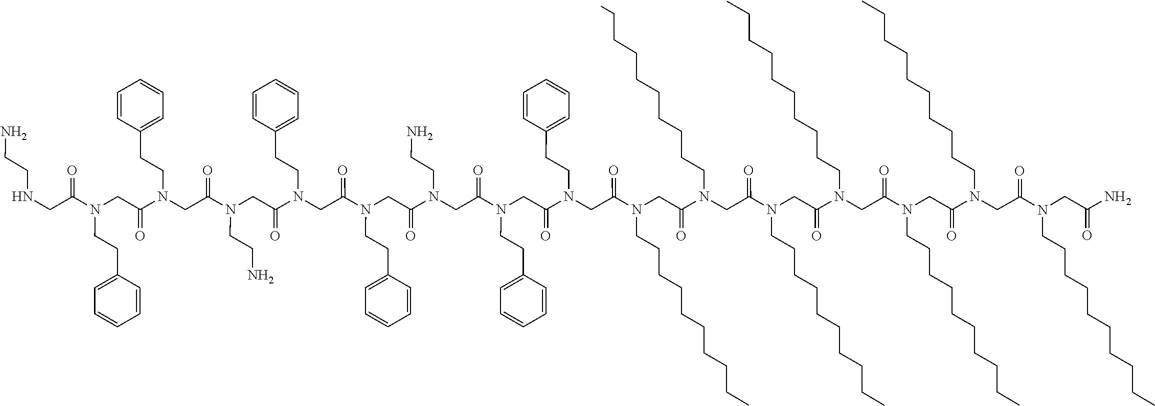 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 24 | 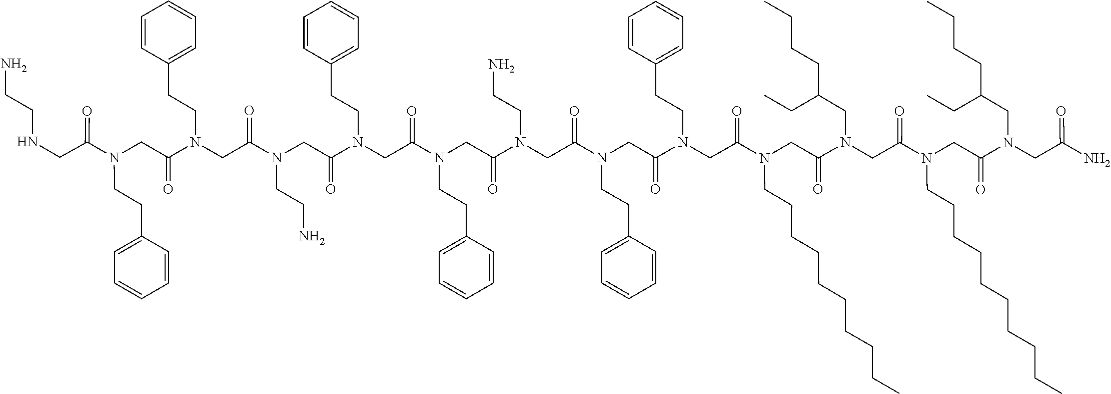 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 25 | 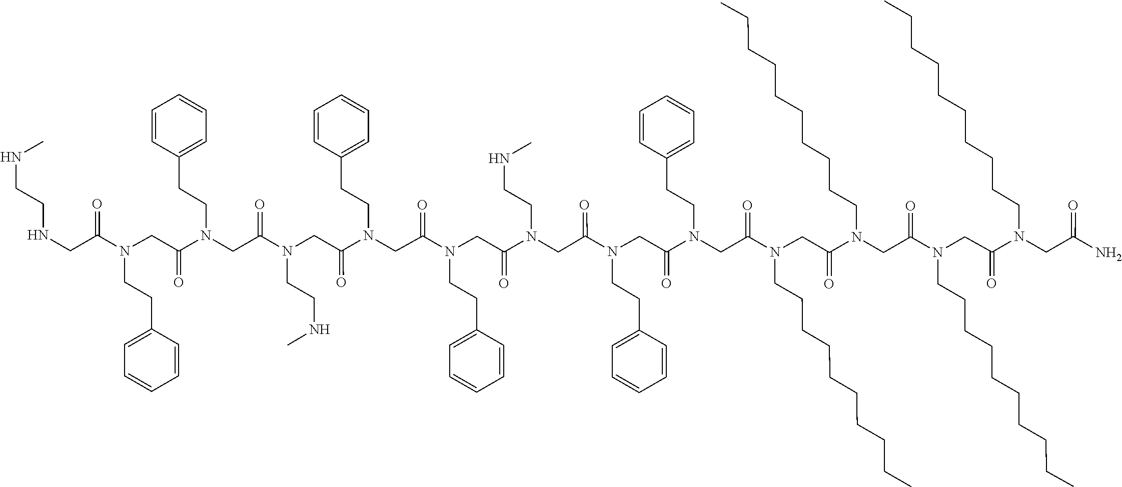 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CNCCNC] |
| 26 | 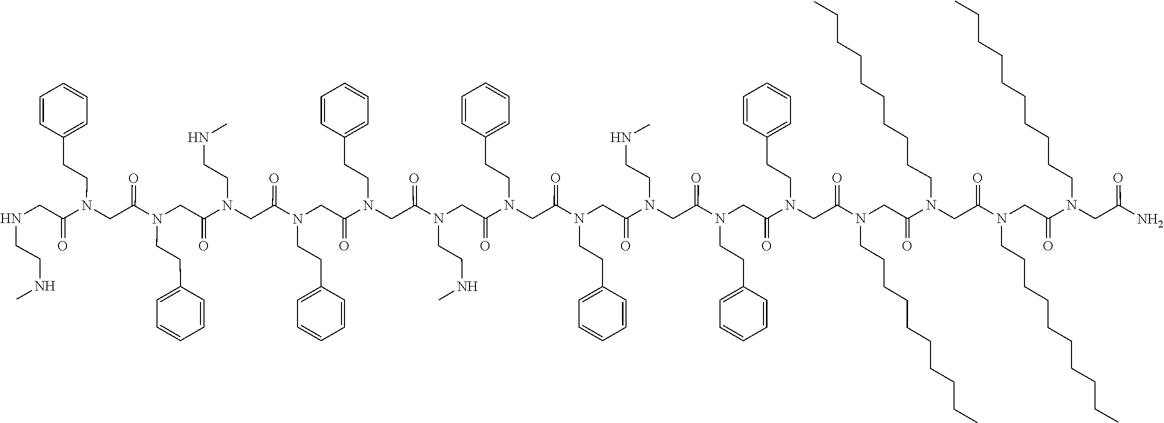 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CNCCNC] |
| 27 | 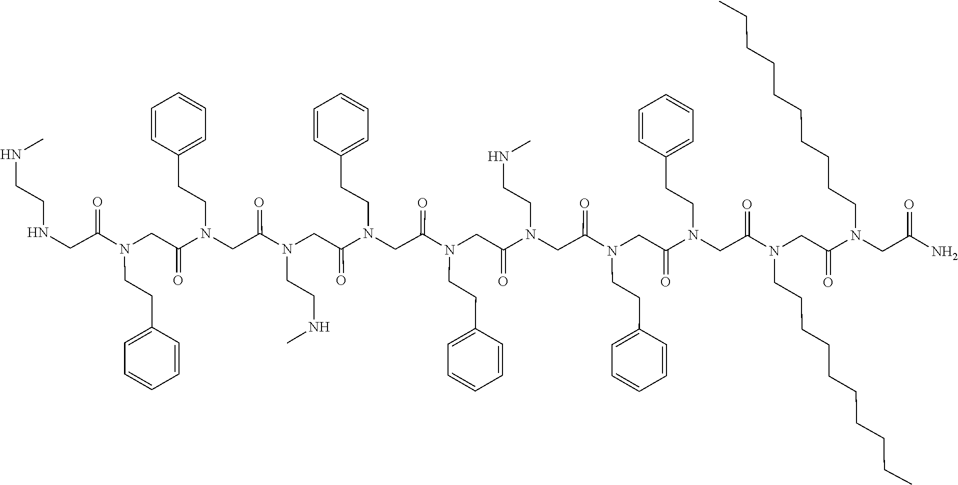 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CNCCNC] |
| 28 | 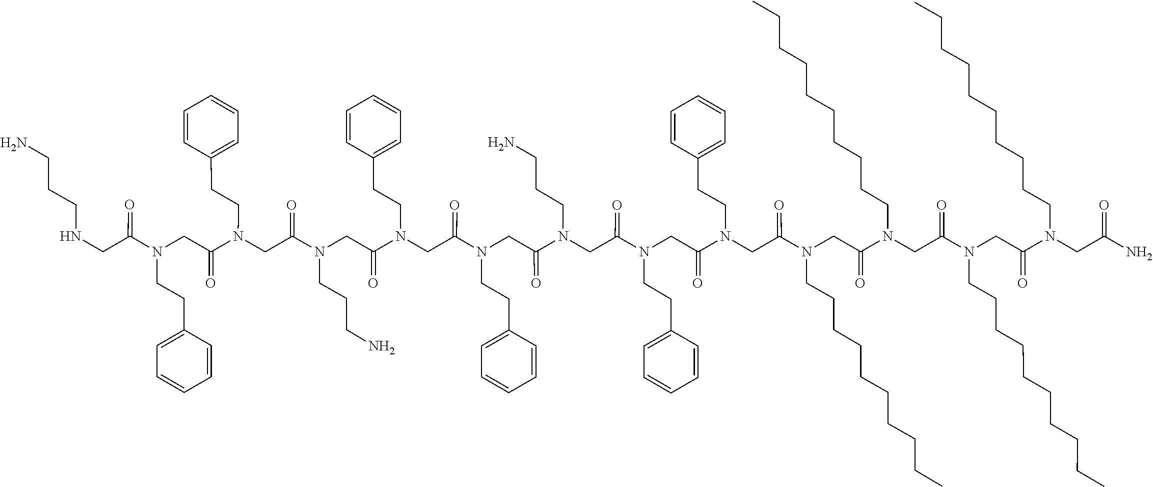 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CNCCCN] |
| 29 | 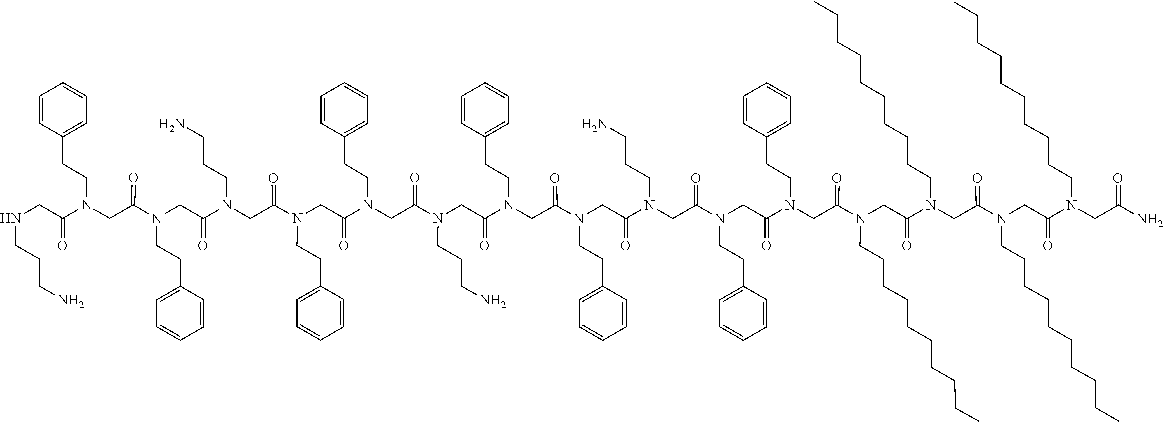 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CNCCCN] |
| 30 | 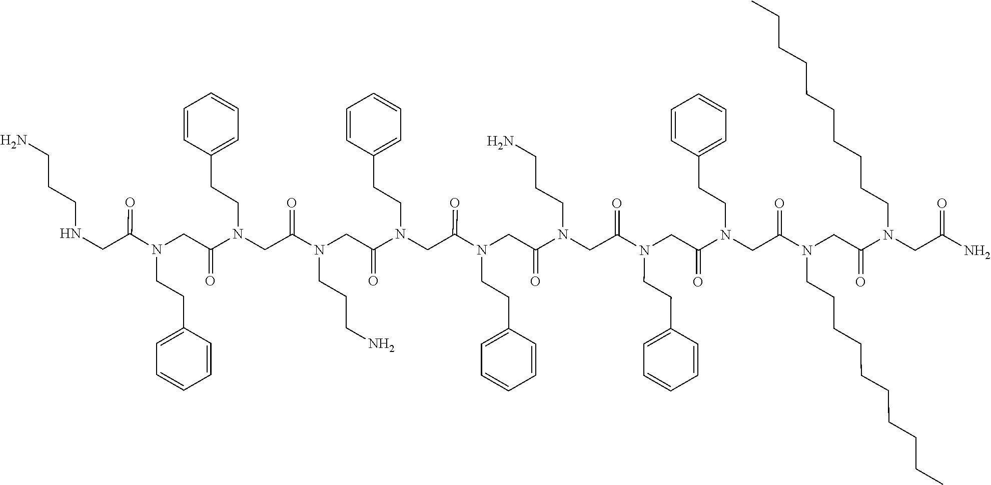 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CNCCCN] |
| 31 | 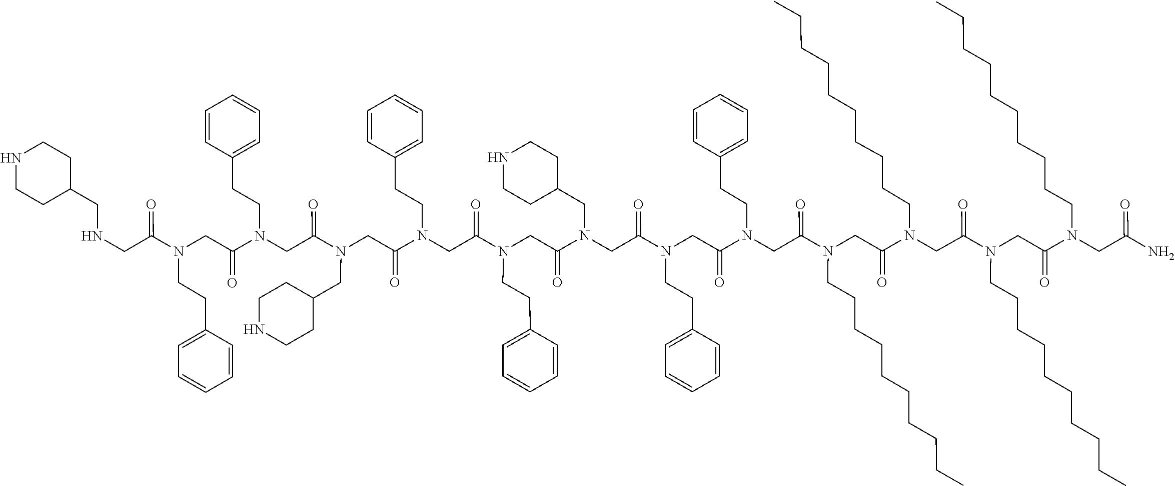 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CNCC1CCNCC1] |
| 32 | 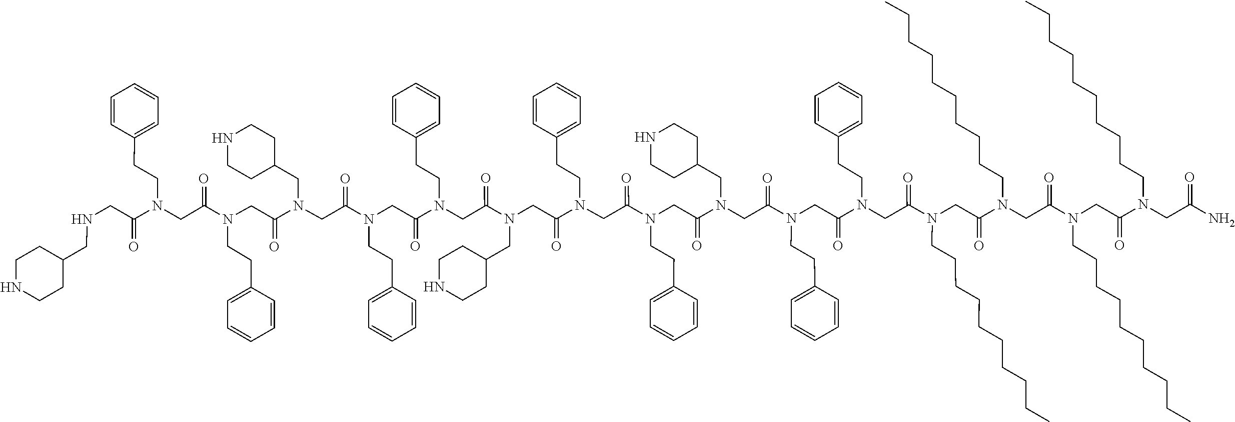 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CNCC1CCNCC1] |
| 33 | 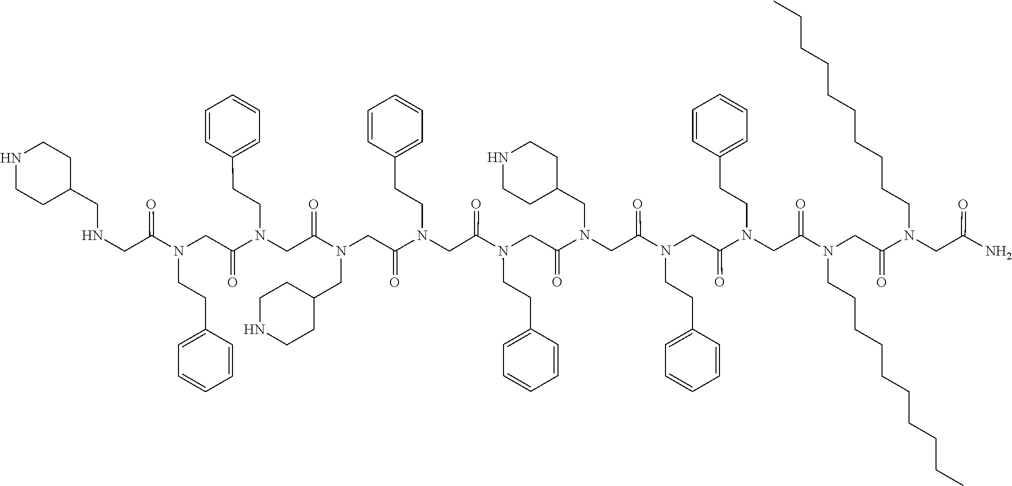 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CNCC1CCNCC1] |
| 34 | 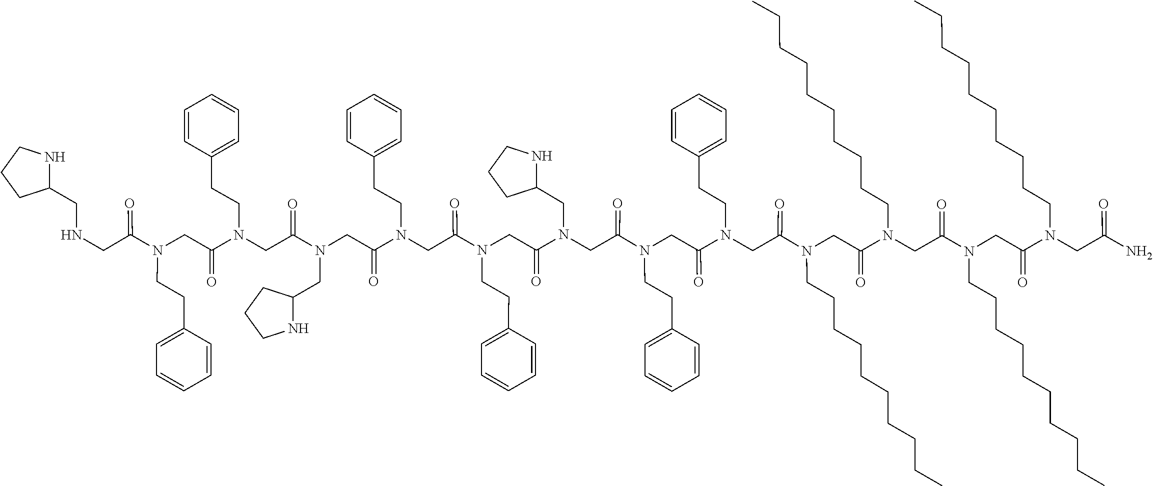 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CNCC1CCCN1] |
| 35 | 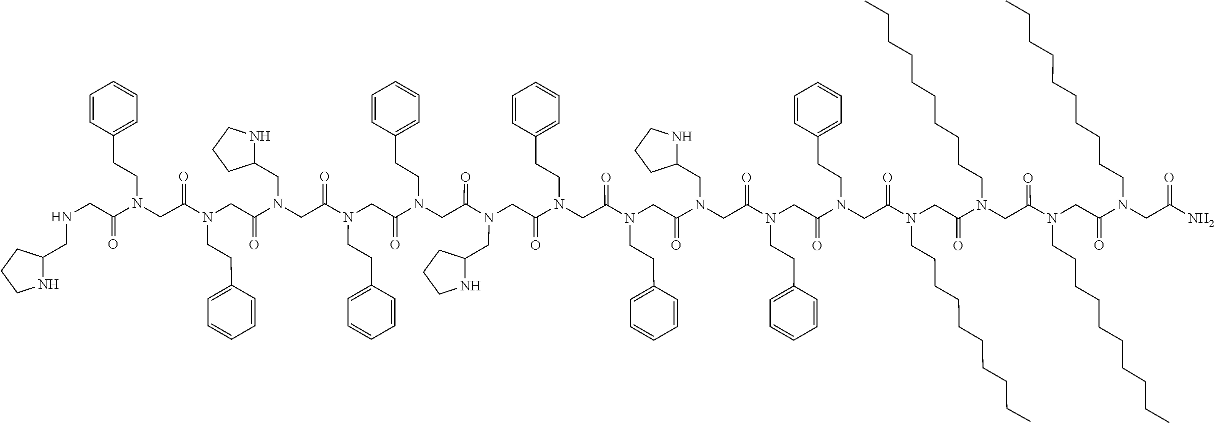 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CNCC1CCCN1] |
| 36 | 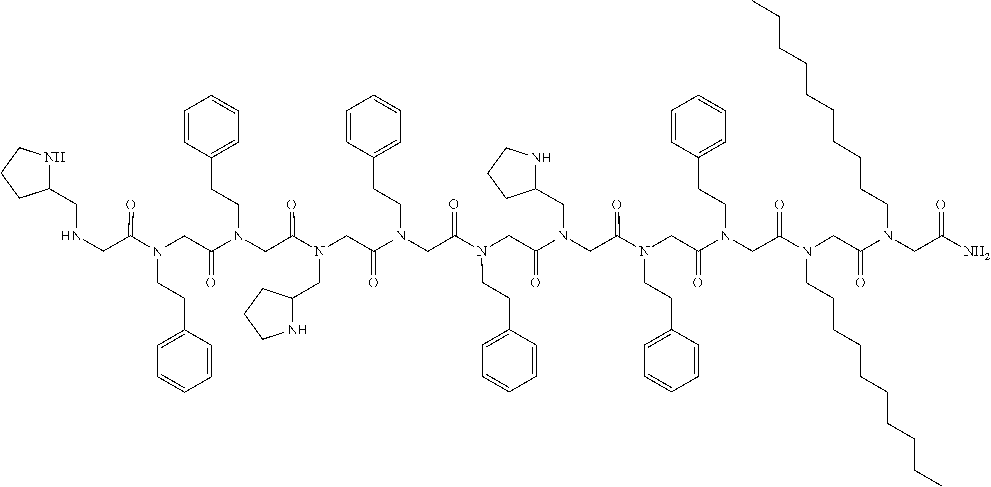 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CNCC1CCCN1] |
| 41 | 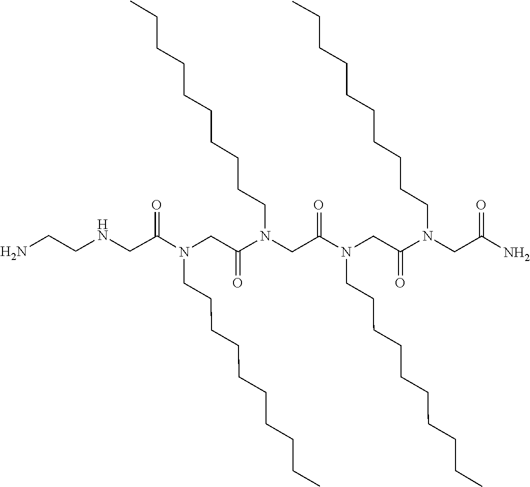 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCN] |
| 42 | 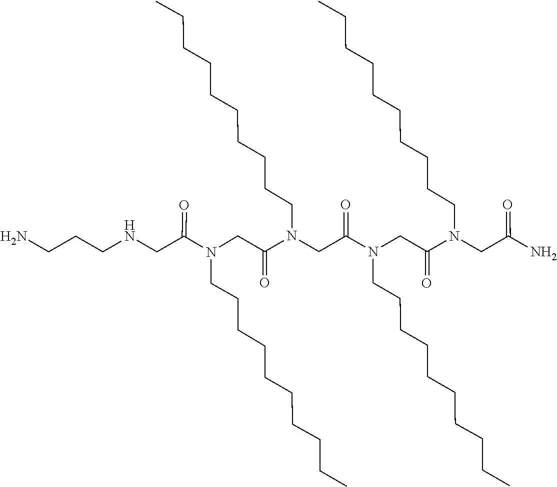 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCN] |
| 43 | 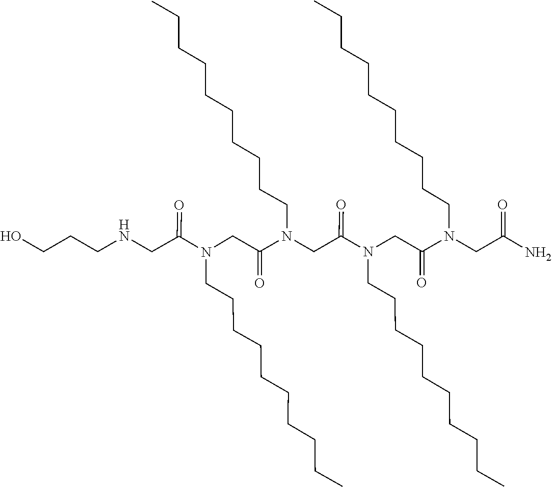 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCO] |
| 44 | 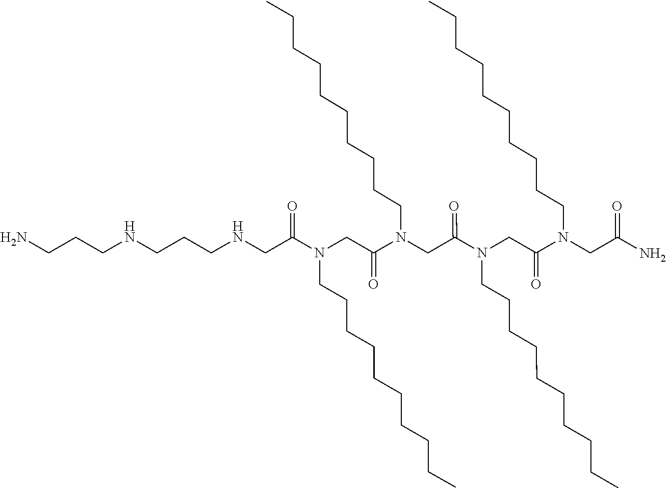 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCNCCCN] |
| 45 | 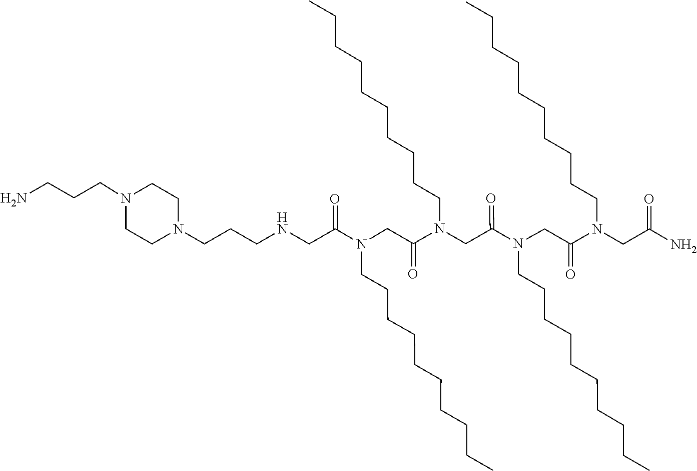 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCN1CCN(CCCN)CC1] |
| 46 | 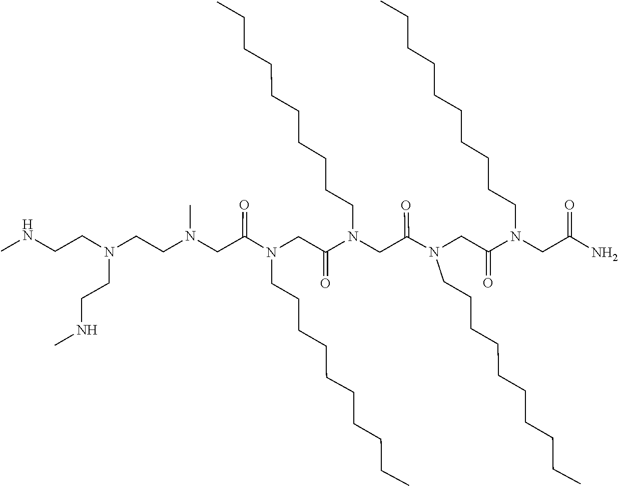 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(C)CCN(CCNC)CCNC] |
| 47 | 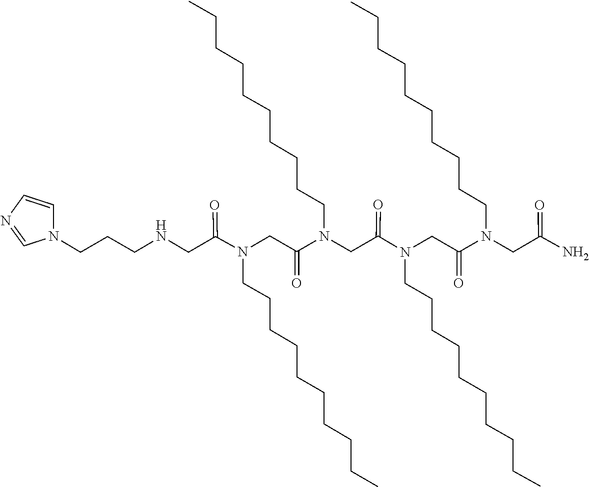 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCn1ccnc1] |
| 49 | 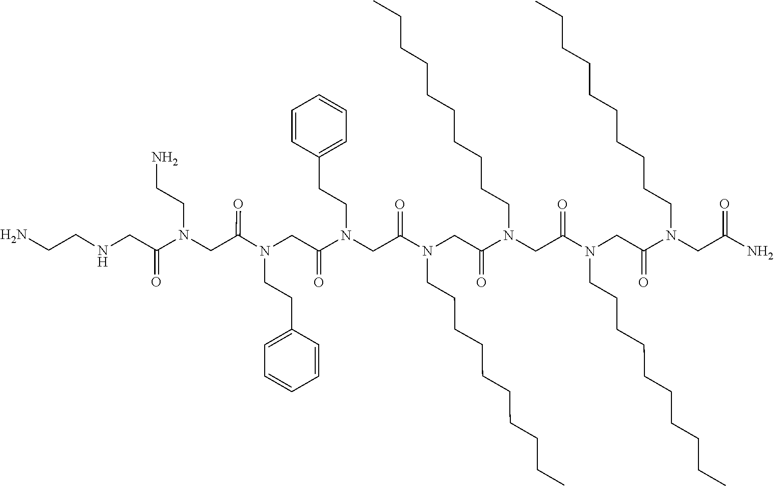 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCN] |
| 50 | 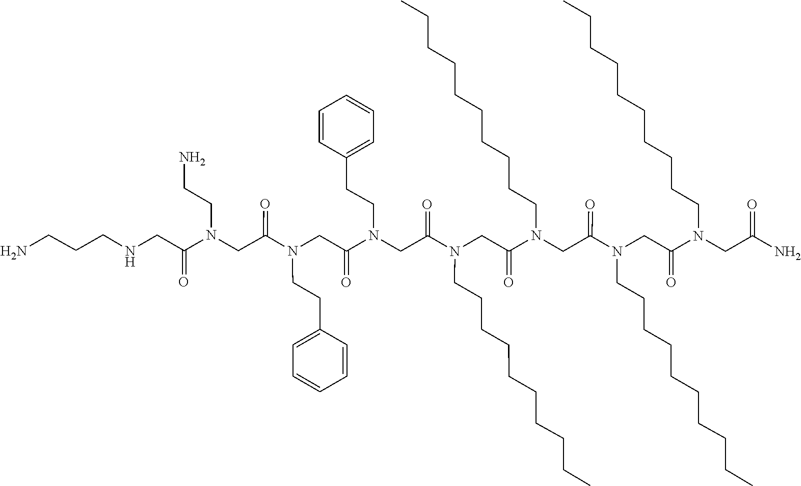 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCCN] |
| 51 | 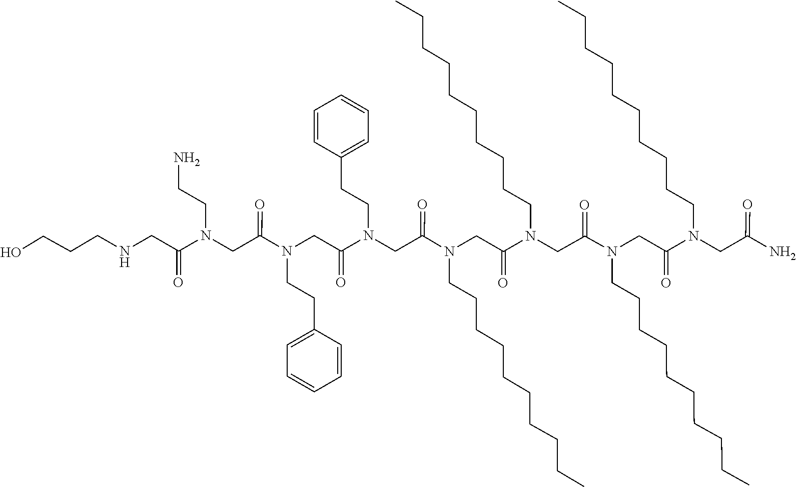 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCCO] |
| 52 | 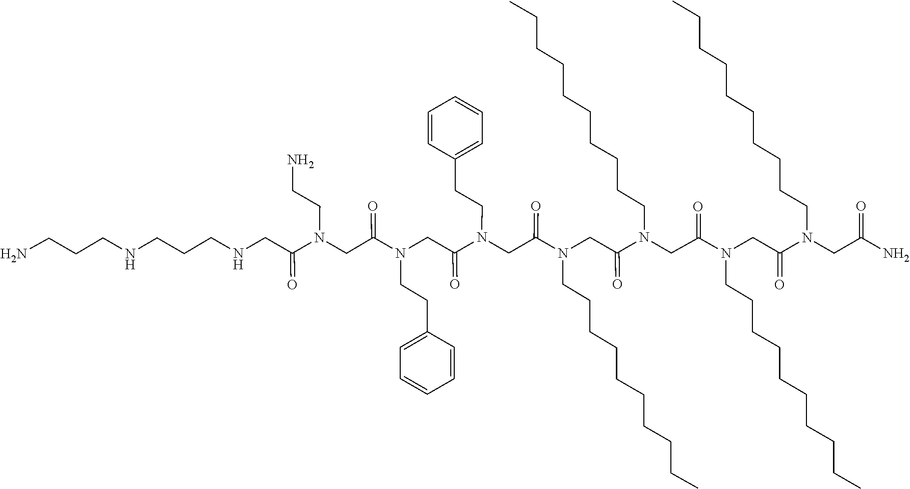 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCCNCCCN] |
| 53 | 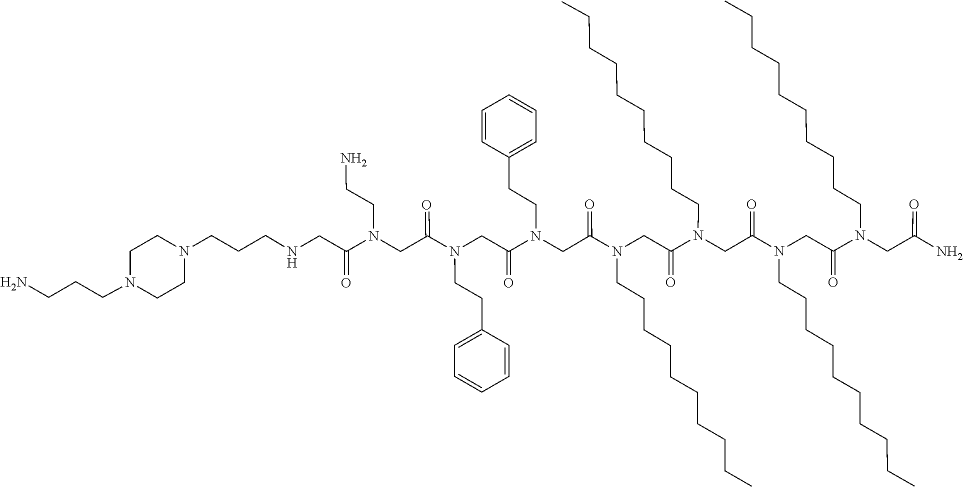 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCCN1CCN(CCCN)CC1] |
| 54 | 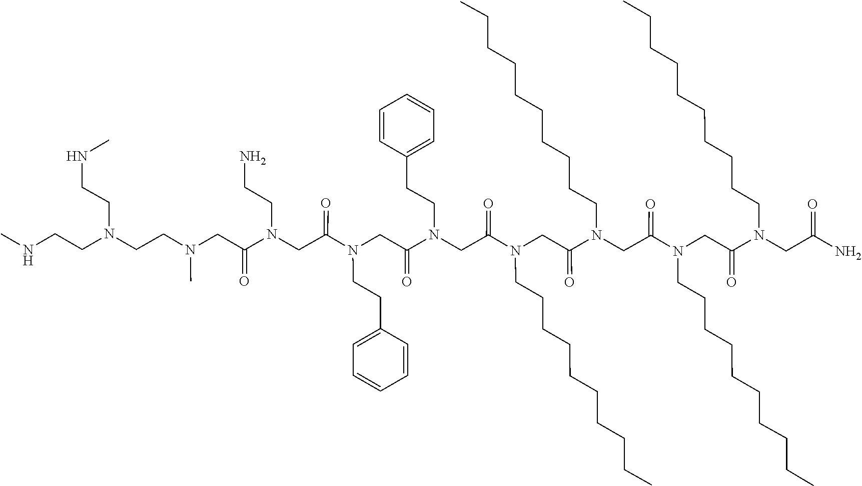 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(C)CCN(CCNC)CCNC] |
| 55 | 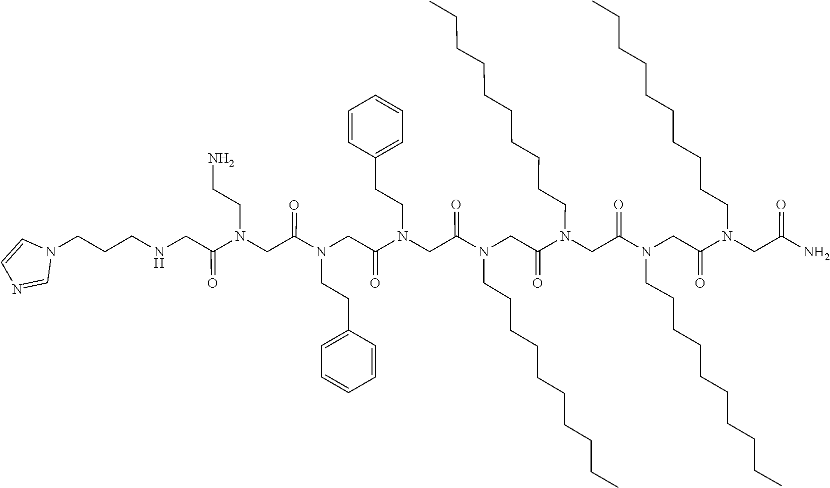 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCCn1ccnc1] |
| 57 | 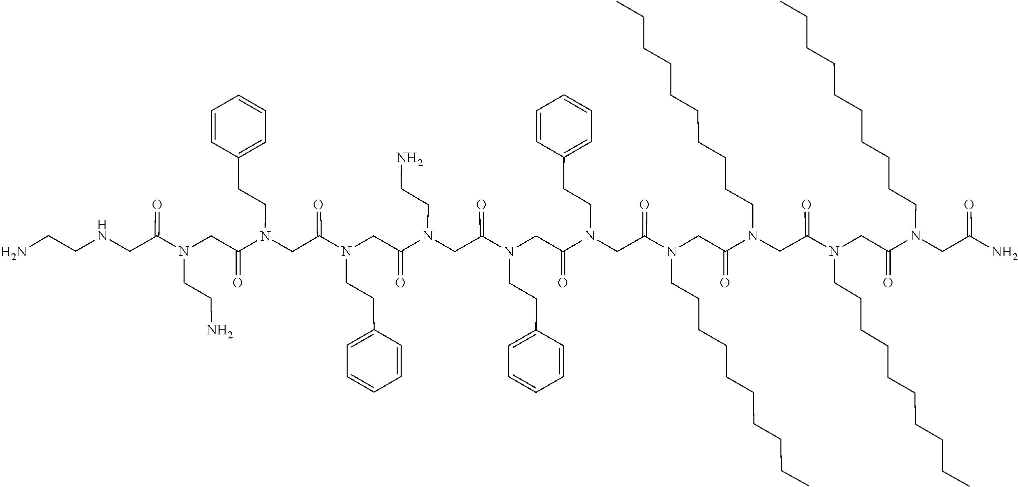 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCN] |
| 58 | 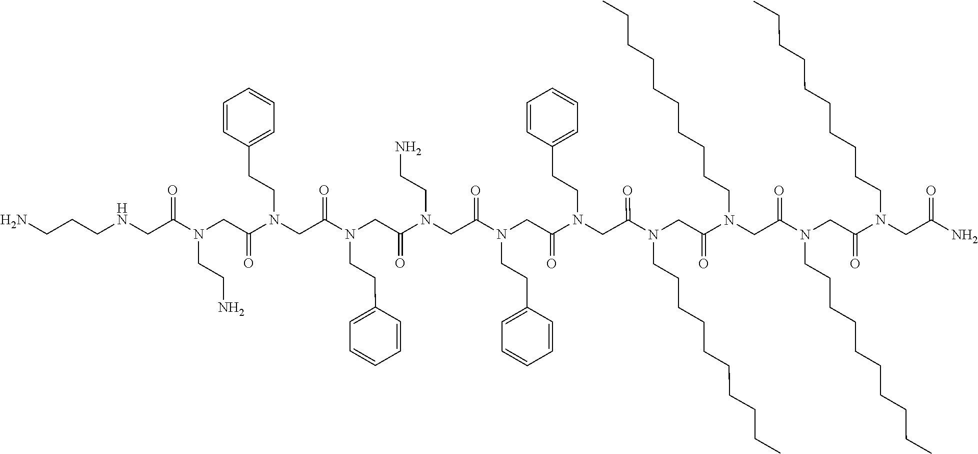 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCN] |
| 59 | 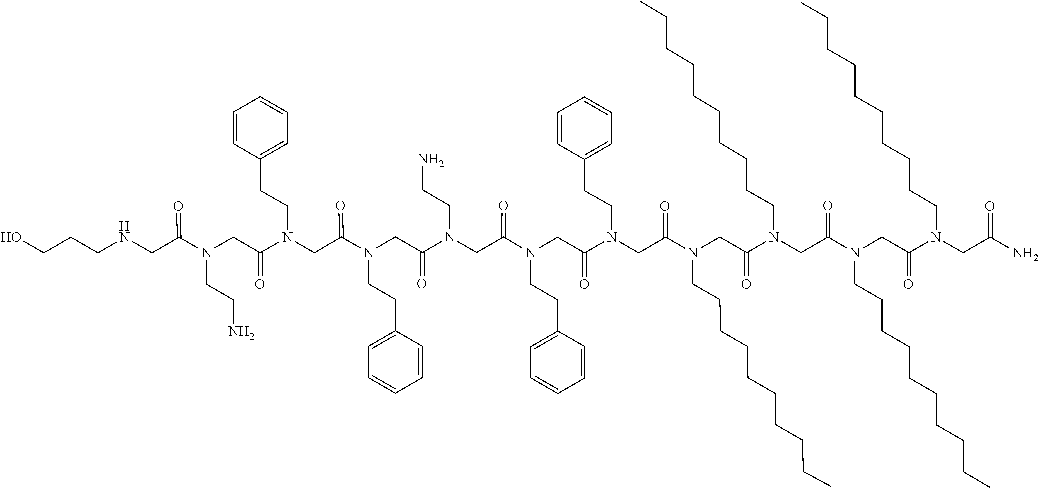 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCO] |
| 60 | 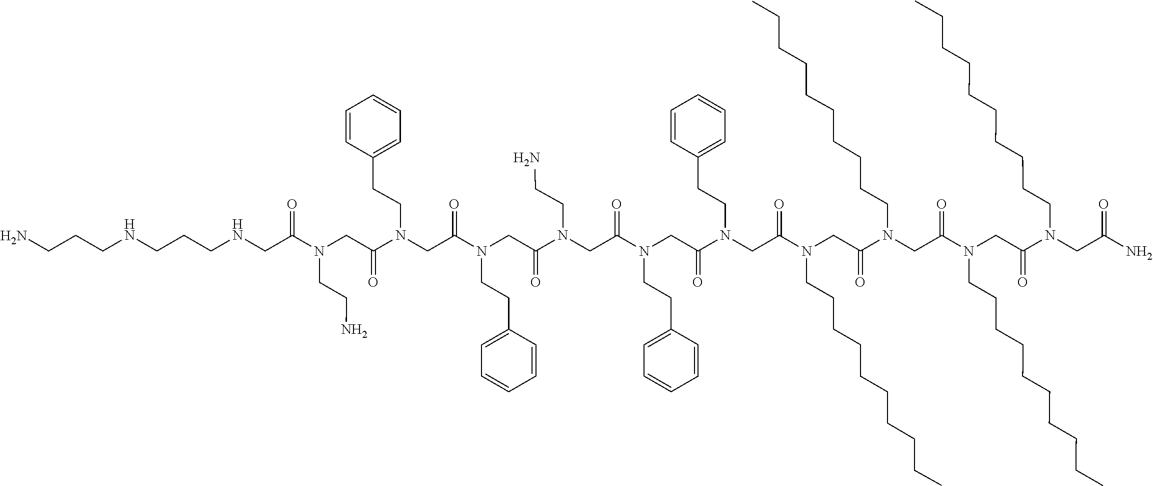 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCNCCCN] |
| 61 | 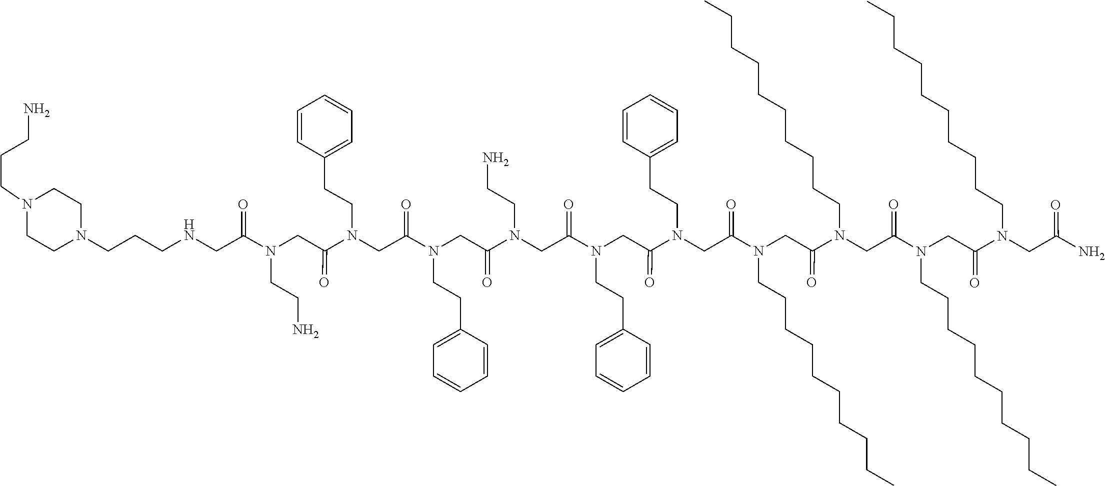 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCN1CCN(CCCN)CC1] |
| 62 | 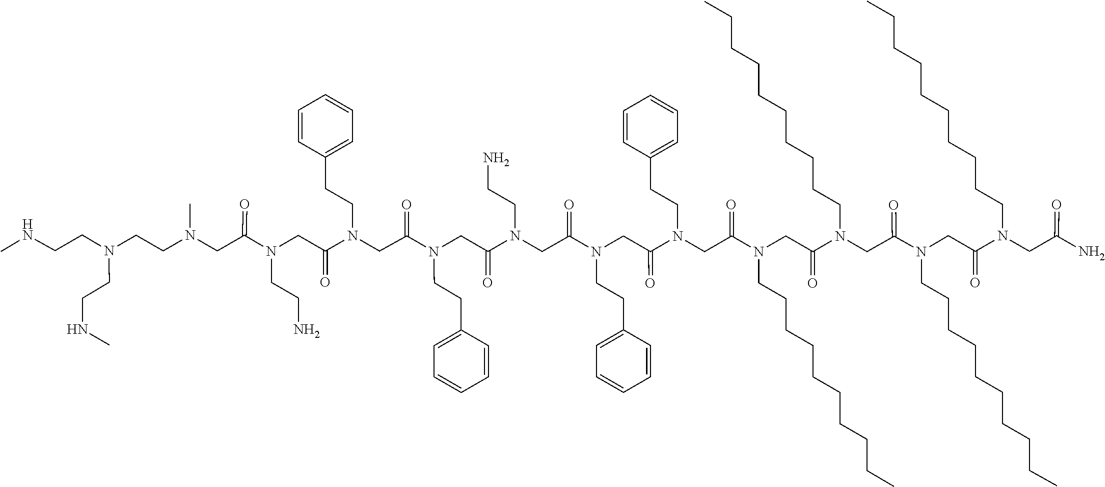 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(C)CCN(CCNC)CCNC] |
| 63 | 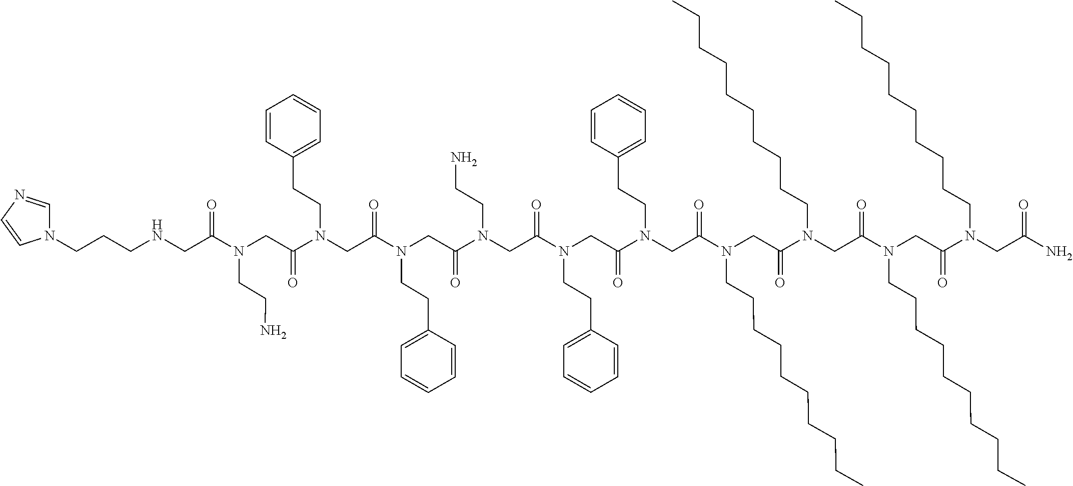 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCn1ccnc1] |
| 65 | 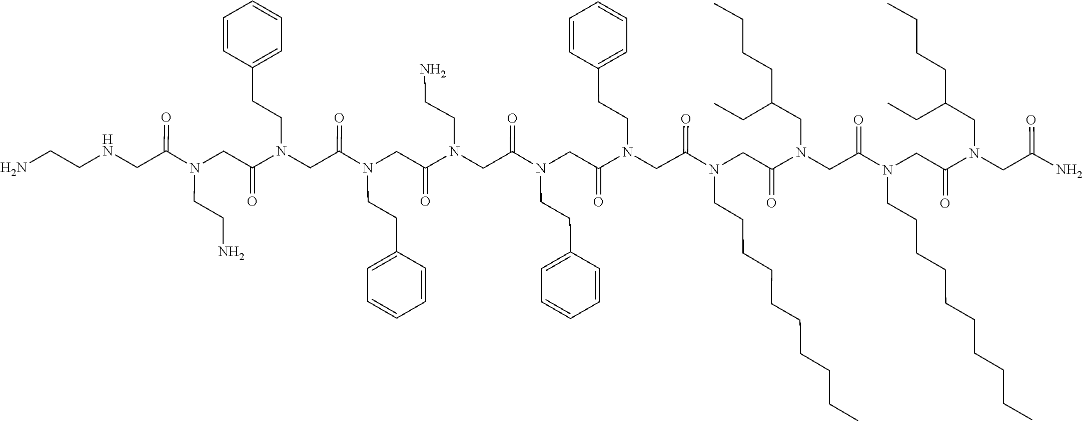 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCN] |
| 66 | 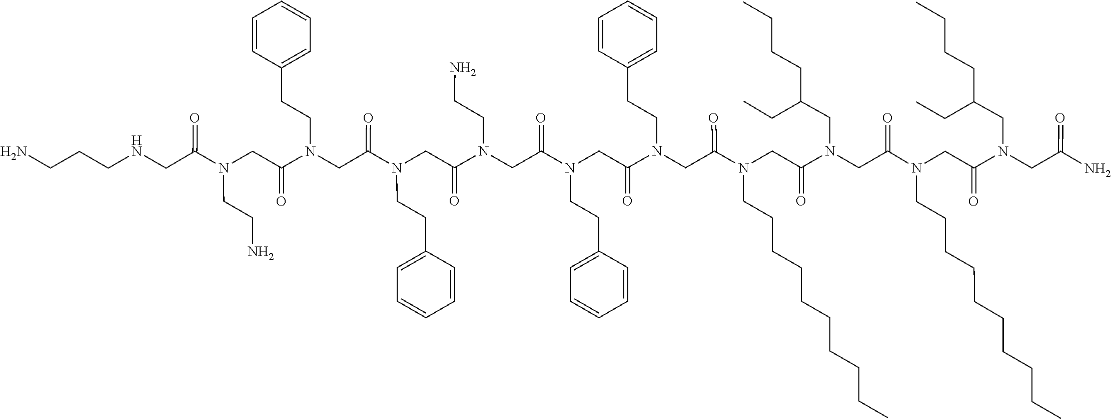 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCN] |
| 67 | 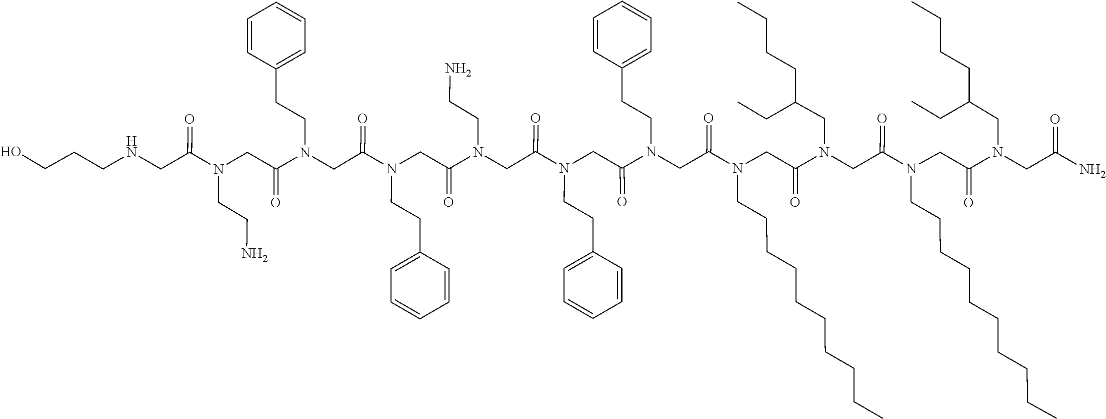 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCO] |
| 68 | 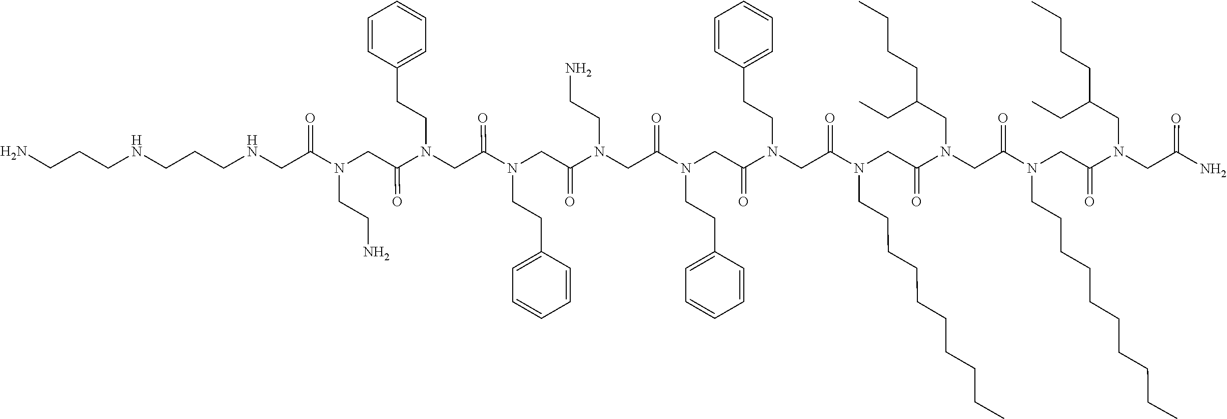 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCNCCCN] |
| 69 | 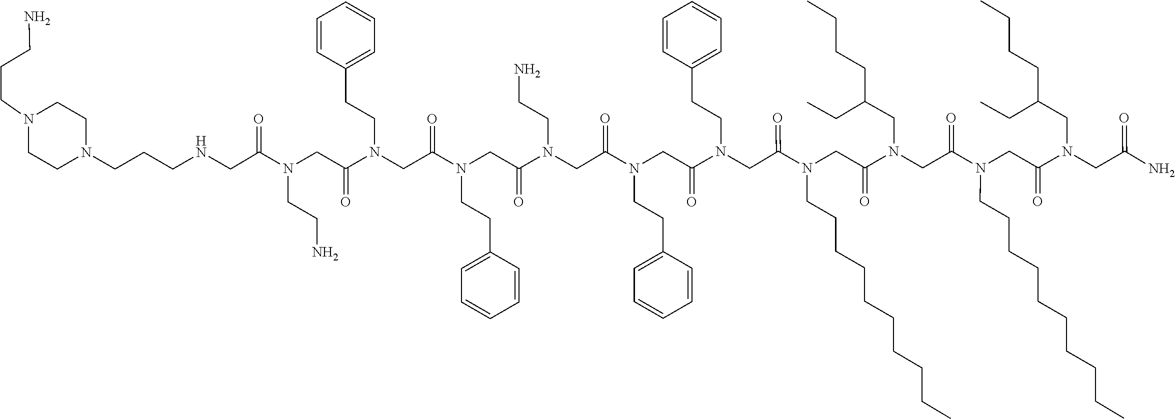 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCN1CCN(CCCN)CC1] |
| 70 | 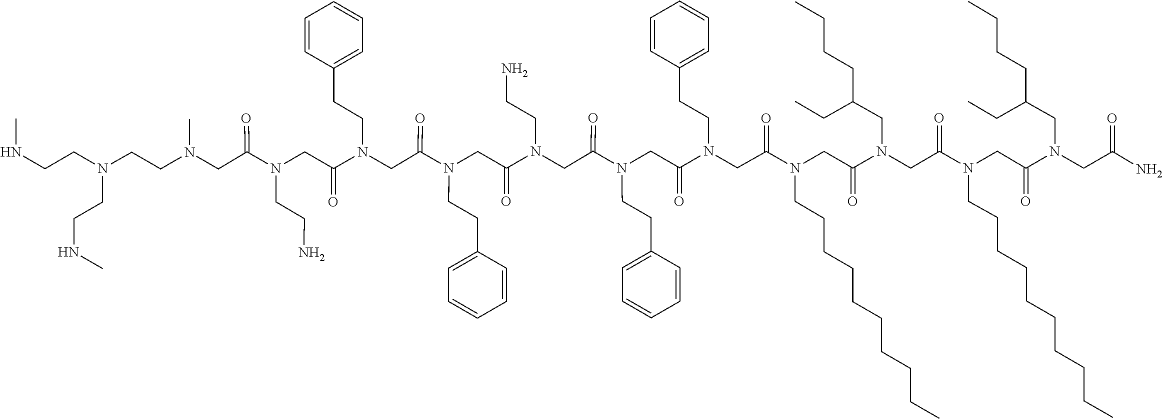 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(C)CCN(CCNC)CCNC] |
| 71 | 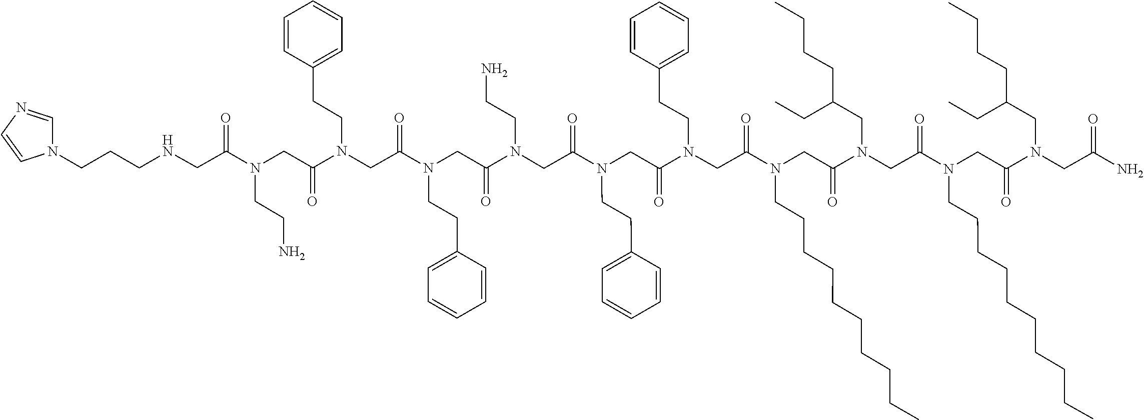 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCn1ccnc1] |
| 73 | 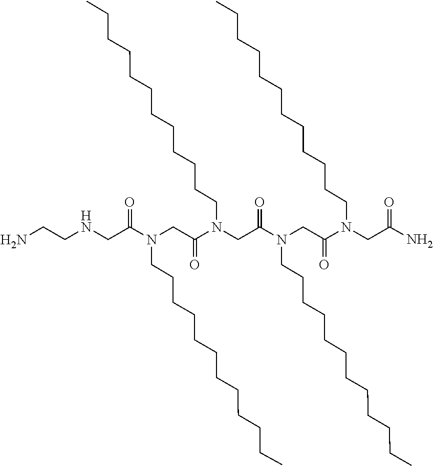 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCN] |
| 74 | 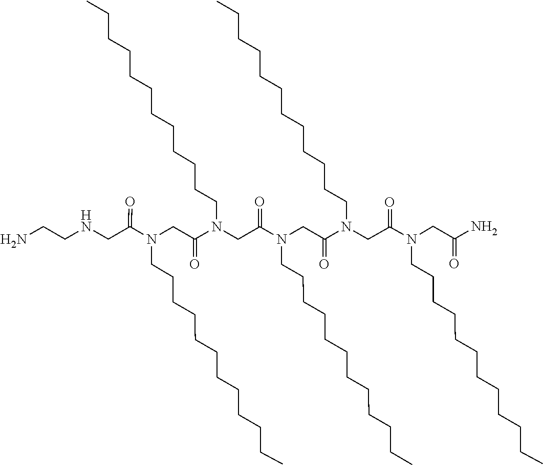 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CNCCN] |
| 75 | 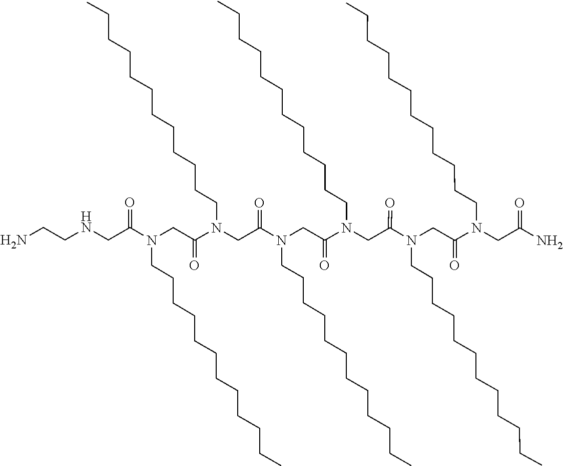 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CNCCN] |
| 76 | 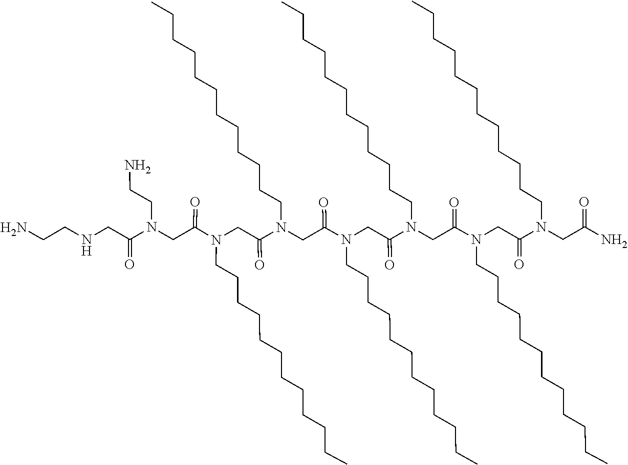 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CN(CCN)C(=O)CNCCN] |
| 77 | 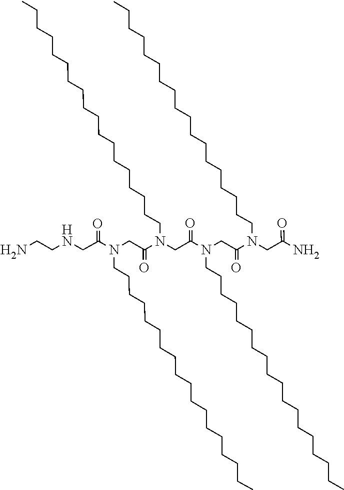 [SMILES: CCCCCCCCCCCCCCCCCCN(CC(N)=O)C(=O)CN(CCCCCCCCCCCCCCCCCC)C(=O)CN(CCCCCCCCCCCCCCCCCC)C(=O)CN(CCCCCCCCCCCCCCCCCC)C(=O)CNCCN] |
| 78 | 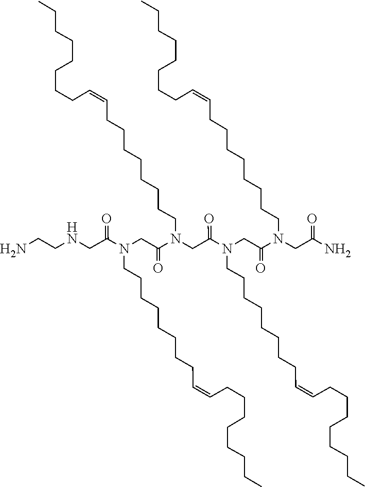 [SMILES: CCCCCCCC/C=C\CCCCCCCCN(CC(N)=O)C(=O)CN(CCCCCCCC/C=C\CCCCCCCC)C(=O)CN(CCCCCCCC/C=C\CCCCCCCC)C(=O)CN(CCCCCCCC/C=C\CCCCCCCC)C(=O)CNCCN] |
| 79 | 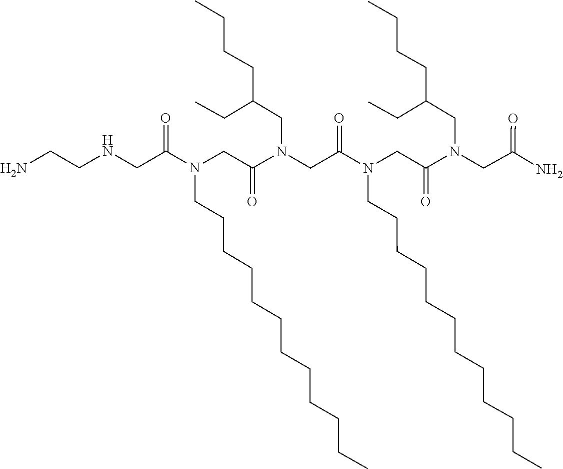 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCN] |
| 80 | 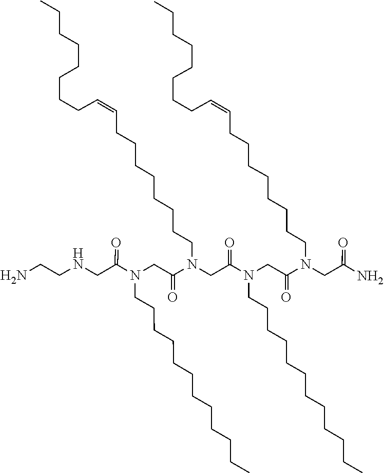 [SMILES: CCCCCCCC/C=C\CCCCCCCCN(CC(N)=O)C(=O)CN(CCCCCCCCCCCC)C(=O)CN(CCCCCCCC/C=C\CCCCCCCC)C(=O)CN(CCCCCCCCCCCC)C(=O)CNCCN] |
| 81 | 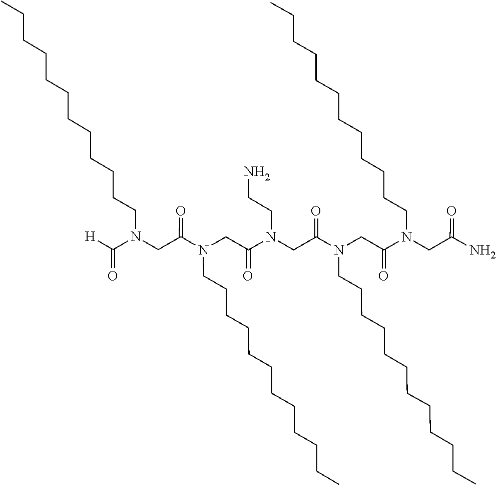 [SMILES: CCCCCCCCCCCCN(C=O)CC(=O)N(CCCCCCCCCCCC)CC(=O)N(CCN)CC(=O)N(CCCCCCCCCCCC)CC(=O)N(CCCCCCCCCCCC)CC(N)=O] |
| 82 | 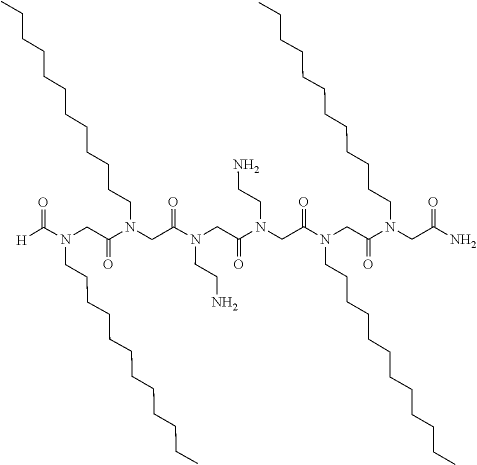 [SMILES: CCCCCCCCCCCCN(C=O)CC(=O)N(CCCCCCCCCCCC)CC(=O)N(CCN)CC(=O)N(CCN)CC(=O)N(CCCCCCCCCCCC)CC(=O)N(CCCCCCCCCCCC)CC(N)=O] |
| 83 | 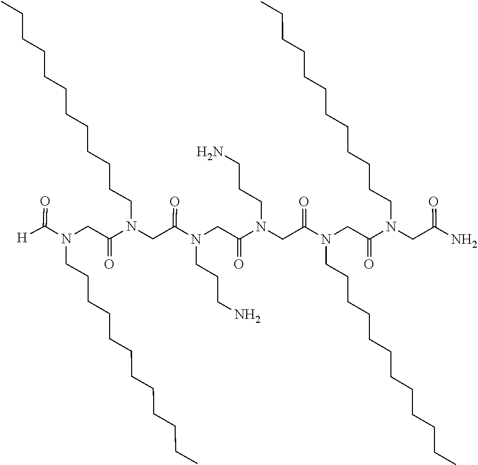 [SMILES: CCCCCCCCCCCCN(C=O)CC(=O)N(CCCCCCCCCCCC)CC(=O)N(CCCN)CC(=O)N(CCCN)CC(=O)N(CCCCCCCCCCCC)CC(=O)N(CCCCCCCCCCCC)CC(N)=O] |
| 84 | 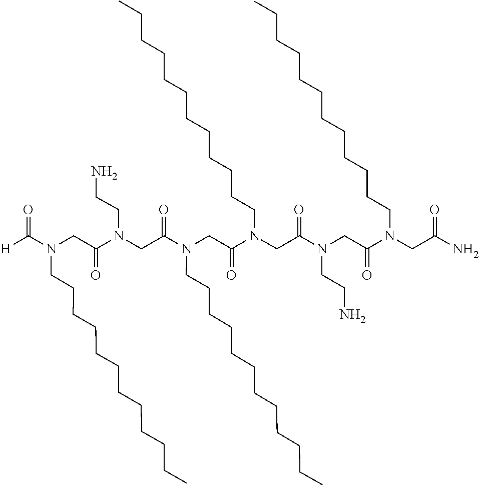 [SMILES: CCCCCCCCCCCCN(C=O)CC(=O)N(CCN)CC(=O)N(CCCCCCCCCCCC)CC(=O)N(CCCCCCCCCCCC)CC(=O)N(CCN)CC(=O)N(CCCCCCCCCCCC)CC(N)=O] |
| 85 | 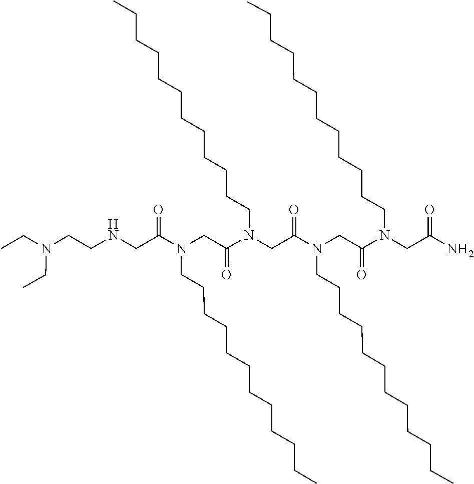 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCN(CC)CC] |
| 86 | 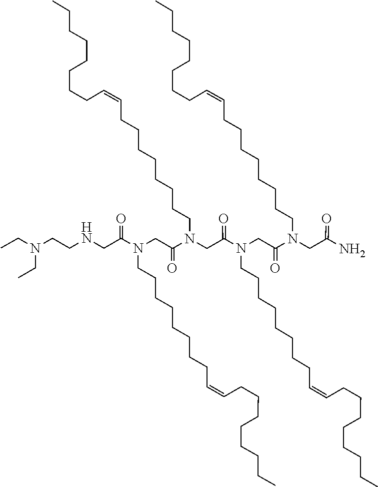 [SMILES: CCCCCCCC/C=C\CCCCCCCCN(CC(N)=O)C(=O)CN(CCCCCCCC/C=C\CCCCCCCC)C(=O)CN(CCCCCCCC/C=C\CCCCCCCC)C(=O)CN(CCCCCCCC/C=C\CCCCCCCC)C(=O)CNCCN(CC)CC] |
| 87 | 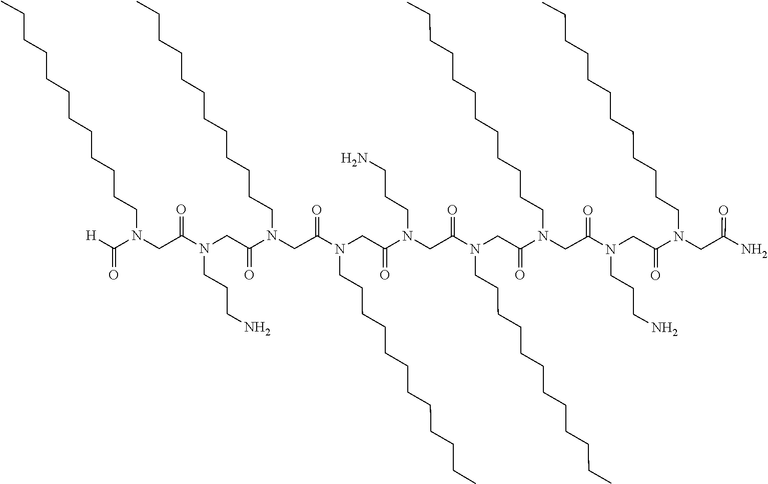 [SMILES: CCCCCCCCCCCCN(C=O)CC(=O)N(CCCN)CC(=O)N(CCCCCCCCCCCC)CC(=O)N(CCCCCCCCCCCC)CC(=O)N(CCCN)CC(=O)N(CCCCCCCCCCCC)CC(=O)N(CCCCCCCCCCCC)CC(=O)N(CCCN)CC(=O)N(CCCCCCCCCCCC)CC(N)=O] |
| 88 | 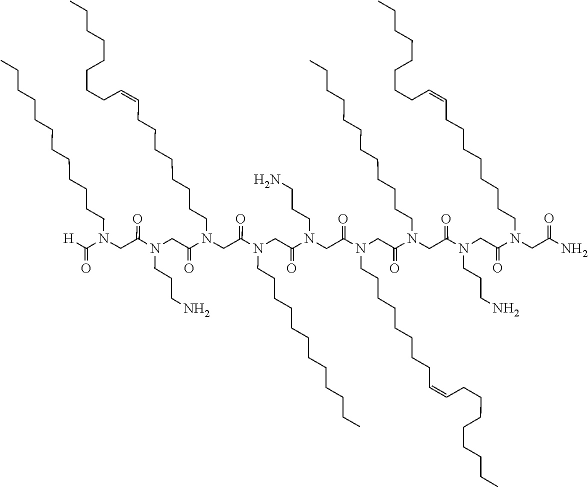 [SMILES: CCCCCCCC/C=C\CCCCCCCCN(CC(N)=O)C(=O)CN(CCCN)C(=O)CN(CCCCCCCCCCCC)C(=O)CN(CCCCCCCC/C=C\CCCCCCCC)C(=O)CN(CCCN)C(=O)CN(CCCCCCCCCCCC)C(=O)CN(CCCCCCCC/C=C\CCCCCCCC)C(=O)CN(CCCN)C(=O)CN(C=O)CCCCCCCCCCCC] |
| 89 | 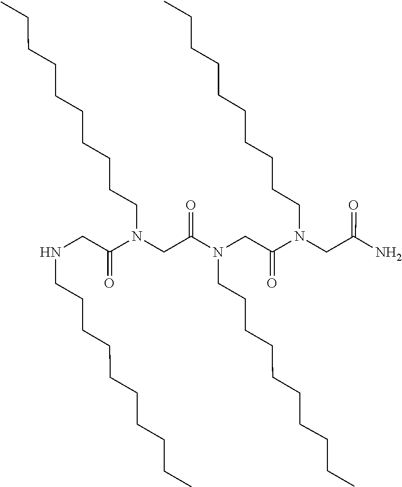 [SMILES: CCCCCCCCCCNCC(=O)N(CCCCCCCCCC)CC(=O)N(CCCCCCCCCC)CC(=O)N(CCCCCCCCCC)CC(N)=O] |
| 91 | 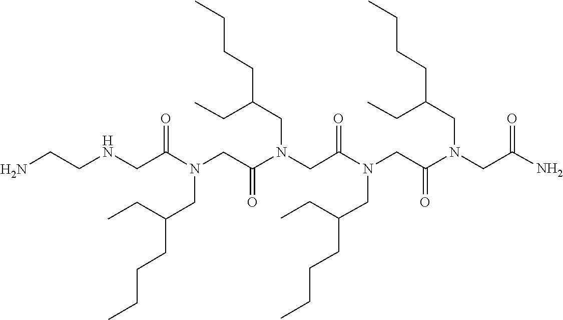 [SMILES: CCCCC(CC)CN(CC(N)=O)C(=O)CN(CC(CC)CCCC)C(=O)CN(CC(CC)CCCC)C(=O)CN(CC(CC)CCCC)C(=O)CNCCN] |
| 92 | 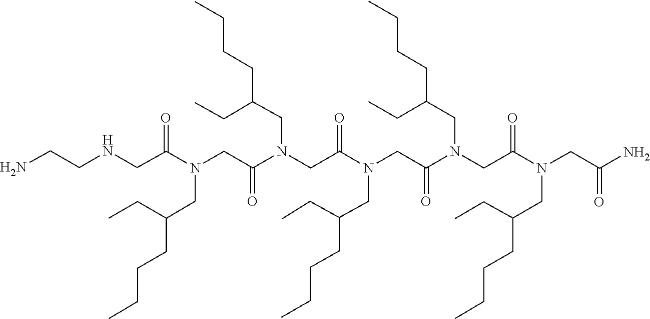 [SMILES: CCCCC(CC)CN(CC(N)=O)C(=O)CN(CC(CC)CCCC)C(=O)CN(CC(CC)CCCC)C(=O)CN(CC(CC)CCCC)C(=O)CN(CC(CC)CCCC)C(=O)CNCCN] |
| 93 | 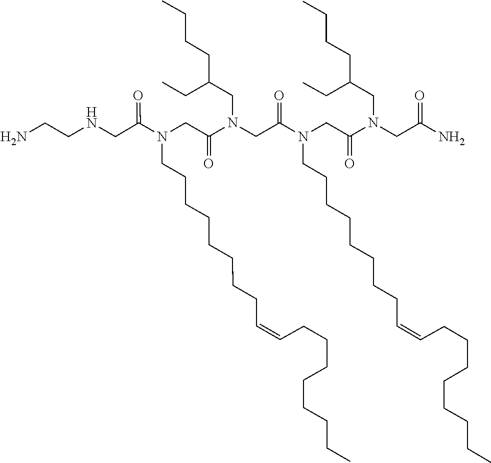 [SMILES: CCCCCCCC/C=C\CCCCCCCCN(CC(=O)N(CC(=O)N(CCCCCCCC/C=C\CCCCCCCC)CC(=O)N(CC(N)=O)CC(CC)CCCC)CC(CC)CCCC)C(=O)CNCCN] |
| 94 | 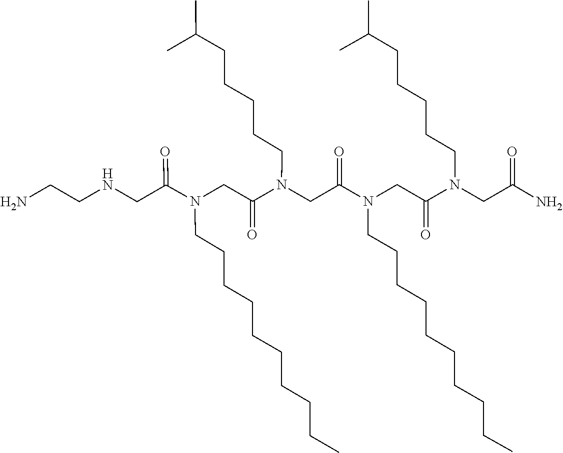 [SMILES: CCCCCCCCCCN(CC(=O)N(CCCCCC(C)C)CC(=O)N(CCCCCCCCCC)CC(=O)N(CCCCCC(C)C)CC(N)=O)C(=O)CNCCN] |
| 95 | 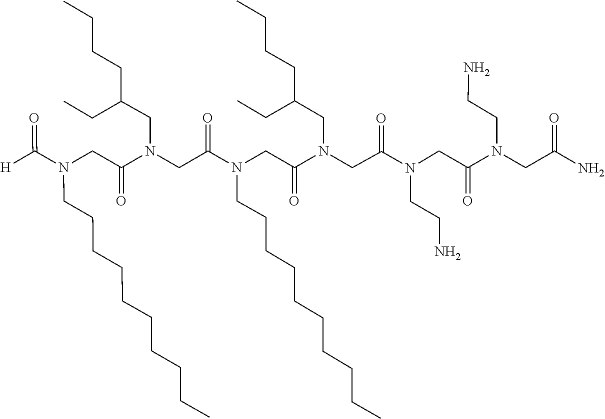 [SMILES: CCCCCCCCCCN(C=O)CC(=O)N(CC(=O)N(CCCCCCCCCC)CC(=O)N(CC(=O)N(CCN)CC(=O)N(CCN)CC(N)=O)CC(CC)CCCC)CC(CC)CCCC] |
| 96 | 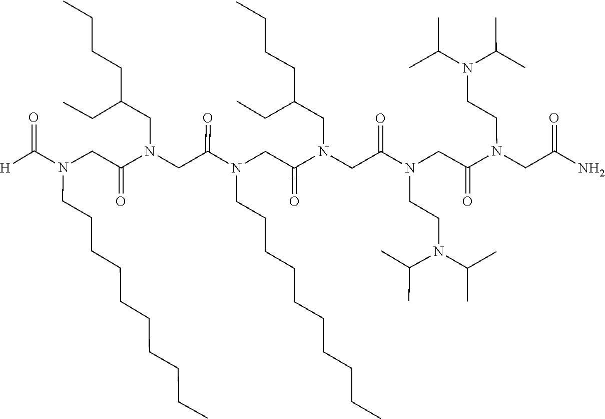 [SMILES: CCCCCCCCCCN(C=O)CC(=O)N(CC(=O)N(CCCCCCCCCC)CC(=O)N(CC(=O)N(CCN(C(C)C)C(C)C)CC(=O)N(CCN(C(C)C)C(C)C)CC(N)=O)CC(CC)CCCC)CC(CC)CCCC] |
| 97 | 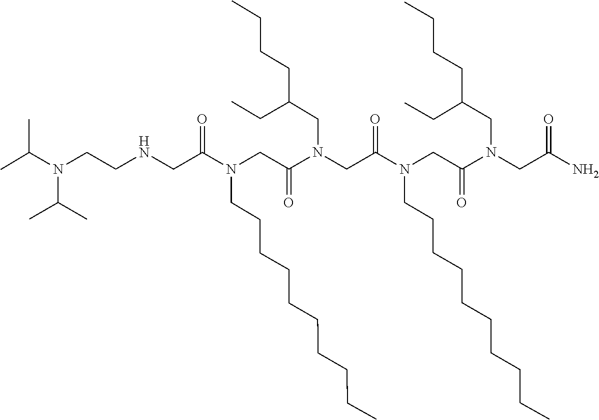 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CNCCN(C(C)C)C(C)C] |
| 98 | 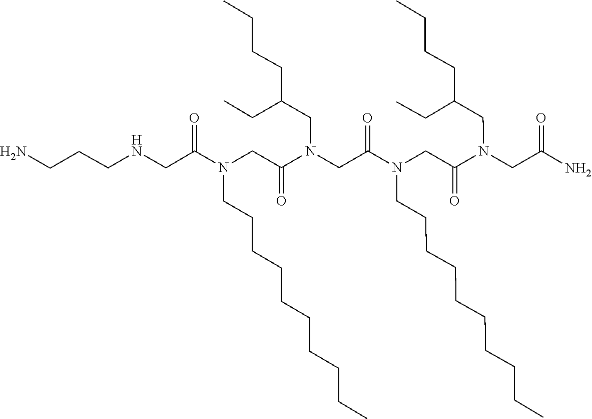 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CNCCCN] |
| 99 | 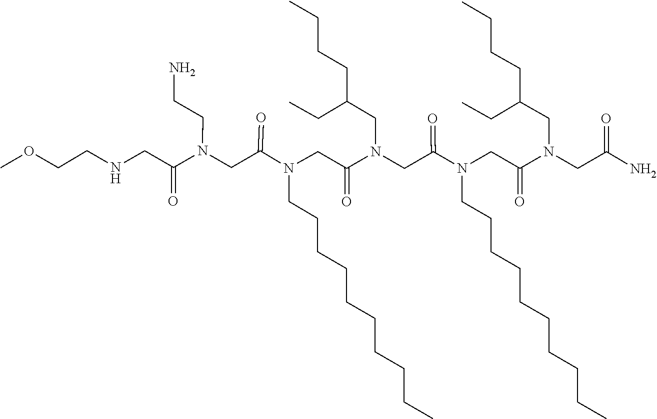 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCN)C(=O)CNCCOC] |
| 100 | 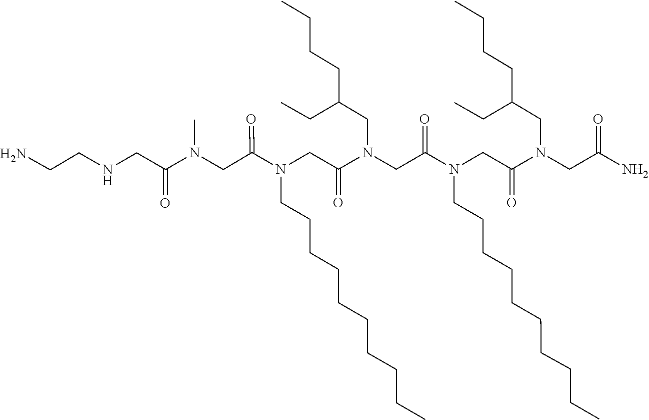 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(C)C(=O)CNCCN] |
| 101 | 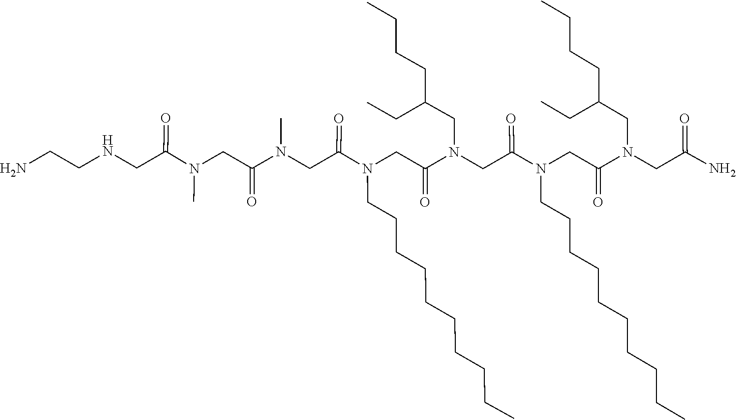 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(C)C(=O)CN(C)C(=O)CNCCN] |
| 102 | 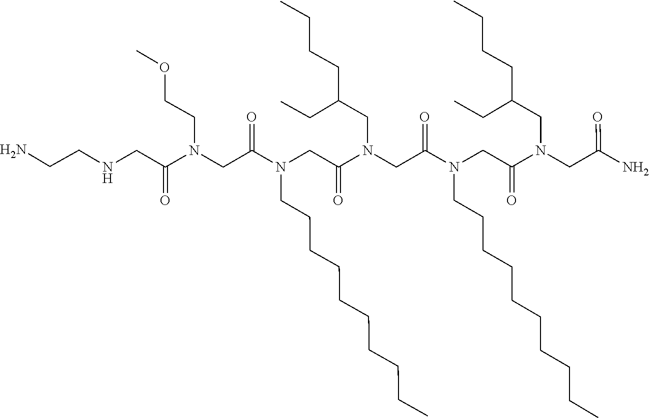 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCOC)C(=O)CNCCN] |
| 103 | 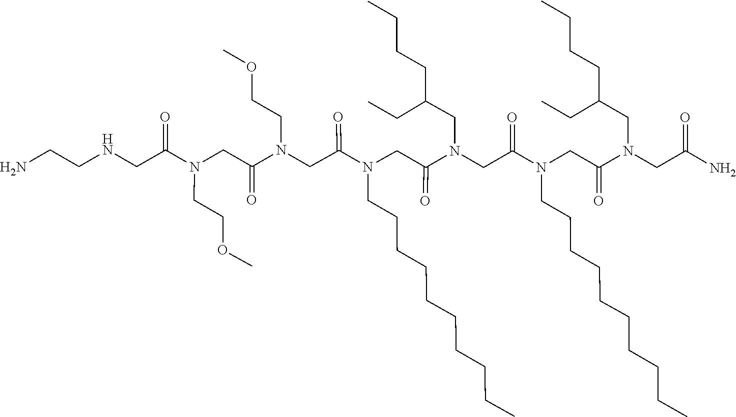 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCOC)C(=O)CN(CCOC)C(=O)CNCCN] |
| 111 | 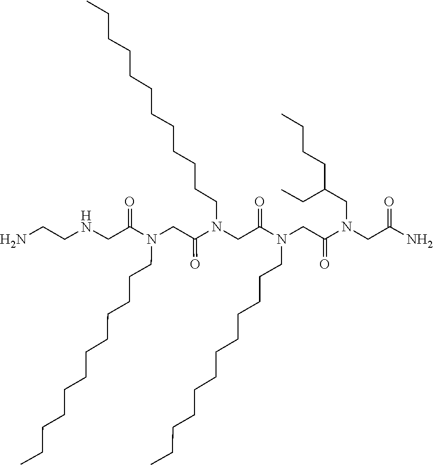 [SMILES: CCCCCCCCCCCCN(CC(=O)N(CCCCCCCCCCCC)CC(=O)N(CCCCCCCCCCCC)CC(=O)N(CC(N)=O)CC(CC)CCCC)C(=O)CNCCN] |
| 112 | 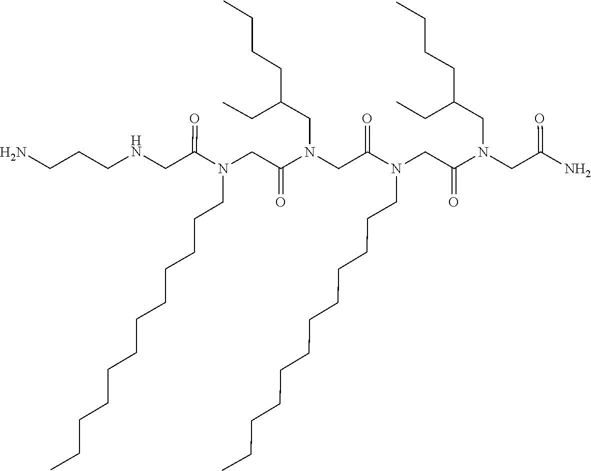 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCCN] |
| 113 | 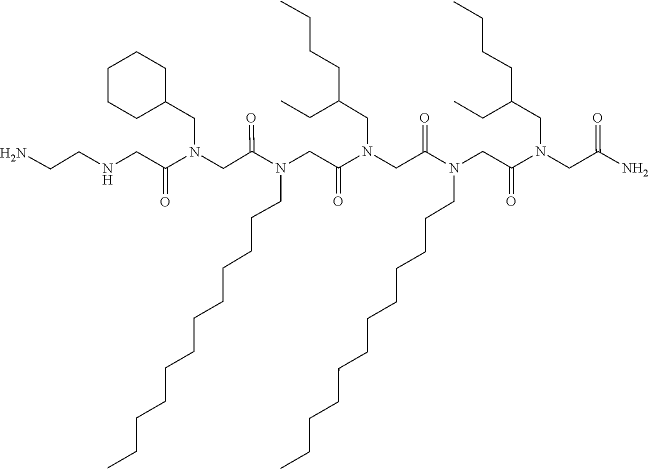 [SMILES: CCCCCCCCCCCCN(CC(=O)N(CC(N)=O)CC(CC)CCCC)C(=O)CN(CC(CC)CCCC)C(=O)CN(CCCCCCCCCCCC)C(=O)CN(CC1CCCCC1)C(=O)CNCCN] |
| 114 | 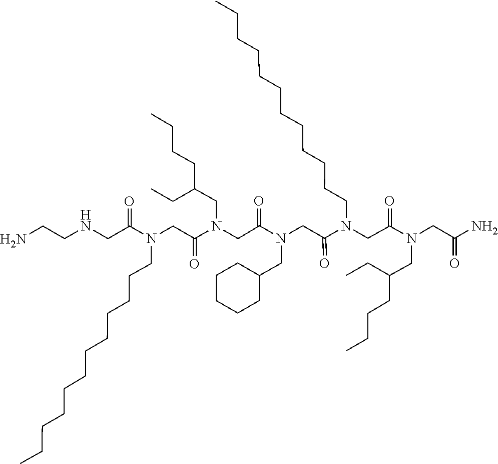 [SMILES: CCCCCCCCCCCCN(CC(=O)N(CC(=O)N(CC(=O)N(CCCCCCCCCCCC)CC(=O)N(CC(N)=O)CC(CC)CCCC)CC1CCCCC1)CC(CC)CCCC)C(=O)CNCCN] |
| 115 | 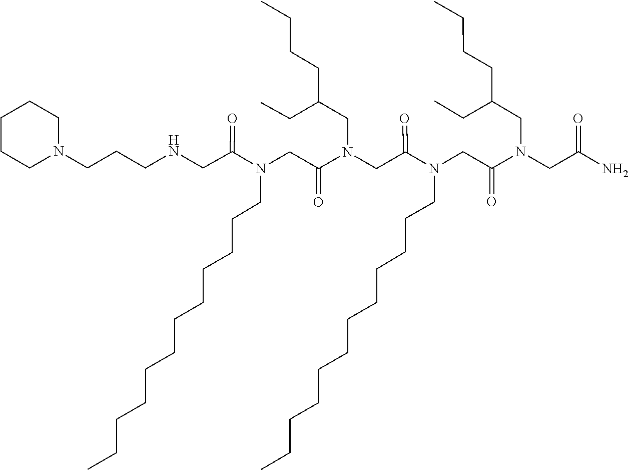 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCCN1CCCCC1] |
| 116 | 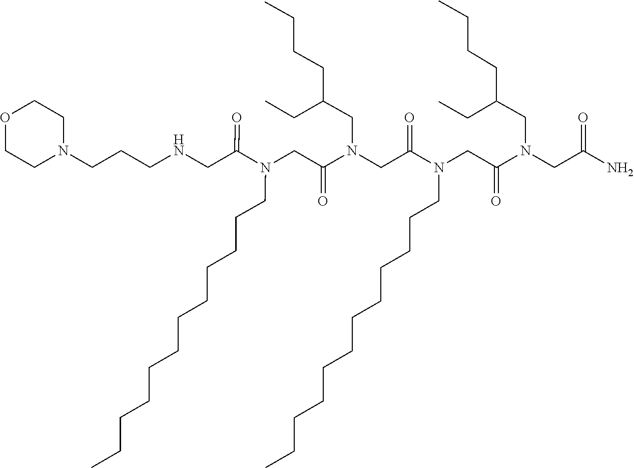 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCCN1CCOCC1] |
| 117 | 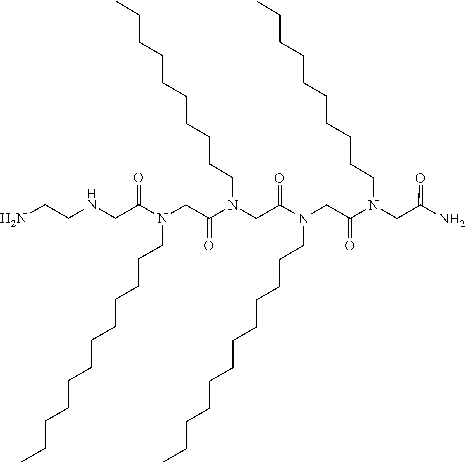 [SMILES: CCCCCCCCCCCCN(CC(=O)N(CCCCCCCCCC)CC(=O)N(CCCCCCCCCCCC)CC(=O)N(CCCCCCCCCC)CC(N)=O)C(=O)CNCCN] |
| 118 | 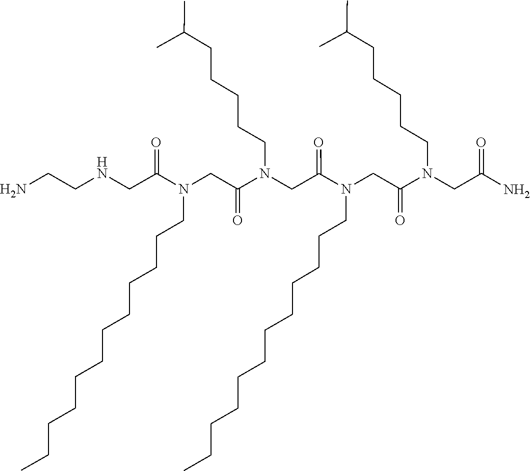 [SMILES: CCCCCCCCCCCCN(CC(=O)N(CCCCCC(C)C)CC(=O)N(CCCCCCCCCCCC)CC(=O)N(CCCCCC(C)C)CC(N)=O)C(=O)CNCCN] |
| 127 | 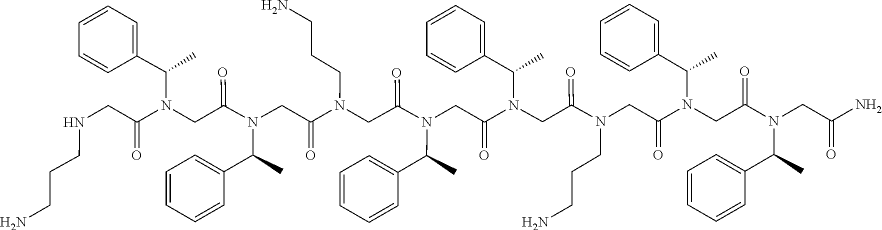 [SMILES: C[C@@H](c1ccccc1)N(CC(N)=O)C(=O)CN(C(=O)CN(CCCN)C(=O)CN(C(=O)CN(C(=O)CN(CCCN)C(=O)CN(C(=O)CN(C(=O)CNCCCN)[C@@H](C)c1ccccc1)[C@@H](C)c1ccccc1)[C@@H](C)c1ccccc1)[C@@H](C)c1ccccc1)[C@@H](C)c1ccccc1] |
| 130 | 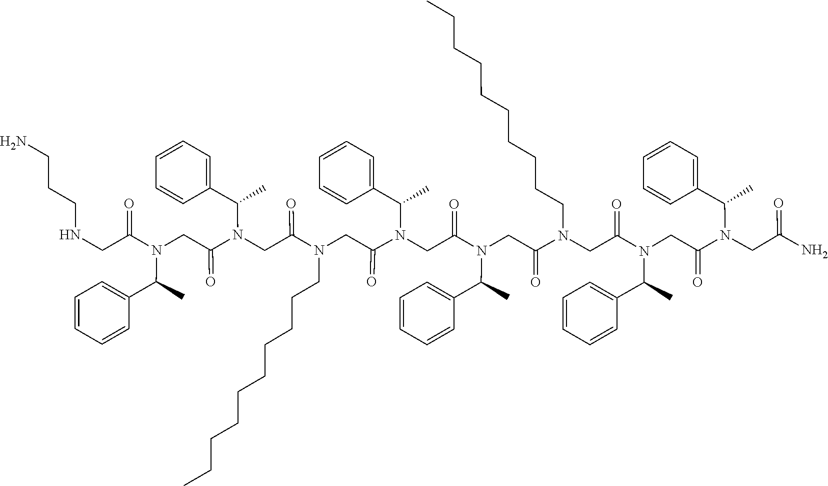 [SMILES: CCCCCCCCCCN(CC(=O)N(CC(=O)N(CC(N)=O)[C@@H](C)c1ccccc1)[C@@H](C)c1ccccc1)C(=O)CN(C(=O)CN(C(=O)CN(CCCCCCCCCC)C(=O)CN(C(=O)CN(C(=O)CNCCCN)[C@@H](C)c1ccccc1)[C@@H](C)c1ccccc1)[C@@H](C)c1ccccc1)[C@@H](C)c1ccccc1] |
| 132 | 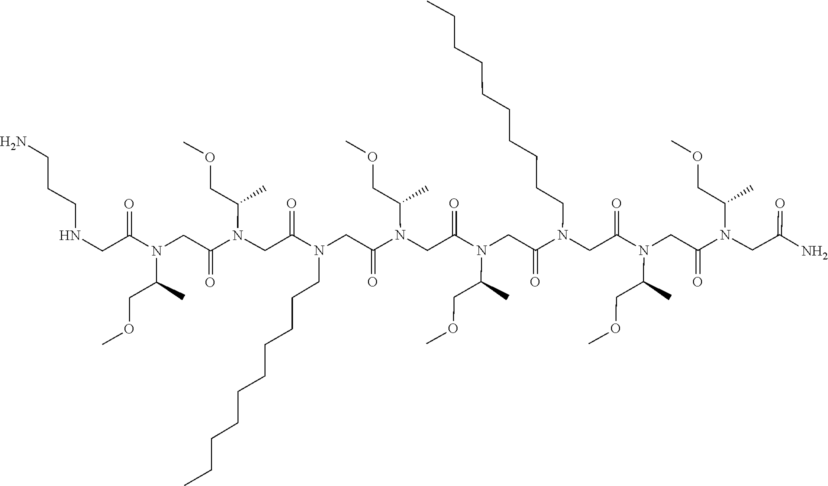 [SMILES: CCCCCCCCCCN(CC(=O)N(CC(=O)N(CC(N)=O)[C@@H](C)COC)[C@@H](C)COC)C(=O)CN(C(=O)CN(C(=O)CN(CCCCCCCCCC)C(=O)CN(C(=O)CN(C(=O)CNCCCN)[C@@H](C)COC)[C@@H](C)COC)[C@@H](C)COC)[C@@H](C)COC] |
| 137 | 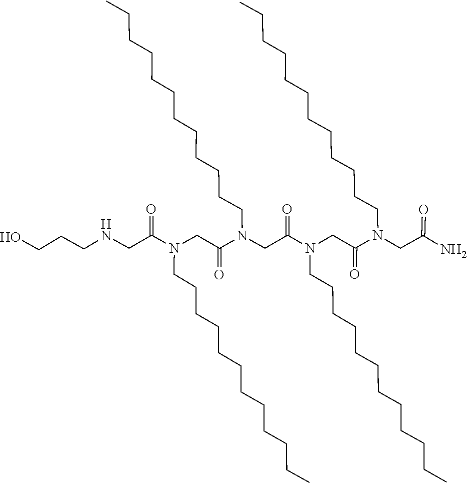 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCCO] |
| 138 | 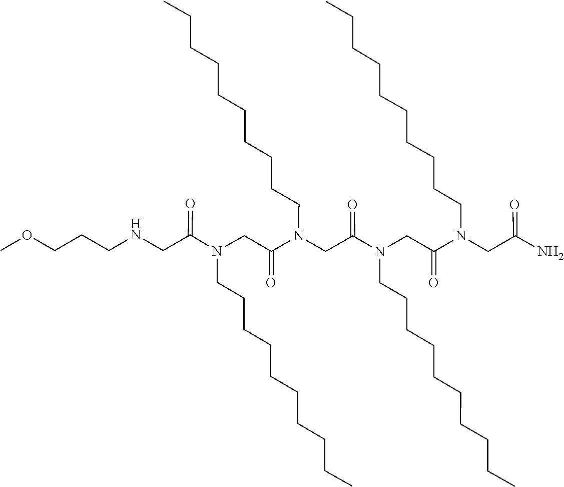 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCOC] |
| 139 | 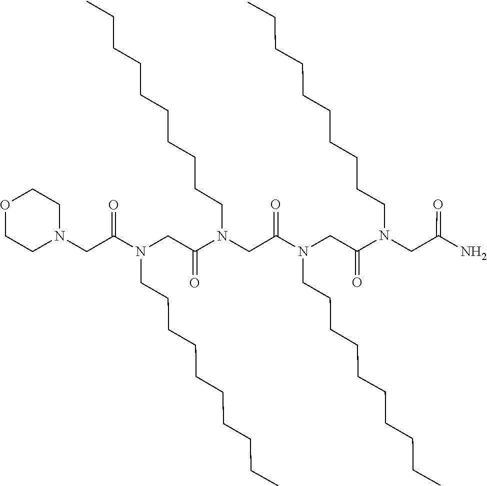 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN1CCOCC1] |
| 140 | 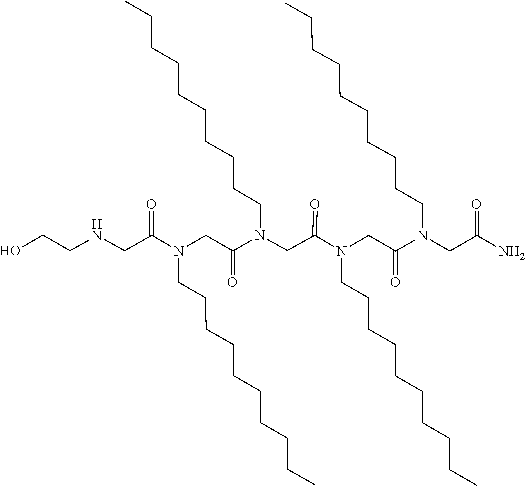 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCO] |
| 141 | 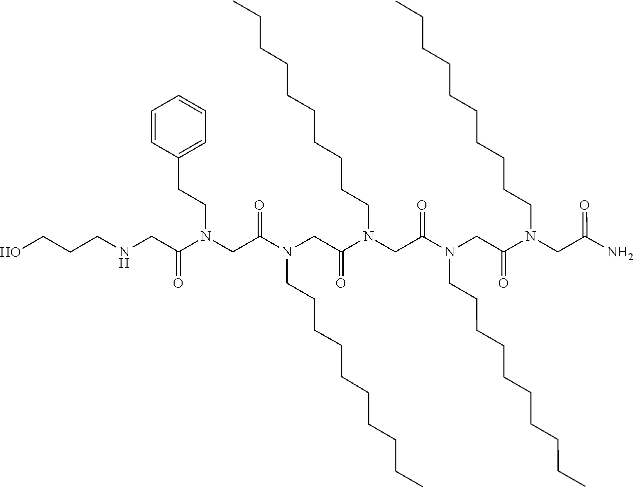 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CNCCCO] |
| 142 | 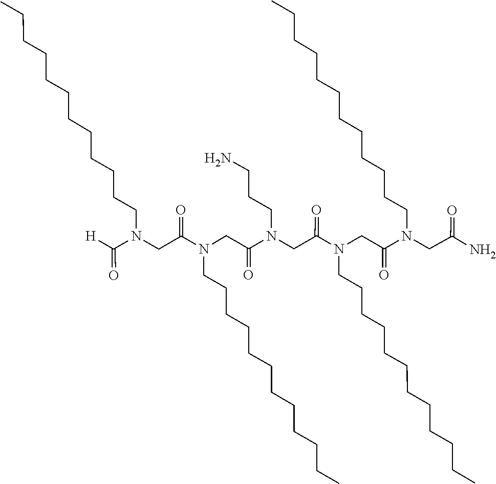 [SMILES: CCCCCCCCCCCCN(C=O)CC(=O)N(CCCCCCCCCCCC)CC(=O)N(CCCN)CC(=O)N(CCCCCCCCCCCC)CC(=O)N(CCCCCCCCCCCC)CC(N)=O] |
| 144 | 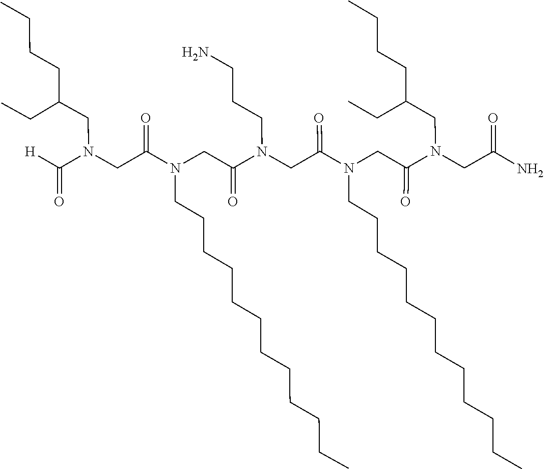 [SMILES: CCCCCCCCCCCCN(CC(=O)N(CCCN)CC(=O)N(CCCCCCCCCCCC)CC(=O)N(CC(N)=O)CC(CC)CCCC)C(=O)CN(C=O)CC(CC)CCCC] |
| 145 | 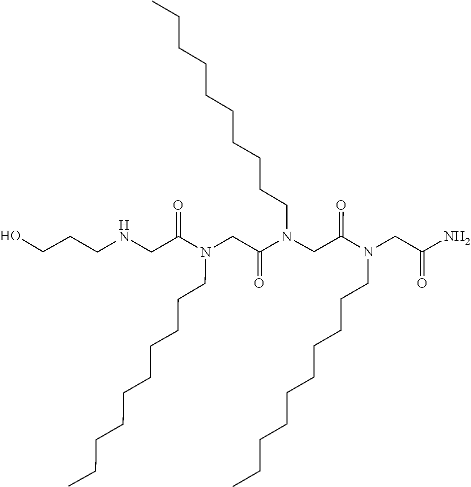 [SMILES: CCCCCCCCCCN(CC(N)=O)C(=O)CN(CCCCCCCCCC)C(=O)CN(CCCCCCCCCC)C(=O)CNCCCO] |
| 146 | 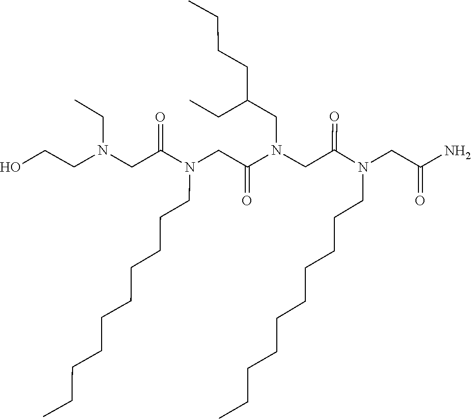 [SMILES: CCCCCCCCCCN(CC(N)=O)C(=O)CN(CC(CC)CCCC)C(=O)CN(CCCCCCCCCC)C(=O)CN(CC)CCO] |
| 147 | 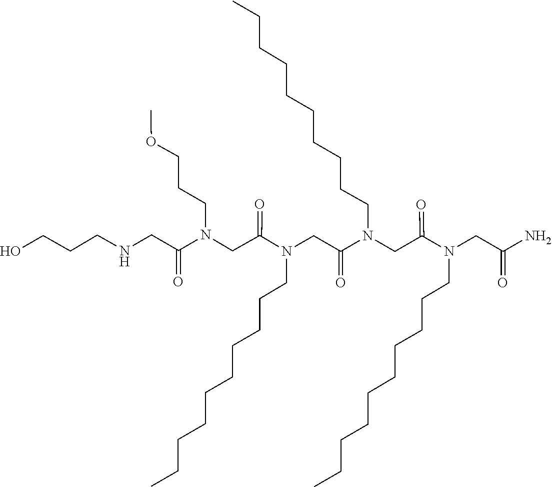 [SMILES: CCCCCCCCCCN(CC(N)=O)C(=O)CN(CCCCCCCCCC)C(=O)CN(CCCCCCCCCC)C(=O)CN(CCCOC)C(=O)CNCCCO] |
| 148 | 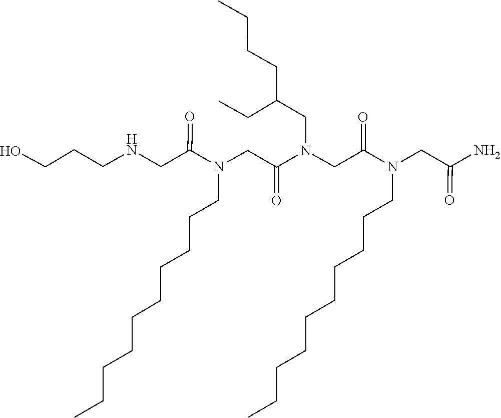 [SMILES: CCCCCCCCCCN(CC(N)=O)C(=O)CN(CC(CC)CCCC)C(=O)CN(CCCCCCCCCC)C(=O)CNCCCO] |
| 149 | 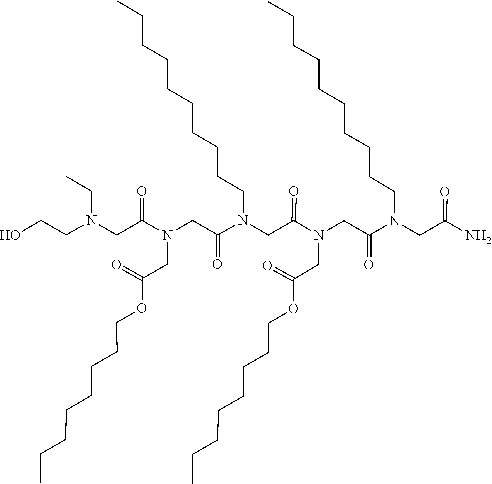 [SMILES: CCCCCCCCCCN(CC(N)=O)C(=O)CN(CC(=O)OCCCCCCCC)C(=O)CN(CCCCCCCCCC)C(=O)CN(CC(=O)OCCCCCCCC)C(=O)CN(CC)CCO] |
| 150 | 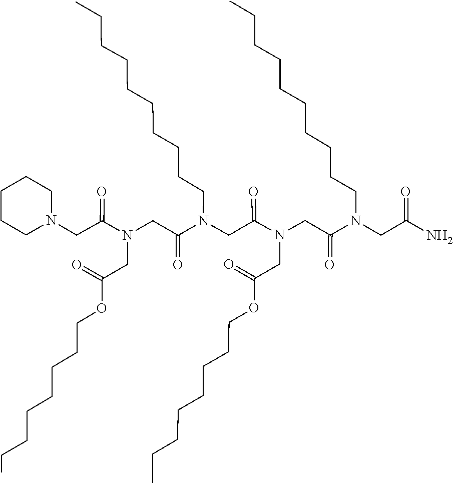 [SMILES: CCCCCCCCCCN(CC(N)=O)C(=O)CN(CC(=O)OCCCCCCCC)C(=O)CN(CCCCCCCCCC)C(=O)CN(CC(=O)OCCCCCCCC)C(=O)CN1CCCCC1] |
| 151 | 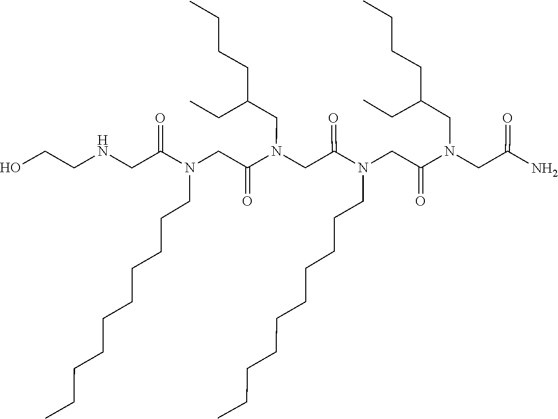 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CNCCO] |
| 152 | 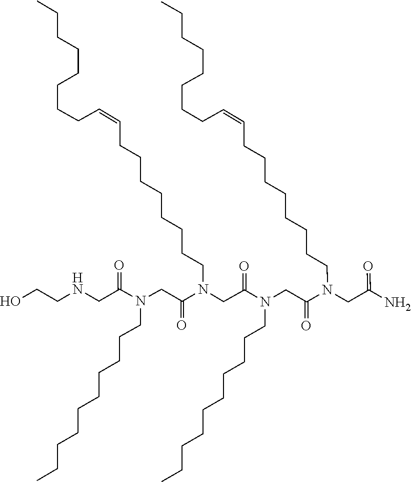 [SMILES: CCCCCCCC/C=C\CCCCCCCCN(CC(N)=O)C(=O)CN(CCCCCCCCCC)C(=O)CN(CCCCCCCC/C=C\CCCCCCCC)C(=O)CN(CCCCCCCCCC)C(=O)CNCCO] |
| 153 | 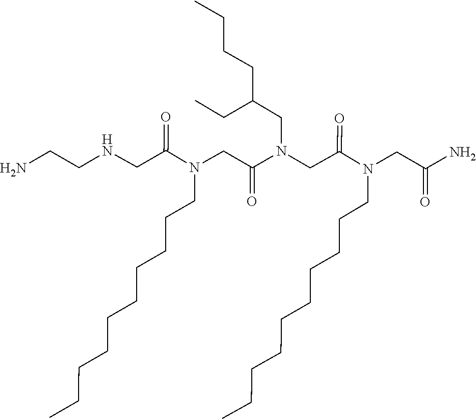 [SMILES: CCCCCCCCCCN(CC(N)=O)C(=O)CN(CC(CC)CCCC)C(=O)CN(CCCCCCCCCC)C(=O)CNCCN] |
| 154 | 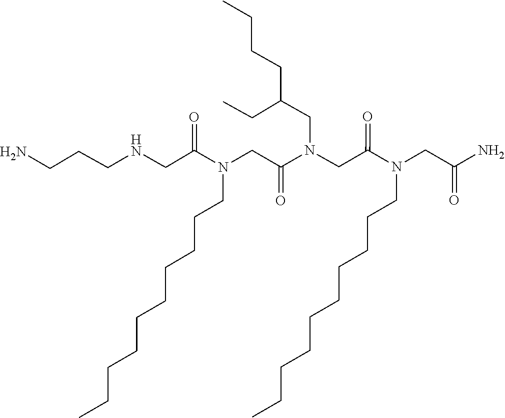 [SMILES: CCCCCCCCCCN(CC(N)=O)C(=O)CN(CC(CC)CCCC)C(=O)CN(CCCCCCCCCC)C(=O)CNCCCN] |
| 155 | 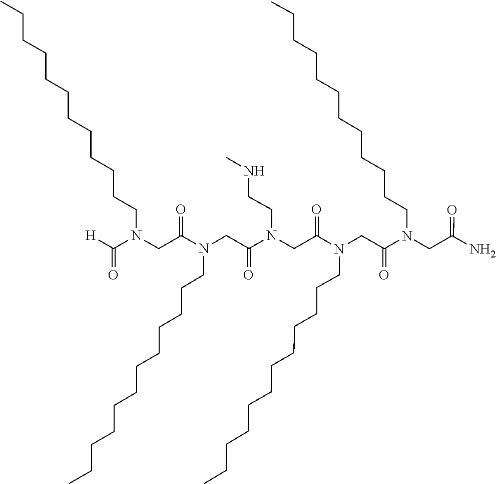 [SMILES: CCCCCCCCCCCCN(C=O)CC(=O)N(CCCCCCCCCCCC)CC(=O)N(CCNC)CC(=O)N(CCCCCCCCCCCC)CC(=O)N(CCCCCCCCCCCC)CC(N)=O] |
| 156 | 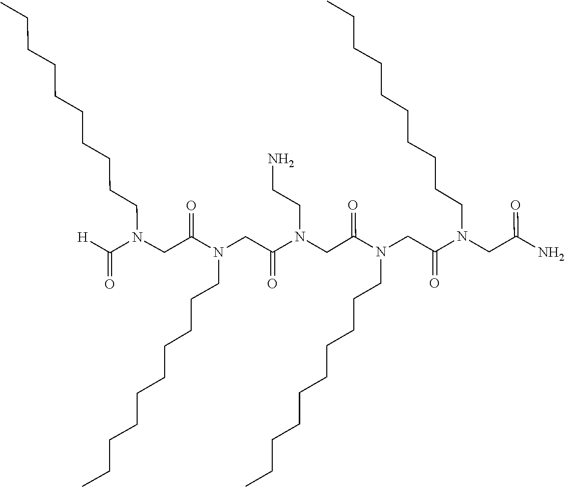 [SMILES: CCCCCCCCCCN(C=O)CC(=O)N(CCCCCCCCCC)CC(=O)N(CCN)CC(=O)N(CCCCCCCCCC)CC(=O)N(CCCCCCCCCC)CC(N)=O] |
| 159 | 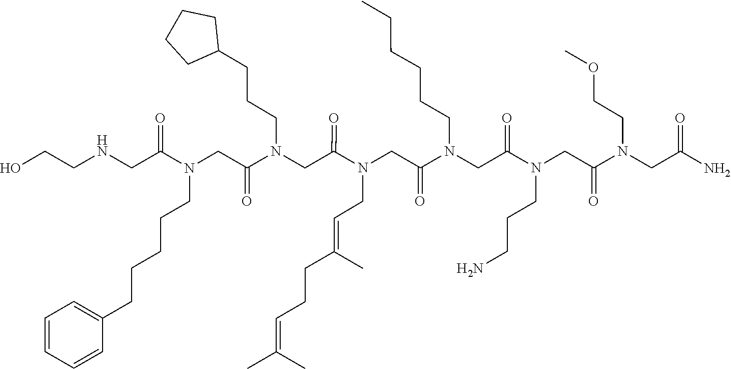 [SMILES: CCCCCCN(CC(=O)N(CCCN)CC(=O)N(CCOC)CC(N)=O)C(=O)CN(C/C=C(\C)CCC=C(C)C)C(=O)CN(CCCC1CCCC1)C(=O)CN(CCCCCc1ccccc1)C(=O)CNCCO] |
| 160 | 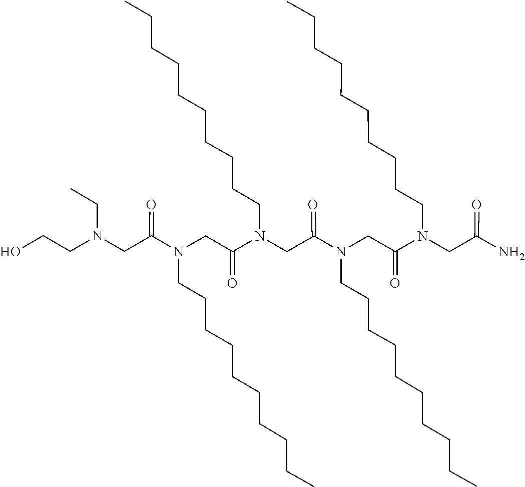 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CC)CCO] |
| 161 | 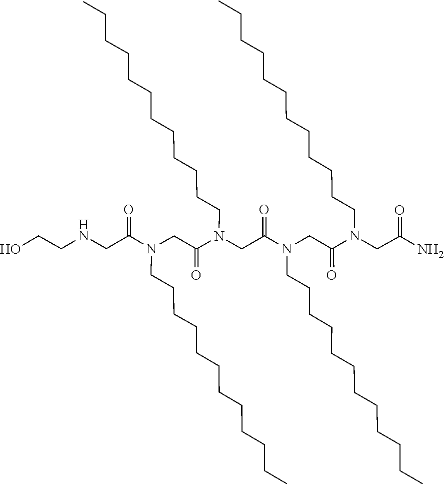 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCO] |
| 162 | 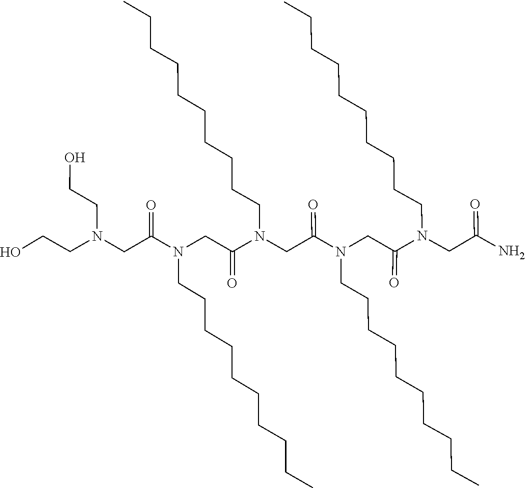 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCO)CCO] |
| 163 | 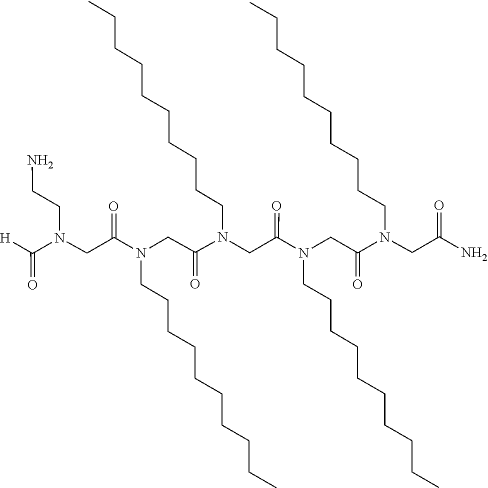 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(C=O)CCN] |
| 164 | 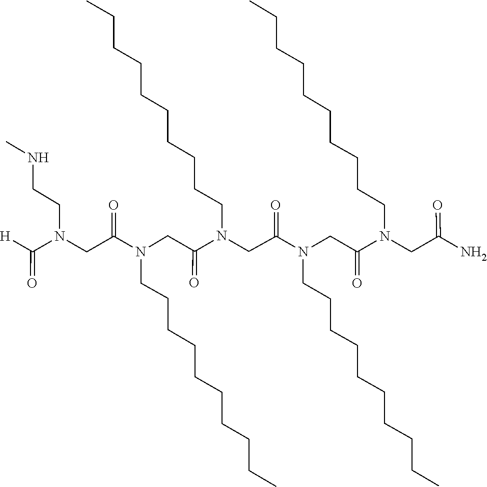 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(C=O)CCNC] |
| 165 | 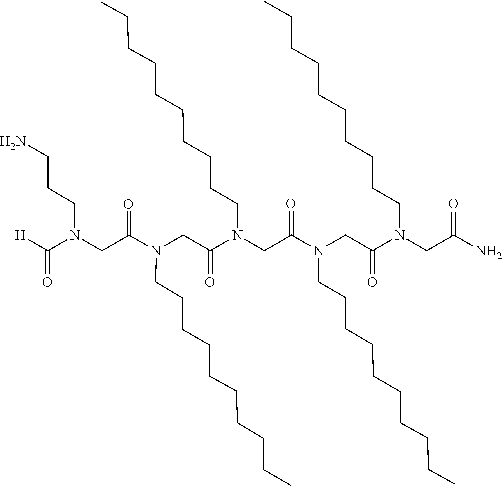 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(C=O)CCCN] |

TABLE 1B
Non-Limiting Zwitterionic/Anionic Lipidated Peptoids

| # | Compound Structure |
|---|---|
| 104 | 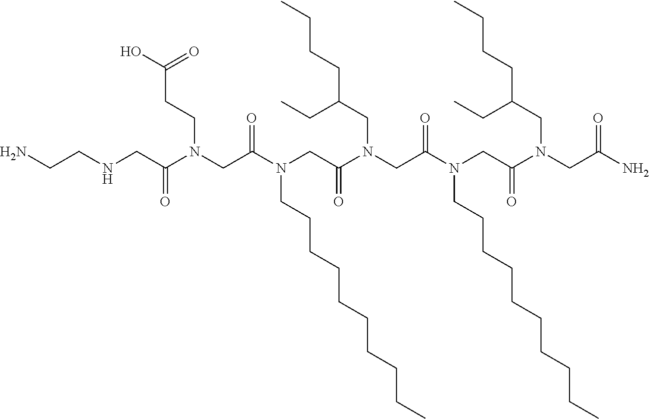 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCC(=O)O)C(=O)CNCCN] |
| 105 | 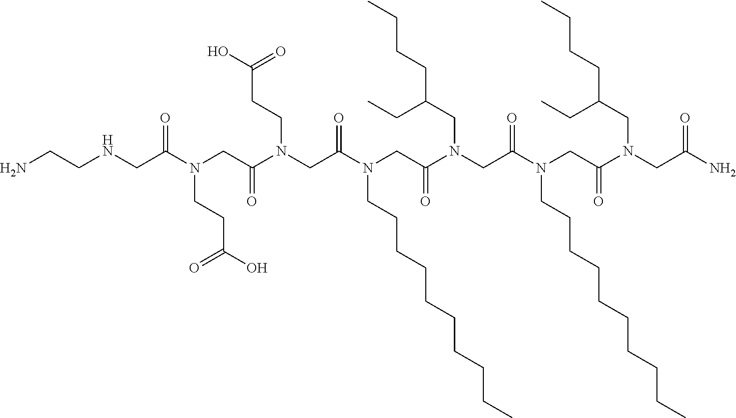 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCC(=O)O)C(=O)CN(CCC(=O)O)C(=O)CNCCN] |
| 124 | 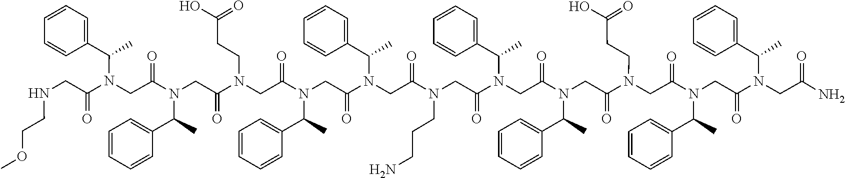 [SMILES: COCCNCC(=O)N(CC(=O)N(CC(=O)N(CCC(=O)O)CC(=O)N(CC(=O)N(CC(=O)N(CCCN)CC(=O)N(CC(=O)N(CC(=O)N(CCC(=O)O)CC(=O)N(CC(=O)N(CC(N)=O)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1] |
| 125 | 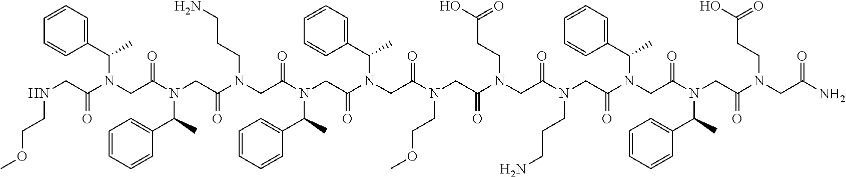 [SMILES: COCCNCC(=O)N(CC(=O)N(CC(=O)N(CCCN)CC(=O)N(CC(=O)N(CC(=O)N(CCOC)CC(=O)N(CCC(=O)O)CC(=O)N(CCCN)CC(=O)N(CC(=O)N(CC(=O)N(CCC(=O)O)CC(N)=O)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1] |
| 126 | 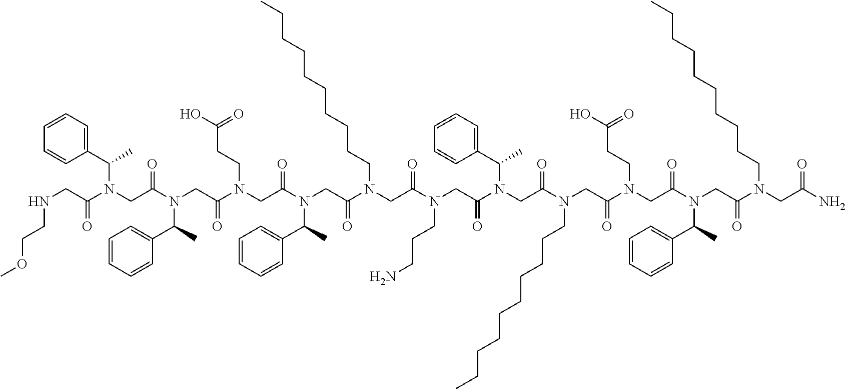 [SMILES: CCCCCCCCCCN(CC(N)=O)C(=O)CN(C(=O)CN(CCC(=O)O)C(=O)CN(CCCCCCCCCC)C(=O)CN(C(=O)CN(CCCN)C(=O)CN(CCCCCCCCCC)C(=O)CN(C(=O)CN(CCC(=O)O)C(=O)CN(C(=O)CN(C(=O)CNCCOC)[C@@H](C)c1ccccc1)[C@@H](C)c1ccccc1)[C@@H](C)c1ccccc1)[C@@H](C)c1ccccc1)[C@@H](C)c1ccccc1] |
| 131 | 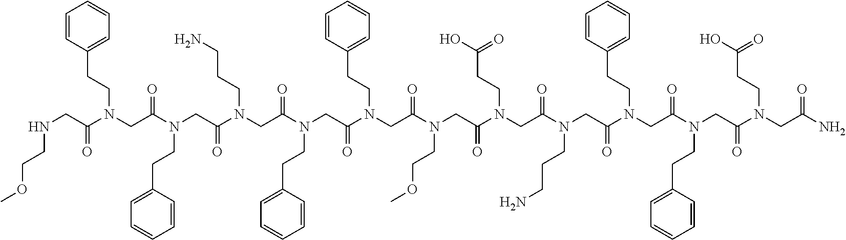 [SMILES: COCCNCC(=O)N(CCc1ccccc1)CC(=O)N(CCc1ccccc1)CC(=O)N(CCCN)CC(=O)N(CCc1ccccc1)CC(=O)N(CCc1ccccc1)CC(=O)N(CCOC)CC(=O)N(CCC(=O)O)CC(=O)N(CCCN)CC(=O)N(CCc1ccccc1)CC(=O)N(CCc1ccccc1)CC(=O)N(CCC(=O)O)CC(N)=O] |
| 133 | 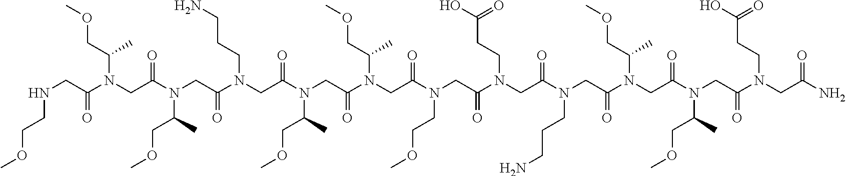 [SMILES: COCCNCC(=O)N(CC(=O)N(CC(=O)N(CCCN)CC(=O)N(CC(=O)N(CC(=O)N(CCOC)CC(=O)N(CCC(=O)O)CC(=O)N(CCCN)CC(=O)N(CC(=O)N(CC(=O)N(CCC(=O)O)CC(N)=O)[C@@H](C)COC)[C@@H](C)COC)[C@@H](C)COC)[C@@H](C)COC)[C@@H](C)COC)[C@@H](C)COC] |
| 134 | 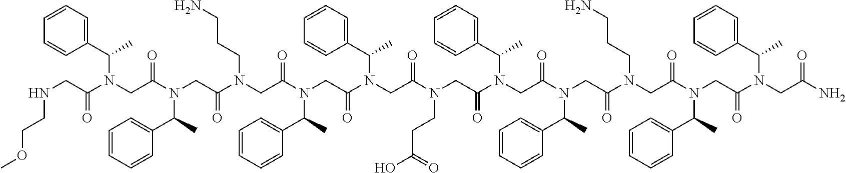 [SMILES: COCCNCC(=O)N(CC(=O)N(CC(=O)N(CCCN)CC(=O)N(CC(=O)N(CC(=O)N(CCC(=O)O)CC(=O)N(CC(=O)N(CC(=O)N(CCCN)CC(=O)N(CC(=O)N(CC(N)=O)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1] |
| 135 | 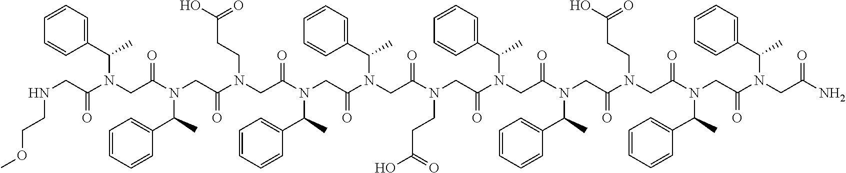 [SMILES: COCCNCC(=O)N(CC(=O)N(CC(=O)N(CCC(=O)O)CC(=O)N(CC(=O)N(CC(=O)N(CCC(=O)O)CC(=O)N(CC(=O)N(CC(=O)N(CCC(=O)O)CC(=O)N(CC(=O)N(CC(N)=O)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1] |
| 136 | 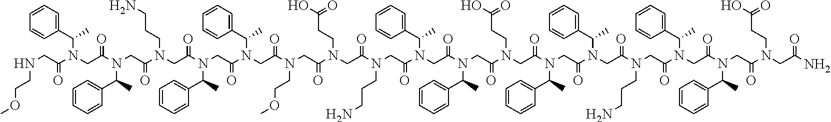 [SMILES: COCCNCC(=O)N(CC(=O)N(CC(=O)N(CCCN)CC(=O)N(CC(=O)N(CC(=O)N(CCOC)CC(=O)N(CCC(=O)O)CC(=O)N(CCCN)CC(=O)N(CC(=O)N(CC(=O)N(CCC(=O)O)CC(=O)N(CC(=O)N(CC(=O)N(CCCN)CC(=O)N(CC(=O)N(CC(=O)N(CCC(=O)O)CC(N)=O)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1] |

TABLE 1C
Non-Limiting Neutral Lipidated Peptoids

| # | Compound Structure |
|---|---|
| 90 | 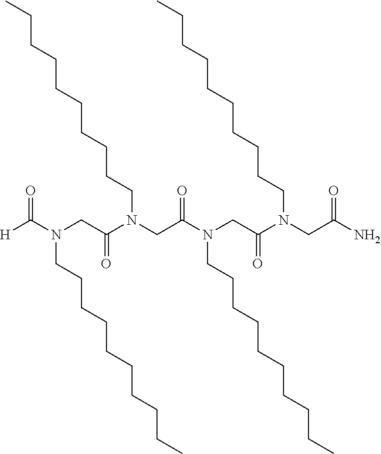 [SMILES: CCCCCCCCCCN(C=O)CC(=O)N(CCCCCCCCCC)CC(=O)N(CCCCCCCCCC)CC(=O)N(CCCCCCCCCC)CC(N)=O] |
| 119 | 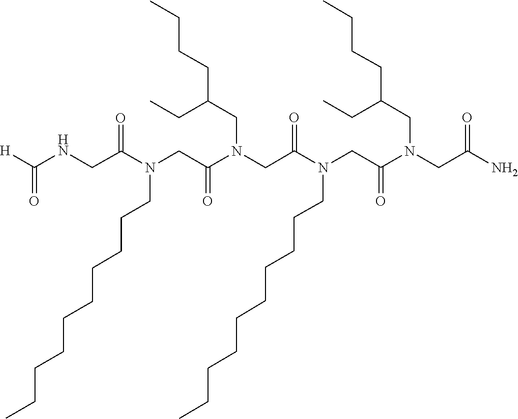 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CNC=O] |
| 120 | 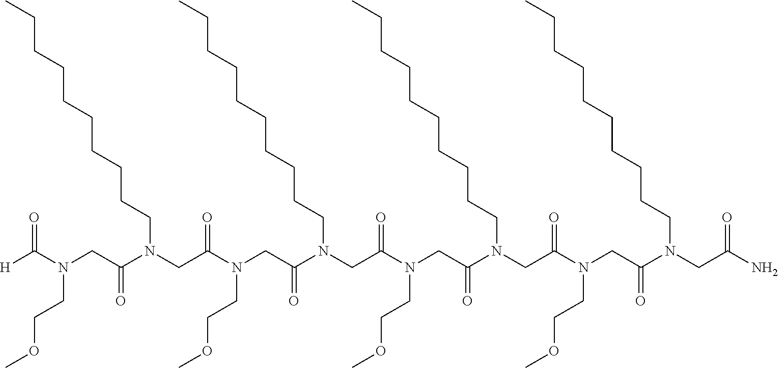 [SMILES: CCCCCCCCCCN(CC(N)=O)C(=O)CN(CCOC)C(=O)CN(CCCCCCCCCC)C(=O)CN(CCOC)C(=O)CN(CCCCCCCCCC)C(=O)CN(CCOC)C(=O)CN(CCCCCCCCCC)C(=O)CN(C=O)CCOC] |
| 121 | 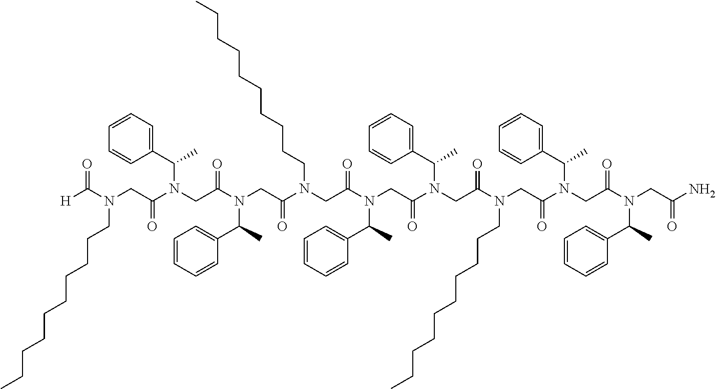 [SMILES: CCCCCCCCCCN(C=O)CC(=O)N(CC(=O)N(CC(=O)N(CCCCCCCCCC)CC(=O)N(CC(=O)N(CC(=O)N(CCCCCCCCCC)CC(=O)N(CC(=O)N(CC(N)=O)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1] |
| 122 | 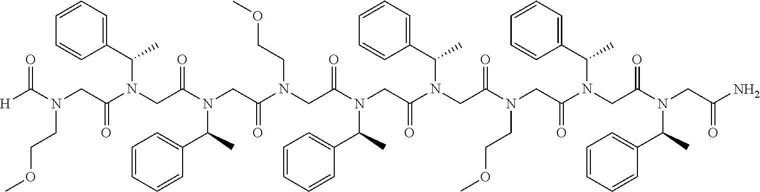 [SMILES: COCCN(C=O)CC(=O)N(CC(=O)N(CC(=O)N(CCOC)CC(=O)N(CC(=O)N(CC(=O)N(CCOC)CC(=O)N(CC(=O)N(CC(N)=O)[C@@H](C)c1ccccc1)[C@@H](C)c1ccccc1)[C@@H](C)c1ccccc1)[C@@H](C)c1ccccc1)[C@@H](C)c1ccccc1)[C@@H](C)c1ccccc1] |
| 123 | 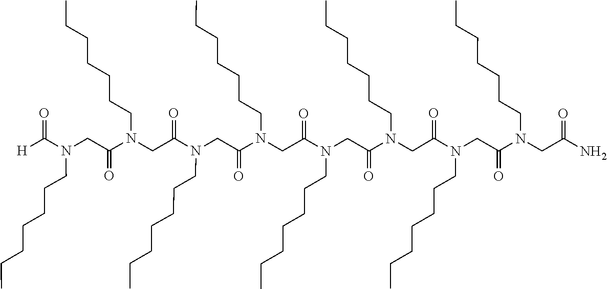 [SMILES: CCCCCCCN(C=O)CC(=O)N(CCCCCCC)CC(=O)N(CCCCCCC)CC(=O)N(CCCCCCC)CC(=O)N(CCCCCCC)CC(=O)N(CCCCCCC)CC(=O)N(CCCCCCC)CC(=O)N(CCCCCCC)CC(N)=O] |
| 128 | 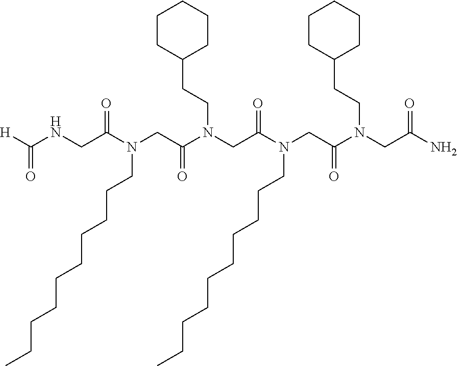 [SMILES: CCCCCCCCCCN(CC(=O)N(CCC1CCCCC1)CC(=O)N(CCCCCCCCCC)CC(=O)N(CCC1CCCCC1)CC(N)=O)C(=O)CNC=O] |
| 129 | 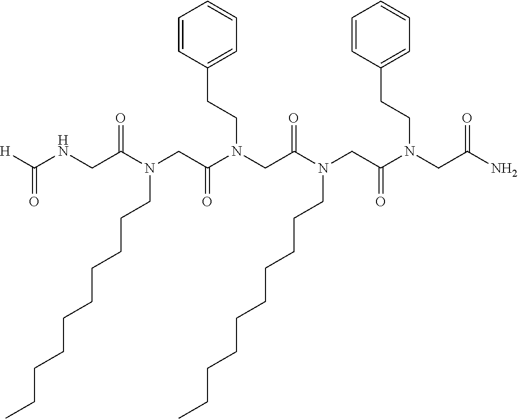 [SMILES: CCCCCCCCCCN(CC(=O)N(CCc1ccccc1)CC(=O)N(CCCCCCCCCC)CC(=O)N(CCc1ccccc1)CC(N)=O)C(=O)CNC=O] |

TABLE 1D
Non-Limiting Shielding Lipidated Peptoids

| # | Compound Structure |
|---|---|
| 48 | 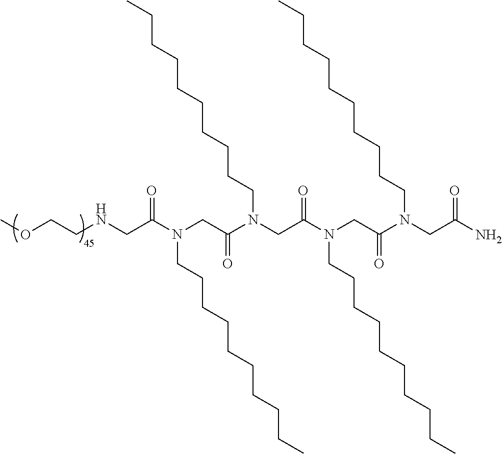 |
| 56 | 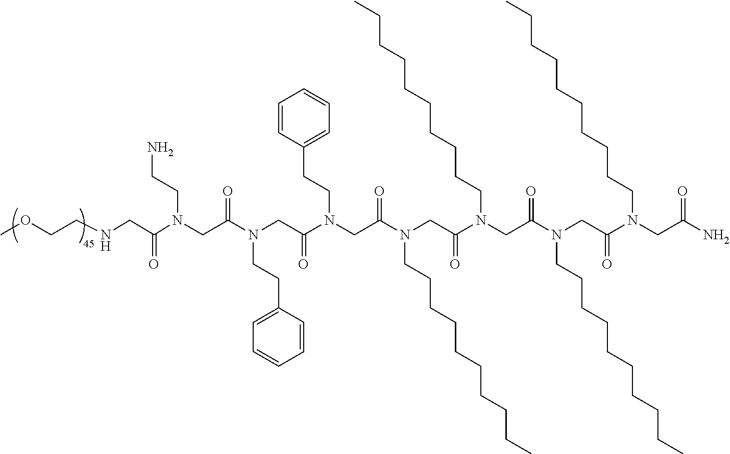 |
| 64 | 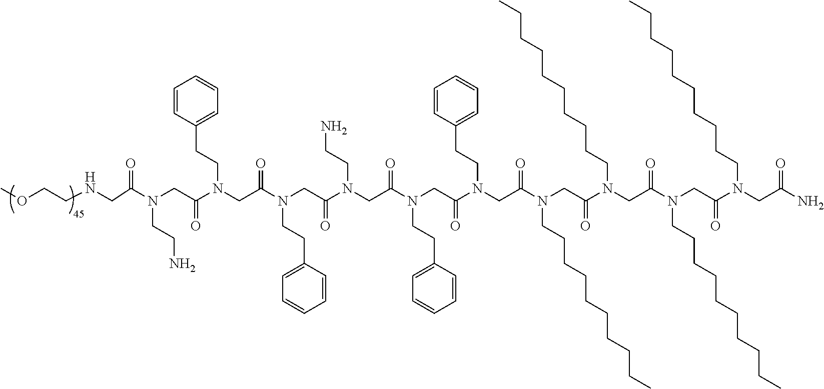 |
| 72 | 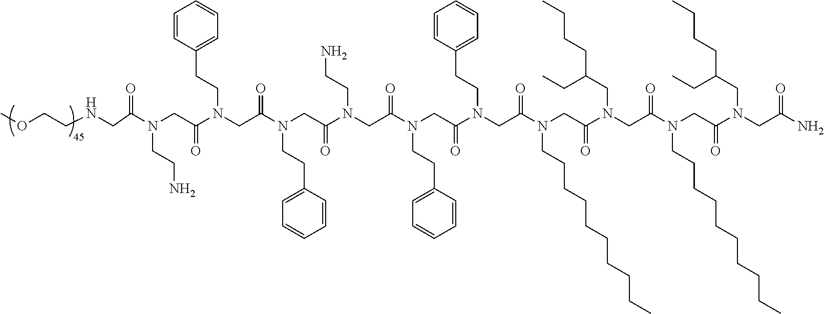 |
| 106 | 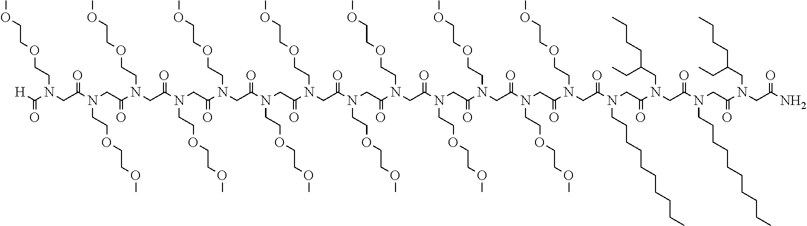 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(C=O)CCOCCOC] |
| 107 | 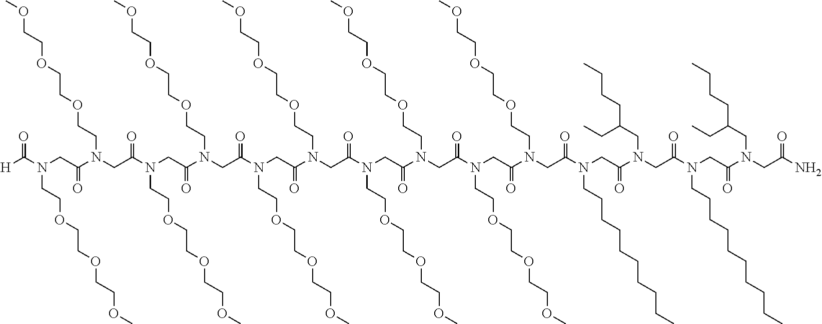 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCOCCOCCOC)C(=O)CN(CCOCCOCCOC)C(=O)CN(CCOCCOCCOC)C(=O)CN(CCOCCOCCOC)C(=O)CN(CCOCCOCCOC)C(=O)CN(CCOCCOCCOC)C(=O)CN(CCOCCOCCOC)C(=O)CN(CCOCCOCCOC)C(=O)CN(CCOCCOCCOC)C(=O)CN(C=O)CCOCCOCCOC] |
| 108 | 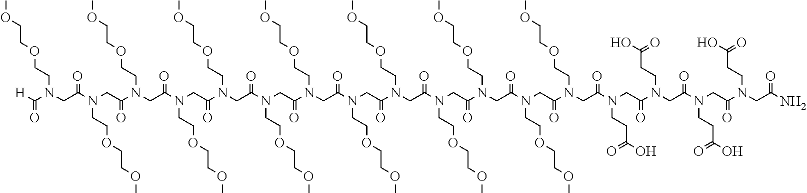 [SMILES: COCCOCCN(C=O)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCC(=O)O)CC(=O)N(CCC(=O)O)CC(=O)N(CCC(=O)O)CC(=O)N(CCC(=O)O)CC(N)=O] |
| 109 | 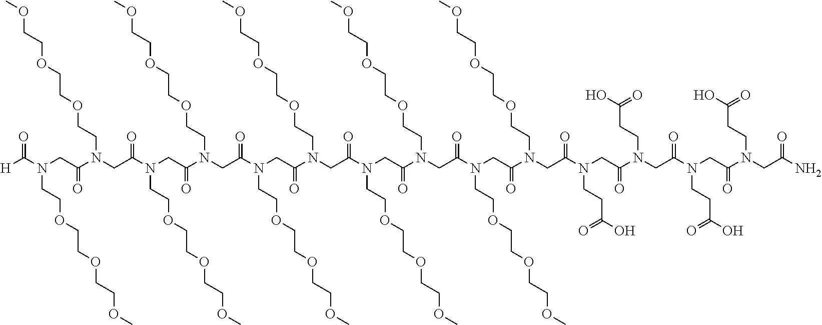 [SMILES: COCCOCCOCCN(C=O)CC(=O)N(CCOCCOCCOC)CC(=O)N(CCOCCOCCOC)CC(=O)N(CCOCCOCCOC)CC(=O)N(CCOCCOCCOC)CC(=O)N(CCOCCOCCOC)CC(=O)N(CCOCCOCCOC)CC(=O)N(CCOCCOCCOC)CC(=O)N(CCOCCOCCOC)CC(=O)N(CCOCCOCCOC)CC(=O)N(CCC(=O)O)CC(=O)N(CCC(=O)O)CC(=O)N(CCC(=O)O)CC(=O)N(CCC(=O)O)CC(N)=O] |

TABLE 1E
Non-Limiting Peptoid Precursors

| # | Compound Structure |
|---|---|
| 37 | 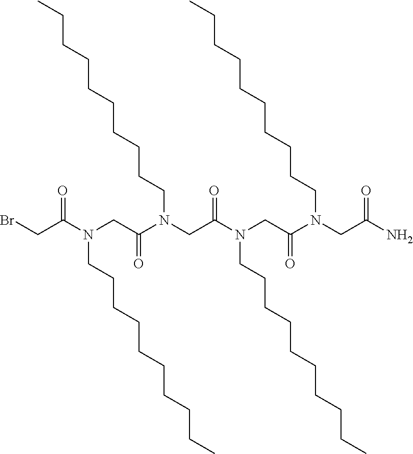 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CBr] |
| 38 | 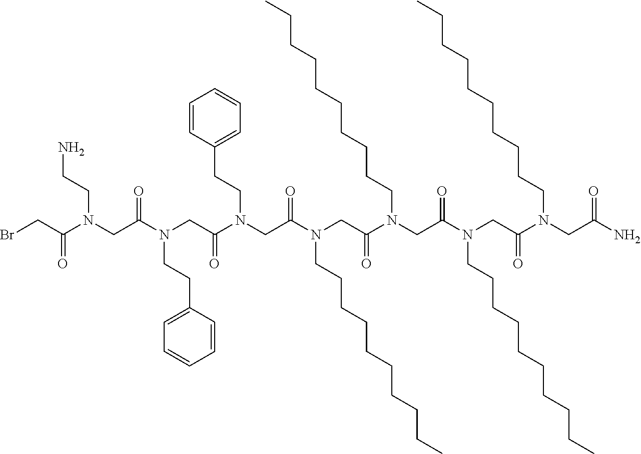 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CBr] |
| 39 | 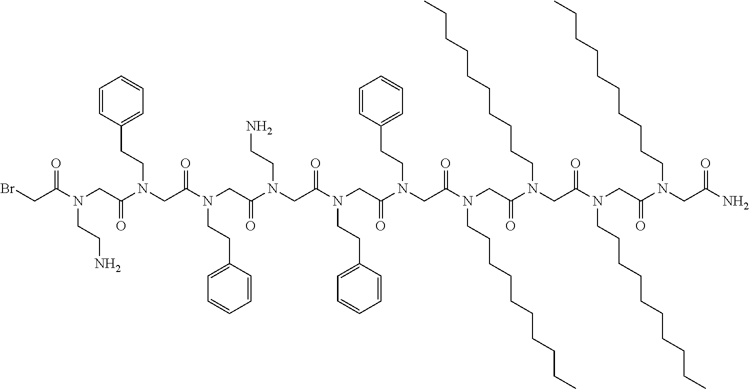 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CBr] |
| 40 | 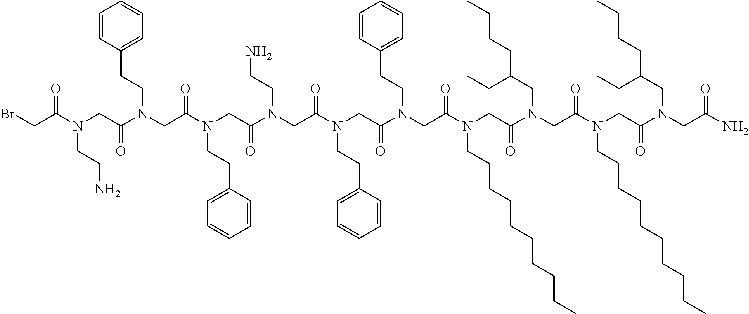 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CBr] |
| 110 | 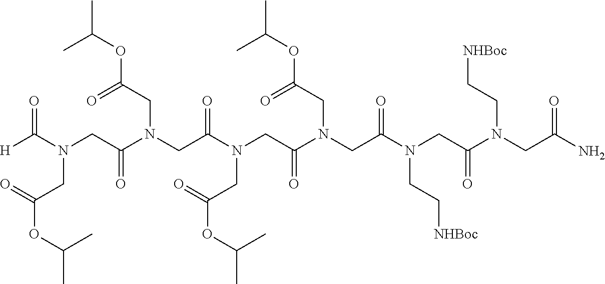 [SMILES: CC(C)OC(=O)CN(C=O)CC(=O)N(CC(=O)OC(C)C)CC(=O)N(CC(=O)OC(C)C)CC(=O)N(CC(=O)OC(C)C)CC(=O)N(CCNC(=O)OC(C)(C)C)CC(=O)N(CCNC(=O)OC(C)(C)C)CC(N)=O] |

In some cases, the multicomponent delivery systems of the disclosure are single peptoid delivery systems (or single peptoid delivery vehicles). Single component delivery systems include one type of peptoid component (e.g., a lipidated cationic peptoid component, a neutral lipidated peptoid component, an anionic/zwitterionic lipidated peptoid component, or a PEGylated peptoid component), while the remaining components are non-peptoids (e.g., a non-peptoid, neutral lipid component, a non-peptoid anionic/zwitterionc component, and a non-peptoid PEGylated lipidated component). In some implementations, the single peptoid delivery systems of the disclosure can include a lipidated cationic peptoid component as the peptoid component. In some cases, the lipidated cationic peptoid has a structure of Formula (Ia), a structure of Formula (Ib), a structure of Formula (Ic), a structure of Formula (Id), a structure of Formula (Ie) (e.g., a structure of Formula (Ie′) or a structure of Formula (Ie″)), or a structure of Formula (If)). In some cases, the single peptoid delivery system comprises one or more of a lipidated cationic peptoid listed in Table 1A. In addition to the cationic lipidated peptoid component, the single component delivery systems can further include a non-peptoid anionic/zwitterionic compound, as previously disclosed herein, such as a phospholipid (e.g., DSPC or DOPE); a non-peptoid neutral lipid, as previously disclosed herein (e.g., cholesterol), and/or a non-peptoid PEGylated lipid, as previously disclosed herein (e.g., DMG-PEG 2000). The single peptoid delivery vehicle compositions of the disclosure can include any combination of components (lipidated cationic peptoid, neutral lipid (e.g., sterol), phospholipid (e.g., DSPC or DOPE), and PEGylated lipid disclosed herein (e.g., DMG-PEG 2000), in any amount or mass ratio disclosed herein. In some cases, the single peptoid delivery systems include a formulation listed in Table 2 or Table 3, below (e.g., Formula F1A, F2A, F3A, F4A, F5A, F1, F2, F3, F4, F5, and F6). Examples of single peptoid systems include FORM-B, 81-F2, 112-F2, 137-F2, 73-F2, 24-F2, 79-F2, 93-F2, 85-F2, 115-F2, 152-F2 (see Table 4).

In some implementations, the multicomponent delivery system of the disclosure includes two or more components that are peptoids. These systems can be referred to as “mixed peptoid delivery systems” or “mixed peptoid delivery vehicles” or “mixed peptoid vehicles” In some implementations, the mixed peptoid delivery systems of the disclosure can include two peptoid components. In some implementations, the mixed peptoid delivery systems of the disclosure can include three peptoid components. In these implementations, one peptoid of the mixed peptoid delivery vehicle can comprise a lipidated cationic peptoid component, as previously described herein. In some cases, the lipidated cationic peptoid comprises a compound of Formula (Ia) (e.g., Compounds 1-12 and 19-36), a compound of Formula (Ib) (e.g., Compounds 1-13-16, 76, and 99), a compound of Formula (Ic) (e.g., Compounds 49-55), a compound of Formula (Id) (e.g., Compounds 57-63 and 65-71), a compound of Formula (Ie) (e.g., Compounds 41-47, 73-75, 77-80, 85, 86, 91-94, 97, 98, 111-118, 137, 138, 140, 145, 146, 148, 149, 151-154, and 160-162), such as a compound of Formula (Ie′) (e.g., Compounds 41, 42, 44-47, 73-75, 77-80, 85, 86, 91-94, 97, 98, 111-118, 153, and 154) or a compound of Formula (Ie″) (e.g., Compounds 43, 137, 138, 140, 145, 146, 148, 149, 151, 152, 154, and 160-162), or a compound of Formula (If) (e.g., Compounds 81, 82, 83, 95, 96, 142, 144, 155, 156, 163, 164, 165). In some cases, the lipidated cationic peptoid can comprise a peptoid listed in Table 1A. In some implementations, the lipidated cationic peptoid is a compound selected from the group consisting of Compound 17, 18, 84, 87, 88, 100, 101, 102, 103, 110, 113, 114, 127, 130, 132, 134, 139, 141, 147, and 159. In some examples, the lipidated cationic comprises Compound 24, 73, 79, 81, 85, 93, 112, 115, 127, 137, 152, 155, 163, 164, or combinations thereof. In some cases, the lipidated cationic peptoid comprises Compound 81, 112, 155, 163, 164, or combinations thereof. In various implementations, the lipidated cationic peptoid comprises Compound 81, 155, 163, 164, or combinations thereof. In some cases, the lipidated cationic peptoid comprises Compound 81, 155, or combinations thereof. In various cases, the lipidated cationic peptoid comprises Compound 81. In some implementations, the lipidated cationic peptoid comprises Compound 155. In various implementations, the lipidated cationic peptoid comprises Compound 163. In various implementations, the lipidated cationic peptoid comprises Compound 164.

The second peptoid of the mixed peptoid delivery system can be a non-cationic lipidated peptoid, as previously described herein. In some implementations, the non-cationic lipidated peptoid can be an anionic/zwitterionic lipidated peptoid, as previously described herein. In some cases, the anionic/zwitterionic lipidated peptoid comprises a compound of Formula (Ma) (e.g., Compound 104 and Compound 105), a compound of Formula (IIIb) (e.g., Compound 124 and Compound 126) or a compound listed in Table 1B (e.g., Compound 125). In some cases, the non-cationic lipidated peptoid comprises an anionic/zwitterionic lipidated peptoid selected from Compound 104, 105, 124, 125, 126, 135, and combinations thereof. In some cases, the non-cationic peptoid comprises an anionic/zwitterionic lipidated peptoid selected from Compound 124, 125, 126, and combinations thereof. In some cases, the anionic/zwitterionic lipidated peptoid comprises Compound 124. In some cases, the anionic/zwitterionic lipidated peptoid comprises Compound 125. In some cases, the anionic/zwitterionic lipidated peptoid comprises Compound 126.

In some implementations, the non-cationic lipidated peptoid can be a neutral lipidated peptoid, as previously described herein. In some implementations, the neutral lipidated peptoid comprises a compound of Formula (IVa) (e.g., Compound 90, 119, 120, 121, 122, 123, 128, and 129). In some cases, the non-cationic lipidated peptoid comprises a neutral lipidated peptoid listed in Table 1C. In some cases, the non-cationic lipidated peptoid comprises a neutral lipidated peptoid selected from Compound 90, 119, 120, 121, 122, 123, 128, 129, and combinations thereof. In some implementations, the non-cationic lipidated peptoid comprises a neutral lipidated peptoid selected from Compound 90, 119, and combinations thereof. In some cases, the neutral lipidated peptoid comprises Compound 90. In various cases, the neutral lipidated peptoid comprises Compound 119.

In some cases the non-cationic lipidated peptoid can be a PEGylated lipidated peptoid, as previously described herein. In some implementations, the PEGylated lipidated peptoid comprises a compound of Formula (Va), a compound of Formula (Vb), or a compound a Formula (Vc). In some cases, the non-cationic lipidated peptoid comprises a PEGylated lipidated peptoid listed in Table 1D. In some cases, the non-cationic lipidated peptoid comprises a PEGylated lipidated peptoid selected from Compound 48, 56, 64, 72, 106, 107, 108, 109, and combinations thereof. In some implementations, the non-cationic lipidated peptoid comprises a PEGylated lipidated peptoid selected from Compound 106, 107, 108, 109, and combinations thereof. In some cases, the non-cationic lipidated peptoid comprises Compound 107.

In some implementations, the mixed peptoid delivery systems do not include an anionic/zwitterionic component. In some cases, the mixed peptoid delivery systems contain a non-peptoid anionic/zwitterionic component, and/or a non-peptoid PEGylated lipid component. In various cases, the mixed peptoid delivery systems contain a non-peptoid anionic/zwitterionic component and a non-peptoid PEGylated lipid component. The non-peptoid anionic/zwitterionic component can be any non-peptoid anionic/zwitterionic component described herein. In some cases, the non-peptoid anionic/zwitterionic component is a phospholipid. In some implementations, the phospholipid is selected from the group consisting of DSPC and DOPE. In some cases, the phospholid is DSPC. In some implementations, the phospholipid is DOPE. In various implementations, the non-peptoid PEGylated lipid is a DMG-PEG, such as DMG-PEG 2000. In some cases, the mixed peptoid delivery systems include a non-peptoid neutral lipid, such as a sterol (e.g., cholesterol).

Nonlimiting examples of components of the mixed peptoid delivery systems disclosed herein are listed in Table 1F, below.

TABLE 1F
Non-limiting components of the mixed peptoid delivery systems

Cationic

| Component | Non-Cationic Component | | |
|---|---|---|---|
| Lipidated | | Anionic/ | |
| Cationic | Neutral Lipid | Zwitterionic | PEGylated Lipid |
| Peptoid | Component | Component | Component |
| Compound 24 | Cholesterol* | DOPE* | DMG-PEG 2000* |
| Compound 73 | Compound 90 | DSPC* | Compound 107 |
| Compound 79 | Compound 119 | Compound 105 | |
| Compound 81 | Compound 120 | Compound 124 | |
| Compound 85 | Compound 121 | Compound 125 | |
| Compound 93 | Compound 122 | Compound 126 | |
| Compound 112 | Compound 123 | Compound 135 | |
| Compound 115 | Compound 128 | | |
| Compound 127 | Compound 129 | | |
| Compound 137 | | | |
| Compound 152 | | | |
| Compound 155 | | | |
| Compound 163 | | | |
| Compound 164 | | | |

*Non-peptoid component

In some implementations, the mixed peptoid delivery system of the disclosure includes at least one lipidated cationic peptoid and at least one non-cationic lipidated peptoid listed in Table 1F. In some cases, the mixed peptoid delivery system of the disclosure does not include an anionic/zwitterionic component. In some implementations, the mixed peptoid delivery system of the disclosure includes one cationic component listed in Table 1F, one neutral lipid component listed in Table 1F, and one PEGylated lipid component listed in Table 1F. Specifically contemplated mixed peptoid delivery systems can include any combination of cationic components, neutral components, anionic/zwitterionic components (or none), and PEGylated lipid components listed in Table 1F.

In some implementations, the mixed peptoid delivery system comprises a lipidated cationic peptoid selected from the group consisting of Compound 24, 73, 79, 81, 85, 93, 112, 115, 127, 137, 152, 155, 163, 164, and combinations thereof; and a non-cationic lipidated that is a neutral lipidated peptoid selected from the group consisting of Compound 90, 119, 120, 121, 122, 123, 128, 129, and combinations thereof. In some implementations, the mixed peptoid delivery system comprises a lipidated cationic peptoid selected from the group consisting of Compound 81, 85, 93, 112, 155, 163, 164, and combinations thereof; and a non-cationic lipidated peptoid that is a neutral lipidated peptoid selected from the group consisting of Compound 90, 119, and combinations thereof. In some implementations, the mixed peptoid delivery system comprises a lipidated cationic peptoid selected from the group consisting of Compound 81, 155, and combinations thereof; and a non-cationic lipidated peptoid that is a neutral lipidated peptoid selected from the group consisting of Compound 90, 119, and combinations thereof. In some cases, the mixed peptoid delivery system comprises Compound 81 and Compound 90. In some cases, the mixed peptoid delivery system comprises Compound 81 and Compound 119. In some cases, the mixed peptoid delivery system comprises Compound 155 and Compound 90. In some cases, the mixed peptoid delivery system comprises Compound 155 and Compound 119. In some cases, the mixed peptoid delivery system comprises Compound 112 as the lipidated cationic peptoid and a neutral lipidated peptoid selected from Compound 90, 119, 120, 121, 122, 123, 128, 129, and combinations thereof. In some cases, the mixed peptoid system comprises Compound 112 and Compound 90. In some cases, the mixed peptoid delivery system comprises Compound 112 and Compound 119. In some cases, the peptoid delivery system comprises Compound 112 and Compound 127; or Compound 112 and Compound 122. In some implementations, the mixed peptoid delivery system further comprises Compound 135. In some implementations, the mixed peptoid delivery system does not contain an anionic/zwitterionic component. In some implementations, the mixed peptoid delivery systems contains an anionic/zwitterionic component. In some cases, the anionic/zwitterionic component is a phospholipid (e.g., DSPC, DOPE). In some implementations, the anionic/zwitterionic component is DSPC. In some implementations, the anionic/zwitterionic component is DOPE. In some implementations, the mixed peptoid delivery system further comprises a PEGyated lipid component (e.g., DMG-PEG 2000 and/or Compound 107). In some cases, the mixed peptoid delivery system further comprises a non-peptoid neutral lipid component, such as a sterol (e.g., cholesterol). Example mixed peptoid delivery systems containing a lipidated cationic peptoid and a neutral lipidated peptoid as the non-cationic peptoid can be found in Table 4.

In some implementations, the mixed peptoid delivery system comprises a lipidated cationic peptoid selected from the group consisting of Compound 24, 73, 79, 81, 85, 93, 112, 115, 127, 137, 152, 155, 163, 164, and combinations thereof; and a non-cationic lipidated that is an anionic/zwitterionic lipidated peptoid selected from the group consisting of Compound 105, 124, 125, 126, 135, and combinations thereof. In some implementations, the mixed peptoid delivery system comprises a lipidated cationic peptoid selected from the group consisting of Compound 81, 85, 93, 112, 155, 163, 164, and combinations thereof; and a non-cationic lipidated peptoid that is an anionic/zwitterionic lipidated peptoid selected from the group consisting of Compound 124, 125, 126, and combinations thereof. In some implementations, the mixed peptoid delivery system comprises a lipidated cationic peptoid selected from the group consisting of Compound 81, 155, and combinations thereof; and a non-cationic lipidated peptoid that is an anionic/zwitterionic lipidated peptoid selected from the group consisting of Compound 124, 125, 126, and combinations thereof. In some cases, the mixed peptoid delivery system comprises Compound 81 and Compound 124. In some cases, the mixed peptoid delivery system comprises Compound 81 and Compound 125. In some cases, the mixed peptoid delivery system comprises Compound 81 and Compound 126. In some cases, the mixed peptoid delivery system comprises Compound 155 and Compound 124. In some cases, the mixed peptoid delivery system comprises Compound 155 and Compound 124. In some cases, the mixed peptoid delivery system comprises Compound 155 and Compound 126. In some cases, the mixed peptoid delivery system comprises Compound 112 as the lipidated cationic peptoid and an anionic/zwitterionic peptoid selected from Compound 105, 124, 125, 126, 135, and combinations thereof. In some cases, the mixed peptoid system comprises Compound 112 and Compound 124. In some cases, the mixed peptoid delivery system comprises Compound 112 and Compound 125. In some cases, the mixed peptoid delivery system comprises Compound 112 and Compound 126. In some implementations, the mixed peptoid delivery system does not contain a further anionic/zwitterionic component. In some implementations, the mixed peptoid delivery systems contains an anionic/zwitterionic component. In some cases, the anionic/zwitterionic component is a phospholipid (e.g., DSPC, DOPE). In some implementations, the anionic/zwitterionic component is DSPC. In some implementations, the anionic/zwitterionic component is DOPE. In some implementations, the mixed peptoid delivery system further comprises a PEGyated lipid component (e.g., DMG-PEG 2000). In some cases, the mixed peptoid delivery system further comprises a neutral lipid component, such as a sterol (e.g., cholesterol). Example mixed peptoid delivery systems containing a lipidated cationic peptoid and an anionic/zwitterionic lipidated peptoid as the non-cationic peptoid can be found in Table 4.

In some implementations, the mixed peptoid delivery system comprises a lipidated cationic peptoid selected from the group consisting of Compound 24, 73, 79, 81, 85, 93, 112, 115, 127, 137, 152, 155, 163, 164, and combinations thereof; and a non-cationic lipidated that is a PEGylated lipid (e.g., Compound 106, 107, 108, and 109). In some implementations, the mixed peptoid delivery system does not contain an anionic/zwitterionic component. In some implementations, the mixed peptoid delivery systems includes an anionic/zwitterionic component. In some cases, the anionic/zwitterionic component is a phospholipid (e.g., DSPC, DOPE). In some implementations, the anionic/zwitterionic component is DSPC. In some implementations, the anionic/zwitterionic component is DOPE. In some implementations, the mixed peptoid delivery system further comprises a PEGyated lipid component (e.g., DMG-PEG 2000). In some cases, the mixed peptoid delivery system further comprises a neutral lipid component, such as a sterol (e.g., cholesterol). Example mixed peptoid delivery systems containing a lipidated cationic peptoid and an anionic/zwitterionic lipidated peptoid as the non-cationic peptoid can be found in Table 4.

In some examples, the delivery vehicle composition comprises about 30 to about 45 mol % of the cationic component; about 5 to about 15 mol % of the anionic/zwitterionic component, about 40 to about 60 mol % of the neutral lipid component, and about 1 to about 5 mol % of the shielding component. In various examples, the delivery vehicle composition comprises about 35 to about 40 mol % of the cationic component; about 8 to about 12 mol % of the anionic/zwitterionic component, about 45 to about 50 mol % of the neutral lipid component, and about 1 to about 3 mol % of the shielding component. In various examples, the delivery vehicle composition comprises about 38.2 mol % of the cationic component; about 11.8 mol % of the anionic/zwitterionic component, about 48.2 mol % of the neutral lipid compound, and about 1.9 mol % of the shielding component.

In some examples, the delivery vehicle composition comprises about 45 to about 55 mol % of the cationic component; about 5 to about 15 mol % of the anionic/zwitterionic component, about 35 to about 55 mol % of the neutral lipid compound, and about 1 to about 5 mol % of the shielding component. In various examples, the delivery vehicle composition comprises about 48 to about 52 mol % of the cationic component; about 5- about 12 mol % of the anionic/zwitterionic component, about 38 to about 42 mol % of the neutral lipid compound, and about 1 to about 3 mol % of the shielding component. In various examples, the delivery vehicle composition comprises about 51.3 mol % of the cationic component; about 9.3 mol % of the anionic/zwitterionic component, about 38.0 mol % of the neutral lipid compound, and about 1.5 mol % of the shielding component.

In various cases, the multicomponent delivery system comprises between about 10 to about 55 mol %, about 15 to about 50 mol %, about 20 to about 45 mol %, about 25 to about 40 mol %, about 30 to about 50 mol %, about 35 to about 45 mol %, about 40 to about 48 mol %, about 43 to about 49.5 mol %, or about 20 to about 35 mol % of lipidated cationic peptoids of the total number of moles of the multicomponent delivery system. In some variations, the multicomponent delivery system comprises between 0 to about 10 mol %, about 5 to about 20 mol %, about 10 to about 30 mol %, about 15 to about 40 mol %, about 20 to about 50 mol %, about 30 to about 60 mol %, about 55 to about 70 mol %, about 60 to about 75 mol %, or about 70 to about 85 mol % of neutral lipidated component of the total number of moles of the multicomponent delivery system. In certain variations, the multicomponent delivery system comprises 0 mol %, about 15 to about 35 mol %, about 20 to about 40.0 mol %, about 20 to about 35 mol %, about 30 to about 60 mol %, about 40 to about 50 mol %, or about 50 to about 60 mol % of one or more of the anionic/zwitterionic compound of the total moles of the multicomponent delivery system. In certain implementations, the multicomponent delivery system comprises 0.5 to about 5 mol % (such as about 0.5 to about 1 mol %, about 1 to about 2 mol %, about 2 to about 3 mol %, about 3 to about 4 mol %, about 4 to about 5 mol %) one or more PEGylated compounds (e.g., DMG-PEG 2000) of the total moles of the multicomponent delivery system.

In one non-limiting example, the multicomponent delivery system comprises at least about 99 mol % cationic component and less than about 1 mol % shielding component (e.g., Formula F1A in Tables 2 and 3). The cationic component may be a lipidated cationic peptoid listed in Table 1A (e.g., Compound 24, 73, 79, 81, 85, 93, 112, 115, 127, 137, 152, 155, 163, and 164). The shielding component may be a peptoid or lipid comprising PEG moieties.

In another non-limiting example, the multicomponent delivery system comprises less than about 20 mol % cationic component, less than about 5 mol % shielding component, and more than about 75 mol % a mixture of anionic/zwitterionic component and lipid component (e.g., Formula F2A and Formula F4A in Tables 2 and 3). The cationic component may be a lipidated cationic peptoid such as a compound listed in in Table 1A (e.g., Compound 24, 73, 79, 81, 85, 93, 112, 115, 127, 137, 152, 155, 163, and 164). The shielding component may be a peptoid or lipid comprising PEG moieties. The lipid component may be a peptoid or lipid comprising phospholipid, cholesterol, or a mixture thereof.

In yet another non-limiting example, the multicomponent delivery system comprises about 30 to about 45 mol % cationic component, about 50 to about 70 mol % a mixture of anionic/zwitterionic component and lipid component, and about 1.5 to about 4.5 mol % shielding component (e.g., Formula F3A and Formula F5A in Tables 2 and 3). The cationic component may be a lipidated cationic peptoid such as a compound listed in in Table 1A (e.g., Compound 24, 73, 79, 81, 85, 93, 112, 115, 127, 137, 152, 155, 163, and 164). The shielding component may be a peptoid or lipid comprising PEG moieties. The anionic/zwitterionic component may be a peptoid or lipid comprising phospholipid, or a mixture thereof. The lipid component may be a peptoid or lipid comprising cholesterol, or a mixture thereof.

In yet another non-limiting example, the multicomponent delivery system comprises about 15 to about 35 mol % cationic component, about 60 to about 80 mol % a mixture of anionic/zwitterionic component and lipid component, and about 1.5 to about 3.0 mol % shielding component (e.g., Formula F2A and Formula F3A in Tables 2 and 3). The cationic component may be a lipidated cationic peptoid such as a compound listed in Table 1A (e.g., Compound 24, 73, 79, 81, 85, 93, 112, 115, 127, 137, 152, 155, 163, and 164). The shielding component may be a peptoid or lipid comprising PEG moieties. The anionic/zwitterionic component may be a peptoid or lipid comprising phospholipid, or a mixture thereof. The lipid component may be a peptoid or lipid comprising cholesterol, or a mixture thereof.

In yet another non-limiting example, the multicomponent delivery system comprises about 15 to about 35 mol % cationic component, about 10 to about 20 mol % an anionic/zwitterionic component, about 50 to about 65 mol % lipid component, and about 1.5-3.0 mol % shielding component (e.g., Formula F2A and Formula F3A in Tables 2 and 3). The cationic component may be a lipidated cationic peptoid such as a compound listed in Table 1A (e.g., Compound 24, 73, 79, 81, 85, 93, 112, 115, 127, 137, 152, 155, 163, and 164). The shielding component may be a peptoid or lipid comprising PEG moieties. The anionic/zwitterionic component may be a peptoid or lipid comprising phospholipid, or a mixture thereof. The lipid component may be a peptoid or lipid comprising cholesterol, or a mixture thereof.

In yet another non-limiting example, the multicomponent delivery system comprises about 10 to about 20 mol % cationic component, about 75 to about 89 mol % lipid component, and about 1 to about 5 mol % shielding component (e.g., Formula F4A in Tables 2 and 3). The cationic component may be a lipidated cationic peptoid listed in Table 1A (e.g., Compound 24, 73, 79, 81, 85, 93, 112, 115, 127, 137, 152, 155, 163, and 164). The shielding component may be a peptoid or lipid comprising PEG moieties. The lipid component may be a peptoid or lipid comprising cholesterol, or a mixture thereof.

In yet another non-limiting example, the multicomponent delivery system comprises about 40 to about 50 mol % cationic component, about 50 to about 59 mol % an anionic/zwitterionic component, and about 1 to about 5 mol % shielding component (e.g., Formula F5A in Tables 2 and 3). The cationic component may be a lipidated cationic peptoid listed in Table 1A (e.g., Compound 24, 73, 79, 81, 85, 93, 112, 115, 127, 137, 152, 155, 163, and 164). The shielding component may be a peptoid or lipid comprising PEG moieties. The anionic/zwitterionic component may be a peptoid or lipid comprising phospholipid, or a mixture thereof.

Non-limiting examples of the multicomponent delivery system formulations are included in Table 2 (molecular percentages) and Table 3 (mass ratios and charge ratios). In Formula F2A, F3A, or F5A, the anionic/zwitterionic component or lipid component comprising phospholipid can be, but is not limited to, DOPE, DSPC or Compound 105. In Formula F2A, F3A or F4A, the lipid component comprising cholesterol can be, but is not limited to, cholesterol, Compound 89, Compound 90, or Compound 119. The shielding component in Formula F1A, F2A, F3A, F4A, F5A, or F6 can be, but is not limited to DMG-PEG (such as DMD-PEG 2000) or Compound 107. In some implementations, the components of Table 2 (cationic component, anionic/zwitterionic component, neutral lipid component, and shielding component) are selected from the components listed in Table 1F.

Table 2, below, provides representative molecular percentages in example multicomponent delivery system formulations that comprise Compound 81 as the cationic component and Compound 90 as the non-cationic component. Similar molecular percentages can be calculated for any multicomponent delivery vehicle system disclosed herein, based on the mass ratios of a particular formulation (e.g., as listed in Table 4) and the molecular weights of the components in the formulation.

TABLE 2
Example molecular percentages of the components
of the multicomponent delivery system

| Molecular | | Anionic or | | Shielding |
|---|---|---|---|---|
| Percentages | Cationic | Zwitterionic | Neutral lipid | (PEGylated) |
| (mol %) | component | component | component | component |
| F1A | 99.1 | 0 | 0 | 0.9 |
| F2A | 17.9 | 16.4 | 62.9 | 2.8 |
| F3A | 32.9 | 13.4 | 51.7 | 2.0 |
| F4A | 17.1 | 0 | 80.2 | 2.7 |
| F5A | 42.3 | 53.3 | 0 | 4.4 |
| F1 | 21.4 | 15.7 | 60.3 | 2.7 |
| F2 | 38.2 | 11.8 | 48.2 | 1.9 |
| F3 | 36.0 | 10.0 | 51.7 | 2.4 |
| F4 | 30.2 | 29.6 | 39.5 | 0.7 |
| F5 | 32.0 | 16.7 | 48.7 | 2.6 |
| F6 | 51.3 | 9.3 | 38.0 | 1.5 |
| MP1 | 38.0 | 0 | 59.7 | 2.3 |
| MP2 | 69.8 | 0 | 26.4 | 3.8 |
| MP3 | 51.4 | 13.9 | 32 | 2.7 |

### Complexes of the Multicomponent Delivery Systems

The multicomponent delivery vehicle compositions disclosed herein can form complexes with one or more polyanionic compounds (e.g., nucleic acids) through an electrostatic interaction between the cationic component of the delivery vehicle composition and the polyanionic compound. The delivery vehicle complexes, in some instances permit a high amount of cargo encapsulation, are stable, and demonstrate excellent efficiency and tolerability in vivo. The multicomponent delivery vehicle complexes, therefore, are useful as delivery vehicles for the transportation of the polyanionic cargo encapsulated therein to a target cell. Additionally or alternatively, the delivery vehicle compositions can include a non-anionic cargo. Accordingly, another aspect of the disclosure relates to a multicomponent delivery system complex comprising: (1) a multicomponent delivery vehicle system, as previously described herein, and (2) a polyanionic compound (or cargo). In some examples, the multicomponent delivery vehicle system complexes with one polyanionic compound (e.g., one RNA). In various examples, the delivery vehicle composition complexes with two different polyanionic compound (e.g., two different RNAs or an RNA and a DNA). In some examples, the multicomponent delivery vehicle system complexes with three or more different polyanionic compounds (e.g., 3, 4, or 5 different RNAs). In some cases, the multicomponent delivery vehicle system complexes with one or more of a nucleic acid selected from DNA and RNA (e.g., an antigenic RNA and adjuvanting DNA, such as CpG).

With regard to the polyanionic cargo compounds (e.g., nucleic acids, such as RNA) present in the complexes of the present disclosure, the quantity of polyanionic cargo compounds within the complexes, may be characterized in a number of ways. In some implementations, the complexes described herein may be characterized by the ratio of the number cationic groups on the cationic component of the multicomponent delivery system to the number of anionic groups on the polyanionic cargo, such as the phosphate groups on the nucleic acid. In some implementations, the complex comprises the lipidated cationic peptoids and the nucleic acid at a cation:anion charge ratio of between about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, or between about 2:1 and about 5:1. In certain implementations, the complex comprises the lipidated cationic peptoids and the nucleic acid at a cation:anion charge ratio of between about 2:1 and about 5:1. In still yet other implementations, the complex comprises the lipidated cationic peptoid compound and the nucleic acid at a cation: anion charge ratio of about 3:1.

The multicomponent delivery vehicle system complexes described herein may be characterized by the relative mass ratio of one of the components of the multicomponent delivery vehicle system to the cargo (e.g., a polyanionic compound) in the complex. Mass ratios of the components in the delivery vehicle complex can be readily calculated based upon the known concentrations and volumes of stock solutions of each component used in preparing the complex. Moreover, if non-anionic cargoes are present in the delivery vehicle complex, mass ratios may provide a more accurate representation of the relative amounts of delivery vehicle components to the overall cargo than cation:anion charge ratios, which do not account for non-anionic material. Specifically, the mass ratio of a component refers to the ratio of the mass of the particular component in the system to the mass of the “cargo” in the system. “Cargo” may refer to the total polyanoic compound(s) present in the system. In one example, the polyanionic compound(s) may refer to nucleic acid(s). In one example, the polyanionic compound(s) refer to mRNA(s) encoding at least one protein. In some cases, the polyanionic compound(s) refer to a combination of RNA and DNA.

In some implementations, the complex comprises the one or more lipidated cationic peptoids and the cargo comprising one or more polyanionic cargo compounds and/or non-anionic cargo compounds at a mass ratio of between about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, or between about 2:1 and about 5:1. In certain implementations, the complex comprises the one or more lipidated cationic peptoids and the cargo comprises one or more polyanionic cargo compounds and/or non-anionic cargo compounds at a mass ratio of between about 2:1 and about 5:1. In still yet other implementations, the complex comprises the one or more lipidated cationic peptoids and the cargo comprises one or more polyanionic cargo compounds and/or non-anionic cargo compounds at a mass ratio of about 3:1.

In certain implementations wherein the complex comprises a nucleic acid as cargo, the complex comprises the lipidated cationic peptoids and the nucleic acid at a mass ratio of between about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, or between about 2:1 and about 5:1. In certain implementations, the complex comprises the lipidated cationic peptoids and the nucleic acid at a mass ratio of between about 2:1 and about 5:1. In still yet other implementations, the complex comprises the lipidated cationic peptoids and the nucleic acid at a mass ratio of about 3:1.

In still other implementations, the amount of polyanionic cargo compounds present in the complexes may be characterized by a mass ratio of the multicomponent delivery system (e.g., lipidated cationic peptoids, non-cationic lipidated peptoids, phospholipid, cholesterol, and/or the shielding component in total) to the one or more polyanionic cargo compounds. In some implementations, the mass ratio of the multicomponent delivery system to the one or more polyanionic cargo compounds is between about 0.5:1 and 30:1, between about 0.5:1 and about 25:1, about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, or between about 2:1 and about 5:1, or between about 2:1 and about 30:1, between about 2:1 and 20:1. In certain implementations, the mass ratio of the multicomponent delivery system to the one or more polyanionic cargo compounds is between about 5:1 and about 8:1 or between about 6:1 and about 7:1.

In some examples, the cationic component (e.g., lipidated cationic peptoid) and the polyanionic compound of the delivery vehicle complex have a mass ratio between about 0.5:1 and about 20:1, between about 5:1 and about 20:1, between about 2:1 and about 15:1, between about 7:1 and about 15:1, between about 8:1 and about 12:1, or between about 10:1 to about 11:1. In some examples, the cationic component (e.g., lipidated cationic peptoid) and the polyanionic compound of the delivery vehicle complex has a mass ratio of about 10:1. In other examples, the cationic component (e.g., lipidated cationic peptoid) and the polyanionic compound of the delivery vehicle complex has a mass ratio of about 10.3:1 or about 10.8:1. In some examples, the cationic component (e.g., lipidated cationic peptoid) can be any cationic component as previously described herein (e.g., Compound 24, 73, 79, 81, 85, 93, 112, 115, 127, 137, 152, 155, 163, 164, and combinations thereof (e.g., Compound 81, 112, 155, 163, 164, and combinations thereof). In some examples, the polyanionic cargo comprises a nucleic acid, such as RNA and/or DNA. In some cases, the polyanionic cargo comprises RNA.

In some cases, the mass ratio of the non-cationic lipidated peptoid component of the delivery vehicle complex (e.g., a neutral lipidated peptoid or an anionic/zwitterionic lipidated peptoid) and the polyanionic compound can be between about about 2:1 to about 7:1, or about 2:1 to about 4:1, or about 4:1 and about 6:1, or about 5:1 to about 6:1, or about 2.5:1 to about 3.5:1. In some examples, the mass ratio of the non-cationic lipidated peptoid component and the polyanionic compound is about 5.4:1. In some examples, the mass ratio of the non-cationic lipidated peptoid component and the polyanionic compound is about 3.1:1. The non-cationic lipidated peptoid component can be any non-cationic lipidated peptoid component, as previously described herein (e.g., Compound 90, 119, 120, 121, 122, 123, 124, 125, 126, 128, 129). In various examples, the non-cationic component is a non-peptoid neutral lipid component, such as a sterol (e.g., cholesterol). In some examples, the polyanionic cargo is a nucleic acid, such as RNA.

In some cases, the delivery vehicle complex does not include a phospholipid. In some cases, the delivery vehicle complex includes a phospholipid, and the mass ratio of the phospholipid and the polyanionic compound is between about 0.5:1 to about 5:1, or about 1:1 to about 3:1, or about 1.5:1 to about 2.5:1. In some examples, the mass ratio of phospholipid and the polyanionic compound is about 2.2:1. In various examples, the phospholipid is DOPE or DSPC. In some examples, the polyanionic cargo is a nucleic acid, such as RNA.

In some cases, delivery vehicle complex includes a shielding component. The shielding component can be, in some examples, a PEGylated lipid, as previously described herein, or a PEGylated lipidated peptoid (e.g., Compound 107), as previously described herein. In some implementations, the mass ratio of the shielding component and the polyanionic compound is between about 0.5:1 to about 2.5:1, or about 1:1 to about 2:1, or about 2:1 to about 7:1, or about 4:1 to about 6:1, or about 5:1 to about 5.5:1 In some examples, the mass ratio of the shielding component and the polyanionic compound is about 1.4:1 or about 5.4:1. In some examples, the shielding component can be a PEGylated lipitoid, as previously described herein (e.g., Compound 107). In various examples, the shielding component is DMG-PEG 2000. In some examples, the polyanionic cargo is a nucleic acid, such as RNA.

In some cases, the mass ratio of the lipidated cationic peptoid to the polyanionic compound is about 10:1, the mass ratio of the non-cationic lipidated peptoid component to the polyanionic compound is about 5.4:1, the mass ratio of the PEGylated lipid to the polyanionic compound is about 1.4:1, and the mass ratio of the phospholipid to the polyanionic compound is 0:1.

Non-limiting examples of delivery vehicle formulations by mass ratio and charge ratio can be found in Table 3, below. Further, non-limiting examples of multicomponent delivery vehicle complexes can be found in Table 4, below. The single component delivery vehicle complexes are designated by the identity of the cationic lipidated peptoid (e.g., Compound 112), and the formulation identifier from Table 3. For example the delivery vehicle complex “112-F2” includes Compound 112 as the lipidated cationic peptoid in formulation F2, which is described in Table 3. The mixed peptoid delivery vehicle complexes are designated by the identity of the cationic lipidated peptoid (e.g., Compound 81 or 112), the identity of the neutral lipidated peptoid (e.g., Compound 90 or 119), and the identity of the mixed peptoid formulation from Table 3 (e.g, MP1, MP2, or MP3). For example, the delivery vehicle complex 81:MP1:90 refers to a complex comprising Compound 81 as the lipidated cationic peptoid and Compound 90 as the neutral lipidated peptoid, in formulation MP 1. The nomenclature for mixed peptoid complexes having three peptoids is similar. For example, 163-MP-135(MP1):119 refers to a mixed peptoid complex comprising lipidated cationic peptoids 163 and 135, and non-cationic peptoid 119, in formulation MP1. In some examples, the mixed peptoid delivery system can be 112:MP1:90, 112:MP1:119, 81:MP1:90, 81:MP1:119, 155-MP1-90, 155-MP1-119, 163-MP1-90, 163-MP1-119, 137-MP1-127, 163-MP-135(MP1):119, or 155-MP-135(MP1):119, as shown in Table 4.

Table 3, below, provides representative mass ratios and charge ratios in example multicomponent delivery system formulations that comprise Compound 81 as the cationic component and Compound 90 as the non-cationic component. Similar mass ratios can be calculated for any multicomponent delivery vehicle system disclosed herein, based on the molecular weights and amounts of the particular components used in a formulation.

TABLE 3
Mass ratios and charge ratios of the components
of the multicomponent delivery system

| | | Anionic or | | | |
|---|---|---|---|---|---|
| | Cationic | Zwitterionic | Neutral lipid | Shielding | |
| | component | component | component | component | Charge |
| | mass ratio | mass ratio | mass ratio | mass ratio | ratios |
| F1A | 5.0 | 0 | 0 | 0.1 | 3.0 |
| F2A | 5.0 | 3.0 | 6.0 | 1.8 | 3.0 |
| F3A | 10 | 2.7 | 5.4 | 1.4 | 6.0 |
| F4A | 5.0 | 0 | 8.0 | 1.8 | 3.0 |
| F5A | 8.5 | 7.0 | 0 | 2.0 | 5.0 |
| F6A | 10 | 0 | 5.4 | 1.4 | 6.0 |
| F1 | 5.0 | 3.0 | 6.0 | 1.8 | 1.87 |
| F2 | 10 | 2.7 | 5.4 | 1.4 | 3.8 |
| F3 | 10 | 2.4 | 6.1 | 1.8 | 3.8 |
| F4 | 10 | 8.0 | 5.6 | 0.69 | 3.8 |
| F5 | 10 | 4.3 | 6.5 | 2.3 | 3.8 |
| F6 | 17 | 2.7 | 5.4 | 1.4 | 6.36 |
| MP1 | 10 | 0 | 5.4 | 1.4 | 6.0 |
| MP2 | 10.3 | 0 | 3.1 | 1.4 | 3.3 |
| MP3 | 10.8 | 2.2 | 5.4 | 5.4 | 3.3 |

### Multicomponent Delivery Vehicle System Characterization

The delivery vehicle complexes disclosed herein can be characterized by various different parameters, such as particle size, polydispersity index, and percent encapsulation of cargo. In one implementation, a delivery vehicle complex resembles a nanoparticle, including at least one polyanionic compound (described further below) being encapsulated by a delivery vehicle composition. In one implementation, such a complex is an mRNA nanoparticle including a delivery vehicle composition encapsulating at least one mRNA.

In some examples, the delivery vehicle complexes disclosed herein can have a mean diameter of less than about 300 nm, or less than about 275 nm, or less than about 250 nm, or less than about 225 nm, or less than about 200 nm, or less than about 175 nm, or less than about 150 nm, or less than about 125 nm, or less than about 100 nm, or less than about 90 nm, or less than about 80 nm, or less than about 70 nm, or less than about 60 nm, or less than about 50 nm, or less than about 40 nm. In some implementations, the delivery vehicle complexes disclosed herein can range in size from about 40 nm to about 200 nm in diameter, or from about 50 nm to about 175 nm, or from about 60 nm to about 150 nm, or from about 70 nm to about 125 nm, or from about 80 nm to about 100 nm, or from about 70 nm to about 90 nm, or from about 75 nm to about 95 nm, or from about 80 nm to about 110 nm, or from about 90 nm to about 125 nm, or from about 70 nm to about 90 nm, or from about 40 nm to 100 nm, or from about 50 nm to 95 nm, or from about 60 nm to about 85 nm. In another implementation, the complex may have a size of greater than about 100 nm in diameter, for example, between about 105 nm and about 250 nm, between about 110 nm and about 220 nm, and between about 150 nm and about 200 nm. In one implementation, the complex may have a size of between about 105 nm and about 200 nm in diameter, or between about 105 nm and about 120 nm, or between about 105 and 110 nm, or between about 140 nm and 190 nm, or between about 120 nm and 150 nm. In some cases, the delivery vehicle complex exhibits a particle size of about 40 nm to about 115 nm, or about 55 nm to about 95 nm, or about 70 to about 80 nm, or about 75 nm. In various cases, the delivery vehicle complex exhibits a particle size of about 135 nm to about 225 nm, or about 155 nm to about 195 nm, or about 170 to about 180 nm, or about 175 nm. In some cases, the particle size depends on the method used to prepare the complex (e.g., via a microfluidic device or by hand). The particle size/diameter can be determined by dynamic light scattering (DLS), and described in Example 3.

In some implementations, the delivery vehicle complexes of the disclosure exhibit a polydispersity index (PDI) of less than about 0.3, about 0.25, about 0.2, about 0.19, about 0.18, about 0.17, about 0.16, about 0.15, about 0.14, about 0.13, about 0.12, about 0.11, or about 0.10.

In some implementations, at least about 40%, about 45%, about 50%, about 55%, about 60%, about 65%, about 66%, about 67%, about 68%, about 69%, about 70%, about 75%, about 80%, about 85%, about 90%, about 95%, about 96%, about 97%, about 98%, or about 99% of the nucleic acid (e.g., RNA) cargo is fully encapsulated in the delivery vehicle complexes. The percentage of mRNA encapsulated within the delivery vehicle complexes can be determined used a modified RiboGreen assay, as described in Example 3.

The delivery vehicle complexes of the disclosure exhibit good storage stability. In some examples, the delivery vehicle complexes of the disclosure retain at least about 70%, or at least about 80%, or at least about 85%, or at least about 90%, or at least about 95%, or at least about 96%, or at least about 97%, or at least about 98%, or at least about 99%, or at least about 99.5%, or about 100% of the polyanionic compound after storage at 4° C.-10° C. for at least 10 days—e.g., at least 20 days, 30 days, 40 days, 50 days, 60 days, 70 days, or more. In one implementation, the delivery vehicle complexes retain the aforementioned level of polyanionic compound at 4° C. for 48 days. Further, in some cases, the delivery vehicle complexes of the disclosure retain at least about 70%, or at least about 80%, or at least about 85%, or at least about 90%, or at least about 95%, or at least about 96%, or at least about 97%, or at least about 98%, or at least about 99%, or at least about 99.5%, or about 100% of their original size after storage at 4° C.-10° C. for at least 10 days—e.g., at least 20 days, 30 days, 40 days, 50 days, 60 days, 70 days, or more. In one implementation, the delivery vehicle complexes retain the aforementioned size after storage at 4° C. or 48 days.

The delivery vehicle complexes disclosed herein have also been found to be well tolerated at high and low doses with no systemic toxicity or adverse events.

### Polyanionic Compound

The delivery vehicle complexes of the disclosure can comprise one or more polyanionic compounds (polyanionic cargo) that can be delivered by the complex to a target in vivo, such as a cell. The polyanionic compound can be complexed to the cationic component (e.g., a lipidated cationic peptoid, as described herein) of the delivery vehicle complex via electrostatic interactions.

In some implementations, the one or more polyanionic cargo compounds comprises a nucleic acid. Nucleic acids, as used herein, include naturally occurring nucleic acids such as DNA, RNA, and/or hybrids thereof, as well as unnaturally occurring nucleic acids having an unnatural backbone, modified backbone linkages such as phosphorothioate, unnatural and modified bases, and/or unnatural and modified termini. Example nucleic acids include genomic DNA, cDNA, mRNA, miRNA, and siRNA. In some cases, the nucleic acid cargo comprises, DNA, RNA, or a combination thereof. In certain implementations, the nucleic acid cargo is RNA including but not limited to modified mRNAs, self-amplifying RNAs, and circular RNAs.

The nucleic acids may be recombinantly produced or chemically synthesized molecules. A nucleic acid may be single-stranded, double-stranded, triple stranded, or quadruple stranded, as well as in more complicated three-dimensional forms including single and double stranded regions.

Depending upon the type of nucleic acid, the length of the nucleic acid (defined in nucleotide units or base pairs (bp) as appropriate) may vary. In some implementations wherein the nucleic acid is mRNA, the mRNA may have from 100 to 10,000 nucleotide units, or from 1,000 to 3,000 nucleotide units. In other implementations wherein the nucleic acid is DNA, the DNA may have from 5,000 bp to 20,000 bp, or about 10,000 bp.

In some implementations wherein the nucleic acid is an mRNA encoding a protein or a peptide. The peptide may be an oligopeptide or a polypeptide. In certain implementations, the mRNA is an mRNA encoding a polypeptide. In yet further implementations, the mRNA is an mRNA encoding a protein. As described above, the mRNA may be naturally-occurring (e.g., isolated tumor RNA) or may be synthetic (e.g., produced by in vitro transcription). For synthetic or unnaturally occurring variations of mRNA, the mRNA may comprise an unnatural backbone with modified backbone linkages such as phosphorothioate, unnatural and modified bases, and/or unnatural and modified termini. In certain implementations wherein the nucleic acid is an mRNA, the mRNA may comprise special sequences such as self-amplifying sequences or internal ribosome entry sites.

According to certain examples of the disclosure, the RNAs for use in the delivery vehicle complexes disclosed herein comprise an RNA comprising at least one region encoding a peptide (e.g., a polypeptide), or protein, or functional fragment of the foregoing. As used herein, “functional fragment” refers to a fragment of a peptide (e.g., a polypeptide), or protein that retains the ability to induce an immune response. In one example, the coding RNA is selected from the group consisting of mRNA, viral RNA, retroviral RNA, and self-replicating RNA. In some examples, the RNA encodes a viral peptide (e.g., a viral polypeptide), a viral protein, or functional fragment of the foregoing.

In some examples, the RNA encodes for adenovirus, alphavirus, calicivirus (e.g., a calicivirus capsid antigen), coronavirus polypeptides, distemper virus, Ebola virus polypeptides, enterovirus, flavivirus, hepatitis virus (AE), herpesvirus, infectious peritonitis virus, leukemia virus, Marburg virus, orthomyxovirus, papilloma virus, parainfluenza virus, paramyxovirus, parvovirus, pestivirus, picorna virus (e.g., a poliovirus), pox virus (e.g., a vaccinia virus), rabies virus, reovirus, retrovirus, and rotavirus. In certain examples, the RNA encodes for SARS-CoV-2, HPV (e.g., E6 and/or E7), or influenza (e.g., influenza hemagglutinin (HA)).

In various cases, the RNA encodes for a human papillomavirus (HPV) protein or a functional fragment thereof. In some cases, the RNA encodes for a HPV E6 protein, a HPV E7 protein, a combination thereof, or a functional fragment any of the foregoing. In some cases, the RNA encodes for a viral spike protein or a functional fragment thereof. In various examples, the RNA encodes for a SARS-CoV spike (S) protein, or a functional fragment thereof. In some cases, the RNA encodes for an influenza protein, or a functional fragment thereof. In various examples, the RNA encodes for influenza hemagglutinin (HA), or a functional fragment thereof. In some examples, the RNA encodes for a combination of the foregoing.

In some implementations, the one or more polyanionic cargo compounds may include anionic or polyanionic cargo compounds that are not nucleic acids. Suitable anionic compounds may include but are not limited to proteins, polyphosphates, or heparins. In some implementations, the one or more polyanionic cargo compounds comprises one or more proteins. In one implementations, the one or more polyanionic cargo compounds comprises Cas9 protein. In other implementations, the one or more polyanionic cargo compounds comprises polyphosphates. In yet other implementations, the one or more polyanionic cargo compounds comprises heparins or other glycosaminoglycan derivatives.

In some implementations, the combined delivery of two or more particular nucleic acids together may be especially useful for therapeutic applications. For example, in some implementations, the one or more polyanionic cargo compounds includes a combination of sgRNA (single guide RNA) as a CRISPR sequence and mRNA encoding Cas9. In still further implementations, the nucleic acids may also be complexed with proteins such as with the CRISPR/Cas9 ribonucleoprotein complex. In some cases, the multicomponent delivery vehicle system complexes with one or more of a nucleic acid selected from DNA and RNA (e.g., an antigenic RNA and adjuvanting DNA, such as CpG).

### Methods of Making the Multicomponent Delivery System

Components of the multicomponent delivery system can be prepared through a variety of physical and/or chemical methods to modulate their physical, chemical, and biological properties. These may involve rapid combination of the lipidated cationic peptoid compound (e.g., a tertiary amino lipidated and/or PEGylated cationic peptoid or a lipitoid) in water or a water-miscible organic solvent with the desired polyanionic cargo compound (e.g., oligonucleotides or nucleic acids) in water or an aqueous buffer solution. These methods can include simple mixing of the components by pipetting, or microfluidic mixing processes such as those involving T-mixers, vortex mixers, or other chaotic mixing structures. In some implementations, the multicomponent delivery system is prepared on a microfluidic platform.

It is to be understood that the particular process conditions for preparing the multicomponent delivery system described herein may be adjusted or selected accordingly to provide the desired physical properties of the compositions. For example, parameters for mixing the components of the multicomponent delivery system that may influence the final compositions may include, but are not limited to, order of mixing, temperature of mixing, mixing speed/rate, flow rate, physical dimensions of the mixing structure, concentrations of starting solutions, molar ratio of components, and solvents used.

Formulation of the multicomponent delivery system can be accomplished in many ways. In some cases, all components can be pre-mixed prior to addition of the nucleic acid cargo, which can result in a uniform distribution of components throughout the delivery particle.

In other cases, the components can be added sequentially to produce a core-shell type structure. For example, a cationic component could be added first to begin particle condensation, followed by a lipid component to allow the particle's surface to associate with target cells, followed by a shielding component to prevent particle aggregation. For example, the lipidated cationic peptoid can be premixed with the nucleic acid cargo to form a core structure. Then, the lipid components (such as lipid components comprising phospholipids and cholesterol) can be added to influence cell/endosomal membrane association. Because the shielding component is primarily useful on the outside of the multicomponent delivery system, this component can be introduced last, so that it does not disrupt the internal structure of the system, but rather provides a coating of the system after it is formed.

Additional components in the complexes and composition, such as the additional components of polymers, surface-active agents, targeting moieties, and/or excipients, may be admixed and combined with the rest of the components before, during, or after the principal components of the nucleic acid cargo, the cationic component, the lipid component and the shielding component have been combined.

### Pharmaceutical Formulations and Modes of Administration

Also provided herein are pharmaceutical compositions that include the delivery vehicle complexes of the disclosure and an effective amount of one or more pharmaceutically acceptable excipients. An “effective amount” includes a “therapeutically effective amount” and a “prophylactically effective amount.” The term “therapeutically effective amount” refers to an amount effective in treating and/or ameliorating a disease or condition in a subject, and/or eliciting an immune response. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females. As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The complexes of the disclosure can be administered to a subject or patient in a therapeutically effective amount. The complexes can be administered alone or as part of a pharmaceutically acceptable composition or formulation. In addition, the complexes can be administered all at once, as for example, by a bolus injection, multiple times, or delivered substantially uniformly over a period of time. It is also noted that the dose of the compound can be varied over time.

The delivery vehicle complexes disclosed herein and other pharmaceutically active compounds, if desired, can be administered to a subject or patient by any suitable route, e.g. orally, rectally, parenterally, (for example, intravenously, intramuscularly, or subcutaneously) intracisternally, intravaginally, intraperitoneally, intravesically, or as a buccal, inhalation, or nasal spray. The administration can be to provide a systemic effect (e.g. enteral or parenteral). All methods that can be used by those skilled in the art to administer a pharmaceutically active agent are contemplated.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compositions for parenteral administrations can be administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

When the composition of the disclosure are used as vaccines, they may comprise one or more immunologic adjuvants. As used herein, the term “immunologic adjuvant” refers to a compound or a mixture of compounds that acts to accelerate, prolong, enhance or modify immune responses when used in conjugation with an immunogen (e.g., neoantigens). Adjuvant may be non-immunogenic when administered to a host alone, but that augments the host's immune response to another antigen when administered conjointly with that antigen. Specifically, the terms “adjuvant” and “immunologic adjuvant” can be used interchangeably in the present disclosure. Adjuvant-mediated enhancement and/or extension of the duration of the immune response can be assessed by any method known in the art including without limitation one or more of the following: (i) an increase in the number of antibodies produced in response to immunization with the adjuvant/antigen combination versus those produced in response to immunization with the antigen alone; (ii) an increase in the number of T cells recognizing the antigen or the adjuvant; and (iii) an increase in the level of one or more cytokines. Adjuvants may be aluminum based adjuvants including but not limiting to aluminum hydroxide and aluminum phosphate; saponins such as steroid saponins and triterpenold saponins; bacterial flagellin and some cytokines such as GM-CSF. Adjuvants selection may depend on antigens, vaccines and routes of administrations.

In some examples, adjuvants improve the adaptive immune response to a vaccine antigen by modulating innate immunity or facilitating transport and presentation. Adjuvants act directly or indirectly on antigen presenting cells (APCs) including dendritic cells (DCs). Adjuvants may be ligands for toll-like receptors (TLRs) and can directly affect DCs to alter the strength, potency, speed, duration, bias, breadth, and scope of adaptive immunity. In other instances, adjuvants may signal via proinflammatory pathways and promote immune cell infiltration, antigen presentation, and effector cell maturation. This class of adjuvants includes mineral salts, oil emulsions, nanoparticles, and polyelectrolytes and comprises colloids and molecular assemblies exhibiting complex, heterogeneous structures. In one example, the composition further comprises pidotimod as an adjuvant. In another example, the composition further comprises CpG as an adjuvant.

The compounds of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

The peptoids described herein can exist in free form, or where appropriate, as a pharmaceutically acceptable salt. As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio. Pharmaceutically acceptable salts are well known in the art. Pharmaceutically acceptable salts of the compounds described herein include those derived from suitable inorganic and organic acids and bases. These salts can be prepared in situ during the final isolation and purification of the compounds. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This disclosure also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Methods of Using the Multicomponent Delivery System

The delivery vehicle complexes disclosed herein can be used to deliver the polyanionic compound of the complex (or cargo) to a cell. Accordingly, disclosed herein are methods of delivering a polyanionic compound, such as a nucleic acid (e.g., RNA) to a cell comprising contacting the cell with the delivery vehicle complex or pharmaceutical composition disclosed herein. In some examples, the cell can be contacted in vitro. In some examples wherein the cell is contacted in vitro, the cell is a HeLa cell. In other examples wherein the cell is contacted in vivo, the multicomponent delivery system of the present disclosure is administered to a mammalian subject. A mammalian subject may include but is not limited to a human or a mouse subject. In yet other examples wherein the cell is contacted ex vivo, the cell is obtained from a human or mouse subject.

In some implementations, the one or more polyanionic cargo compounds may be delivered for therapeutic uses. Non-limiting therapeutic uses include cancer, infectious diseases, autoimmune disorders, and neurological disorders. In certain implementations, the complex comprising the multicomponent delivery system and the polyanionic cargo compound is used as a vaccine. Genetic vaccination, or the administration of nucleic acid molecules (e.g., RNA) to a patient and subsequent transcription and/or translation of the encoded genetic information, is useful in the treatment and/or the prevention of inherited genetic diseases but also autoimmune diseases, infectious diseases, cancerous or tumor-related diseases as well as inflammatory diseases. Genetic vaccination is useful for treating or preventing coronavirus. The vaccine target of the majority of these entities is the coronavirus' spike (S) protein, a heavily glycosylated trimeric class I fusion protein that coats the outside of the virus and is responsible for host cell entry. The S protein of SARS-CoV-2 shares high structural homology with SARS-CoV-1 and contains several subunits vital for viral entry into host cells through the angiotensin converting enzyme 2 (ACE2) receptor, including the 51 domain, the S2 domain, and the receptor binding domain (RBD). Thus, the S protein and its subunits, as well as accessible peptide sequences within these domains, are attractive vaccine antigen targets. Further, genetic vaccination is particularly use in the treatment of cancer because cancer cells express antigens, tumors are generally not readily recognized and eliminated by the host, as evidenced by the development of disease

In some implementations of the foregoing methods wherein the cell is contacted in vivo, the complex comprising the multicomponent delivery system and the nucleic acid cargos as described herein may be administered by injection (intravenous (IV), subcutaneous (SC), intramuscular (MI), or intrathecal injection. In other implementations, the complex comprising multicomponent delivery system and the nucleic acid cargos as described herein are administered by bolus injection or intravenous infusion. In other implementations wherein the cell is contacted in vivo, the complexes are administered by nasal or oral inhalation. In some implementations wherein the cell is contacted in vivo, the complexes are administered orally. In still other implementations wherein the cell is contacted in vivo, the complexes are administered via absorption into the mucous membrane (including topical, intra-anal, buccal, intravaginal, etc.).

It should be understood that clinical applications, such as the diagnostic, prophylactic and therapeutic examples disclosed above, may involve dosing regimens (e.g., dosage levels and time courses for administration) which may be varied as appropriate to the specific multicomponent delivery system and the nucleic acid cargos being used, the route of administration, the subject to which the complexes and compositions are being administered, and/or the desired physiological effect. For example, in some implementations, the methods of the present disclosure comprise administering the multicomponent delivery system and the nucleic acid cargos at a dose of 0.001 mg/kg to about 2 mg/kg of bodyweight.

For clinical applications, the complex comprising the multicomponent delivery system and the polyanionic cargo compound is further mixed with at least one pharmaceutically acceptable carrier. “Pharmaceutically acceptable carrier” as used herein refers to a carrier for administration of a therapeutic agent. Such carriers include, but are not limited to, saline, buffered saline, dextrose, water, glycerol, ethanol, and combinations thereof. The term specifically excludes cell culture medium. For the complex administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the oral composition, such as tablets, may be coated with a material such as glyceryl monostearate or glyceryl distearate, to delay absorption in the gastrointestinal tract.

### Vaccines

The delivery vehicle complexes of the disclosure are also useful as vaccines, in which the polyanionic compound is an RNA that may encode an immunogen, antigen or neoantigen. The immune system of a host provides the means for quickly and specifically mounting a protective response to pathogenic microorganisms and also for contributing to rejection of malignant tumors. Immune responses have been generally described as including humoral responses, in which antibodies specific for antigens are produced by differentiated B lymphocytes, and cell mediated responses, in which various types of T lymphocytes eliminate antigens by a variety of mechanisms. For example, CD4 (also called CD4+) helper T cells that are capable of recognizing specific antigens may respond by releasing soluble mediators such as cytokines to recruit additional cells of the immune system to participate in an immune response. CD8 (also called CD8+) cytotoxic T cells are also capable of recognizing specific antigens and may bind to and destroy or damage an antigen-bearing cell or particle. In particular, cell mediated immune responses that include a cytotoxic T lymphocyte (CTL) response can be important for elimination of tumor cells and cells infected by a microorganism, such as virus, bacteria, or parasite.

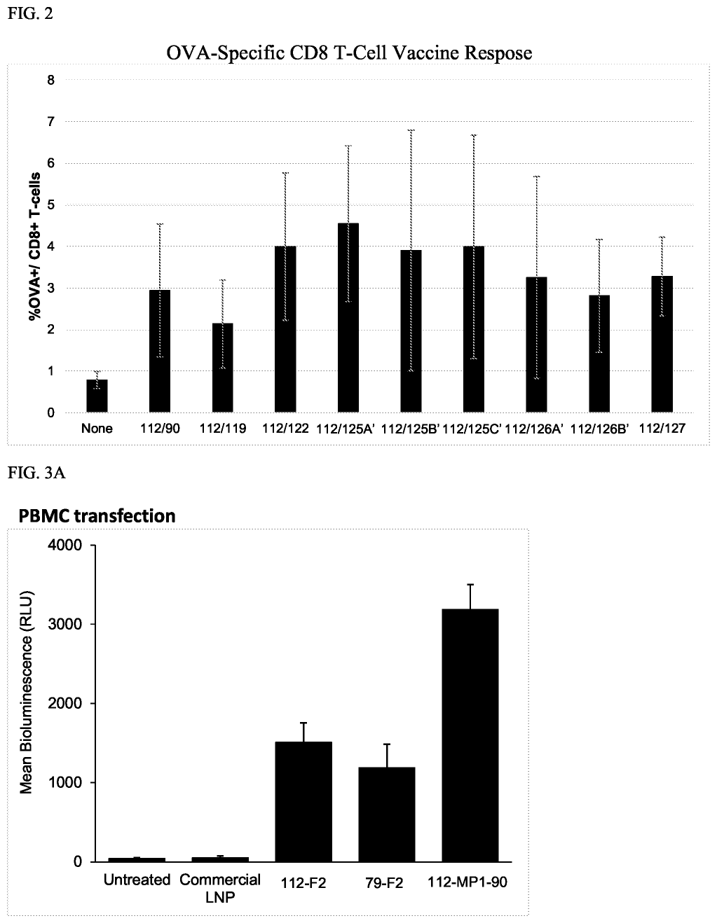

The delivery vehicle complexes of the disclosure have been found to induce immune responses when one or more of the polyanionic compound of the complex encodes a viral peptide (e.g., a viral polypeptide), a viral protein, or functional fragment of the foregoing. For example, delivery vehicle complexes 112:MP1:90, 112:MP1:119, 112:MP1:122, 112:MP1:125, 112:MP1:126; and 112:MP1:127 complexed with mRNA encoding Ovalbumin (OVA) elicited strong immune responses. See, e.g., Table 4, Example 5 and FIG. 2.

Thus, the disclosure includes methods for inducing an immune response in a subject in need thereof, comprising administering to the subject an effective amount of the delivery vehicle complex (e.g., formulated as an antigenic composition) of the disclosure. Also disclosed herein is a method of treating a viral infection in a subject in need thereof, comprising administering to the subject an effective amount of the delivery vehicle complex of the disclosure. In some examples, the administering is by intramuscular, intratumoral, intravenous, intraperitoneal, or subcutaneous delivery.

In various examples, administering the delivery vehicle complexes of the disclosure (e.g., formulated as a composition, pharmaceutical formulation, or antigenic composition) to a subject can result in an increase in the amount of antibodies (e.g., neutralizing antibodies) against the viral antigen that is produced in the subject relative to the amount of antibodies that is produced in a subject who was not administered the delivery vehicle complex. In some examples, the increase is a 2-fold increase, a 5-fold increase, a 10-fold increase, a 50-fold increase, a 100-fold increase, a 200-fold increase, a 500-fold increase, a 700-fold increase, or a 1000-fold increase.

The immune response raised by the methods of the present disclosure generally includes an antibody response, preferably a neutralizing antibody response, maturation and memory of T and B cells, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the delivery vehicle complexes comprising RNA that encodes a viral antigen as disclosed herein generates an immune response that recognizes, and preferably ameliorates and/or neutralizes, a viral infection as described herein. Methods for assessing antibody responses after administration of an antigenic composition (immunization or vaccination) are known in the art and/or described herein. In some examples, the immune response comprises a T cell-mediated response (e.g., peptide-specific response such as a proliferative response or a cytokine response). In some examples, the immune response comprises both a B cell and a T cell response. Antigenic compositions can be administered in a number of suitable ways, such as intramuscular injection, intratumoral injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

### Cancer

Various cancers may be treated with the polyanionic cargo compounds delivered by the multicomponent system of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

As a non-limiting example, the carcinoma which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

In some implementations, the delivery vehicle complexes of the disclosure are used to treat a cancer is selected from the group consisting of cervical cancer, head and neck cancer, B-cell lymphoma, T-cell lymphoma, prostate cancer, and lung cancer. In some implementations, the delivery vehicle complexes can be used to treat cervical cancer.

### Other Diseases and Disorders

### Infectious Diseases

In some implementations, the complex comprising the polyanionic cargo compounds and the multicomponent system of the present disclosure is used to treat infectious diseases, such as microbial infection, e.g., a viral infection, a bacterial infection, a fungal infection, or a parasitic infection. Non-limiting examples of infectious diseases include hepatitis (such as HBV infection or HCV infection), RSV, influenza, adenovirus, rhinovirus, or other viral infections.

### Autoimmune Diseases

Various autoimmune diseases and autoimmune-related diseases may be treated with the polyanionic compounds delivered by the multicomponent system of the present disclosure. As used herein, the term “autoimmune disease” refers to a disease in which the body produces antibodies that attack its own tissues. As a non-limiting example, the autoimmune disease may be Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome**, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed ryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia**, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosis, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA).

### Neurological Diseases

Various neurological diseases may be treated with the polyanionic compounds delivered by the multicomponent delivery system of the present disclosure. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Post infectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Shortlasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

### Definitions

“Alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

The term “alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Complex” as used herein includes any chemical association between two or more molecules, which may be mediated by ionic interactions, hydrogen bonding, van der Waals interactions, metal-ligand coordination, other chemical forces, and combinations of one or more of the foregoing. The complexes may form higher order structures including, for example, polyplexes, coacervate complexes, nanocomplexes, nanoparticles, and microparticles.

The term “cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. A preferred cycloalkyl is a cyclic hydrocarbon having from 3 to 13 annular carbon atoms. A more preferred cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Halo” or “halogen” refers to elements of the Group 17 series having atomic number 9 to 85. Preferred halo groups include fluoro, chloro, bromo and iodo. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl. Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

The term “heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms can be, in some instances, oxidized, and the nitrogen atom(s) can be, in some instances, quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to, pyridyl, pyrimidyl, thiophenyl, furanyl, thiazolyl, and the like.

The term “heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are, in some cases, y oxidized, and the nitrogen atom(s) are, in some cases quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, tetrahydrothiophenyl, 2,3-dihydrobenzo[b]thiophen-2-yl, 4-amino-2-oxopyrimidin-1(2H)-yl, and the like.

“Oxo” refers to the moiety ═O.

“Thiocarbonyl” refers to the group C═S.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some implementations, an optionally substituted group has 1 to 2, 2 to 5, 3 to 5, 2 to 3, 2 to 4, 3 to 4, 1 to 3, 1 to 4 or 1 to 5 substituents.

The term “substituted” refers to the replacement of one or more hydrogen atoms of a moiety with a monovalent or divalent radical. “Optionally substituted” indicates that the moiety may be substituted or unsubstituted. Suitable substituent groups include, for example, hydroxyl, nitro, amino (e.g., —NH₂ or dialkyl amino), imino, cyano, halo (such as F, Cl, Br, I), haloalkyl (such as —CCl₃ or —CF₃), thio, sulfonyl, thioamido, amidino, imidino, oxo, oxamidino, methoxamidino, imidino, guanidino, sulfonamido, carboxyl, formyl, alkyl, alkoxy, alkoxy-alkyl, alkylcarbonyl, alkylcarbonyloxy (—OCOR), aminocarbonyl, arylcarbonyl, aralkylcarbonyl, carbonylamino, heteroarylcarbonyl, heteroaralkyl-carbonyl, alkylthio, aminoalkyl, cyanoalkyl, carbamoyl (—NHCOOR— or —OCONHR—), urea (—NHCONHR—), aryl and the like, where R is any suitable group, e.g., alkyl or alkylene. In some implementations, the optionally substituted moiety is optionally substituted only with select radicals, as described. In some implementations, the above groups (e.g., alkyl groups) are optionally substituted with, for example, alkyl (e.g., methyl or ethyl), haloalkyl (e.g., —CCl₃, —CH₂CHCl₂ or —CF₃), cycloalkyl (e.g., —C₃H₅, —C₄H₇, —C₅H9), amino (e.g., —NH₂ or dialkyl amino), alkoxy (e.g., methoxy), heterocycloalkyl (e.g., as morpholine, piperazine, piperidine, azetidine), hydroxyl, and/or heteroaryl (e.g., oxazolyl). In some implementations, a substituent group is itself optionally substituted. In some implementations, a substituent group is not itself substituted. The group substituted onto the substitution group can be, for example, carboxyl, halo, nitro, amino, cyano, hydroxyl, alkyl, alkenyl, alkynyl, alkoxy, aminocarbonyl, —SR, thioamido, —SO₃H, —SO₂R or cycloalkyl, where R is any suitable group, e.g., a hydrogen or alkyl.

### Implementations

1. In one aspect, the disclosure provides a multicomponent delivery system comprising at least one cationic component, wherein the cationic component is a lipidated peptoid.
2. The multicomponent delivery system of paragraph [0222], wherein the lipidated peptoid is a tertiary amino lipidated and/or PEGylated cationic peptoid or a lipitoid.
3. The multicomponent delivery system of paragraph [0222], wherein the molar percentage of the lipidated peptoid is 10.0-99.5 mol %.
4. The multicomponent delivery system of any of paragraphs [0222]-[0224], further comprising at least one shielding component.
5. The multicomponent delivery system of paragraph [0225], wherein the shielding component comprises a poly(ethylene glycol) (PEG) moiety.
6. The multicomponent delivery system of paragraph [0225], wherein the shielding component is a PEGylated lipid or a PEGylated lipidated peptoid.
7. The multicomponent delivery system of paragraph [0225], wherein the shielding component is DMG-PEG or Compound 107.
8. The multicomponent delivery system of any of paragraphs [0225]-[0228], wherein the molar percentage of the shielding component is 1-5 mol %.
9. The multicomponent delivery system of any of paragraphs [0222]-[0229], further comprises at least one anionic or zwitterionic component.
10. The multicomponent delivery system of paragraph [0230], wherein the zwitterionic component comprises a phospholipid.
11. The multicomponent delivery system of paragraph [0230], wherein the zwitterionic component is selected from a phospholipid, a lipitoid, or a mixture thereof.
12. The multicomponent delivery system of paragraph [0230], wherein the zwitterionic component is a DOPE, DSPC or Compound 105.
13. The multicomponent delivery system of any of paragraphs [0230]-[0233], wherein the molar percentage of the anionic or zwitterionic component is 10-60 mol %.
14. The multicomponent delivery system of any of paragraphs [0222]-[0234], further comprises at least one lipid component.
15. The multicomponent delivery system of paragraph [0235], wherein the lipid component comprises a sterol.
16. The multicomponent delivery system of paragraph [0235], wherein the lipid component is a cholesterol, Compound 89 or Compound 90.
17. The multicomponent delivery system of any of paragraphs [0235]-[0237], wherein the molar percentage of the lipid component is 50-85 mol %.
18. The multicomponent delivery system of any of paragraphs [0222]-[0238], comprising a mixture of an anionic or zwitterionic component and a lipid component.
19. The multicomponent delivery system of paragraph [0239], wherein the molar percentage of the mixture of the anionic or zwitterionic component and the lipid component is 60-80 mol %.
20. The multicomponent delivery system of paragraph [0222], comprising at least 99 mol % cationic component and less than 1 mol % shielding component.
21. The multicomponent delivery system of paragraph [0241], comprising Formula F1.
22. The multicomponent delivery system of paragraph [0222], comprising less than 20 mol % cationic component, less than 5 mol % shielding component, and more than 75 mol % a mixture of a zwitterionic component and a lipid component.
23. The multicomponent delivery system of paragraph [0243], comprising Formula F2 or Formula F4.
24. The multicomponent delivery system of paragraph [0222] comprising between 30-45 mol % cationic component, between 50-70 mol % a mixture of a zwitterionic component and a lipid component, and between 1.5-4.5 mol % shielding component.
25. The multicomponent delivery system of paragraph [0245], comprising Formula F3 or Formula F5.
26. The multicomponent delivery system of paragraph [0222], comprising between 15-35 mol % cationic component, between 60-80 mol % a mixture of a zwitterionic component and a lipid component, and between 1.5-3.0 mol % shielding component.
27. The multicomponent delivery system of paragraph [0247], comprising Formula F2 or Formula F3.
28. A complex comprising the multicomponent delivery system of any of paragraphs [0222]-[0248] and a polyanionic compound.
29. The complex of paragraph [0249], wherein the polyanionic compound is a nucleic acid.
30. The complex of paragraph [0250], wherein the nucleic acid is an mRNA encoding a protein.
31. A method of delivering a polyanionic compound to a cell comprising contacting the cell with the complex of paragraphs [0249]-[0251].
1. In another aspect, the disclosure provides a system for the delivery of polyanionic compounds such as nucleic acids to target cells, the delivery system comprising at least one cationic component, wherein the at least one cationic component comprises a lipidated peptoid.
2. The system of paragraph [0253], further comprising one or more of the following: an anionic or zwitterionic component; a non-cationic lipid component; a shielding component.
3. The system of paragraph [0254], wherein the anionic or zwitterionic component comprises a phospholipid, a lipitoid, or a mixture thereof, preferably wherein the anionic or zwitterionic component is a DOPE or DSPC.
4. The system of paragraphs [0254] or [0224], wherein the non-cationic lipid component comprises a sterol, for example cholesterol, and/or a neutral peptoid.
5. The system of any of the preceding paragraphs [0254]-[0256], wherein the shielding component comprises a poly(ethylene glycol) (PEG) moiety, preferably wherein the shielding component is a PEGylated lipid or a PEGylated lipidated peptoid, more preferably wherein the shielding component is DMG-PEG, for example DMGPEG2k.
6. The system of paragraph [0256] or [0257], wherein the non-cationic lipid component comprises Compound 90 or wherein the non-cationic lipid component comprises Compound 89.
7. The system according to any of the preceding paragraphs [0254]-[0258], wherein the cationic component comprises compound 112 and the non-cationic lipid component comprises Compound 90.
8. The system according to paragraph [0259], wherein the mol % of the cationic component is between about 20 to about 50; the mol % of the anionic or zwitterionic component is between about 0 to about 30; the mol % of the non-cationic lipid component is between about 30 to about 80; and the mol % of the shielding component is between about 0 to about 10.
9. The system according to paragraph [0259] or [0260], wherein the mol % of the cationic component is between about 30 to about 50; the mol % of the anionic or zwitterionic component is between about 0 to about 10; the mol % of the non-cationic lipid component is between about 40 to about 70; and the mol % of the shielding component is between about 0 to about 5.
10. The system according to any of the paragraphs [0259]-[0261], wherein the mol % of the cationic component is 38; the mol % of the anionic or zwitterionic component is 0; the mol % of the non-cationic lipid component is 59.7; and the mol % of the shielding component is 2.3.
11. The system according to paragraph [0262], wherein the anionic or zwitterionic component comprises DOPE, and wherein the shielding component comprises DMG-PEG2K.
12. The system according to any of the paragraphs [0253]-[0257], wherein the cationic component comprises Compound 93 and the non-cationic lipid component comprises cholesterol, Compound 90, Compound 89, or any combination thereof.
13. The system according to any of the paragraphs [0253]-[0257] wherein the cationic component comprises Compound 79 and the non-cationic lipid component comprises Compound 90.
14. The system according to any of the paragraphs [0253]-[0257], wherein the cationic component comprises Compound 24, and the non-cationic lipid component comprises cholesterol, Compound 89, Compound 90, or any combination thereof.
15. The system of any of the preceding clams [0254]-[0257], comprising at least about 99 mol % cationic component and less than about 1 mol % shielding component.
16. The system according to paragraph [0267], wherein the cationic component comprises Compound 24, Compound 79, Compound 93, Compound 112, or any combination thereof.
17. The system according to paragraph [0267] or [0268] wherein the mol % of the cationic component is 99.1, and the mol % of the shielding component comprising PEG is 0.9.
18. The system according to any of the preceding paragraphs [0254]-[0257] comprising less than about 20 mol % of the cationic component, less than about 5 mol % of the shielding component, and more than about 75 mol % of a mixture of the zwitterionic component and the non-cationic lipid component.
19. The system of paragraph [0270], wherein the cationic component comprises Compound 24, Compound 79, Compound 93, Compound 112, or any combination thereof.
20. The system of paragraph [0270] or [0271], wherein the mol % of the cationic component is about 17.9, the mol % of the shielding component comprising PEG is about 2.8, the mol % of the non-cationic lipid component is about 62.9, and the mol % of the anionic or zwitterionic component is about 16.4.
21. The system of paragraph [0270] or [0271], wherein the mol % of the cationic component is about 17.1, the mol % of the shielding component comprising PEG is about 2.7, the mol % of the non-cationic lipid component is about 80.2.
22. The system according to any of the paragraphs [0254]-[0257], comprising between about 30 and about 45 mol % of the cationic component, between about 50 and about 70 mol % of a mixture of the anionic or zwitterionic component and the non-cationic lipid component, and between about 1.5 about 4.5 mol % of the shielding component.
23. The system of paragraph [0274], wherein the cationic component comprises Compound 24, Compound 79, Compound 93, Compound 112, or any combination thereof.
24. The system of paragraph [0274] or [0275], wherein the mol % of the cationic component is about 32.9, the mol % of the shielding component comprising PEG is about 2.0, the mol % of the non-cationic lipid component is about 51.7, and the mol % of the anionic or zwitterionic component is about 13.4.
25. The system of paragraph [0274] or [0275], wherein the mol % of the cationic component is about 42.3, the mol % of the shielding component comprising PEG is about 4.4, the mol % of the non-cationic lipid component is 0, and the mol % of the anionic or zwitterionic component is about 53.3.
26. The system according to any of the paragraphs [0254]-[0257], comprising between about 15 and about 35 mol % of the cationic component, between about 60 and about 80 mol % of a mixture of the zwitterionic component and the non-cationic lipid component, and between about 1.5 and about 3.0 mol % of the shielding component.
27. The system of paragraph [0278], wherein the cationic component comprises Compound 24, Compound 79, Compound 93, Compound 112, or any combination thereof.
28. The system of paragraph [0278] or [0279], wherein the mol % of the cationic component is about 17.9, the mol % of the shielding component comprising PEG is about 2.8, the mol % of the non-cationic lipid component is about 62.9, and the mol % of the anionic or zwitterionic component is about 16.4.
29. The system of paragraph [0278] or [0279], wherein the mol % of the cationic component is about 32.9, the mol % of the shielding component comprising PEG is about 2.0, the mol % of the non-cationic lipid component is about 51.7, and the mol % of the anionic or zwitterionic component is about 13.4.
30. A complex comprising the system of any of the preceding paragraphs of this aspect and a polyanionic compound, preferably a nucleic acid.
31. The complex of paragraph [0282], wherein the polyanionic compound is an mRNA encoding a protein.
32. The complex according to paragraphs [0282] or [0283], wherein the mass ratio of the cationic component to the nucleic acid is between about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, between about 2:1 and about 5:1, for example wherein the complex comprises the cationic components and the nucleic acid at a mass ratio of about 3:1,
33. The complex according to paragraph [0284], wherein the cationic component is Compound 112, and the non-cationic lipid component is Compound 90.
34. The complex according to paragraph [0285], wherein the shielding component comprises DMG-PEG 2000.
35. The complex according to any of the paragraphs [0283]-[0286], wherein mass ratio of the cationic component to the nucleic acid is about 10:1.
36. The complex according to paragraph [0287], wherein the mass ratio of the shielding component to the nucleic acid is 1.4:1.
37. The complex according to paragraphs [0287] or [0288] wherein the mass ratio of the non-cationic lipid component to the nucleic acid is about 5.4:1.
38. The complex according to any of the paragraphs [0287]-[0289], wherein a charge ratio between the cationic component and the nucleic acid is about 6:1.
39. The complex according to any of the paragraphs [0282]-[0290], wherein mass ratio of the cationic component to the nucleic acid is about 10:1, the mass ratio of the shielding component to the nucleic acid is about 1.4:1, and the mass ratio component of the non-cationic lipid component to the nucleic acid is about 5.4:1, and the mass ratio of the anionic or zwitterionic component to the nucleic acid is 0.
40 The complex according to any of the paragraphs [0282]-[0291], comprising the system according to any of the paragraphs [0259]-[0263].
41. The system according to any of the paragraphs [0259]-[0263] for use in delivering a therapeutic nucleic acid, preferably an mRNA.
42. The system according to any of the paragraphs [0253]-[0281], or the complex according to any of the paragraphs [0282]-[0292] for use as a medicament.
43. The system according to any of the paragraphs [0253]-[0281] or the complex according to any of the paragraphs [0282]-[0292] for use for use in the treatment of any of the conditions referred to in the description herein.
44. A method of forming the complex of any of the paragraphs [0282]-[0292], comprising contacting a system according to any of the paragraphs [0253]-[0281], with a polyanionic compound, for example the nucleic acid, preferably an mRNA encoding a protein.

### EXAMPLES

The presently disclosed subject matter will be better understood by reference to the following Examples, which are provided as example of the invention, and not by way of limitation.

### Example 1— Synthesis of Example Tertiary Amino Lipidated Cationic Peptoids

General protocols for synthesizing the tertiary amino lipidated cationic peptoids disclosed herein can be found in WO2020/069442 and WO2020/069445, each of which is incorporated herein by reference in its entirety.

The following example describes the general protocol for synthesis of the tertiary amino lipidated and/or PEGylated cationic peptoids of formula (I) as described herein.

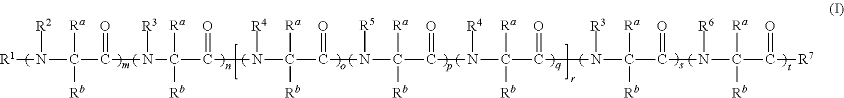

In the description provided below, all R^{a} and R^{b} are —H. All polymers were synthesized using bromoacetic acid and primary amines. An Fmoc-Rink amide resin was used as the solid support. The Fmoc group on the resin was deprotected with 20% (v/v) piperidine-dimethylformamide (DMF). The amino resin was then amidated with bromoacetic acid. The amidation was followed by amination of the α-carbon by nucleophilic displacement of the bromide with a primary amine. The two steps were successively repeated to produce the desired cationic peptide sequence.

All reactions and washings were performed at room temperature unless otherwise noted. Washing of the resin refers to the addition of a wash solvent (usually DMF or dimethylsulfoxide (DMSO)) to the resin, agitating the resin so that a uniform slurry was obtained, followed by thorough draining of the solvent from the resin. Solvents were removed by vacuum filtration through the fritted bottom of the reaction vessel until the resin appeared dry. In all the syntheses, resin slurries were agitated via bubbling argon up through the bottom of the flitted vessel.

Initial Resin Deprotection. A fritted reaction vessel was charged with Fmoc-Rink amide resin. DMF was added to the resin and this solution was agitated to swell the resin. The DMF was then drained. The Fmoc group was removed by adding 20% piperidine in DMF to the resin, agitating the resin, and draining the resin. 20% piperidine in DMF is added to the resin and agitated for 15 minutes and then drained. The resin is then washed with DMF, six times.

Acylation/Amidation. The deblocked amine was then acylated by adding bromoacetic acid in DMF to the resin followed by N,N-diisoprooplycarbodiimide (DIC) in DMF. This solution is agitated for 30 minutes at room temperature and then drained. This step was repeated a second time. The resin was then washed with DMF twice and DMSO once. This was one completed reaction cycle.

Nucleophilic Displacement/Amination. The acylated resin was treated with the desired primary or secondary amine to undergo nucleophilic displacement at the bromine leaving group on the α-carbon. This acylation/displacement cycle was repeated until the desired peptide sequence is obtained.

Peptide Cleavage from Resin. The dried resin was placed in a glass scintillation vial containing a teflon-coated micro stir bar, and 95% trifluoroacetic acid (TFA) in water was added. The solution was stirred for 20 minutes and then filtered through solid-phase extraction (SPE) column fitted with a polyethylene frit into a polypropylene conical centrifuge tube.

The resin was washed with 1 mL 95% TFA. The combined filtrates were then lyophilized three times from 1:1 acetonitrile:water. The lyophilized peptide) was redissolved to a concentration of 5 mM in 5% acetonitrile in water.

Purification and Characterization. The redissolved crude peptide was purified by preparative HPLC. The purified peptide was characterized by LC-MS analysis.

### Example 2—Synthesis and Characterization of Representative Amino Lipidated Peptoids

Amino lipidated peptoids were synthesized by the submonomer method described above in Example 1 with bromoacetic acid and N,N′-diisopropylcarbodiimide (DIC). Polystyrene-supported MBHA Fmoc-protected Rink amide (200 mg representative scale, 0.64 mmol/g loading, Protein Technologies) resin was used as a solid support. For bromoacetylation, resin was combined with a 1:1 mixture of 2 M bromoacetic acid and 2M N,N′-diisopropylcarbodiimide (DIC) for 5 minutes. Amine displacement was carried out using a 1M solution of amine in DMF for 1 hour. Following synthesis, crude peptoids were cleaved from resin using 5 mL of a mixture of 95:2.5:2.5 trifluoroacetic acid (TFA):water:triisopropylsilane for 40 minutes at room temperature. Resin was removed by filtration and the filtrate concentrated using a Biotage V10 evaporator. The crude peptoids were further concentrated by lyophilization from a 25% solution of MeCN in water. Purity and identity were assayed with a Waters Acquity UPLC system with Acquity Diode Array UV detector and Waters SQD2 mass spectrometer on a Waters Acquity UPLC Peptide BEH C₄ Column over a 5-95% gradient. Select crude peptoids were purified by preparative Waters Prep 150LC system with Waters 2489 UV/Visible Detector on a Waters XBridge BEH300 Prep C₄ column using a 5-40% acetonitrile in water with 0.1% TFA gradient over 30 minutes.

Alternatively, the bromoacetylation, resin was combined with a 1:1 mixture of 0.8 M bromoacetic acid and 0.8 M N,N′-diisopropylcarbodiimide (DIC) for 15 minutes. Amine displacement was carried out using a 1M solution of amine in DMF for 45 minutes. Following synthesis, crude peptoids were cleaved from resin using 5 mL of a mixture of 95:2.5:2.5 trifluoroacetic acid (TFA):water:triisopropylsilane for 40 minutes at room temperature. Resin was removed by filtration and the filtrate concentrated using a vacuum centrifuge. The crude peptoids were further purified by reverse-phase flash chromatography (Biotage Selekt) using a C4 column and a gradient from 60-95% ACN/H₂O+0.1% TFA. Purity and identity were assayed with a Waters Acquity UPLC system with Acquity Diode Array UV detector and Waters SQD2 mass spectrometer on a Waters Acquity UPLC Peptide BEH C₄ Column over a 5-95% gradient. Select peptoids were further purified by preparative Waters Prep150LC system with Waters 2489 UV/Visible Detector on a Waters XBridge BEH300 Prep C4 column using a 40-85% acetonitrile in water with 0.1% TFA gradient over 30 minutes.

Tables 1A, 1B, 1C, 1D, and 1E show representative amino lipidated peptoids prepared by the method described in Example 2. Examples of peptoid precursors are shown in Table 1F.

### Example 3—Synthesis and Characterization of Representative Multicomponent Delivery Systems

Synthesis. Amino-lipidated peptoids can be combined with polyanionic compounds, such as nucleic acids, to form nanoparticle compositions that can be evaluated for therapeutic and/or prophylactic purposes in vitro or in vivo. Without being bound to any particular theory, the cationic portion(s) of the amino-lipidated peptoids binds to the negatively-charged phosphodiester backbone of the polyanionic cargo (e.g., nucleic acid cargo) through primarily electrostatic interactions, forming a mixed coacervate complex. Hydrophobic interactions between lipid chains on the amino-lipidated peptoids can act to stabilize particle formation and assist with membrane association.

Formulations can be prepared through any physical and/or chemical methods known in the art to modulate their physical, chemical, and biological properties. These methods typically involve rapid combination of the amino-lipidate peptoid in water or a water-miscible organic solvent with the oligonucleotide in water or an aqueous buffer solution. These methods can include simple mixing of the components by pipetting, or microfluidic mixing processes such as those involving T-mixers, vortex mixers, or other chaotic mixing structures.

In standard formulations, the amino-lipidated peptoid and additional lipids are dissolved in anhydrous ethanol at a concentration of 10 mg/mL to result in solutions that are stable at room temperature. In some implementations, the solutions are stored at −20° C. The nucleic acid cargo is dissolved in DNAse or RNAse-free water at a final concentration of 1-2 mg/mL. These solutions can be stored at −20° C. or −78° C. for extended time periods.

To prepare the delivery systems disclosed herein, amino-lipidated peptoid and additional lipid components are first pre-mixed in an ethanol phase at the required mass ratios. Nucleic acid cargo(s) are diluted in ethanol and acidic buffer (10 mM phosphate/citrate, pH 5.0). Ethanol and aqueous phases are mixed at a 3:1 volume ratio, and then immediately diluted with a 1:1 volume ratio of PBS, resulting in a final mRNA concentration of 0.1 μg/uL.

Non-limiting example multicomponent delivery system formulations are described in Table 4. The multicomponent delivery systems of the disclosure were combined with firefly luciferase (Fluc) mRNA at ratios detailed in Table 4 to form nanoparticle compositions to be evaluated for therapeutic and/or prophylactic purposes in vitro or in vivo. The w/w in Table 4 is the ratio of that component to the mRNA by mass.

Characterization. The resulting delivery vehicles were evaluated by dynamic light scattering (DLS) in order to determine the volume average particle size/diameter (nm) and the size polydispersity index (PDI) within the formulation. Particle sizes and size distributions were measured using a Wyatt DynaPro Plate Reader III. In general, formulated samples were diluted to 2 ng/uL in 100 uL phosphate buffered saline (PBS). Data is reported as the hydrodynamic diameter (in nm) of the cumulant fit of the correlation function, and the polydispersity of that measurement. Table 5 shows the DLS-based size measurements, demonstrating that the multicomponent delivery systems disclosed herein advantageously allow the formation of nanoparticles that are stable and monodisperse.

The percentage of mRNA encapsulated within amino-lipidated peptoid formulations was determined using a modified RiboGreen assay. In general, formulated mRNA samples were diluted to 500 ng/mL in Tris-EDTA buffer with or without Triton-X. RiboGreen (Invitrogen) was added at a 200-fold dilution, and plate was incubated for 5 minutes. Fluorescence was measured at Ex. 840 nm/Em. 520 nm and encapsulated mRNA was calculated by taking the ratio of fluorescence for non-lysed particles versus lysed particles.

TABLE 4
Compositions of non-limiting examples of multicomponent delivery system formulations

| | Lipidated Cationic | | Anionic/ | | | | | |
|---|---|---|---|---|---|---|---|---|
| | Peptoid | | Zwitterionic | | Neutral (lipid) | | Shielding | |
| | Component | | Component | | Component | | Component | |
| Name | (w/w) | | (w/w) | | (w/w) | | (w/w) | |
| MC3-F2 | DLIN-MC3- | 10 | DSPC | 2.7 | Cholesterol | 5.4 | DMG-PEG 2000 | 1.4 |
| (Commercial) | DMA | | | | | | | |
| FORM-A | None | 0 | None | 0 | None | 0 | None | 0 |
| FORM-B | Compound 93 | 10 | DSPC | 2.7 | Cholesterol | 5.4 | DMG-PEG 2000 | 1.4 |
| FORM-C | Compound 93 | 5 | DOPE | 3 | Compound 90 | 6 | DMG-PEG 2000 | 1.8 |
| FORM-D | Compound 93 | 5 | Compound 105 | 3 | Cholesterol | 6 | DMG-PEG 2000 | 1.8 |
| FORM-E | Compound 93 | 5 | DOPE | 3 | Cholesterol | 6 | Compound 107 | 1.8 |
| FORM-F | Compound 79 | 5 | DOPE | 3 | Compound 90 | 6 | DMG-PEG 2000 | 1.8 |
| FORM-G | Compound 24 | 5 | DOPE | 3 | Compound 90 | 6 | DMG-PEG 2000 | 1.8 |
| FORM-H | Compound 24 | 5 | DOPE | 3 | Cholesterol | 6 | Compound 107 | 1.8 |
| FORM-I | Compound 93 | 5 | DOPE | 3 | Compound 89 | 6 | DMG-PEG 2000 | 1.8 |
| FORM-J | Compound 112 | 5 | DOPE | 3 | Compound 90 | 6 | DMG-PEG 2000 | 1.8 |
| FORM-K | Compound 112 | 10 | None | 0 | Compound 90 | 5.4 | DMG-PEG 2000 | 1.4 |
| FORM-L | Compound 81 | 10 | Compound 135 | 1 | Compound 119 | 5.4 | DMG-PEG 2000 | 1.4 |
| FORM-M | Compound 163 | 10 | Compound 135 | 1 | Compound 119 | 5.4 | DMG-PEG 2000 | 1.4 |
| FORM-N | Compound 155 | 10 | Compound 135 | 1 | Compound 119 | 5.4 | DMG-PEG 2000 | 1.4 |
| FORM-P | Compound 81 | 10 | Compound 135 | 3 | Compound 119 | 5.4 | DMG-PEG 2000 | 1.4 |
| FORM-Q | Compound 163 | 10 | Compound 135 | 3 | Compound 119 | 5.4 | DMG-PEG 2000 | 1.4 |
| FORM-R | Compound 155 | 10 | Compound 135 | 3 | Compound 119 | 5.4 | DMG-PEG 2000 | 1.4 |
| FORM-T | Compound 81 | 10 | Compound 135 | 5.4 | Compound 119 | 5.4 | DMG-PEG 2000 | 1.4 |
| FORM-U | Compound 163 | 10 | Compound 135 | 5.4 | Compound 119 | 5.4 | DMG-PEG 2000 | 1.4 |
| FORM-V | Compound 155 | 10 | Compound 135 | 5.4 | Compound 119 | 5.4 | DMG-PEG 2000 | 1.4 |
| 81/90A | Compound 81 | 10 | None | 0 | Compound 90 | 3 | DMG-PEG 2000 | 2 |
| 81/119A | Compound 81 | 10 | None | 0 | Compound 119 | 3 | DMG-PEG 2000 | 2 |
| 81-F2 | Compound 81 | 10 | DSPC | 2.7 | Cholesterol | 5.4 | DMG-PEG 2000 | 1.4 |
| 137-MP1-135 | Compound 137 | 10 | None | 0 | Compound 135 | 3 | DMG-PEG 2000 | 2 |
| 137-MP1-90 | Compound 137 | 10 | None | 0 | Compound 90 | 3 | DMG-PEG 2000 | 2 |
| 137-MP1-127 | Compound 137 | 10 | None | 0 | Compound 127 | 3 | DMG-PEG 2000 | 2 |
| 137-F2 | Compound 137 | 10 | DSPC | 2.7 | Cholesterol | 5.4 | DMG-PEG 2000 | 1.4 |
| 73-MP1-127 | Compound 73 | 10 | None | 0 | Compound 127 | 3 | DMG-PEG 2000 | 2 |
| 73-MP1-131 | Compound 73 | 10 | None | 0 | Compound 131 | 3 | DMG-PEG 2000 | 2 |
| 24-F2 | Compound 24 | 10 | DSPC | 2.7 | Cholesterol | 5.4 | DMG-PEG 2000 | 1.4 |
| 73-F2 | Compound 73 | 10 | DSPC | 2.7 | Cholesterol | 5.4 | DMG-PEG 2000 | 1.4 |
| 79-F2 | Compound 79 | 10 | DSPC | 2.7 | Cholesterol | 5.4 | DMG-PEG 2000 | 1.4 |
| 93-F2 | Compound 93 | 10 | DSPC | 2.7 | Cholesterol | 5.4 | DMG-PEG 2000 | 1.4 |
| 85-F2 | Compound 85 | 10 | DSPC | 2.7 | Cholesterol | 5.4 | DMG-PEG 2000 | 1.4 |
| 115-F2 | Compound 115 | 10 | DSPC | 2.7 | Cholesterol | 5.4 | DMG-PEG 2000 | 1.4 |
| 152-F2 | Compound 152 | 10 | DSPC | 2.7 | Cholesterol | 5.4 | DMG-PEG 2000 | 1.4 |
| 81-MP1-119 | Compound 81 | 10 | None | 0 | Compound 119 | 5.4 | DMG-PEG 2000 | 1.4 |
| 155-MP1-119 | Compound 155 | 10 | None | 0 | Compound 119 | 5.4 | DMG-PEG 2000 | 1.4 |
| 152-MP1-119 | Compound 152 | 10 | None | 0 | Compound 119 | 5.4 | DMG-PEG 2000 | 1.4 |
| 163-MP1-119 | Compound 163 | 10 | None | 0 | Compound 119 | 5.4 | DMG-PEG 2000 | 1.4 |
| 164-MP1-119 | Compound 164 | 10 | None | 0 | Compound 119 | 5.4 | DMG-PEG 2000 | 1.4 |
| 81-MP1-90 | Compound 81 | 10 | None | 0 | Compound 90 | 5.4 | DMG-PEG 2000 | 1.4 |
| 81-MP1-119 | Compound 81 | 10 | None | 0 | Compound 119 | 5.4 | DMG-PEG 2000 | 1.4 |
| 81-MP1-125 | Compound 81 | 10 | None | 0 | Compound 125 | 5.4 | DMG-PEG 2000 | 1.4 |
| 81-MP1-126 | Compound 81 | 10 | None | 0 | Compound 126 | 5.4 | DMG-PEG 2000 | 1.4 |
| 112-MP1-90 | Compound 112 | 10 | None | 0 | Compound 90 | 5.4 | DMG-PEG 2000 | 1.4 |
| 112-MP1-119 | Compound 112 | 10 | None | 0 | Compound 119 | 5.4 | DMG-PEG 2000 | 1.4 |
| 112-MP1-125 | Compound 112 | 10 | None | 0 | Compound 125 | 5.4 | DMG-PEG 2000 | 1.4 |
| 112-MP1-126 | Compound 112 | 10 | None | 0 | Compound 126 | 5.4 | DMG-PEG 2000 | 1.4 |
| 81-MP1-123 | Compound 81 | 10 | None | 0 | Compound 123 | 5.4 | DMG-PEG 2000 | 1.4 |
| 81/90B | Compound 81 | 10 | None | 0 | Compound 90 | 8 | DMG-PEG 2000 | 1.4 |
| 81/90C | Compound 81 | 15 | None | 0 | Compound 90 | 8 | DMG-PEG 2000 | 1.4 |
| 81/119B | Compound 81 | 15 | None | 0 | Compound 119 | 8 | DMG-PEG 2000 | 1.4 |
| 81-MP1-119 | Compound 81 | 10 | None | 0 | Compound 119 | 5.4 | DMG-PEG 2000 | 1.4 |
| 81/119C | Compound 81 | 8 | None | 0 | Compound 119 | 5.4 | DMG-PEG 2000 | 1.4 |
| 81/119D | Compound 81 | 10 | None | 0 | Compound 119 | 8 | DMG-PEG 2000 | 1.4 |
| 81/119E | Compound 81 | 15 | None | 0 | Compound 119 | 10 | DMG-PEG 2000 | 1.4 |
| 81/90D | Compound 81 | 10 | None | 0 | Compound 90 | 10 | DMG-PEG 2000 | 1.4 |
| 112/90 | Compound 112 | 10 | None | 0 | Compound 90 | 3 | DMG-PEG 2000 | 1.4 |
| 112/119 | Compound 112 | 10 | None | 0 | Compound 119 | 3 | DMG-PEG 2000 | 1.4 |
| 112/122 | Compound 112 | 10 | None | 0 | Compound 122 | 3 | DMG-PEG 2000 | 1.4 |
| 112/125 A' | Compound 112 | 10 | None | 0 | Compound 125 | 3 | DMG-PEG 2000 | 1.4 |
| 112/125 B' | Compound 112 | 15 | None | 0 | Compound 125 | 1 | DMG-PEG 2000 | 1.4 |
| 112/125 C' | Compound 112 | 10 | None | 0 | Compound 125 | 5.4 | DMG-PEG 2000 | 1.4 |
| (112-MP1-125) | | | | | | | | |
| 112/126 A' | Compound 112 | 10 | None | 0 | Compound 126 | 3 | DMG-PEG 2000 | 1.4 |
| 112/126 B' | Compound 112 | 10 | DSPC | 2 | Compound 126 | 6 | DMG-PEG 2000 | 1.4 |
| 112/127 | Compound 112 | 10 | None | 0 | Compound 127 | 3 | DMG-PEG 2000 | 1.4 |
| 112-F2* | Compound 112 | 10 | DSPC | 2.2 | Cholesterol | 5.4 | DMG-PEG 2000 | 1.4 |
| 112/125A | Compound 112 | 13.3 | None | 0 | Compound 125 | 4.5 | DMG-PEG 2000 | 1.4 |
| 112/125B | Compound 112 | 8 | None | 0 | Compound 125 | 1 | DMG-PEG 2000 | 1.4 |
| 112/125C | Compound 112 | 15 | None | 0 | Compound 125 | 1 | DMG-PEG 2000 | 1.4 |
| 112/125D | Compound 112 | 8 | None | 0 | Compound 122 | 6 | DMG-PEG 2000 | 1.4 |
| 112/125E | Compound 112 | 15 | None | 0 | Compound 125 | 6 | DMG-PEG 2000 | 1.4 |
| 112/125F | Compound 112 | 10.8 | None | 0 | Compound 125 | 6 | DMG-PEG 2000 | 1.4 |
| 112/125G | Compound 112 | 9.2 | None | 0 | Compound 125 | 3.5 | DMG-PEG 2000 | 1.4 |
| 112/125H | Compound 112 | 13.3 | None | 0 | Compound 125 | 2 | DMG-PEG 2000 | 1.4 |
| 112/125I | Compound 112 | 13.3 | DSPC | 2 | Compound 125 | 4.5 | DMG-PEG 2000 | 0.75 |
| 112/125J | Compound 112 | 13.3 | None | 0 | Compound 125 | 4.5 | DMG-PEG 2000 | 2 |

TABLE 5
Physical properties of the formulations

| Name | Z-Average (d · nm) | PdI |
|---|---|---|
| FORM-A | | |
| FORM-B | 122.8 | 0.14 |
| FORM-C | 183.2 | 0.13 |
| FORM-D | 201.8 | 0.29 |
| FORM-E | 242.6 | 0.24 |
| FORM-F | 169.5 | 0.13 |
| FORM-G | 172.1 | 0.14 |
| FORM-H | 163.1 | 0.13 |
| FORM-I | 190.4 | 0.18 |
| FORM-J | 159.0 | 0.14 |
| 112/90 | 122.9 | 0.113 |
| 112/119 | 118.4 | 0.14 |
| 112/122 | 101.7 | 0.158 |
| 112/125 A′ | 92 | 0.174 |
| 112/125 B′ | 106.3 | 0.154 |
| 112/125 C′ | 109.9 | 0.135 |
| 112/126 A′ | 118 | 0.224 |
| 112/126 B′ | 128.3 | 0.172 |
| 112/127 | 120.2 | 0.185 |
| 112/125A | 102.8 | 0.151 |
| 112/125B | 108.9 | 0.199 |
| 112/125C | 84.3 | 0.141 |
| 112/125D | 98.9 | 0.145 |
| 112/125E | 87.2 | 0.176 |
| 112/125F | 128.5 | 0.195 |
| 112/125G | 93.8 | 0.162 |
| 112/125H | 76.4 | 0.143 |
| 112/125I | 90.8 | 0.167 |
| 112/125J | 102.2 | 0.199 |
| 112/125A | 95.7 | 0.141 |
| 81-MP1-119 | 159.1 | 0.276 |
| 155-MP1-119 | 141.5 | 0.228 |
| 81-MP1-90 | 174.8 | 0.196 |
| 155-MP1-90 | 169.8 | 0.295 |

### Example 4—In Vitro Luciferase Assay

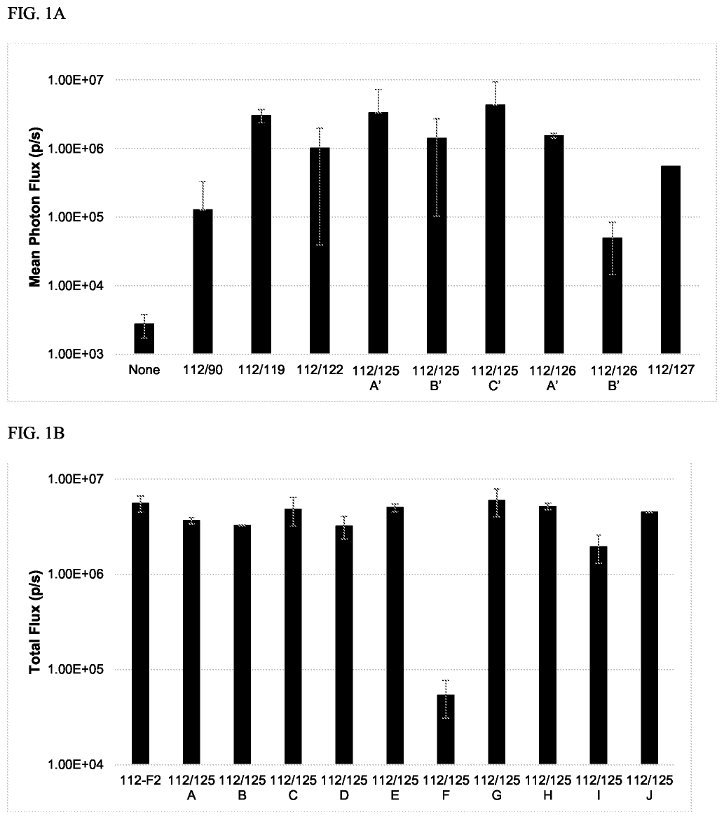

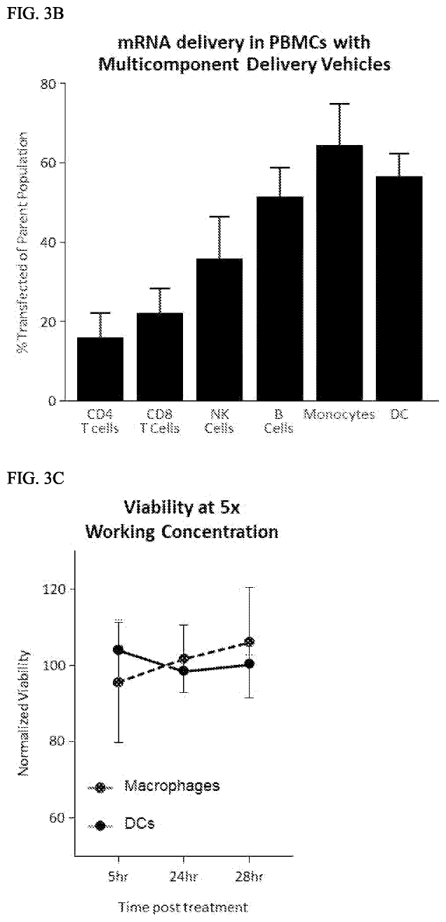

The efficacy of mRNA delivered in the multicomponent delivery systems was evaluated in vitro based on their ability to deliver the Fluc reporter gene to cultured cells. The multicomponent delivery systems were combined with Fluc mRNA at the given w/w ratios, and the resulting particles were added to cultured cells (e.g., HEK-293 cells, hPBMC) at a dose of 50 ng/well (in 150 μL total volume). The resulting luciferase expression (RLU) was measured by a luminescence plate reader after 6, 8, and/or 18 hours of treatment. See FIGS. 1A, 1B, and 3A. Table 6 shows the observed in vitro Fluc expression. These data demonstrate that the mixed peptoid delivery systems of the disclosure allow for efficient intake, expression, and viability of mRNA cargo in cells. The mixed peptoid systems show promise for non-viral and ex vivo therapies, such as gene editing technologies, cell therapies for solid tumors, and cellular reprogramming, including generation of autologous iPSCs.

TABLE 6
In vitro expression from formulations

| | HEK-293 | RL | hPBMC |
|---|---|---|---|
| Name | Expression | Expression | Expression |
| FORM-A | 3800.5 | 11 | 44 |
| FORM-B | 1754 | 382.5 | 148.5 |
| FORM-C | 36547.5 | 203 | 1047.5 |
| FORM-D | 1287.5 | 16 | 70.5 |
| FORM-E | 43617 | 12 | 2997.5 |
| FORM-F | 17374.5 | 584 | 1418.5 |
| FORM-G | 46475 | 652 | 4083 |
| FORM-H | 33839 | 437.5 | 2349 |
| FORM-I | 37383.5 | 711.5 | 2418.5 |
| FORM-J | 15334 | 320 | 1189 |

Multicomponent delivery vehicle systems comprising varying amounts of each of: Compound 112 as the cationic component (Table 7A), Compound 90 as the non-cationic component (Table 7B), DSPC (Table 7C), and DMG-PEG 2000 (Table 7D) were complexed with Fluc mRNA, and added to cultured HEK-293 cells, and the resulting luciferase expression was measured (Table 7E), as previously described. The components of the multicomponent delivery vehicle systems tested can be found in Tables 7A-7D, and the luciferase expression is shown in Table 7E. For example, Formula A1 includes 11.0 w/w of Compound 112, 0 w/w of Compound 90, 0 w/w of DSPC, and 0.9 w/w/of DMG-PEG, and resulted in an HEK expression of 14100. As another example, Formula G8 includes 10.1 w/w of Compound 112, 5.0 w/w of Compound 90, 5.0 w/w of DSPC, and 1.2 w/w of DMG-PEG, and resulted in an HEK expression of 50900.

TABLE 7A
Amount of Compound 112 (w/w relative to cargo)

| | 1 | 2 | 3 | 4 | 5 | 6 | 7 | 8 | 9 | 10 | 11 | 12 |
|---|---|---|---|---|---|---|---|---|---|---|---|---|
| A | 11.0 | 10.1 | 9.4 | 9.0 | 8.4 | 7.9 | 18.1 | 16.9 | 15.8 | 15.3 | 14.4 | 13.6 |
| B | 9.0 | 8.4 | 7.9 | 7.7 | 7.2 | 6.8 | 15.3 | 14.4 | 13.6 | 13.3 | 12.6 | 12.0 |
| C | 7.7 | 7.2 | 6.8 | 6.6 | 6.3 | 6.0 | 13.3 | 12.6 | 12.0 | 11.7 | 11.2 | 10.7 |
| D | 6.5 | 6.1 | 5.9 | 5.7 | 5.5 | 5.2 | 11.5 | 10.9 | 10.5 | 10.3 | 9.9 | 9.5 |
| E | 7.7 | 7.2 | 6.8 | 6.5 | 6.1 | 5.9 | 13.3 | 12.6 | 12.0 | 11.5 | 10.9 | 10.5 |
| F | 6.6 | 6.3 | 6.0 | 5.7 | 5.5 | 5.2 | 11.7 | 11.2 | 10.7 | 10.3 | 9.9 | 9.5 |
| G | 5.9 | 5.6 | 5.4 | 5.1 | 4.9 | 4.7 | 10.5 | 10.1 | 9.7 | 9.3 | 9.0 | 8.7 |
| H | 5.1 | 4.9 | 4.7 | 4.6 | 4.4 | 4.3 | 9.3 | 9.0 | 8.7 | 8.4 | 8.1 | 7.9 |

TABLE 7B
Amount of Compound 90 (w/w relative to cargo)

| | 1 | 2 | 3 | 4 | 5 | 6 | 7 | 8 | 9 | 10 | 11 | 12 |
|---|---|---|---|---|---|---|---|---|---|---|---|---|
| A | 0.0 | 0.0 | 0.0 | 4.5 | 4.2 | 3.9 | 0.0 | 0.0 | 0.0 | 3.8 | 3.6 | 3.4 |
| B | 0.0 | 0.0 | 0.0 | 3.8 | 3.6 | 3.4 | 0.0 | 0.0 | 0.0 | 3.3 | 3.2 | 3.0 |
| C | 0.0 | 0.0 | 0.0 | 3.3 | 3.2 | 3.0 | 0.0 | 0.0 | 0.0 | 2.9 | 2.8 | 2.7 |
| D | 0.0 | 0.0 | 0.0 | 2.9 | 2.7 | 2.6 | 0.0 | 0.0 | 0.0 | 2.6 | 2.5 | 2.4 |
| E | 7.7 | 7.2 | 6.8 | 10.3 | 9.8 | 9.4 | 6.6 | 6.3 | 6.0 | 9.2 | 8.8 | 8.4 |
| F | 6.6 | 6.3 | 6.0 | 9.2 | 8.8 | 8.4 | 5.9 | 5.6 | 5.4 | 8.2 | 7.9 | 7.6 |
| G | 5.9 | 5.6 | 5.4 | 8.2 | 7.9 | 7.6 | 5.2 | 5.0 | 4.8 | 7.5 | 7.2 | 6.9 |
| H | 5.1 | 4.9 | 4.7 | 7.3 | 7.1 | 6.8 | 4.7 | 4.5 | 4.3 | 6.7 | 6.5 | 6.3 |

TABLE 7C
Amount of DSPC (w/w relative to cargo)

| | 1 | 2 | 3 | 4 | 5 | 6 | 7 | 8 | 9 | 10 | 11 | 12 |
|---|---|---|---|---|---|---|---|---|---|---|---|---|
| A | 0 | 0 | 0 | 0 | 0 | 0 | 0 | 0 | 0 | 0 | 0 | 0 |
| B | 4.5 | 4.2 | 3.9 | 3.8 | 3.6 | 3.4 | 3.8 | 3.6 | 3.4 | 3.3 | 3.2 | 3.0 |
| C | 7.7 | 7.2 | 6.8 | 6.6 | 6.3 | 6.0 | 6.6 | 6.3 | 6.0 | 5.9 | 5.6 | 5.4 |
| D | 10.3 | 9.8 | 9.4 | 9.2 | 8.8 | 8.4 | 9.2 | 8.8 | 8.4 | 8.2 | 7.9 | 7.6 |
| E | 0.0 | 0.0 | 0.0 | 0.0 | 0.0 | 0.0 | 0.0 | 0.0 | 0.0 | 0.0 | 0.0 | 0.0 |
| F | 3.3 | 3.2 | 3.0 | 2.9 | 2.7 | 2.6 | 2.9 | 2.8 | 2.7 | 2.6 | 2.5 | 2.4 |
| G | 5.9 | 5.6 | 5.4 | 5.1 | 4.9 | 4.7 | 5.2 | 5.0 | 4.8 | 4.7 | 4.5 | 4.3 |
| H | 8.2 | 7.9 | 7.6 | 7.3 | 7.1 | 6.8 | 7.5 | 7.2 | 6.9 | 6.7 | 6.5 | 6.3 |

TABLE 7D
Amount of DMG-PEG 2000 (w/w relative to cargo)

| | 1 | 2 | 3 | 4 | 5 | 6 | 7 | 8 | 9 | 10 | 11 | 12 |
|---|---|---|---|---|---|---|---|---|---|---|---|---|
| A | 0.9 | 2.4 | 3.8 | 0.7 | 2.0 | 3.2 | 0.7 | 2.0 | 3.2 | 0.6 | 1.7 | 2.7 |
| B | 0.7 | 2.0 | 3.2 | 0.6 | 1.7 | 2.7 | 0.6 | 1.7 | 2.7 | 0.5 | 1.5 | 2.4 |
| C | 0.6 | 1.7 | 2.7 | 0.5 | 1.5 | 2.4 | 0.5 | 1.5 | 2.4 | 0.5 | 1.3 | 2.1 |
| D | 0.5 | 1.5 | 2.3 | 0.5 | 1.3 | 2.1 | 0.5 | 1.3 | 2.1 | 0.4 | 1.2 | 1.9 |
| E | 0.6 | 1.7 | 2.7 | 0.5 | 1.5 | 2.3 | 0.5 | 1.5 | 2.4 | 0.5 | 1.3 | 2.1 |
| F | 0.5 | 1.5 | 2.4 | 0.5 | 1.3 | 2.1 | 0.5 | 1.3 | 2.1 | 0.4 | 1.2 | 1.9 |
| G | 0.5 | 1.3 | 2.1 | 0.4 | 1.2 | 1.9 | 0.4 | 1.2 | 1.9 | 0.4 | 1.1 | 1.7 |
| H | 0.4 | 1.2 | 1.9 | 0.4 | 1.1 | 1.7 | 0.4 | 1.1 | 1.7 | 0.3 | 1.0 | 1.6 |

As another example, multicomponent delivery vehicle systems comprising varying amounts of each of: Compound 112 as the cationic component (Table 8A), the indicated non-cationic lipidated peptoid component (Table 8B), DSPC (Table 8C), and DMG-PEG 2000 (Table 8D) were complexed with Fluc mRNA, and added to cultured HEK-293 cells, and the resulting luciferase expression was measured (Table 8E), as previously described. The components of the multicomponent delivery vehicle systems tested can be found in Tables 8A-8D, and the luciferase expression is shown in Table 8E. For example, Formula 112/119A-1 includes 11.2 w/w of Compound 112, 1.1 w/w of Compound 119, 0 w/w of DSPC, and 1.7 w/w of DMG-PEG, and resulted in an HEK expression of 4750. As another example, Formula 112/124B-6 includes 11.5 w/w of Compound 112, 2.3 w/w of Compound 124, 4.6 w/w of DSPC, and 1.1 w/w of DMG-PEG, and resulted in an HEK expression of 3730.

Table 7E and 8E demonstrate that the multicomponent delivery vehicle systems of the disclosure elicit strong Fluc expression.

TABLE 8A
Amount of Compound 112 (w/w relative to cargo)

| Cmpd | 1 | 2 | 3 | 4 | 5 | 6 | 7 | 8 | 9 | 10 | 11 | 12 |
|---|---|---|---|---|---|---|---|---|---|---|---|---|
| 119A | 11.2 | 9.9 | 8.8 | 10.3 | 9.2 | 8.3 | 9.2 | 8.3 | 7.5 | 8.5 | 7.8 | 7.1 |
| 119B | 15.2 | 13.5 | 12.2 | 14.0 | 12.6 | 11.5 | 12.6 | 11.5 | 10.5 | 11.8 | 10.8 | 9.9 |
| 121 | 11.2 | 9.9 | 8.8 | 10.3 | 9.2 | 8.3 | 9.2 | 8.3 | 7.5 | 8.5 | 7.8 | 7.1 |
| 122 | 15.2 | 13.5 | 12.2 | 14.0 | 12.6 | 11.5 | 12.6 | 11.5 | 10.5 | 11.8 | 10.8 | 9.9 |
| 124A | 11.2 | 9.9 | 8.8 | 10.3 | 9.2 | 8.3 | 9.2 | 8.3 | 7.5 | 8.5 | 7.8 | 7.1 |
| 124B | 15.2 | 13.5 | 12.2 | 14.0 | 12.6 | 11.5 | 12.6 | 11.5 | 10.5 | 11.8 | 10.8 | 9.9 |
| 127A | 11.2 | 9.9 | 8.8 | 10.3 | 9.2 | 8.3 | 9.2 | 8.3 | 7.5 | 8.5 | 7.8 | 7.1 |
| 127B | 15.2 | 13.5 | 12.2 | 14.0 | 12.6 | 11.5 | 12.6 | 11.5 | 10.5 | 11.8 | 10.8 | 9.9 |

TABLE 8B
Amount of Non-Cationic Component (w/w relative to cargo)

| Cmpd | 1 | 2 | 3 | 4 | 5 | 6 | 7 | 8 | 9 | 10 | 11 | 12 |
|---|---|---|---|---|---|---|---|---|---|---|---|---|
| 119A | 1.1 | 1.0 | 0.9 | 3.1 | 2.8 | 2.5 | 5.5 | 5.0 | 4.5 | 6.8 | 6.2 | 5.7 |
| 119B | 1.0 | 0.9 | 0.8 | 2.8 | 2.5 | 2.3 | 5.0 | 4.6 | 4.2 | 6.3 | 5.8 | 5.3 |
| 121 | 1.1 | 1.0 | 0.9 | 3.1 | 2.8 | 2.5 | 5.5 | 5.0 | 4.5 | 6.8 | 6.2 | 5.7 |
| 122 | 1.0 | 0.9 | 0.8 | 2.8 | 2.5 | 2.3 | 5.0 | 4.6 | 4.2 | 6.3 | 5.8 | 5.3 |
| 124A | 1.1 | 1.0 | 0.9 | 3.1 | 2.8 | 2.5 | 5.5 | 5.0 | 4.5 | 6.8 | 6.2 | 5.7 |
| 124B | 1.0 | 0.9 | 0.8 | 2.8 | 2.5 | 2.3 | 5.0 | 4.6 | 4.2 | 6.3 | 5.8 | 5.3 |
| 127A | 1.1 | 1.0 | 0.9 | 3.1 | 2.8 | 2.5 | 5.5 | 5.0 | 4.5 | 6.8 | 6.2 | 5.7 |
| 127B | 1.0 | 0.9 | 0.8 | 2.8 | 2.5 | 2.3 | 5.0 | 4.6 | 4.2 | 6.3 | 5.8 | 5.3 |

TABLE 8C
Amount of DSPC (w/w relative to cargo)

| Cmpd | 1 | 2 | 3 | 4 | 5 | 6 | 7 | 8 | 9 | 10 | 11 | 12 |
|---|---|---|---|---|---|---|---|---|---|---|---|---|
| 119A | 0.0 | 3.0 | 5.3 | 0.0 | 2.8 | 5.0 | 0.0 | 2.5 | 4.5 | 0.0 | 2.3 | 4.3 |
| 119B | 0.0 | 2.7 | 4.9 | 0.0 | 2.5 | 4.6 | 0.0 | 2.3 | 4.2 | 0.0 | 2.2 | 4.0 |
| 121 | 0.0 | 3.0 | 5.3 | 0.0 | 2.8 | 5.0 | 0.0 | 2.5 | 4.5 | 0.0 | 2.3 | 4.3 |
| 122 | 0.0 | 2.7 | 4.9 | 0.0 | 2.5 | 4.6 | 0.0 | 2.3 | 4.2 | 0.0 | 2.2 | 4.0 |
| 124A | 0.0 | 3.0 | 5.3 | 0.0 | 2.8 | 5.0 | 0.0 | 2.5 | 4.5 | 0.0 | 2.3 | 4.3 |
| 124B | 0.0 | 2.7 | 4.9 | 0.0 | 2.5 | 4.6 | 0.0 | 2.3 | 4.2 | 0.0 | 2.2 | 4.0 |
| 127A | 0.0 | 3.0 | 5.3 | 0.0 | 2.8 | 5.0 | 0.0 | 2.5 | 4.5 | 0.0 | 2.3 | 4.3 |
| 127B | 0.0 | 2.7 | 4.9 | 0.0 | 2.5 | 4.6 | 0.0 | 2.3 | 4.2 | 0.0 | 2.2 | 4.0 |

TABLE 8D
Amount of DMG-PEG 2000 (w/w relative to cargo)

| Cmpd | 1 | 2 | 3 | 4 | 5 | 6 | 7 | 8 | 9 | 10 | 11 | 12 |
|---|---|---|---|---|---|---|---|---|---|---|---|---|
| 119A | 1.7 | 1.5 | 1.3 | 1.5 | 1.4 | 1.2 | 1.4 | 1.2 | 1.1 | 1.3 | 1.2 | 1.1 |
| 119B | 1.5 | 1.4 | 1.2 | 1.4 | 1.3 | 1.1 | 1.3 | 1.1 | 1.0 | 1.2 | 1.1 | 1.0 |
| 121 | 1.7 | 1.5 | 1.3 | 1.5 | 1.4 | 1.2 | 1.4 | 1.2 | 1.1 | 1.3 | 1.2 | 1.1 |
| 122 | 1.5 | 1.4 | 1.2 | 1.4 | 1.3 | 1.1 | 1.3 | 1.1 | 1.0 | 1.2 | 1.1 | 1.0 |
| 124A | 1.7 | 1.5 | 1.3 | 1.5 | 1.4 | 1.2 | 1.4 | 1.2 | 1.1 | 1.3 | 1.2 | 1.1 |
| 124B | 1.5 | 1.4 | 1.2 | 1.4 | 1.3 | 1.1 | 1.3 | 1.1 | 1.0 | 1.2 | 1.1 | 1.0 |
| 127A | 1.7 | 1.5 | 1.3 | 1.5 | 1.4 | 1.2 | 1.4 | 1.2 | 1.1 | 1.3 | 1.2 | 1.1 |
| 127B | 1.5 | 1.4 | 1.2 | 1.4 | 1.3 | 1.1 | 1.3 | 1.1 | 1.0 | 1.2 | 1.1 | 1.0 |

TABLE 7E
Resulting Fluc Expression (in vitro in HEK)

| | 1 | 2 | 3 | 4 | 5 | 6 | 7 | 8 | 9 | 10 | 11 | 12 |
|---|---|---|---|---|---|---|---|---|---|---|---|---|
| A | 1.41E+04 | 2.35E+04 | 1.08E+04 | 2.53E+04 | 2.39E+04 | 2.38E+04 | 2.62E+04 | 1.72E+04 | 9.46E+02 | 1.14E+03 | 5.38E+02 | 4.21E+02 |
| B | 1.91E+03 | 1.90E+03 | 1.50E+03 | 1.55E+04 | 9.85E+03 | 1.34E+04 | 4.09E+04 | 3.69E+04 | 2.79E+04 | 4.68E+04 | 2.85E+04 | 2.51E+04 |
| C | 3.03E+03 | 6.27E+02 | 1.12E+04 | 3.51E+04 | 1.38E+04 | 1.30E+04 | 2.69E+04 | 3.15E+04 | 2.55E+04 | 5.56E+04 | 2.14E+04 | 1.82E+04 |
| D | 1.79E+03 | 1.01E+03 | 2.30E+03 | 2.30E+04 | 8.30E+03 | 7.10E+03 | 1.82E+04 | 1.35E+04 | 1.39E+04 | 1.74E+04 | 2.36E+04 | 7.34E+02 |
| E | 6.70E+02 | 5.66E+02 | 5.81E+02 | 1.39E+03 | 8.02E+02 | 6.84E+02 | 1.83E+03 | 1.07E+03 | 1.14E+03 | 3.17E+03 | 1.79E+03 | 5.74E+02 |
| F | 4.69E+04 | 4.29E+04 | 3.81E+04 | 2.51E+04 | 3.31E+04 | 3.51E+04 | 3.35E+04 | 3.09E+04 | 5.12E+04 | 6.76E+04 | 3.33E+04 | 3.67E+04 |
| G | 3.08E+04 | 2.53E+04 | 2.35E+04 | 2.23E+04 | 2.75E+04 | 2.03E+04 | 4.54E+04 | 5.09E+04 | 5.69E+04 | 4.77E+04 | 4.50E+04 | 4.06E+04 |
| H | 3.02E+04 | 2.26E+04 | 1.79E+04 | 1.90E+04 | 1.95E+04 | 1.73E+04 | 2.85E+04 | 3.21E+04 | 3.11E+04 | 3.79E+04 | 2.08E+04 | 3.23E+04 |

TABLE 8E
Resulting Fluc Expression (in vitro in HEK)

| CMPD | 1 | 2 | 3 | 4 | 5 | 6 | 7 |
|---|---|---|---|---|---|---|---|
| 119A | 4.75E+03 | 5.18E+03 | 5.38E+03 | 4.97E+03 | 3.29E+03 | 4.03E+03 | 4.70E+03 |
| 119B | 3.77E+03 | 4.42E+03 | 5.39E+03 | 4.67E+03 | 4.80E+03 | 4.61E+03 | 3.12E+03 |
| 121 | 4.09E+03 | 4.07E+03 | 2.88E+03 | 4.04E+03 | 3.90E+03 | 3.51E+03 | 4.39E+03 |
| 122 | 4.17E+03 | 4.00E+03 | 4.44E+03 | 3.83E+03 | 4.39E+03 | 3.67E+03 | 5.06E+03 |
| 124A | 5.15E+03 | 4.93E+03 | 4.21E+03 | 4.86E+03 | 4.36E+03 | 3.27E+03 | 4.30E+03 |
| 124B | 4.72E+03 | 3.73E+03 | 2.96E+03 | 3.77E+03 | 3.96E+03 | 3.73E+03 | 3.62E+03 |
| 127A | 2.20E+03 | 4.34E+03 | 3.18E+03 | 9.32E+02 | 3.94E+03 | 3.54E+03 | 3.34E+03 |
| 127B | 4.24E+03 | 4.27E+03 | 4.03E+03 | 4.70E+03 | 5.13E+03 | 3.78E+03 | 5.66E+03 |
| | | CMPD | 8 | 9 | 10 | 11 | 12 |
| | | 119A | 5.24E+03 | 5.01E+03 | 2.17E+03 | 4.00E+03 | 3.15E+03 |
| | | 119B | 3.76E+03 | 2.27E+04 | 3.33E+03 | 4.27E+03 | 2.58E+03 |
| | | 121 | 4.00E+03 | 3.46E+03 | 4.49E+03 | 4.38E+03 | 3.20E+03 |
| | | 122 | 4.55E+03 | 4.89E+03 | 4.64E+03 | 3.96E+03 | 2.70E+03 |
| | | 124A | 4.63E+03 | 2.74E+03 | 4.79E+03 | 4.95E+03 | 5.14E+03 |
| | | 124B | 3.90E+03 | 4.11E+03 | 2.87E+03 | 4.20E+03 | 5.00E+03 |
| | | 127A | 3.53E+03 | 3.24E+03 | 2.75E+03 | 3.30E+03 | 4.06E+03 |
| | | 127B | 6.45E+03 | 1.74E+04 | 3.83E+03 | 3.88E+03 | 6.44E+0. [symbol] |

[symbol]

indicates data missing or illegible when filed

### Example 5—Cellular Viability Assay

Cellular viability was measured following treatment with the multicomponent delivery systems described herein (e.g., the Fluc mRNA/aminolipidated peptoid formulations) by a traditional MTT viability assay. In this assay, cells were first treated with the multicomponent delivery systems at a final mRNA concentration of 50 ng/well for 8 hours. Cells were then left to grow for 48 hours, after which time they were exposed to 1.5 mg/mL 3-(4,5-dimethylthiazol-2-yl)-2,5-diphenyltetrazolium bromide (MTT) for 2 hours, then lysed using a solubilizing solution of 10% Triton-X 100 in acidic isopropanol (0.1N HCl). The signal corresponding to reduced formazan was normalized to untreated cells to provide a relative viability of each treated condition. See FIG. 3C.

### Example 6—In Vivo Luciferase Expression

Generally, In vivo local expression of Fluc following IM administration of the resulting particles to mice at a dose of 0.05 mg/kg via a tail-vein injection. The resulting bioluminescence was quantified after 6 hours.

General methods. Prior to all studies, animals (e.g., mice and/or rats) were allowed to acclimate for a minimum of 3 days prior to use. The animals were maintained on a 12 hour light cycle in a temperature and humidity controlled room. A daily health check was performed as well as food and water check.

Injections were done subcutaneous (50-200 μL), intraperitoneal (up to 1000 μL) intravenous (50-200 μL), intramuscular (50-μL), or intratumoral (50-μL), using a 26-30 gauge needle depending on the site of injection. Animals were under isoflurane anesthesia for all injections/implants.

For imaging, animals were put under anesthesia, injected intraperitoneal with D-Luciferin (15 mg/mL) at a dose of 10 μL per gram of body weight, and placed into the camera chamber and imaged for up to 30 minutes. Typically images were performed 15 minutes following substrate injection.

Intravenous administration. The delivery vehicle complexes disclosed herein were effective for in vivo administration of Fluc mRNA to Balb/c mice through multiple routes of administration. In general, delivery system complexes were administered at a dose of 0.5 mg/kg via a tail-vein injection, and the resulting bioluminescence quantified after 6 hours. Organ-specific bioluminescence was quantified by sacrificing the treated animal, dissecting out the organs of interest, and separately quantifying the resulting bioluminescence

Local administration. In addition to IV administration, the delivery vehicle complexes described herein are also effective for local administration of mRNA through intratumoral (IT), subcutaneous (SC) or intramuscular (IM) routes of administration. For these examples, mRNA was administered at a dose of 0.1 mpk for intratumoral, or 0.01 mpk for subcutaneous and intramuscular administration, and the resulting bioluminescence quantified after 6 hours.

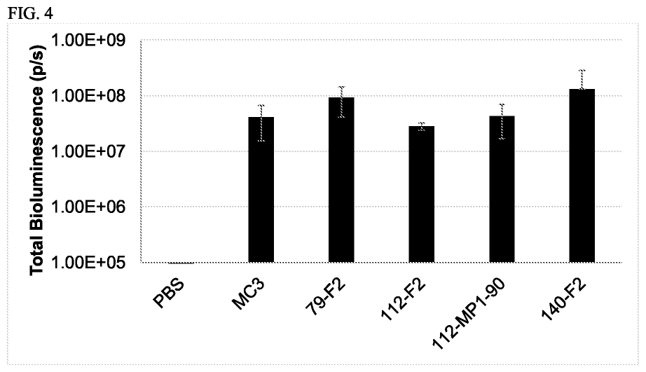

The multicomponent delivery vehicles of the disclosure show high luciferase expression when administered via intramuscular injection (see FIG. 1A, FIG. 1B, and Table 9) and via intratumoral injection (see FIG. 4).

### Example 7—mRNA Vaccination Models

Cellular responses. The efficacy of multicomponent delivery systems described herein in a disease model was evaluated by formulating mRNA coding for Ovalbumin (OVA) or with the peptoids of the disclosure and administering this vaccine to C57Bl/6 mice. Vaccine candidates were administered twice, with a prime on Day 0 off the study and a boost on Day 7. The resulting immune response to characterized epitopes was determined on Day 14 by measuring levels of antigen-specific CD8+ T-cells in peripheral blood and spleen with a fluorescent MHC-I tetramer conjugate (MBL International). The multicomponent delivery vehicle systems of the disclosure elicted a strong T cell response. See FIG. 2 and Table 9.

Humoral responses. Humoral responses to the vaccine candidates were evaluated by E7-IgG ELISA. Briefly, MaxiSorp ELISA plates (Thermo Scientific) were coated overnight at 4C with 1 ug/mL E7-his protein (Abcam). Plates were then washed and blocked with 10% FBS. Plasma samples were diluted in blocking buffer (10% FCS) at a 1:5 dilution with 5 10-fold dilutions down plate. Samples were added to plate and incubated at 4C overnight. Detection used a Donkey anti-mouse IgG-HRP (Jackson Immunology) at 1:1000 in blocking buffer for 1 hour, then detected with HRP substrate and read at 450 nm.

TABLE 9
In vivo expression from formulations

| | Total Fluc | OVA + | OVA Binding |
|---|---|---|---|
| | Photon | CD8 | Antibodies |
| Name | Flux (p/s) | T-Cells | (endpoint titer) |
| FORM-A | 7373 | 0.59 | 2.4 |
| FORM-B | 15240500 | 16.47 | 6967.0 |
| FORM-C | 41972667 | 15.24 | 6525.7 |
| FORM-D | 7954500 | 38.67 | 7737.3 |
| FORM-E | 8202667 | 18.82 | 12077.8 |
| FORM-F | 9313000 | 6.69 | 6446.8 |
| FORM-G | 4078000 | 2.76 | 44.4 |
| FORM-H | 11454000 | 5.39 | 3588.7 |
| FORM-I | 10211500 | 4.73 | 5361.4 |
| FORM-J | 9089500 | 3.25 | 9641.3 |

It should be appreciated that all combinations of the foregoing concepts and implementations and additional concepts and implementations discussed in greater detail below are contemplated as being part of the inventive subject matter disclosed herein, and may be employed in any suitable combination to achieve the benefits as described here. In particular, all combinations of claimed subject matter appearing at the end of this disclosure are contemplated as being part of the inventive subject matter disclosed herein.

The foregoing description is given for clearness of understanding only, and no unnecessary limitations should be understood therefrom, as modifications within the scope of the invention may be apparent to those having ordinary skill in the art.

The terms “substantially” and “about” used throughout this Specification are used to describe and account for small fluctuations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular examples, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

What is claimed is:

1. A system for the delivery of polyanionic compounds to target cells, the delivery system comprising at least one cationic component, wherein the at least one cationic component comprises a lipidated peptoid (“lipidated cationic peptoid”).

2. The system of claim 1 further comprising one or more of the following: (i) an anionic or zwitterionic component, (ii) a non-cationic lipid component, and (iii) a shielding component.

3. The system of claim 2, wherein the anionic or zwitterionic component comprises a phospholipid, a lipitoid, or a mixture thereof.

4. The system of claim 3, wherein the anionic or zwitterionic component is a DOPE or DSPC

5. The system of any one of claims 2-4, wherein the non-cationic lipid component comprises a sterol and/or a neutral peptoid.

6. The system of claim 5, wherein the sterol is cholesterol.

7. The system of any one of claims 2-6, wherein the shielding component comprises a poly(ethylene glycol) (PEG) moiety.

8. The system of claim 7, wherein the shielding component is a PEGylated lipid or a PEGylated lipidated peptoid.

9. The system of claim 8, wherein the shielding component is DMG-PEG.

10. The system of claim 9, wherein the shielding component is DMG-PEG 2000.

11. The system of any one of claims 5-10, wherein the non-cationic lipid component comprises Compound 90, or wherein the cationic lipid component comprises Compound 89:

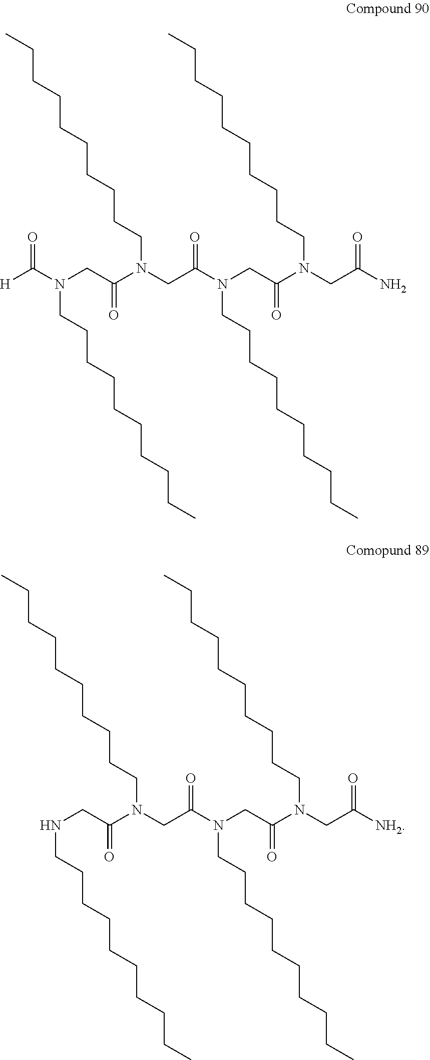 [SMILES: CCCCCCCCCCN(C=O)CC(=O)N(CCCCCCCCCC)CC(=O)N(CCCCCCCCCC)CC(=O)N(CCCCCCCCCC)CC(N)=O ; CCCCCCCCCCNCC(=O)N(CCCCCCCCCC)CC(=O)N(CCCCCCCCCC)CC(=O)N(CCCCCCCCCC)CC(N)=O]

12. The system according to any of the preceding claims 2-6 wherein the cationic component comprises Compound 112, and the non-cationic lipid component comprises Compound 90:

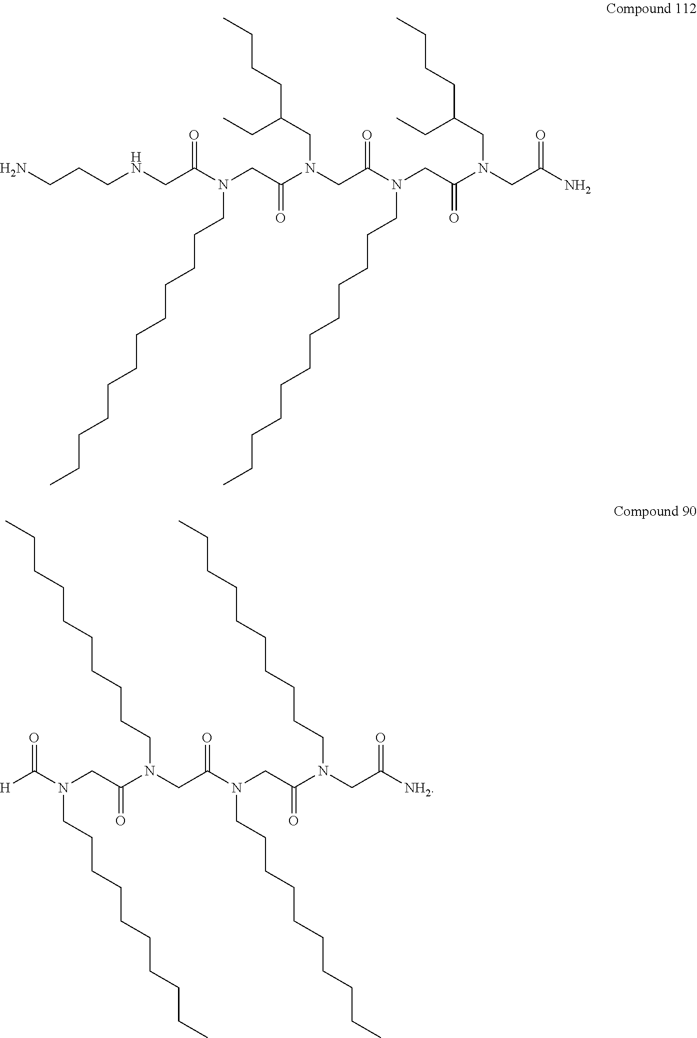 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCCN ; CCCCCCCCCCN(C=O)CC(=O)N(CCCCCCCCCC)CC(=O)N(CCCCCCCCCC)CC(=O)N(CCCCCCCCCC)CC(N)=O]

13. The system according to claim 12, wherein the mol % of the cationic component is between about 20 to about 50; the mol % of the anionic or zwitterionic component is between about 0 to about 30; the mol % of the non-cationic lipid component is between about 30 to about 80; and the mol % of the shielding component is between about 0 to about 10.

14. The system according to claim 12 or 13, wherein the mol % of the cationic component is between about 30 to about 50; the mol % of the anionic or zwitterionic component is between about 0 to about 10; the mol % of the non-cationic lipid component is between about 40 to about 70; and the mol % of the shielding component is between about 0 to about 5.

15. The system according to any of the claims 13-14 wherein the mol % of the cationic component is about 38; the mol % of the anionic or zwitterionic component is 0; the mol % of the non-cationic lipid component is about 59.7; and the mol % of the shielding component is about 2.3.

16. The system according to claim 15, wherein the anionic or zwitterionic component comprises DOPE, and wherein the shielding component comprises DMG-PEG2K.

17. The system according to any of the claims 2-16, wherein the cationic component comprises Compound 89 or Compound 93, and the non-cationic lipid component comprises cholesterol, Compound 90, or any combination thereof:

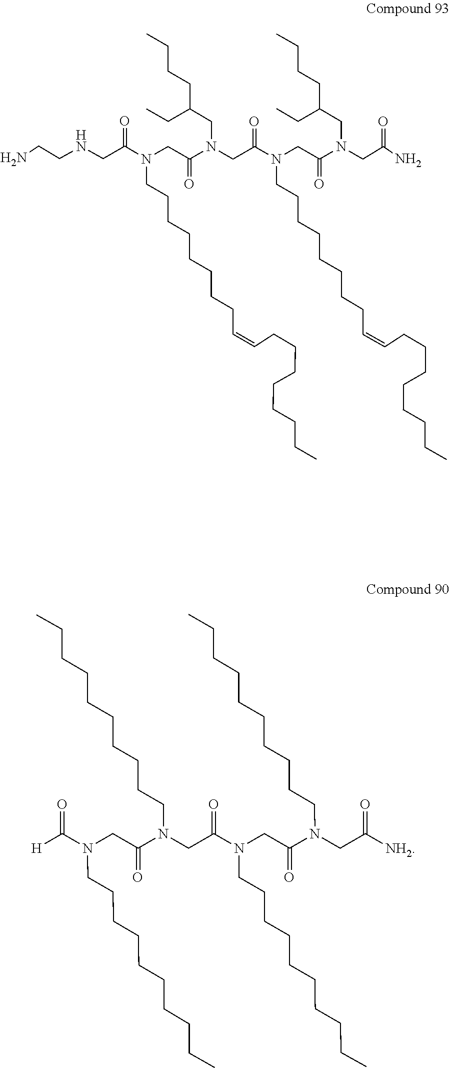

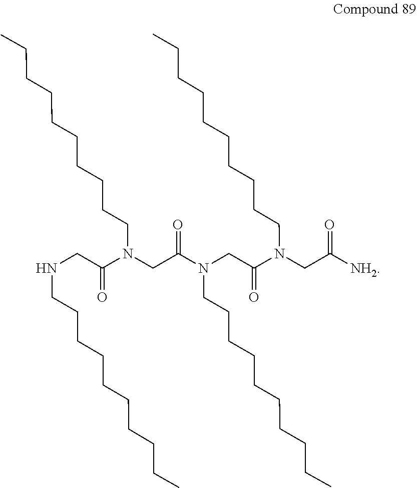 [SMILES: CCCCCCCCCCNCC(=O)N(CCCCCCCCCC)CC(=O)N(CCCCCCCCCC)CC(=O)N(CCCCCCCCCC)CC(N)=O]

18. The system according to any of the claims 1-10, wherein the cationic component comprises Compound 79, and the non-cationic lipid component comprises Compound 90:

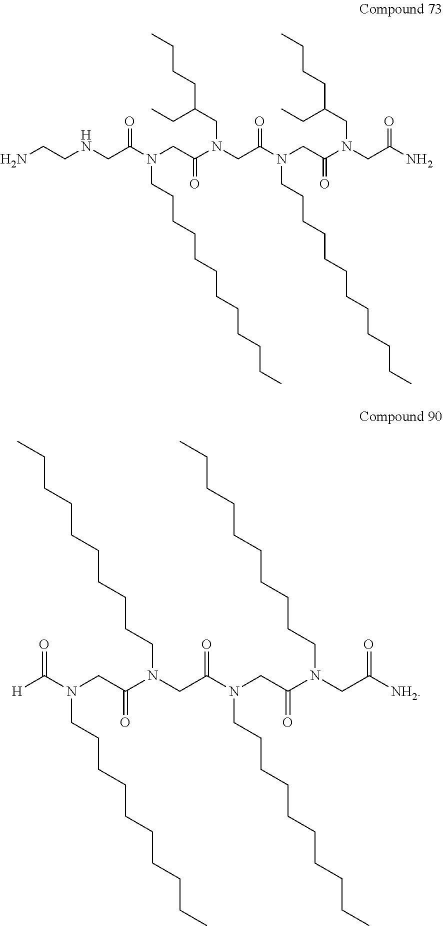 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCN ; CCCCCCCCCCN(C=O)CC(=O)N(CCCCCCCCCC)CC(=O)N(CCCCCCCCCC)CC(=O)N(CCCCCCCCCC)CC(N)=O]

19. The system according to any of the claims 1-10, wherein the cationic component comprises Compound 24 or Compound 89, and the non-cationic lipid component comprises cholesterol, Compound 90, or any combination thereof.

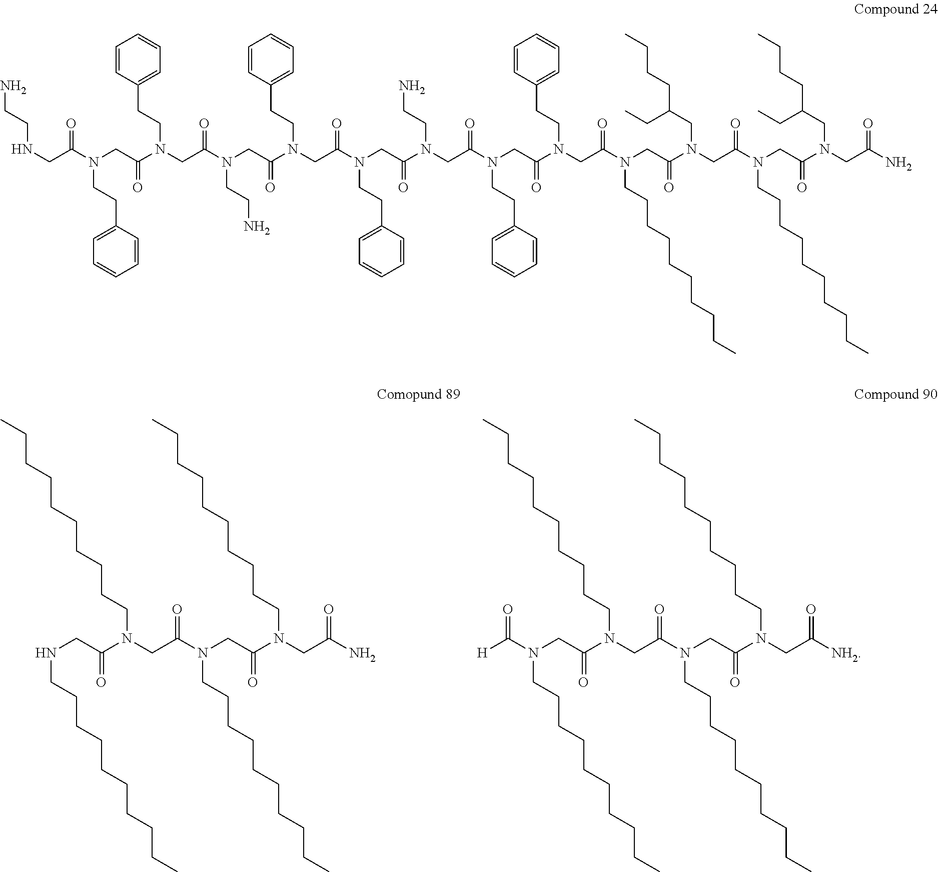 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN ; CCCCCCCCCCNCC(=O)N(CCCCCCCCCC)CC(=O)N(CCCCCCCCCC)CC(=O)N(CCCCCCCCCC)CC(N)=O ; CCCCCCCCCCN(C=O)CC(=O)N(CCCCCCCCCC)CC(=O)N(CCCCCCCCCC)CC(=O)N(CCCCCCCCCC)CC(N)=O]

20. The system of any of the preceding claims 2-10 comprising at least about 99 mol % cationic component and less than about 1 mol % shielding component.

21. The system according to claim 20 wherein the cationic component comprises Compound 24, Compound 79, Compound 93, Compound 112, or any combination thereof:

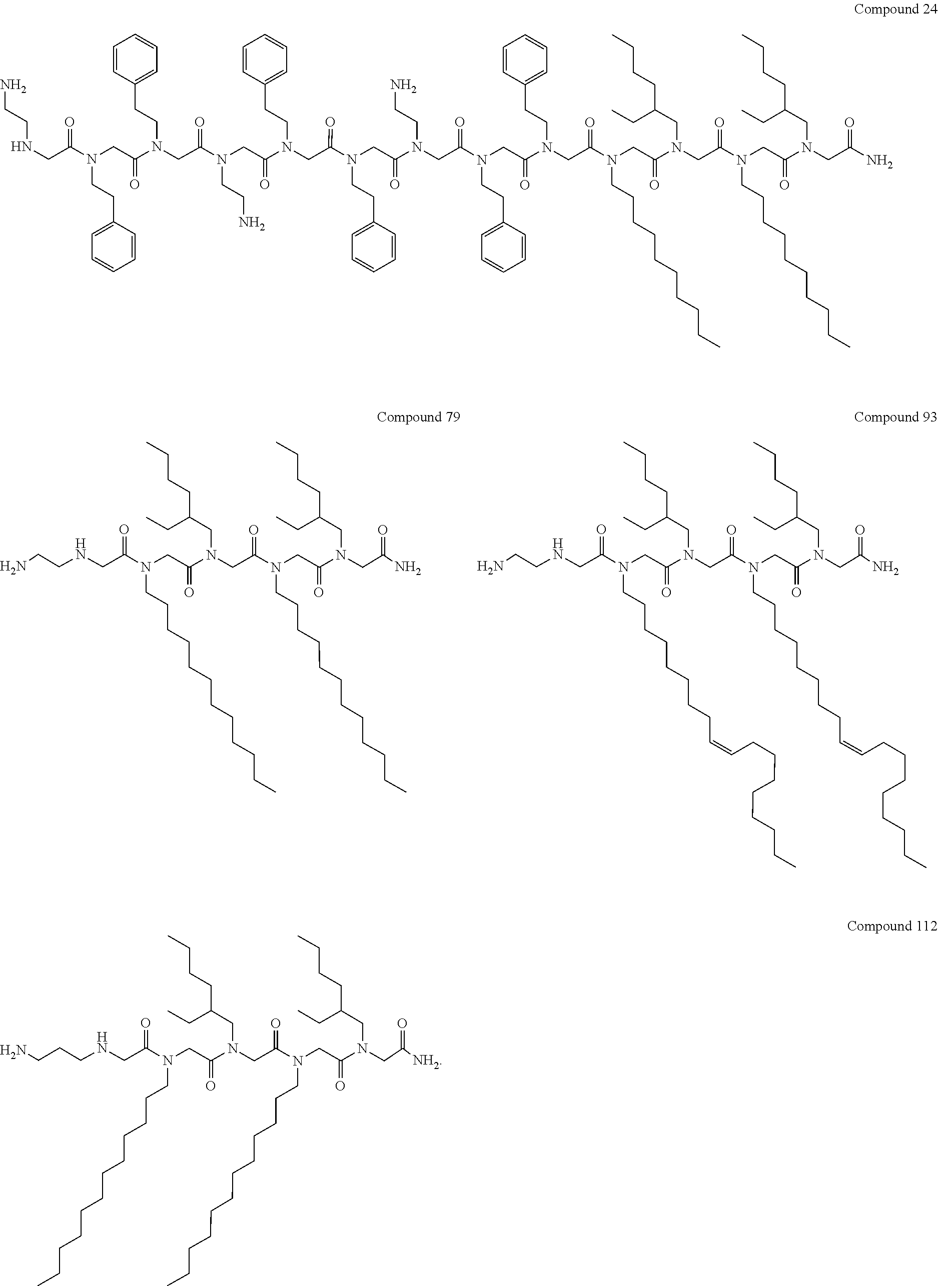

22. The system according to claim 20 or 21 wherein the mol % of the cationic component is about 99.1, and the mol % of the shielding component comprising PEG is about 0.9.

23. The system according to any of the preceding claims 2-10 comprising less than about 20 mol % of the cationic component, less than about 5 mol % of the shielding component, and more than about 75 mol % of a mixture of the zwitterionic component and the non-cationic lipid component.

24. The system of claim 23, wherein the cationic component comprises Compound 24, Compound 79, Compound 93, Compound 112, or any combination thereof.

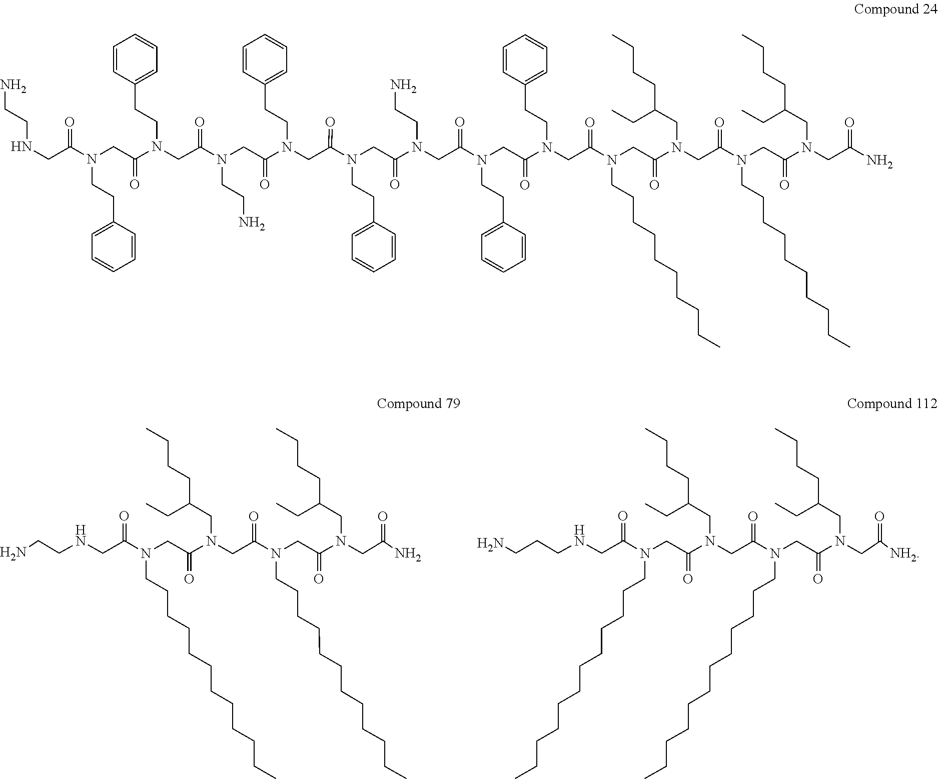 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN ; CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCN ; CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCCN]

25. The system of claim 23 or 24, wherein the mol % of the cationic component is about 17.9, the mol % of the shielding component comprising PEG is about 2.8, the mol % of the non-cationic lipid component is about 62.9, and the mol % of the anionic or zwitterionic component is about 16.4.

26. The system of claim 23 or 24, wherein the mol % of the cationic component is about 17.1, the mol % of the shielding component comprising PEG is about 2.7, the mol % of the non-cationic lipid component is about 80.2.

27. The system according to any of the claims 2-10 comprising between about 30 and about 45 mol % of the cationic component, between about 50 and about 70 mol % of a mixture of the anionic or zwitterionic component and the non-cationic lipid component, and between about 1.5 about 4.5 mol % of the shielding component.

28. The system of claim 27 wherein the cationic component comprises Compound 24, Compound 79, Compound 93, Compound 112, or any combination thereof.

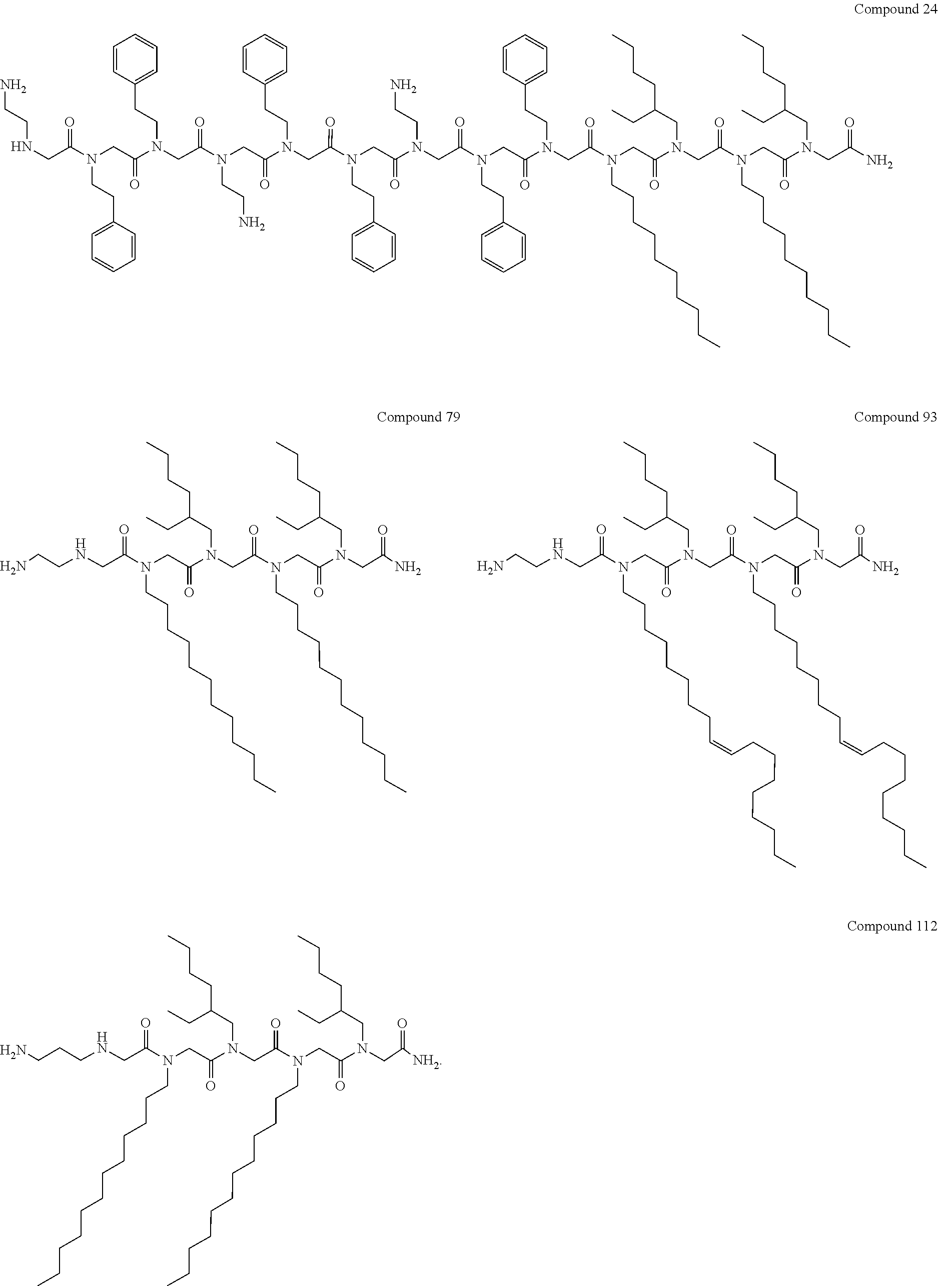

29. The system of claim 27 or 28 wherein the mol % of the cationic component is about 32.9, the mol % of the shielding component comprising PEG is about 2.0, the mol % of the non-cationic lipid component is about 51.7, and the mol % of the anionic or zwitterionic component is about 13.4.

30. The system of claim 27 or 28 wherein the mol % of the cationic component is about 42.3, the mol % of the shielding component comprising PEG is about 4.4, the mol % of the non-cationic lipid component is 0, and the mol % of the anionic or zwitterionic component is about 53.3.

31. The system according to any of the claims 2-10 comprising between about 15 and about 35 mol % of the cationic component, between about 60 and about 80 mol % of a mixture of the zwitterionic component and the non-cationic lipid component, and between about 1.5 and about 3.0 mol % of the shielding component.

32. The system of claim 31 wherein the cationic component comprises Compound 24, Compound 79, Compound 93, Compound 112, or any combination thereof.

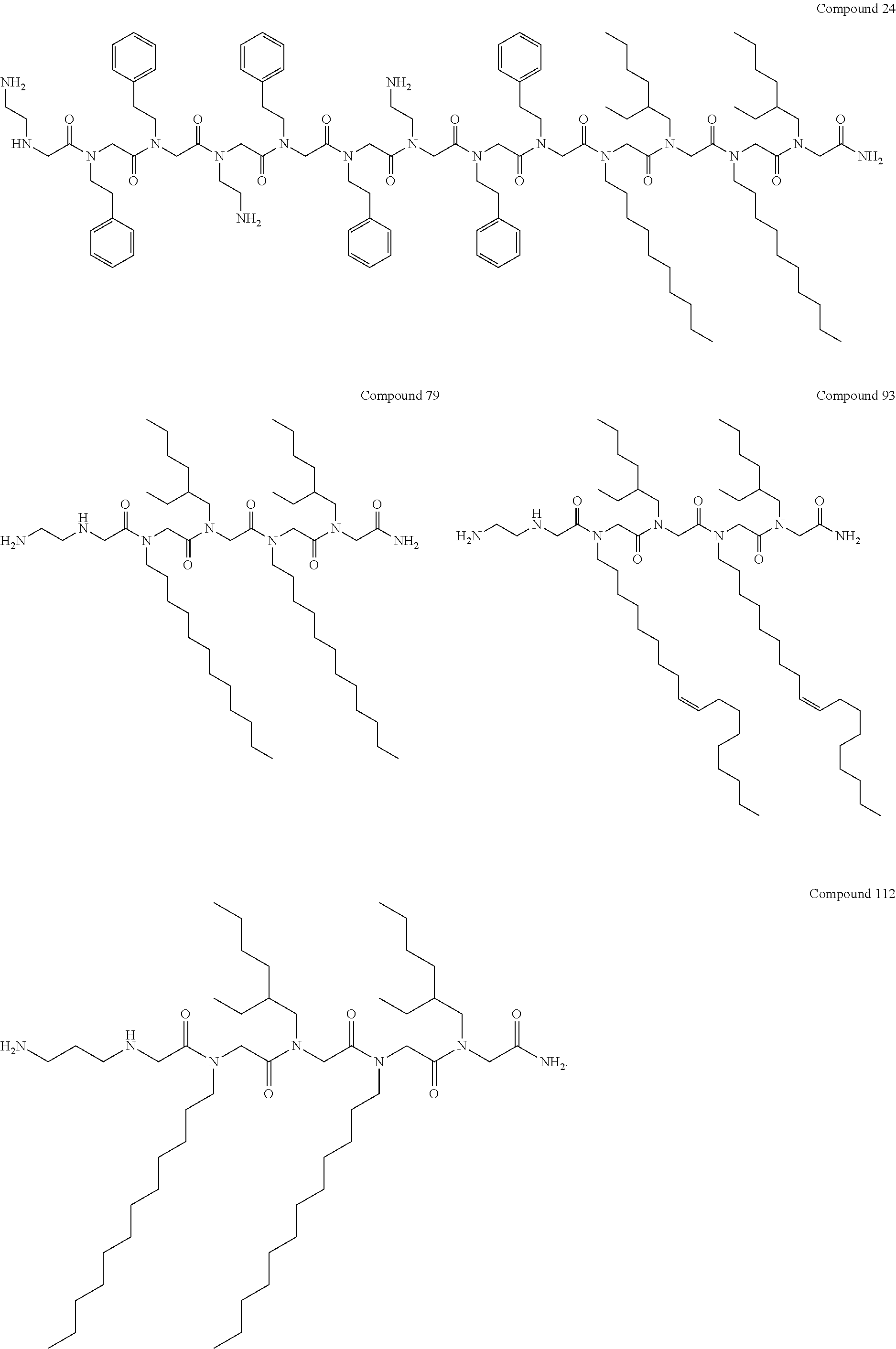

33. The system of claim 31 or 32, wherein the mol % of the cationic component is about 17.9, the mol % of the shielding component comprising PEG is about 2.8, the mol % of the non-cationic lipid component is about 62.9, and the mol % of the anionic or zwitterionic component is about 16.4.

34. The system of claim 31 or 32 wherein the mol % of the cationic component is about 32.9, the mol % of the shielding component comprising PEG is about 2.0, the mol % of the non-cationic lipid component is about 51.7, and the mol % of the anionic or zwitterionic component is about 13.4.

35. A complex comprising the system of any one of claims 1 to 34 and a polyanionic compound.

36. A delivery vehicle composition comprising a lipidated cationic peptoid component.

37. The delivery vehicle composition of claim 36, wherein the lipidated cationic peptoid component comprises a compound of Formula (Ia): 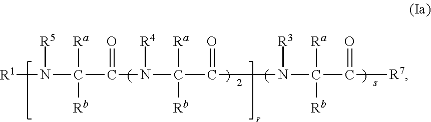
wherein:
r is an integer from 1 to 5;
s is an integer from 1 to 8;
R¹ is alkyl or CH₃;
each R³ independently is C₈-C₂₄alkyl or C₈-C₂₄-alkenyl;
each R⁴ independently is selected from the group consisting of C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalky is: (i) unsubstituted or (ii) substituted with one or more substituents selected from —OH, halo, or alkoxy;
each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl;
R⁷ is NH₂; and
each of R^{a} and R^{b} independently is H.

38. The delivery vehicle composition of claim 37, wherein the lipidated cationic peptoid component comprises one or more compounds selected from Compounds 1-12 and 19-36.

39. The delivery vehicle composition of claim 38, wherein the lipidated cationic peptoid component comprises Compound 24.

40. The delivery vehicle composition of claim 36, wherein the lipidated cationic peptoid component comprises a compound of Formula (Ib): 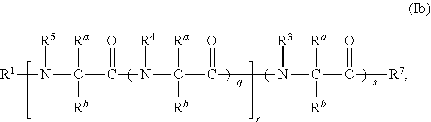
wherein:
q is 0 or 1;
r is an integer from 1 to 10;
s is an integer from 1 to 8;
R¹ is alkyl or CH₃;
each R³ independently is C₈-C₂₄alkyl or C₈-C₂₄-alkenyl;
each R⁴ independently is selected from the group consisting of C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalky is: (i) unsubstituted or (ii) substituted with one or more substituents selected from —OH, halo, or alkoxy;
each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl;
R⁷ is NH₂; and
each of R^{a} and R^{b} independently is H.

41. The delivery vehicle composition of claim 40, wherein the lipidated cationic peptoid component comprises one or more compounds selected from Compounds 13-16, 76, and 99.

42. The delivery vehicle composition of claim 36, wherein the lipidated cationic peptoid component comprises a compound of Formula (Ic): 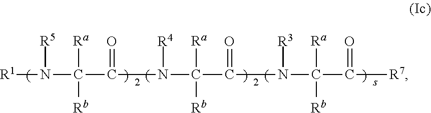
wherein:
s is 3 or 4;
R¹ is alkyl or CH₃;
each R³ independently is C₈-C₂₄alkyl or C₈-C₂₄-alkenyl;
each R⁴ independently is selected from the group consisting of C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalky is: (i) unsubstituted or (ii) substituted with one or more substituents selected from —OH, halo, or alkoxy;
each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl;
R⁷ is NH₂; and
each of R^{a} and R^{b} independently is H.

43. The delivery vehicle composition of claim 42, wherein the lipidated cationic peptoid component comprises one or more compounds selected from Compounds 49-55.

44. The delivery vehicle composition of claim 36, wherein the lipidated cationic peptoid component comprises a compound of Formula (Id): 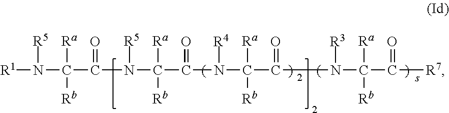
wherein:
s is 3 or 4;
R¹ is alkyl or CH₃;
each R³ independently is C₈-C₂₄alkyl or C₈-C₂₄-alkenyl;
each R⁴ independently is selected from the group consisting of C₁-C₄alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalky is: (i) unsubstituted or (ii) substituted with one or more substituents selected from —OH, halo, or alkoxy;
each R⁵ independently is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl;
R⁷ is NH₂; and
each of R^{a} and R^{b} independently is H.

45. The delivery vehicle composition of claim 44, wherein the lipidated cationic peptoid component comprises one or more compounds selected from Compounds 57-63 and 65-71.

46. The delivery vehicle composition of claim 36, wherein the lipidated cationic peptoid component comprises a compound of Formula (Ie): 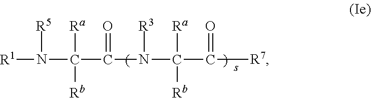
wherein:
s is 1 to 8;
R¹ is H or alkyl;
each R³ independently is C₈-C₂₄alkyl or C₈-C₂₄-alkenyl;
R⁵ is selected from the group consisting of aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, and hydroxylheteroalkyl;
R⁷ is NH₂; and
each of R^{a} and R^{b} independently is H.

47. The delivery vehicle composition of claim 46, wherein the lipidated cationic peptoid component comprises one or more compounds selected from Compounds 41-47, 73-75, 77-80, 85, 86, 91-94, 97, 98, 111-118, 137, 138, 140, 145, 146, 148, 149, 151-154, and 160-162.

48. The delivery vehicle composition of claim 47, wherein the lipidated cationic peptoid component comprises Compound 73, Compound 79, Compound 85, Compound 93, Compound 112, Compound 115, Compound 137, Compound 152, or combinations thereof.

49. The delivery vehicle composition of claim 48, wherein the lipidated cationic peptoid component comprises Compound 112.

50. The delivery vehicle composition of claim 36, wherein the lipidated cationic peptoid component comprises a compound of Formula (If): 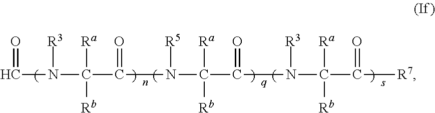
wherein:
n is an integer from 0 to 4;
q is 1 or 2;
s is an integer from 1 to 4;
each R³ independently is C₈-C₂₄alkyl or C₈-C₂₄-alkenyl;
R⁵ is selected from the group consisting of aminoalkyl, alkylaminoalkyl, aminoalkylaminoakyl, guanidinoalkyl, N-heterocyclylalkyl, and N-heteroaryl;
R⁷ is NH₂; and
each of R^{a} and R^{b} independently is H.

51. The delivery vehicle composition of claim 50, wherein the lipidated cationic peptoid component comprises one or more compounds selected from Compounds 81, 82, 83, 95, 96, 142, 144, 155, 156, 163, 164, and 165.

52. The delivery vehicle composition of claim 51, wherein the lipidated cationic peptoid component comprises Compound 81, Compound 155, Compound 163, Compound 164, or combinations thereof.

53. The delivery vehicle composition of claim 52, wherein the lipidated cationic peptoid component comprises Compound 81, Compound 155, or combinations thereof.

54. The delivery vehicle composition of claim 36, wherein the lipidated cationic peptoid component comprises one or more compounds selected from Compounds 17, 18, 84, 87-89, 100, 101, 102, 103, 113, 114, 127, 130, 132, 134, 139, 141, 147, and 159.

55. The delivery vehicle composition of claim 36, wherein the lipidated cationic peptoid component comprises Compound 127.

56. The delivery vehicle composition of claim 36, wherein the lipidated cationic peptoid component is selected from a compound listed in Table 1A.

57. The delivery vehicle composition of any one of claims 36-56 further comprising a non-cationic lipidated peptoid component.

58. The delivery vehicle composition of claim 57, wherein the non-cationic lipidated peptoid component comprises a neutral lipidated peptoid component.

59. The delivery vehicle composition of claim 58, wherein the neutral lipidated peptoid component comprises a compound Formula (IVa): 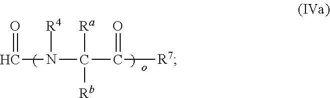
wherein o is integer from 3 to 10;
each R⁴ independently is C₈-C₂₄alkyl, or C₁-C₄-alkyl substituted with cycloalkyl, heterocyclylalkyl, aryl, alkylaryl, arylalkyl, alkylheteroaryl, heteroarylalkyl, alkoxy, alkoxyalkyl, or hydroxyalkyl;
R⁷ is —NH₂; and
each R^{a} and R^{b} independently is —H.

60. The delivery vehicle composition of claim 59, wherein the neutral lipidated peptoid component comprises Compound 90, Compound 119, Compound 120, Compound 121, Compound 122, Compound 123, Compound 128, Compound 129, or combinations thereof.

61. The delivery vehicle composition of claim 60, wherein the neutral lipidated peptoid component comprises Compound 90, Compound 119, or a combination thereof.

62. The delivery vehicle composition of claim 58, wherein the neutral lipidated peptoid component comprises Compound 150.

63. The delivery vehicle composition of claim 58, wherein the neutral lipidated peptoid component is selected from a compound listed in Table 1C.

64. The delivery vehicle composition of any one of claims 36-63, further comprising an anionic or zwitterionic (“anionic/zwitterionic”) lipidated peptoid component.

65. The delivery vehicle composition of claim 64, wherein the anionic/zwitterionic lipidated peptoid component comprises a compound of Formula (IIIa) 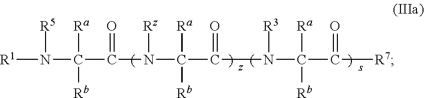
wherein:
s is an integer from 1 to 6;
z is 1 or 2;
R¹ is H or alkyl;
each R³ independently is C₈-C₂₄alkyl or C₈-C₂₄-alkenyl;
R⁵ is selected from the group consisting of aminoalkyl, alkylaminoalkyl, aminoalkylaminoakyl, guanidinoalkyl, N-heterocyclylalkyl, and N-heteroaryl;
R⁷ is —NH₂;
each R^{a} and R^{b} independently is —H; and
each R^{z} independently is C₂₋₅ alkylenecarboxylic acid.

66. The delivery vehicle composition of claim 65, wherein the anionic/zwitterionic lipidated peptoid component comprises Compound 104, Compound 105, or a combination thereof.

67. The delivery vehicle composition of claim 64, wherein the anionic/zwitterionic lipidated peptoid component comprises a compound of Formula (IIIb): 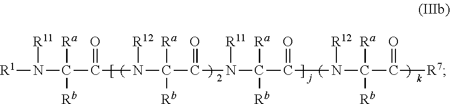
wherein:
j is 1, 2, 3, 4, 5, or 6;
k is 1, 2, 3, or 4;
R¹ is —H, alkyl, alkylaryl, —COR^{1a} or a lipid moiety, wherein R^{1a} is —H, —OH, alkyl, aryl, alkylaryl, —O-alkyl, or —O-alkylaryl;
each R¹¹ independently is aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, hydroxylheteroalkyl, or C₂₋₅ alkylenecarboxylic acid;
each R¹² independently is C₈-C₂₄alkyl, C₈-C₂₄-alkenyl, C₁-C₄aralkyl, or C₁₋₄heteroaralkyl;
R⁷ is —H, alkyl, acyl, —OH, —OR^{7a}, —NH₂, —NHR^{7a}, or a lipid moiety, wherein R^{7a} is alkyl, acyl, or a lipid moiety; and
each R^{a} and R^{b} independently is —H, C₁-C₄-alkyl, or a side chain moiety found on a naturally- or non-naturally-occurring amino acid.

68. The delivery vehicle composition of claim 67, wherein the non-cationic lipidated peptoid component comprises Compound 124, Compound 126, Compound 134, or a combination thereof.

69. The delivery vehicle composition of claim 64, wherein the anionic/zwitterionic lipidated peptoid component comprises one or more compounds selected from Compounds 125, 131, 133, 135, 136, and combinations thereof.

70. The delivery vehicle composition of claim 64, wherein the non-cationic lipidated peptoid component comprises Compound 124, Compound 125, Compound 126, or combinations thereof.

71. The delivery vehicle composition of claim 64, wherein the anionic/zwitterionic lipidated peptoid component is selected from a compound listed in Table 1B.

72. The delivery vehicle composition of any one of claims 36-71, further comprising a PEGylated lipidated peptoid component.

73. The delivery vehicle composition of claim 72, wherein the PEGylated lipidated peptoid component comprises a compound of Formula (Va), a compound of Formula (Vb), or a combination thereof: 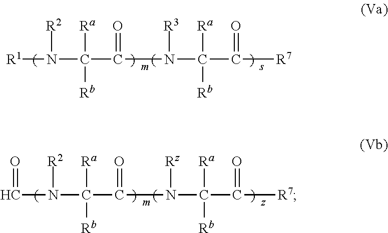
wherein:
m is an integer from 1 to 15;
s is an integer from 1 to 6;
z is an integer from 1 to 6;
R¹ is H or alkyl;
each R² independently is an ethylene glycol moiety of the formula —CH₂CH₂O(CH₂CH₂O)ᵤCH₃, and wherein each u is independently an integer from 2 to 200;
each R³ independently is C₈-C₂₄alkyl or C₈-C₂₄-alkenyl;
R⁷ is —NH₂;
each R^{a} and R^{b} independently is —H; and
each R^{z} independently is C₂₋₅alkylenecarboxylic acid.

74. The delivery vehicle composition of claim 73, wherein the PEGylated lipidated peptoid component comprises Compound 106, Compound 107, Compound 108, Compound 109, or combinations thereof.

75. The delivery vehicle composition of claim 72, wherein the PEGylated lipidated peptoid component comprises one or more of Compounds 56, 64, 72.

76. The delivery vehicle composition of claim 72, wherein the PEGylated lipidated peptoid component is selected from a compound listed in Table 1D.

77. The delivery vehicle composition of any one of claims 36-76, further comprising Compound 135.

78. The delivery vehicle composition of claim 36, wherein:
(a) the cationic lipidated peptoid component comprises Compound 24, Compound 73, Compound 79, Compound 81, Compound 85, Compound 93, Compound 112, Compound 115, Compound 127, Compound 137, Compound 152, Compound 155, Compound 163, Compound 164, or combinations thereof; and
(b) the non-cationic lipidated peptoid component comprises Compound 90, Compound 119, Compound 120, Compound 121, Compound 122, Compound 123, Compound 124, Compound 125, Compound 126, Compound 128, Compound 129, or combinations thereof.

79. The delivery vehicle composition of claim 78, wherein:
(a) the cationic lipidated peptoid component comprises Compound 81, Compound 112, Compound 155, Compound 163, Compound 164, or combinations thereof; and
(b) the non-cationic lipidated peptoid component comprises Compound 90, Compound 119, Compound 124, Compound 125, Compound 126, or combinations thereof.

80. The delivery vehicle composition of claim 79, wherein:
(a) the cationic lipidated peptoid component comprises Compound 81, Compound 155, or combinations thereof; and
(b) the non-cationic lipidated peptoid component comprises Compound 90, Compound 119, or combinations thereof.

81. The delivery vehicle composition of any one of claims 36-80, further comprising a PEGylated lipid.

82. The delivery vehicle composition of claim 81, wherein the PEGylated lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, a PEG-modified sterol, a PEG-modified phospholipid, and combinations thereof.

83. The delivery vehicle composition of claim 82, wherein the PEGylated lipid comprises dimyristoylglycerol-polyethylene glycol 2000 (DMG-PEG 2000).

84. The delivery vehicle composition of any one of claim 36-83, wherein the delivery vehicle composition further comprises a phospholipid.

85. The delivery vehicle composition of claim 84, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE),1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and combinations thereof.

86. The delivery vehicle composition of claim 85, wherein the phospholipid comprises DOPE, DSPC, or combinations thereof.

87. The delivery vehicle composition of any one of claims 36-86, further comprising a sterol.

88. The delivery vehicle composition of claim 87, wherein the sterol comprises cholesterol.

89. The delivery vehicle composition of any one of claims 57-88, wherein the composition comprises about 20 mol % to about 50 mol % of the lipidated cationic component; about 40 mol % to about 80 mol % of the non-lipidated peptoid component, about 1 mol % to about 5 mol % of the PEGylated lipid, and 0 mol % to about 20 mol % of the phospholipid.

90. The delivery vehicle composition of claim 89, wherein the composition comprises about 30 mol % to about 45 mol % of the lipidated cationic component; about 50 mol % to about 70 mol % of the non-cationic lipidated peptoid component, about 1 mol % to about 3 mol % of the PEGylated lipid, and 0 mol % of the phospholipid.

91. The delivery vehicle composition of claim 90, wherein the composition comprises about 35 mol % to about 40 mol % of the lipidated cationic component; about 55 mol % to about 65 mol % of the non-cationic lipidated peptoid component, about 1.5 mol % to about 2.5 mol % of the PEGylated lipid, and 0 mol % of the phospholipid.

92. The delivery vehicle composition of claim 91, wherein the composition comprises about 38 mol % of the lipidated cationic component; about 60 mol % of the non-cationic lipidated peptoid component, about 2.3 mol % of the PEGylated lipid, and 0 mol % of the phospholipid.

93. A delivery vehicle complex comprising the delivery vehicle composition of any one of claims 36-92, and a polyanionic compound.

94. The delivery vehicle complex of claim 93, wherein the lipidated cationic peptoid component is complexed to the polyanionic compound.

95. The delivery vehicle complex of claim 93 or 94, wherein the mass ratio of the lipidated cationic peptoid component to the polyanionic compound is between about 5:1 and about 20:1.

96. The delivery vehicle complex of claim 95, wherein the mass ratio of the lipidated cationic peptoid component to the polyanionic compound is between about 7:1 and about 15:1.

97. The delivery vehicle complex of claim 96, wherein the mass ratio of the lipidated cationic peptoid component to the polyanionic compound is between about 10:1 and about 11:1.

98. The delivery vehicle complex of claim 97, wherein the mass ratio of the lipidated cationic peptoid component to the polyanionic compound is about 10:1.

99. The delivery vehicle complex of any one of claims 93-98, wherein the mass ratio of the non-cationic lipidated peptoid to the polyanionic compound is between about 4:1 and about 6.5:1.

100. The delivery vehicle complex of claim 99, wherein the mass ratio of the non-cationic lipidated peptoid to the polyanionic compound is between about 5:1 and about 6:1.

101. The delivery vehicle complex of claim 100, wherein the mass ratio of the non-cationic lipidated peptoid to the polyanionic compound is about 5.4:1.

102. The delivery vehicle complex of any one of claims 93-101, wherein the mass ratio of the PEGylated lipid to the polyanionic compound is between about 1:1 to about 2:1.

103. The delivery vehicle complex of claim 102, wherein the mass ratio of the PEGylated lipid to the polyanionic compound is about 1.4:1.

104. The delivery vehicle complex of any one of claims 93-103, wherein the mass ratio of the lipidated cationic peptoid to the polyanionic compound is about 10:1, the mass ratio of the non-cationic lipidated peptoid to the polyanionic compound is about 5.4:1, and the mass ratio of the PEGylated lipid to the polyanionic compound is about 1.4:1.

105. The delivery vehicle complex of any one of claims 93-104, wherein:
(a) the cationic lipidated peptoid component comprises Compound 24, Compound 73, Compound 79, Compound 81, Compound 85, Compound 93, Compound 112, Compound 115, Compound 137, Compound 152, Compound 155, Compound 163, Compound 164, or combinations thereof;
(b) the non-cationic lipidated peptoid component comprises Compound 90, Compound 119, Compound 120, Compound 121, Compound 122, Compound 123, Compound 124, Compound 125, Compound 126, Compound 128, Compound 129, or combinations thereof; and
(c) the PEGylated lipid is DMG-PEG 2000.

106. The delivery vehicle composition of claim 105, wherein:
(a) the cationic lipidated peptoid component comprises Compound 81, Compound 112, Compound 155, Compound 163, Compound 164, or combinations thereof; and
(b) the non-cationic lipidated peptoid component comprises Compound 90, Compound 119, Compound 124, Compound 125, Compound 126, or combinations thereof.

107. The delivery vehicle composition of claim 106, wherein:
(a) the cationic lipidated peptoid component comprises Compound 81, Compound 155, or combinations thereof; and
(b) the non-cationic lipidated peptoid component comprises Compound 90, Compound 119, or combinations thereof.

108. The delivery vehicle complex of claim 107, wherein the lipidated cationic component comprises Compound 81, and the non-cationic lipidated peptoid component comprises Compound 90.

109. The delivery vehicle complex of claim 107, wherein the lipidated cationic component comprises Compound 81, and the non-cationic lipidated peptoid component comprises Compound 119.

110. The delivery vehicle complex of claim 107, wherein the lipidated cationic component comprises Compound 155, and the non-cationic lipidated peptoid component comprises Compound 90.

111. The delivery vehicle complex of claim 107, wherein the lipidated cationic component comprises Compound 155, and the non-cationic lipidated peptoid component comprises Compound 119.

112. The delivery vehicle complex of any one of claims 93-111, further comprising Compound 135.

113. The delivery vehicle complex of any one of claims 93-112, wherein the complex exhibits a particle size of less than about 200 nm and/or a polydispersity index (PDI) of less than 0.25.

114. The delivery vehicle complex of claim 113, wherein the complex exhibits a particle size of about 50 nm to about 95 nm.

115. The delivery vehicle complex of claim 113, wherein the complex exhibits a particle size of about 105 nm to about 200 nm.

116. The delivery vehicle complex of any one of claims 93-115, wherein at least 80% of the polyanionic compound is retained after storage at 4° C. for 48 days, or the delivery vehicle complex retains at least 80% of its original size after storage at 4° C. for 48 days, or both.

117. The delivery vehicle complex of any one of claims 93-116, wherein the polyanionic compound comprises at least one nucleic acid.

118. The delivery vehicle complex of claim 117, wherein the at least one nucleic acid comprises RNA, DNA, or a combination thereof.

119. The delivery vehicle complex of claim 118, wherein the at least one nucleic acid comprises RNA.

120. The delivery vehicle complex of claim 119, wherein the RNA is mRNA encoding a peptide, a protein, or a functional fragment of any the foregoing.

121. The delivery vehicle complex of claim 120, wherein the mRNA encodes for a viral peptide, a viral protein, or functional fragment of any of the foregoing.

122. The delivery vehicle complex of claim 121, wherein the mRNA encodes for a human papillomavirus (HPV) protein or a functional fragment thereof.

123. The delivery vehicle complex of claim 122, wherein the mRNA encodes for the HPV E6 protein and/or the HPV E7 protein, or a functional fragment of any of the foregoing.

124. The delivery vehicle complex of claim 121, wherein the mRNA encodes for a viral spike protein or a functional fragment thereof.

125. The delivery vehicle complex of claim 124, wherein the mRNA encodes for a SARS-CoV spike (S) protein or a functional fragment thereof.

126. The delivery vehicle complex of claim 121, wherein the mRNA encodes for influenza hemagglutinin (HA), or a functional fragment thereof.

127. The delivery vehicle complex of claim 121, comprising an mRNA that encodes for a SARS-CoV spike (S) protein and an mRNA that encodes for influenza hemagglutinin (HA), or a functional fragment of the foregoing.

128. A pharmaceutical composition comprising the delivery vehicle complex of any one of claims 93-127, and a pharmaceutically acceptable excipient.

129. The pharmaceutical composition of claim 128 as an intratumoral (IT) or intramuscular (MI) composition.

130. A method of inducing an immune response in a subject in need thereof, comprising administering to the subject in need thereof an effective amount of the delivery vehicle complex of any one of claims 93-127, or the pharmaceutical formulation of claim 128 or 129, thereby inducing an immune response in the subject.

131. A method of treating a viral infection in a subject in need thereof, comprising administering to the subject an effective amount of the delivery vehicle complex of any one of claims 93-127, or the pharmaceutical formulation of any of claim 128 or 129, thereby treating the viral infection in the subject.

132. A method of treating cancer in a subject in need thereof, comprising administering to the subject an effective amount of the delivery vehicle complex of any one of claims 93-127, or the pharmaceutical formulation of any of claim 128 or 129, thereby treating the cancer in the subject.

133. The method of claim 132, wherein the cancer is cervical cancer, head and neck cancer, B-cell lymphoma, T-cell lymphoma, prostate cancer, lung cancer, or a combination thereof.

134. The method of any one of claims 130-132, wherein the administering is by intramuscular, intratumoral, intravenous, intraperitoneal, or subcutaneous delivery.

135. A method of delivering a polyanionic compound to a cell comprising contacting the cell with the delivery vehicle complex of any one of claims 93-127 or the pharmaceutical composition of claim 128 or 129.

136. The method of claim 135, wherein the polyanionic compound is an mRNA that encodes for a peptide, a protein, or a fragment of any of the foregoing, and the cell expresses the peptide, the protein, or the fragment after being contacted with the delivery vehicle complex.

137. A method of forming the delivery vehicle complex of any one of claims 93-127, comprising contacting the lipidated cationic peptoid component with the polyanionic compound.

138. The method of claim 137, comprising admixing a solution comprising the lipidated cationic peptoid component with a solution comprising the polyanionic compound.

139. A compound of Formula (If): 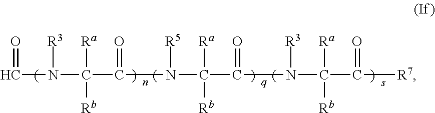
wherein:
n is an integer from 0 to 4;
q is 1 or 2;
s is an integer from 1 to 4;
each R³ independently is C₈-C₂₄alkyl or C₈-C₂₄-alkenyl;
R⁵ is selected from the group consisting of aminoalkyl, alkylaminoalkyl, aminoalkylaminoakyl, guanidinoalkyl, N-heterocyclylalkyl, and N-heteroaryl;
R⁷ is NH₂; and
each of R^{a} and R^{b} independently is H.

140. The compound of claim 139 selected from Compounds 81, 82, 83, 95, 96, 142, 144, 155, 156, 163, 164, and 165.

141. A compound of Formula (IVa): 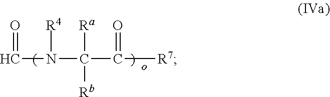
wherein o is integer from 3 to 10;
each R⁴ independently is C₈-C₂₄alkyl, or C₁-C₄-alkyl substituted with cycloalkyl, heterocyclylalkyl, aryl, alkylaryl, arylalkyl, alkylheteroaryl, heteroarylalkyl, alkoxy, alkoxyalkyl, or hydroxyalkyl;
R⁷ is —NH₂; and
each R^{a} and R^{b} independently is —H.

142. The compound of claim 141 selected from Compounds 90, 119, 120, 121, 122, 123, 128, and 129.

143. A compound of Formula (IIIa) 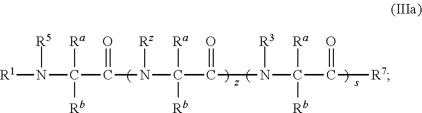
wherein:
s is an integer from 1 to 6;
z is 1 or 2;
R¹ is H or alkyl;
each R³ independently is C₈-C₂₄alkyl or C₈-C₂₄-alkenyl;
R⁵ is selected from the group consisting of aminoalkyl, alkylaminoalkyl, aminoalkylaminoakyl, guanidinoalkyl, N-heterocyclylalkyl, and N-heteroaryl;
R⁷ is —NH₂;
each R^{a} and R^{b} independently is —H; and
each R^{z} independently is C₂₋₅ alkylenecarboxylic acid.

144. The compound of claim 143 selected from Compound 104 and Compound 105.

145. A compound of Formula (IIIb): 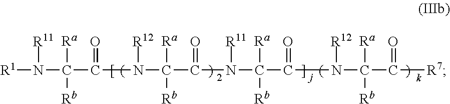
wherein:
j is 1, 2, 3, 4, 5, or 6;
k is 1, 2, 3, or 4;
R¹ is —H, alkyl, alkylaryl, —COR^{1a} or a lipid moiety, wherein R^{1a} is —H, —OH, alkyl, aryl, alkylaryl, —O-alkyl, or —O-alkylaryl;
each R¹¹ independently is aminoalkyl, alkylaminoalkyl, aminoalkylaminoakyl, guanidinoalkyl, N-heterocyclylalkyl, N-heteroaryl, hydroxyalkyl, hydroxyether, alkoxyalkyl, hydroxylheteroalkyl, or C₂₋₅ alkylenecarboxylic acid;
each R¹² independently is C₈-C₂₄alkyl, C₅-C₂₄-alkenyl, C₁-C₄aralkyl, or C₁₋₄heteroaralkyl;
R⁷ is —H, alkyl, acyl, —OH, —OR^{7a}, —NH₂, —NHR^{7a}, or a lipid moiety, wherein R^{7a} is alkyl, acyl, or a lipid moiety; and
each R^{a} and R^{b} independently is —H, C₁-C₄-alkyl, or a side chain moiety found on a naturally- or non-naturally-occurring amino acid.

146. The compound of claim 145 selected from Compound 124, Compound 126, and Compound 134.

147. A compound listed in Table 1A, 1B, 1C, or 1D selected from Compound 73-165.
